SEQUENCE LISTING (314 sequences)

SEQ ID NO: 1 - protein, 121 aa, Artificial Sequence
  source: /note=“Description of Artificial Sequence Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 2 - protein, 107 aa, Artificial Sequence
  source: /note=“Description of Artificial Sequence Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 3 - protein, 451 aa, Artificial Sequence
  source: /note=“Description of Artificial Sequence Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  source: /note=“Description of Artificial Sequence Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 363 bp, Artificial Sequence
  source: /note=“Description of Artificial Sequence Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 6 - DNA, 321 bp, Artificial Sequence
  source: /note=“Description of Artificial Sequence Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 7 - DNA, 1353 bp, Artificial Sequence
  source: /note=“Description of Artificial Sequence Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 8 - DNA, 642 bp, Artificial Sequence
  source: /note=“Description of Artificial Sequence Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 9 - protein, 5 aa, Artificial Sequence
  source: /note=“Description of Artificial Sequence Synthetic peptide”
SYGVD

SEQ ID NO: 10 - protein, 7 aa, Artificial Sequence
  source: /note=“Description of Artificial Sequence Synthetic peptide”
GFSLSSY

SEQ ID NO: 11 - protein, 16 aa, Artificial Sequence
  source: /note=“Description of Artificial Sequence Synthetic peptide”
VIWGGGGTYYASSLMG

SEQ ID NO: 12 - protein, 5 aa, Artificial Sequence
  source: /note=“Description of Artificial Sequence Synthetic peptide”
WGGGG

SEQ ID NO: 13 - protein, 13 aa, Artificial Sequence
  source: /note=“Description of Artificial Sequence Synthetic peptide”
HAYGHDGGFAMDY

SEQ ID NO: 14 - protein, 11 aa, Artificial Sequence
  source: /note=“Description of Artificial Sequence Synthetic peptide”
RASESVSSNVA

SEQ ID NO: 15 - protein, 7 aa, Artificial Sequence
  source: /note=“Description of Artificial Sequence Synthetic peptide”
SESVSSN

SEQ ID NO: 16 - protein, 7 aa, Artificial Sequence
  source: /note=“Description of Artificial Sequence Synthetic peptide”
GASNRAT

SEQ ID NO: 17 - protein, 3 aa, Artificial Sequence
  source: /note=“Description of Artificial Sequence Synthetic peptide”
GAS

SEQ ID NO: 18 - protein, 9 aa, Artificial Sequence
  source: /note=“Description of Artificial Sequence Synthetic peptide”
GQSYSYPFT

SEQ ID NO: 19 - protein, 6 aa, Artificial Sequence
  source: /note=“Description of Artificial Sequence Synthetic peptide”
SYSYPF

SEQ ID NO: 20 - protein, 124 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 21 - protein, 107 aa, Artificial Sequence
  source: /note=“Description of Artificial Sequence Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 22 - protein, 5 aa, Artificial Sequence
  source: /note=“Description of Artificial Sequence Synthetic peptide”
TYWMH

SEQ ID NO: 23 - protein, 17 aa, Artificial Sequence
  source: /note=“Description of Artificial Sequence Synthetic peptide”
NIYPGTGGSNFDEKFKN

SEQ ID NO: 24 - protein, 8 aa, Artificial Sequence
  source: /note=“Description of Artificial Sequence Synthetic peptide”
WTTGTGAY

SEQ ID NO: 25 - protein, 7 aa, Artificial Sequence
  source: /note=“Description of Artificial Sequence Synthetic peptide”
GYTFTTY

SEQ ID NO: 26 - protein, 6 aa, Artificial Sequence
  source: /note=“Description of Artificial Sequence Synthetic peptide”
YPGTGG

SEQ ID NO: 27 - protein, 117 aa, Artificial Sequence
  source: /note=“Description of Artificial Sequence Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 28 - DNA, 351 bp, Artificial Sequence
  source: /note=“Description of Artificial Sequence Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 29 - protein, 443 aa, Artificial Sequence
  source: /note=“Description of Artificial Sequence Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - DNA, 1329 bp, Artificial Sequence
  source: /note=“Description of Artificial Sequence Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 31 - protein, 17 aa, Artificial Sequence
  source: /note=“Description of Artificial Sequence Synthetic peptide”
KSSQSLLDSGNQKNFLT

SEQ ID NO: 32 - protein, 7 aa, Artificial Sequence
  source: /note=“Description of Artificial Sequence Synthetic peptide”
WASTRES

SEQ ID NO: 33 - protein, 22 aa, Artificial Sequence
  source: /note=“Description of Artificial Sequence Synthetic peptide”
YGRKKRRQRRRLYRSPAMPENL

SEQ ID NO: 34 - protein, 13 aa, Artificial Sequence
  source: /note=“Description of Artificial Sequence Synthetic peptide”
SQSLLDSGNQKNF

SEQ ID NO: 35 - protein, 3 aa, Artificial Sequence
  source: /note=“Description of Artificial Sequence Synthetic peptide”
WAS

SEQ ID NO: 36 - protein, 6 aa, Artificial Sequence
  source: /note=“Description of Artificial Sequence Synthetic peptide”
DYSYPY

SEQ ID NO: 37 - protein, 113 aa, Artificial Sequence
  source: /note=“Description of Artificial Sequence Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 38 - DNA, 339 bp, Artificial Sequence
  source: /note=“Description of Artificial Sequence Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 39 - protein, 220 aa, Artificial Sequence
  source: /note=“Description of Artificial Sequence Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 660 bp, Artificial Sequence
  source: /note=“Description of Artificial Sequence Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 41 - protein, 113 aa, Artificial Sequence
  source: /note=“Description of Artificial Sequence Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 42 - DNA, 339 bp, Artificial Sequence
  source: /note=“Description of Artificial Sequence Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 43 - protein, 220 aa, Artificial Sequence
  source: /note=“Description of Artificial Sequence Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 660 bp, Artificial Sequence
  source: /note=“Description of Artificial Sequence Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 45 - DNA, 15 bp, Artificial Sequence
  source: /note=“Description of Artificial Sequence Synthetic oligonucleotide”
acctactggatgcac

SEQ ID NO: 46 - DNA, 51 bp, Artificial Sequence
  source: /note=“Description of Artificial Sequence Synthetic oligonucleotide”
aatatctaccccggcaccggcggctctaacttcgacgagaagtttaagaat

SEQ ID NO: 47 - DNA, 24 bp, Artificial Sequence
  source: /note=“Description of Artificial Sequence Synthetic oligonucleotide”
tggactaccggcacaggcgcctac

SEQ ID NO: 48 - DNA, 21 bp, Artificial Sequence
  source: /note=“Description of Artificial Sequence Synthetic oligonucleotide”
ggctacaccttcactacctac

SEQ ID NO: 49 - DNA, 18 bp, Artificial Sequence
  source: /note=“Description of Artificial Sequence Synthetic oligonucleotide”
taccccggcaccggcggc

SEQ ID NO: 50 - DNA, 51 bp, Artificial Sequence
  source: /note=“Description of Artificial Sequence Synthetic oligonucleotide”
aaatctagtcagtcactgctggatagcggtaatcagaagaacttcctgacc

SEQ ID NO: 51 - DNA, 21 bp, Artificial Sequence
  source: /note=“Description of Artificial Sequence Synthetic oligonucleotide”
tgggcctctactagagaatca

SEQ ID NO: 52 - DNA, 27 bp, Artificial Sequence
  source: /note=“Description of Artificial Sequence Synthetic oligonucleotide”
cagaacgactatagctacccctacacc

SEQ ID NO: 53 - DNA, 39 bp, Artificial Sequence
  source: /note=“Description of Artificial Sequence Synthetic oligonucleotide”
agtcagtcactgctggatagcggtaatcagaagaacttc

SEQ ID NO: 54 - DNA, 9 bp, Artificial Sequence
  source: /note=“Description of Artificial Sequence Synthetic oligonucleotide”
tgggcctct

SEQ ID NO: 55 - DNA, 18 bp, Artificial Sequence
  source: /note=“Description of Artificial Sequence Synthetic oligonucleotide”
gactatagctacccctac

SEQ ID NO: 56 - protein, 440 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 214 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 447 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 218 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 447 aa, Artificial Sequence
  source: /note=“Description of Artificial Sequence Synthetic polypeptide”
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 213 aa, Artificial Sequence
  source: /note=“Description of Artificial Sequence Synthetic polypeptide”
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 5 aa, Artificial Sequence
  source: /note=“Description of Artificial Sequence Synthetic peptide”
SYWMY

SEQ ID NO: 63 - protein, 17 aa, Artificial Sequence
  source: /note=“Description of Artificial Sequence Synthetic peptide”
RIDPNSGSTKYNEKFKN

SEQ ID NO: 64 - protein, 11 aa, Artificial Sequence
  source: /note=“Description of Artificial Sequence Synthetic peptide”
DYRKGLYAMDY

SEQ ID NO: 65 - protein, 7 aa, Artificial Sequence
  source: /note=“Description of Artificial Sequence Synthetic peptide”
GYTFTSY

SEQ ID NO: 66 - protein, 6 aa, Artificial Sequence
  source: /note=“Description of Artificial Sequence Synthetic peptide”
DPNSGS

SEQ ID NO: 67 - protein, 120 aa, Artificial Sequence
  source: /note=“Description of Artificial Sequence Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 68 - DNA, 360 bp, Artificial Sequence
  source: /note=“Description of Artificial Sequence Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 69 - protein, 446 aa, Artificial Sequence
  source: /note=“Description of Artificial Sequence Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 70 - protein, 11 aa, Artificial Sequence
  source: /note=“Description of Artificial Sequence Synthetic peptide”
KASQDVGTAVA

SEQ ID NO: 71 - protein, 7 aa, Artificial Sequence
  source: /note=“Description of Artificial Sequence Synthetic peptide”
WASTRHT

SEQ ID NO: 72 - protein, 9 aa, Artificial Sequence
  source: /note=“Description of Artificial Sequence Synthetic peptide”
QQYNSYPLT

SEQ ID NO: 73 - protein, 7 aa, Artificial Sequence
  source: /note=“Description of Artificial Sequence Synthetic peptide”
SQDVGTA

SEQ ID NO: 74 - protein, 3 aa, Artificial Sequence
  source: /note=“Description of Artificial Sequence Synthetic peptide”
WAS

SEQ ID NO: 75 - protein, 6 aa, Artificial Sequence
  source: /note=“Description of Artificial Sequence Synthetic peptide”
YNSYPL

SEQ ID NO: 76 - DNA, 1338 bp, Artificial Sequence
  source: /note=“Description of Artificial Sequence Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 77 - protein, 107 aa, Artificial Sequence
  source: /note=“Description of Artificial Sequence Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 78 - DNA, 321 bp, Artificial Sequence
  source: /note=“Description of Artificial Sequence Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 79 - protein, 214 aa, Artificial Sequence
  source: /note=“Description of Artificial Sequence Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 642 bp, Artificial Sequence
  source: /note=“Description of Artificial Sequence Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 81 - protein, 120 aa, Artificial Sequence
  source: /note=“Description of Artificial Sequence Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 82 - DNA, 360 bp, Artificial Sequence
  source: /note=“Description of Artificial Sequence Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 83 - protein, 446 aa, Artificial Sequence
  source: /note=“Description of Artificial Sequence Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 84 - DNA, 1338 bp, Artificial Sequence
  source: /note=“Description of Artificial Sequence Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 85 - protein, 107 aa, Artificial Sequence
  source: /note=“Description of Artificial Sequence Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 86 - DNA, 321 bp, Artificial Sequence
  source: /note=“Description of Artificial Sequence Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 87 - protein, 214 aa, Artificial Sequence
  source: /note=“Description of Artificial Sequence Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - DNA, 642 bp, Artificial Sequence
  source: /note=“Description of Artificial Sequence Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 89 - DNA, 15 bp, Artificial Sequence
  source: /note=“Description of Artificial Sequence Synthetic oligonucleotide”
agctactggatgtac

SEQ ID NO: 90 - DNA, 51 bp, Artificial Sequence
  source: /note=“Description of Artificial Sequence Synthetic oligonucleotide”
agaatcgaccctaatagcggctctactaagtataacgagaagtttaagaat

SEQ ID NO: 91 - DNA, 33 bp, Artificial Sequence
  source: /note=“Description of Artificial Sequence Synthetic oligonucleotide”
gactatagaaagggcctgtacgctatggactac

SEQ ID NO: 92 - DNA, 21 bp, Artificial Sequence
  source: /note=“Description of Artificial Sequence Synthetic oligonucleotide”
ggctacaccttcactagctac

SEQ ID NO: 93 - DNA, 18 bp, Artificial Sequence
  source: /note=“Description of Artificial Sequence Synthetic oligonucleotide”
gaccctaatagcggctct

SEQ ID NO: 94 - DNA, 33 bp, Artificial Sequence
  source: /note=“Description of Artificial Sequence Synthetic oligonucleotide”
aaagcctctcaggacgtgggcaccgccgtggcc

SEQ ID NO: 95 - DNA, 21 bp, Artificial Sequence
  source: /note=“Description of Artificial Sequence Synthetic oligonucleotide”
tgggcctctactagacacacc

SEQ ID NO: 96 - DNA, 27 bp, Artificial Sequence
  source: /note=“Description of Artificial Sequence Synthetic oligonucleotide”
cagcagtataatagctaccccctgacc

SEQ ID NO: 97 - DNA, 21 bp, Artificial Sequence
  source: /note=“Description of Artificial Sequence Synthetic oligonucleotide”
tctcaggacgtgggcaccgcc

SEQ ID NO: 98 - DNA, 9 bp, Artificial Sequence
  source: /note=“Description of Artificial Sequence Synthetic oligonucleotide”
tgggcctct

SEQ ID NO: 99 - DNA, 18 bp, Artificial Sequence
  source: /note=“Description of Artificial Sequence Synthetic oligonucleotide”
tataatagctaccccctg

SEQ ID NO: 100 - protein, 448 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 214 aa, Artificial Sequence
  source: /note=“Description of Artificial Sequence Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 450 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 216 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 104 - protein, 451 aa, Artificial Sequence
  source: /note=“Description of Artificial Sequence Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 215 aa, Artificial Sequence
  source: /note=“Description of Artificial Sequence Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 123 aa, Artificial Sequence
  source: /note=“Description of Artificial Sequence Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 107 - protein, 106 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 108 - protein, 5 aa, Artificial Sequence
  source: /note=“Description of Artificial Sequence Synthetic peptide”
NYGMN

SEQ ID NO: 109 - protein, 17 aa, Artificial Sequence
  source: /note=“Description of Artificial Sequence Synthetic peptide”
WINTDTGEPTYADDFKG

SEQ ID NO: 110 - protein, 16 aa, Artificial Sequence
  source: /note=“Description of Artificial Sequence Synthetic peptide”
NPPYYYGTNNAEAMDY

SEQ ID NO: 111 - protein, 7 aa, Artificial Sequence
  source: /note=“Description of Artificial Sequence Synthetic peptide”
GFTLTNY

SEQ ID NO: 112 - protein, 6 aa, Artificial Sequence
  source: /note=“Description of Artificial Sequence Synthetic peptide”
NTDTGE

SEQ ID NO: 113 - protein, 125 aa, Artificial Sequence
  source: /note=“Description of Artificial Sequence Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 114 - DNA, 375 bp, Artificial Sequence
  source: /note=“Description of Artificial Sequence Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 115 - DNA, 375 bp, Artificial Sequence
  source: /note=“Description of Artificial Sequence Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 116 - protein, 451 aa, Artificial Sequence
  source: /note=“Description of Artificial Sequence Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 117 - protein, 11 aa, Artificial Sequence
  source: /note=“Description of Artificial Sequence Synthetic peptide”
SSSQDISNYLN

SEQ ID NO: 118 - protein, 7 aa, Artificial Sequence
  source: /note=“Description of Artificial Sequence Synthetic peptide”
YTSTLHL

SEQ ID NO: 119 - protein, 9 aa, Artificial Sequence
  source: /note=“Description of Artificial Sequence Synthetic peptide”
QQYYNLPWT

SEQ ID NO: 120 - protein, 7 aa, Artificial Sequence
  source: /note=“Description of Artificial Sequence Synthetic peptide”
SQDISNY

SEQ ID NO: 121 - protein, 3 aa, Artificial Sequence
  source: /note=“Description of Artificial Sequence Synthetic peptide”
YTS

SEQ ID NO: 122 - protein, 6 aa, Artificial Sequence
  source: /note=“Description of Artificial Sequence Synthetic peptide”
YYNLPW

SEQ ID NO: 123 - DNA, 1353 bp, Artificial Sequence
  source: /note=“Description of Artificial Sequence Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 124 - DNA, 1353 bp, Artificial Sequence
  source: /note=“Description of Artificial Sequence Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 125 - protein, 107 aa, Artificial Sequence
  source: /note=“Description of Artificial Sequence Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 126 - DNA, 321 bp, Artificial Sequence
  source: /note=“Description of Artificial Sequence Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 127 - DNA, 321 bp, Artificial Sequence
  source: /note=“Description of Artificial Sequence Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 128 - protein, 214 aa, Artificial Sequence
  source: /note=“Description of Artificial Sequence Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - DNA, 642 bp, Artificial Sequence
  source: /note=“Description of Artificial Sequence Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 130 - DNA, 642 bp, Artificial Sequence
  source: /note=“Description of Artificial Sequence Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 131 - protein, 125 aa, Artificial Sequence
  source: /note=“Description of Artificial Sequence Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 132 - DNA, 375 bp, Artificial Sequence
  source: /note=“Description of Artificial Sequence Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 133 - DNA, 375 bp, Artificial Sequence
  source: /note=“Description of Artificial Sequence Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 134 - protein, 451 aa, Artificial Sequence
  source: /note=“Description of Artificial Sequence Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 135 - DNA, 1353 bp, Artificial Sequence
  source: /note=“Description of Artificial Sequence Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 136 - DNA, 1353 bp, Artificial Sequence
  source: /note=“Description of Artificial Sequence Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 137 - protein, 107 aa, Artificial Sequence
  source: /note=“Description of Artificial Sequence Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 138 - DNA, 321 bp, Artificial Sequence
  source: /note=“Description of Artificial Sequence Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 139 - DNA, 321 bp, Artificial Sequence
  source: /note=“Description of Artificial Sequence Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 140 - protein, 214 aa, Artificial Sequence
  source: /note=“Description of Artificial Sequence Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - DNA, 642 bp, Artificial Sequence
  source: /note=“Description of Artificial Sequence Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 142 - DNA, 642 bp, Artificial Sequence
  source: /note=“Description of Artificial Sequence Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 143 - DNA, 15 bp, Artificial Sequence
  source: /note=“Description of Artificial Sequence Synthetic oligonucleotide”
aattacgggatgaac

SEQ ID NO: 144 - DNA, 15 bp, Artificial Sequence
  source: /note=“Description of Artificial Sequence Synthetic oligonucleotide”
aactacggcatgaac

SEQ ID NO: 145 - DNA, 51 bp, Artificial Sequence
  source: /note=“Description of Artificial Sequence Synthetic oligonucleotide”
tggattaacaccgacaccggggagcctacctacgcggacgatttcaaggga

SEQ ID NO: 146 - DNA, 51 bp, Artificial Sequence
  source: /note=“Description of Artificial Sequence Synthetic oligonucleotide”
tggatcaacaccgacaccggcgagcctacctacgccgacgacttcaagggc

SEQ ID NO: 147 - DNA, 48 bp, Artificial Sequence
  source: /note=“Description of Artificial Sequence Synthetic oligonucleotide”
aacccgccctactactacggaaccaacaacgccgaagccatggactac

SEQ ID NO: 148 - DNA, 48 bp, Artificial Sequence
  source: /note=“Description of Artificial Sequence Synthetic oligonucleotide”
aaccccccttactactacggcaccaacaacgccgaggccatggactat

SEQ ID NO: 149 - DNA, 21 bp, Artificial Sequence
  source: /note=“Description of Artificial Sequence Synthetic oligonucleotide”
ggattcaccctcaccaattac

SEQ ID NO: 150 - DNA, 21 bp, Artificial Sequence
  source: /note=“Description of Artificial Sequence Synthetic oligonucleotide”
ggcttcaccctgaccaactac

SEQ ID NO: 151 - DNA, 18 bp, Artificial Sequence
  source: /note=“Description of Artificial Sequence Synthetic oligonucleotide”
aacaccgacaccggggag

SEQ ID NO: 152 - DNA, 18 bp, Artificial Sequence
  source: /note=“Description of Artificial Sequence Synthetic oligonucleotide”
aacaccgacaccggcgag

SEQ ID NO: 153 - DNA, 33 bp, Artificial Sequence
  source: /note=“Description of Artificial Sequence Synthetic oligonucleotide”
agctctagtcaggatatctctaactacctgaac

SEQ ID NO: 154 - DNA, 33 bp, Artificial Sequence
  source: /note=“Description of Artificial Sequence Synthetic oligonucleotide”
tcctccagccaggacatctccaactacctgaac

SEQ ID NO: 155 - DNA, 21 bp, Artificial Sequence
  source: /note=“Description of Artificial Sequence Synthetic oligonucleotide”
tacactagcaccctgcacctg

SEQ ID NO: 156 - DNA, 21 bp, Artificial Sequence
  source: /note=“Description of Artificial Sequence Synthetic oligonucleotide”
tacacctccaccctgcacctg

SEQ ID NO: 157 - DNA, 27 bp, Artificial Sequence
  source: /note=“Description of Artificial Sequence Synthetic oligonucleotide”
cagcagtactataacctgccctggacc

SEQ ID NO: 158 - DNA, 27 bp, Artificial Sequence
  source: /note=“Description of Artificial Sequence Synthetic oligonucleotide”
cagcagtactacaacctgccctggacc

SEQ ID NO: 159 - DNA, 21 bp, Artificial Sequence
  source: /note=“Description of Artificial Sequence Synthetic oligonucleotide”
agtcaggatatctctaactac

SEQ ID NO: 160 - DNA, 21 bp, Artificial Sequence
  source: /note=“Description of Artificial Sequence Synthetic oligonucleotide”
agccaggacatctccaactac

SEQ ID NO: 161 - DNA, 9 bp, Artificial Sequence
  source: /note=“Description of Artificial Sequence Synthetic oligonucleotide”
tacactagc

SEQ ID NO: 162 - DNA, 9 bp, Artificial Sequence
  source: /note=“Description of Artificial Sequence Synthetic oligonucleotide”
tacacctcc

SEQ ID NO: 163 - DNA, 18 bp, Artificial Sequence
  source: /note=“Description of Artificial Sequence Synthetic oligonucleotide”
tactataacctgccctgg

SEQ ID NO: 164 - DNA, 18 bp, Artificial Sequence
  source: /note=“Description of Artificial Sequence Synthetic oligonucleotide”
tactacaacctgccctgg

SEQ ID NO: 165 - DNA, 15 bp, Artificial Sequence
  source: /note=“Description of Artificial Sequence Synthetic oligonucleotide”
aactacgggatgaac

SEQ ID NO: 166 - DNA, 51 bp, Artificial Sequence
  source: /note=“Description of Artificial Sequence Synthetic oligonucleotide”
tggattaacaccgacaccggcgagcctacctacgccgacgactttaagggc

SEQ ID NO: 167 - DNA, 48 bp, Artificial Sequence
  source: /note=“Description of Artificial Sequence Synthetic oligonucleotide”
aaccccccctactactacggcactaacaacgccgaggctatggactac

SEQ ID NO: 168 - DNA, 21 bp, Artificial Sequence
  source: /note=“Description of Artificial Sequence Synthetic oligonucleotide”
ggcttcaccctgactaactac

SEQ ID NO: 169 - protein, 447 aa, Artificial Sequence
  source: /note=“Description of Artificial Sequence Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 170 - protein, 214 aa, Artificial Sequence
  source: /note=“Description of Artificial Sequence Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 446 aa, Artificial Sequence
  source: /note=“Description of Artificial Sequence Synthetic polypeptide”
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 220 aa, Artificial Sequence
  source: /note=“Description of Artificial Sequence Synthetic polypeptide”
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 10 aa, Artificial Sequence
  source: /note=“Description of Artificial Sequence Synthetic peptide”
GFTLTNYGMN

SEQ ID NO: 174 - protein, 5 aa, Artificial Sequence
  source: /note=“Description of Artificial Sequence Synthetic peptide”
SYNMH

SEQ ID NO: 175 - protein, 17 aa, Artificial Sequence
  source: /note=“Description of Artificial Sequence Synthetic peptide”
DIYPGNGDTSYNQKFKG

SEQ ID NO: 176 - protein, 9 aa, Artificial Sequence
  source: /note=“Description of Artificial Sequence Synthetic peptide”
VGGAFPMDY

SEQ ID NO: 177 - protein, 7 aa, Artificial Sequence
  source: /note=“Description of Artificial Sequence Synthetic peptide”
GYTFTSY

SEQ ID NO: 178 - protein, 6 aa, Artificial Sequence
  source: /note=“Description of Artificial Sequence Synthetic peptide”
YPGNGD

SEQ ID NO: 179 - protein, 118 aa, Artificial Sequence
  source: /note=“Description of Artificial Sequence Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 180 - DNA, 354 bp, Artificial Sequence
  source: /note=“Description of Artificial Sequence Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 181 - protein, 444 aa, Artificial Sequence
  source: /note=“Description of Artificial Sequence Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 182 - DNA, 1332 bp, Artificial Sequence
  source: /note=“Description of Artificial Sequence Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 183 - protein, 15 aa, Artificial Sequence
  source: /note=“Description of Artificial Sequence Synthetic peptide”
RASESVEYYGTSLMQ

SEQ ID NO: 184 - protein, 7 aa, Artificial Sequence
  source: /note=“Description of Artificial Sequence Synthetic peptide”
AASNVES

SEQ ID NO: 185 - protein, 9 aa, Artificial Sequence
  source: /note=“Description of Artificial Sequence Synthetic peptide”
QQSRKDPST

SEQ ID NO: 186 - protein, 11 aa, Artificial Sequence
  source: /note=“Description of Artificial Sequence Synthetic peptide”
SESVEYYGTSL

SEQ ID NO: 187 - protein, 3 aa, Artificial Sequence
  source: /note=“Description of Artificial Sequence Synthetic peptide”
AAS

SEQ ID NO: 188 - protein, 6 aa, Artificial Sequence
  source: /note=“Description of Artificial Sequence Synthetic peptide”
SRKDPS

SEQ ID NO: 189 - protein, 111 aa, Artificial Sequence
  source: /note=“Description of Artificial Sequence Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 190 - DNA, 333 bp, Artificial Sequence
  source: /note=“Description of Artificial Sequence Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 191 - protein, 218 aa, Artificial Sequence
  source: /note=“Description of Artificial Sequence Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - DNA, 654 bp, Artificial Sequence
  source: /note=“Description of Artificial Sequence Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 193 - protein, 17 aa, Artificial Sequence
  source: /note=“Description of Artificial Sequence Synthetic peptide”
DIYPGQGDTSYNQKFKG

SEQ ID NO: 194 - protein, 6 aa, Artificial Sequence
  source: /note=“Description of Artificial Sequence Synthetic peptide”
YPGQGD

SEQ ID NO: 195 - protein, 118 aa, Artificial Sequence
  source: /note=“Description of Artificial Sequence Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 196 - DNA, 354 bp, Artificial Sequence
  source: /note=“Description of Artificial Sequence Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 197 - protein, 444 aa, Artificial Sequence
  source: /note=“Description of Artificial Sequence Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 198 - DNA, 1332 bp, Artificial Sequence
  source: /note=“Description of Artificial Sequence Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 199 - protein, 111 aa, Artificial Sequence
  source: /note=“Description of Artificial Sequence Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 200 - DNA, 333 bp, Artificial Sequence
  source: /note=“Description of Artificial Sequence Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 201 - protein, 218 aa, Artificial Sequence
  source: /note=“Description of Artificial Sequence Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - DNA, 654 bp, Artificial Sequence
  source: /note=“Description of Artificial Sequence Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 203 - protein, 114 aa, Artificial Sequence
  source: /note=“Description of Artificial Sequence Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 204 - protein, 108 aa, Artificial Sequence
  source: /note=“Description of Artificial Sequence Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 205 - protein, 120 aa, Artificial Sequence
  source: /note=“Description of Artificial Sequence Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 206 - protein, 113 aa, Artificial Sequence
  source: /note=“Description of Artificial Sequence Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 207 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 208 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 209 - protein, 114 aa, Artificial Sequence
  source: /note=“Description of Artificial Sequence Synthetic polypeptide”
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 210 - protein, 297 aa, Artificial Sequence
  source: /note=“Description of Artificial Sequence Synthetic polypeptide”
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 114 aa, Homo sapiens; identical to SEQ ID NO: 207
  VARIANT (93)..(93): /replace=“Lys”
  SITE (1)..(114): /note=“Variant residues given in the sequence have no preference with respect to those in the annotations for variant positions”

SEQ ID NO: 212 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 213 - protein, 10 aa, Artificial Sequence
  source: /note=“Description of Artificial Sequence Synthetic peptide”
GYTFTTYWMH

SEQ ID NO: 214 - protein, 10 aa, Artificial Sequence
  source: /note=“Description of Artificial Sequence Synthetic peptide”
GYTFTSYWMY

SEQ ID NO: 215 - protein, 20 aa, Artificial Sequence
  source: /note=“Description of Artificial Sequence Synthetic peptide”
GGGGSGGGGSGGGGSGGGGS

SEQ ID NO: 216 - protein, 15 aa, Artificial Sequence
  source: /note=“Description of Artificial Sequence Synthetic peptide”
GGGGSGGGGSGGGGS

SEQ ID NO: 217 - protein, 5 aa, Artificial Sequence
  source: /note=“Description of Artificial Sequence Synthetic peptide”
GGGGS

SEQ ID NO: 218 - protein, 242 aa, Artificial Sequence
  source: /note=“Description of Artificial Sequence Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 219 - protein, 242 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 220 - protein, 242 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 221 - protein, 242 aa, Artificial Sequence
  source: /note=“Description of Artificial Sequence Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 222 - protein, 242 aa, Artificial Sequence
  source: /note=“Description of Artificial Sequence Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 223 - protein, 247 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 224 - protein, 247 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 225 - protein, 247 aa, Artificial Sequence
  source: /note=“Description of Artificial Sequence Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 226 - protein, 247 aa, Artificial Sequence
  source: /note=“Description of Artificial Sequence Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 227 - protein, 247 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 228 - protein, 247 aa, Artificial Sequence
  source: /note=“Description of Artificial Sequence Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 229 - protein, 242 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 230 - protein, 242 aa, Artificial Sequence
  source: /note=“Description of Artificial Sequence Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 231 - protein, 12 aa, Artificial Sequence
  source: /note=“Description of Artificial Sequence Synthetic peptide”
HYYYGGSYAMDY

SEQ ID NO: 232 - protein, 9 aa, Artificial Sequence
  source: /note=“Description of Artificial Sequence Synthetic peptide”
QQGNTLPYT

SEQ ID NO: 233 - protein, 18 aa, Artificial Sequence
  source: /note=“Description of Artificial Sequence Synthetic peptide”
GSTSGSGKPGSGEGSTKG

SEQ ID NO: 234 - protein, 119 aa, Artificial Sequence
  source: /note=“Description of Artificial Sequence Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVT

SEQ ID NO: 235 - protein, 111 aa, Artificial Sequence
  source: /note=“Description of Artificial Sequence Synthetic polypeptide”
ELVLTQSPKFMSTSVGDRVSVTCKASQNVGTNVAWYQQKPGQSPKPLIYSATYRNSGVPDRFTGSGSGTDFTLTITNVQSKDLADYFYFCQYNRYPYTSGGGTKLEIKRRS

SEQ ID NO: 236 - protein, 248 aa, Artificial Sequence
  source: /note=“Description of Artificial Sequence Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGSTSGSGKPGSGEGSTKGELVLTQSPKFMSTSVGDRVSVTCKASQNVGTNVAWYQQKPGQSPKPLIYSATYRNSGVPDRFTGSGSGTDFTLTITNVQSKDLADYFYFCQYNRYPYTSGGGTKLEIKRRS

SEQ ID NO: 237 - protein, 486 aa, Artificial Sequence
  source: /note=“Description of Artificial Sequence Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 238 - protein, 486 aa, Artificial Sequence
  source: /note=“Description of Artificial Sequence Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 239 - protein, 486 aa, Artificial Sequence
  source: /note=“Description of Artificial Sequence Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 240 - protein, 486 aa, Artificial Sequence
  source: /note=“Description of Artificial Sequence Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 241 - protein, 486 aa, Artificial Sequence
  source: /note=“Description of Artificial Sequence Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 242 - protein, 491 aa, Artificial Sequence
  source: /note=“Description of Artificial Sequence Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 243 - protein, 491 aa, Artificial Sequence
  source: /note=“Description of Artificial Sequence Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 244 - protein, 491 aa, Artificial Sequence
  source: /note=“Description of Artificial Sequence Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 245 - protein, 491 aa, Artificial Sequence
  source: /note=“Description of Artificial Sequence Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 246 - protein, 491 aa, Artificial Sequence
  source: /note=“Description of Artificial Sequence Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 247 - protein, 491 aa, Artificial Sequence; identical to SEQ ID NO: 246
  source: /note=“Description of Artificial Sequence Synthetic polypeptide”

SEQ ID NO: 248 - protein, 491 aa, Artificial Sequence
  source: /note=“Description of Artificial Sequence Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 249 - protein, 486 aa, Artificial Sequence
  source: /note=“Description of Artificial Sequence Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 250 - protein, 45 aa, Artificial Sequence
  source: /note=“Description of Artificial Sequence Synthetic polypeptide”
TTTPAPRPPTPAPTIASQPLSLRPEACRPAAGGAVHTRGLDFACD

SEQ ID NO: 251 - protein, 24 aa, Artificial Sequence
  source: /note=“Description of Artificial Sequence Synthetic peptide”
IYIWAPLAGTCGVLLLSLVITLYC

SEQ ID NO: 252 - DNA, 72 bp, Artificial Sequence
  source: /note=“Description of Artificial Sequence Synthetic oligonucleotide”
atctacatctgggcgcccttggccgggacttgtggggtccttctcctgtcactggttatcaccctttactgc

SEQ ID NO: 253 - protein, 230 aa, Artificial Sequence
  source: /note=“Description of Artificial Sequence Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 254 - DNA, 135 bp, Artificial Sequence
  source: /note=“Description of Artificial Sequence Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 255 - DNA, 690 bp, Artificial Sequence
  source: /note=“Description of Artificial Sequence Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 256 - protein, 282 aa, Artificial Sequence
  source: /note=“Description of Artificial Sequence Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 257 - DNA, 847 bp, Artificial Sequence
  source: /note=“Description of Artificial Sequence Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 258 - protein, 10 aa, Artificial Sequence
  source: /note=“Description of Artificial Sequence Synthetic peptide”
GGGGSGGGGS

SEQ ID NO: 259 - DNA, 30 bp, Artificial Sequence
  source: /note=“Description of Artificial Sequence Synthetic oligonucleotide”
ggtggcggaggttctggaggtggaggttcc

SEQ ID NO: 260 - protein, 112 aa, Artificial Sequence
  source: /note=“Description of Artificial Sequence Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 261 - protein, 112 aa, Artificial Sequence
  source: /note=“Description of Artificial Sequence Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 262 - DNA, 336 bp, Artificial Sequence
  source: /note=“Description of Artificial Sequence Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 263 - DNA, 336 bp, Artificial Sequence
  source: /note=“Description of Artificial Sequence Synthetic polynucleotide”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 264 - protein, 42 aa, Artificial Sequence
  source: /note=“Description of Artificial Sequence Synthetic polypeptide”
KRGRKKLLYIFKQPFMRPVQTTQEEDGCSCRFPEEEEGGCEL

SEQ ID NO: 265 - protein, 48 aa, Artificial Sequence
  source: /note=“Description of Artificial Sequence Synthetic polypeptide”
QRRKYRSNKGESPVEPAEPCRYSCPREEEGSTIPIQEDYRKPEPACSP

SEQ ID NO: 266 - DNA, 126 bp, Artificial Sequence
  source: /note=“Description of Artificial Sequence Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 267 - DNA, 144 bp, Artificial Sequence
  source: /note=“Description of Artificial Sequence Synthetic polynucleotide”
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 268 - protein, 21 aa, Artificial Sequence
  source: /note=“Description of Artificial Sequence Synthetic peptide”
MALPVTALLLPLALLLHAARP

SEQ ID NO: 269 - DNA, 2000 bp, Artificial Sequence
  source: /note=“Description of Artificial Sequence Synthetic polynucleotide”
  misc_feature (1)..(2000): /note=“This sequence may encompass 50-2000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 270 - DNA, 1184 bp, Artificial Sequence
  source: /note=”Description of Artificial Sequence Synthetic polynucleotide“
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 271 - protein, 448 aa, Artificial Sequence
  source: /note=”Description of Artificial Sequence Synthetic polypeptide“
QVQLQESGPGLVKPSQTLSLTCTVSDYSITSDYAWNWIRQFPGKGLEWMGYISYSGSTSYNPSLKSRITISRDTSKNQFSLQLNSVTAADTAVYYCASFDYAHAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 214 aa, Artificial Sequence
  source: /note=”Description of Artificial Sequence Synthetic polypeptide“
DIVLTQSPAFLSVTPGEKVTFTCQASQSIGTSIHWYQQKTDQAPKLLIKYASESISGIPSRFSGSGSGTDFTLTISSVEAEDAADYYCQQINSWPTTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 6 aa, Artificial Sequence
  source: /note=”Description of Artificial Sequence Synthetic peptide“
SDYAWN

SEQ ID NO: 274 - protein, 16 aa, Artificial Sequence
  source: /note=”Description of Artificial Sequence Synthetic peptide“
YISYSGSTSYNPSLKS

SEQ ID NO: 275 - protein, 9 aa, Artificial Sequence
  source: /note=”Description of Artificial Sequence Synthetic peptide“
FDYAHAMDY

SEQ ID NO: 276 - protein, 11 aa, Artificial Sequence
  source: /note=”Description of Artificial Sequence Synthetic peptide“
QASQSIGTSIH

SEQ ID NO: 277 - protein, 7 aa, Artificial Sequence
  source: /note=”Description of Artificial Sequence Synthetic peptide“
YASESIS

SEQ ID NO: 278 - protein, 9 aa, Artificial Sequence
  source: /note=”Description of Artificial Sequence Synthetic peptide“
QQINSWPTT

SEQ ID NO: 279 - protein, 143 aa, Artificial Sequence
  source: /note=”Description of Artificial Sequence Synthetic polypeptide“
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDITSASYTMI

SEQ ID NO: 280 - protein, 447 aa, Artificial Sequence
  source: /note=”Description of Artificial Sequence Synthetic polypeptide“
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 281 - protein, 215 aa, Artificial Sequence
  source: /note=”Description of Artificial Sequence Synthetic polypeptide“
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 137 aa, Artificial Sequence
  source: /note=”Description of Artificial Sequence Synthetic polypeptide“
MEFGLSWVFLVALLRGVQCQVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSS

SEQ ID NO: 283 - protein, 126 aa, Artificial Sequence
  source: /note=”Description of Artificial Sequence Synthetic polypeptide“
MLPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIK

SEQ ID NO: 284 - protein, 121 aa, Artificial Sequence
  source: /note=”Description of Artificial Sequence Synthetic polypeptide“
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 285 - protein, 109 aa, Artificial Sequence
  source: /note=”Description of Artificial Sequence Synthetic polypeptide“
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 286 - protein, 9 aa, Artificial Sequence
  source: /note=”Description of Artificial Sequence Synthetic peptide“
QNDYSYPYT

SEQ ID NO: 287 - DNA, 63 bp, Artificial Sequence
  source: /note=”Description of Artificial Sequence Synthetic oligonucleotide“
atggccctgcctgtgacagccctgctgctgcctctggctctgctgctgcatgccgctagaccc

SEQ ID NO: 288 - DNA, 63 bp, Artificial Sequence
  source: /note=”Description of Artificial Sequence Synthetic oligonucleotide“
atggccctccctgtcaccgccctgctgcttccgctggctcttctgctccacgccgctcggccc

SEQ ID NO: 289 - DNA, 72 bp, Artificial Sequence
  source: /note=”Description of Artificial Sequence Synthetic oligonucleotide“
atctacatttgggcccctctggctggtacttgcggggtcctgctgctttcactcgtgatcactctttactgt

SEQ ID NO: 290 - DNA, 126 bp, Artificial Sequence
  source: /note=”Description of Artificial Sequence Synthetic polynucleotide“
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 291 - DNA, 336 bp, Artificial Sequence
  source: /note=”Description of Artificial Sequence Synthetic polynucleotide“
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 292 - protein, 5 aa, Artificial Sequence
  source: /note=”Description of Artificial Sequence Synthetic peptide“
GGGGS

SEQ ID NO: 293 - protein, 150 aa, Artificial Sequence [sequence omitted]

SEQ ID NO: 294 - DNA, 450 bp, Artificial Sequence
  source: /note=”Description of Artificial Sequence Synthetic polynucleotide“
cccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtc

SEQ ID NO: 295 - protein, 394 aa, Artificial Sequence
  source: /note=”Description of Artificial Sequence Synthetic polypeptide“
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 296 - DNA, 1182 bp, Artificial Sequence
  source: /note=”Description of Artificial Sequence Synthetic polynucleotide“
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 297 - protein, 4 aa, Artificial Sequence
  source: /note=”Description of Artificial Sequence Synthetic peptide“
GGGS

SEQ ID NO: 298 - DNA, 5000 bp, Artificial Sequence
  source: /note=”Description of Artificial Sequence Synthetic polynucleotide“
  misc_feature (1)..(5000): /note=”This sequence may encompass 50-5000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 299 - protein, 373 aa, Artificial Sequence
  source: /note=“Description of Artificial Sequence Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 300 - protein, 35 aa, Artificial Sequence
  source: /note=“Description of Artificial Sequence Synthetic polypeptide”
TKKKYSSSVHDPNGEYMFMRAVNTAKKSRLTDVTL

SEQ ID NO: 301 - DNA, 105 bp, Artificial Sequence
  source: /note=“Description of Artificial Sequence Synthetic polynucleotide”
acaaaaaagaagtattcatccagtgtgcacgaccctaacggtgaatacatgttcatgagagcagtgaacacagccaaaaaatccagactcacagatgtgacccta

SEQ ID NO: 302 - protein, 69 aa, Artificial Sequence
  source: /note=“Description of Artificial Sequence Synthetic polypeptide”
TTTPAPRPPTPAPTIASQPLSLRPEACRPAAGGAVHTRGLDFACDFWLPIGCAAFVVVCILGCILICWL

SEQ ID NO: 303 - DNA, 207 bp, Artificial Sequence
  source: /note=“Description of Artificial Sequence Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggttacccataggatgtgcagcctttgttgtagtctgcattttgggatgcatacttatttgttggctt

SEQ ID NO: 304 - protein, 41 aa, Artificial Sequence
  source: /note=“Description of Artificial Sequence Synthetic polypeptide”
RSKRSRLLHSDYMNMTPRRPGPTRKHYQPYAPPRDFAAYRS

SEQ ID NO: 305 - DNA, 123 bp, Artificial Sequence
  source: /note=“Description of Artificial Sequence Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 306 - protein, 10 aa, Artificial Sequence
  source: /note=“Description of Artificial Sequence Synthetic peptide”
GVSLPDYGVS

SEQ ID NO: 307 - protein, 16 aa, Artificial Sequence
  source: /note=“Description of Artificial Sequence Synthetic peptide”
VIWGSETTYYNSALKS

SEQ ID NO: 308 - protein, 16 aa, Artificial Sequence
  source: /note=“Description of Artificial Sequence Synthetic peptide”
VIWGSETTYYSSSLKS

SEQ ID NO: 309 - protein, 16 aa, Artificial Sequence
  source: /note=“Description of Artificial Sequence Synthetic peptide”
VIWGSETTYYQSSLKS

SEQ ID NO: 310 - protein, 16 aa, Artificial Sequence
  source: /note=“Description of Artificial Sequence Synthetic peptide”
VIWGSETTYYNSSLKS

SEQ ID NO: 311 - protein, 11 aa, Artificial Sequence
  source: /note=“Description of Artificial Sequence Synthetic peptide”
RASQDISKYLN

SEQ ID NO: 312 - protein, 7 aa, Artificial Sequence
  source: /note=“Description of Artificial Sequence Synthetic peptide”
HTSRLHS

SEQ ID NO: 313 - protein, 40 aa, Artificial Sequence
  source: /note=“Description of Artificial Sequence Synthetic polypeptide”
  SITE (1)..(40): /note=“This sequence may encompass 1-10 ′Gly Gly Gly Ser′ repeating units”
GGGSGGGSGGGSGGGSGGGSGGGSGGGSGGGSGGGSGGGS

SEQ ID NO: 314 - protein, 4 aa, Artificial Sequence
  source: /note=“Description of Artificial Sequence Synthetic peptide”
RGDS

(19) United States
(12) Patent Application Publication
(10) Pub. No.: US 2024/0245670 A1
(43) Pub. Date: Jul. 25, 2024

(54) DOSING REGIMEN AND PHARMACEUTICAL COMBINATION COMPRISING 3-(1-OXOISOINDOLIN-2-YL)PIPERIDINE-2,6-DIONE DERIVATIVES

(71) Applicant: Novartis AG, Basel (CH)

(72) Inventors: Simone BONAZZI, Cambridge, MA (US); Adam Crystal, Newton, MA (US); John Scott Cameron, Cambridge, MA (US); Eva Marie Genevieve D'Hennezel, Cambridge, MA (US); Glenn Dranoff, Cambridge, MA (US); Ry Roger Forseth, West Orange, NJ (US); Dominik Johannes Hainzl, Cambridge, MA (US); Jacqueline Kinyamu-Akunda, East Hanover, NJ (US); Guiqing Liang, Cambridge, MA (US); Lilly Mary Petruzzelli, Cambridge, MA (US)

(21) Appl. No.: 17/415,923

(22) PCT Filed: Dec. 19, 2019
(86) PCT No.: PCT/IB2019/061130

§ 371 Date: Jun. 18, 2021

Related U.S. Application Data
(60) Provisional application No. 62/782,421, filed on Dec. 20, 2018.
(60) Provisional application No. 62/806,136, filed on Feb. 15, 2019.

(51) Int. Cl. A61K 31/4545; A61K 9/00; A61K 31/55; A61P 35/00

(52) U.S. Cl. CPC A61K 31/4545; A61K 9/0053; A61K 31/55; A61P 35/00

(57) ABSTRACT

The present disclosure relates to dosing regimens, formulations, and combinations comprising 3-(1-oxoisoindolin-2-yl)piperidine-2,6-dione compounds or pharmaceutical compositions comprising the same; and methods of using such combinations and compositions in the treatment or prevention IKAROS Family Zinc Finger 2 (IKZF2)-dependent diseases or disorders or where reduction of IKZF2 or IKZF4 protein levels can ameliorate a disease, for example, the treatment of cancers.

### RELATED APPLICATIONS

This application claims the benefit of and priority to U.S. Provisional application Nos. 62/782,421, filed Dec. 20, 2018, and 62/806,136, filed Feb. 15, 2019, the entire contents of each of which are incorporated herein by reference in their entireties.

### FIELD OF THE DISCLOSURE

The present disclosure relates to dosing regimens, formulations, and combinations comprising 3-(1-oxoisoindolin-2-yl)piperidine-2,6-dione compound, and their use for the treatment of IKAROS Family Zinc Finger 2 (IKZF2)-dependent diseases or disorders or where reduction of IKZF2 or IKZF4 protein levels can treat, prevent, or ameliorate a disease.

### BACKGROUND OF THE DISCLOSURE

IKAROS Family Zinc Finger 2 (IKZF2) (also known as Helios) is one of the five members of the Ikaros family of transcription factors found in mammals. IKZF2 contains four zinc finger domains near the N-terminus, which are involved in DNA binding, and two zinc finger domains at the C-terminus, which are involved in protein dimerization. IKZF2 is about 50% identical with Ikaros family members, Ikaros (IKZF1), Aiolos (IKZF3), and Eos (IKZF4) with highest homology in the zinc finger regions (80%+identity). These four Ikaros family transcription factors bind to the same DNA consensus site and can heterodimerize with each other when co-expressed in cells. The fifth Ikaros family protein, Pegasus (IKZF5), is only 25% identical to IKZF2, binds a different DNA site than other Ikaros family members and does not readily heterodimerize with the other Ikaros family proteins. IKZF2, IKZF1 and IKZF3 are expressed mainly in hematopoietic cells while IKZF4 and IKZF5 are expressed in a wide variety of tissues. (John, L. B., et al., (2011), Mol. Immunol. 48:1272-1278; Perdomo, J., et al., (2000), J. Biol. Chem. 275:38347-38354.)

IKZF2 is believed to have an important role in the function and stability of regulatory T cells (Tregs). IKZF2 is highly expressed at the mRNA and protein level by regulatory T-cell populations. Knockdown of IKZF2 by siRNA has been shown to result in downregulation of FoxP3 and to impair the ability of isolated human CD4+CD25+ Tregs to block T-cell activation in vitro. Moreover, overexpression of IKZF2 in isolated murine Tregs has been shown to increase expression of Treg related markers such as CD103 and GITR and the IKZF2 overexpressing cells showed increased suppression of responder T-cells. IKZF2 has also been found to bind the promoter of FoxP3, the defining transcription factor of the regulatory T-cell lineage, and to affect FoxP3 expression.

Knockout of IKZF2 within FoxP3-expressing Tregs in mice has been shown to cause activated Tregs to lose their inhibitory properties, to express T-effector cytokines, and to take on T-effector functions. IKZF2 knockout mutant mice develop autoimmune disease by 6-8 months of age, with increased numbers of activated CD4 and CD8 T cells, follicular helper T cells and germinal center B cells. This observed effect is believed to be cell intrinsic, as Rag2−/− mice given bone marrow from IKZF2 knockout mice, but not bone marrow from IKZF2+/+ develop autoimmune disease. Direct evidence that IKZF2 affects regulatory T-cell function has been shown in the analysis of mice in which IKZF2 was deleted only in FoxP3 expressing cells (FoxP3-YFP-Cre Heliosfl/fl). The results showed that the mice also develop autoimmune disease with similar features as observed in the whole animal IKZF2 knockout. Moreover, pathway analysis of a CHIP-SEQ experiment has also suggested that IKZF2 is affecting expression of genes in the STAT5/IL-2Rα pathway in regulatory T-cells. This effect of IKZF2 loss was shown to be more apparent after an immune challenge (viral infection or injection with sheep's blood), and it was noted that after immune stimulation, the IKZF2 negative regulatory T cells began to take on features of effector T cells. (Getnet, D., et al., Mol. Immunol. (2010), 47:1595-1600; Bin Dhuban, K., et al., (2015), J. Immunol. 194: 3687-96; Kim, H-J., et al., (2015), Science 350: 334-339; Nakawaga, H., et al., (2016) PNAS, 113: 6248-6253)

Overexpression of Ikaros isoforms, which lack the DNA binding regions, have been shown to be associated with multiple human haematological malignancies. Recently, mutations in the IKZF2 gene, which lead to abnormal splicing variants, have been identified in adult T-cell leukemias and low hypodiploid acute lymphoblastic leukemia. It has been proposed that these isoforms, which are capable of dimerization, have a dominant negative effect on Ikaros family transcription factors, which primes the development of lymphomas. IKZF2 knockout mutants that survive into adulthood do not develop lymphomas, supporting this hypothesis (Asanuma, S., et al., (2013), Cancer Sci. 104:1097-1106; Zhang, Z., et al., (2007), Blood 109:2190-2197; Kataoka, D., et al., (2015), Nature Genetics 47:1304-1315.)

Currently, anti-CTLA4 antibodies are used in the clinic to target Tregs in tumors. However, targeting CTLA4 often causes systemic activation of T-effector cells, resulting in excessive toxicity and limiting therapeutic utility. Up to ¾ of patients treated with a combination of anti-PD1 and anti-CTLA4 have reported grade 3 or higher adverse events. Thus, a strong need exists to provide compounds that target Tregs in tumors without causing systemic activation of T-effector cells.

An IKZF2-specific degrader has the potential to focus the enhanced immune response to areas within or near tumors providing a potentially more tolerable and less toxic therapeutic agent for the treatment of cancer.

### SUMMARY OF THE DISCLOSURE

Disclosed herein, inter alia, are methods, formulations, combinations, and compositions comprising a compound or a combination comprising a compound that has degrader activity for IKZF2 (a first therapeutic agent) and a second therapeutic agent disclosed herein. The second therapeutic agent can be chosen from one or more of: an inhibitor of an inhibitory molecule (e.g., an inhibitor of a checkpoint inhibitor), an activator of a costimulatory molecule, a chemotherapeutic agent, a targeted anti-cancer therapy, an oncolytic drug, a cytotoxic agent, or any of the therapeutic agents disclosed herein. In some embodiments, the therapeutic agent can be chosen from a PD-1 inhibitor, a LAG-3 inhibitor, a cytokine, an A2A antagonist, a GITR agonist, a TIM-3 inhibitor, a STING agonist, and a TLR7 agonist.

A first aspect of the present disclosure relates a combination comprising,
(a) a compound (or first therapeutic agent) of Formula (Ic):

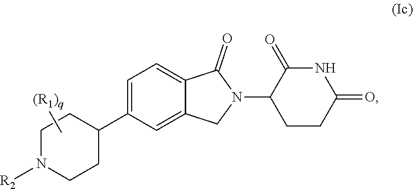

or a pharmaceutically acceptable salt, hydrate, solvate, prodrug, stereoisomer, or tautomer thereof:
wherein:
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R_{6′} are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S; and
q is 0, 1, 2, 3, or 4; and
(b) a second therapeutic agent.

In another aspect, the present disclosure relates to pharmaceutical formulation comprising a compound (or first therapeutic agent) (a) a compound (or first therapeutic agent) of Formula (Ic):

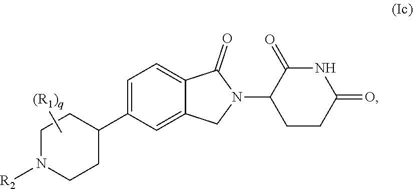

or a pharmaceutically acceptable salt, hydrate, solvate, prodrug, stereoisomer, or tautomer thereof:
wherein:
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₃-C₇)cycloalkyl ring or a 5- to 7-membered heterocycloalkyl ring comprising 1 to 3 heteroatoms selected from O, N, and S optionally substituted with one or more R₁₀;
R₆ and R_{6′} are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more Rn, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S; and
q is 0, 1, 2, 3, or 4; and
(b) a second therapeutic agent.

In another aspect, the present disclosure relates to pharmaceutical formulation comprising: (a) a compound (or first therapeutic agent) of Formula (Ic), or a pharmaceutically acceptable salt, hydrate, solvate, prodrug, stereoisomer, or tautomer thereof; and (b) a second agent.

In another aspect, the present disclosure relates to pharmaceutical formulation comprising: (a) a compound (or first therapeutic agent) of Formula (Ic), or a pharmaceutically acceptable salt, hydrate, solvate, prodrug, stereoisomer, or tautomer thereof; and (b) one or more therapeutic agent(s).

Another aspect of the present disclosure relates to pharmaceutical formulation comprising a compound (or first therapeutic agent) selected from:

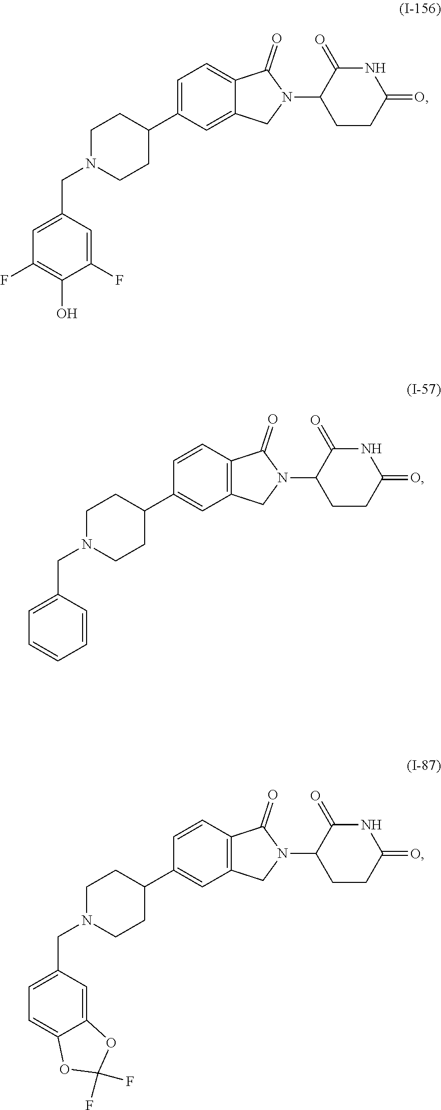 [SMILES: O=C1CCC(N2Cc3cc(C4CCN(Cc5cc(F)c(O)c(F)c5)CC4)ccc3C2=O)C(=O)N1 ; O=C1CCC(N2Cc3cc(C4CCN(Cc5ccccc5)CC4)ccc3C2=O)C(=O)N1 ; O=C1CCC(N2Cc3cc(C4CCN(Cc5ccc6c(c5)OC(F)(F)O6)CC4)ccc3C2=O)C(=O)N1]

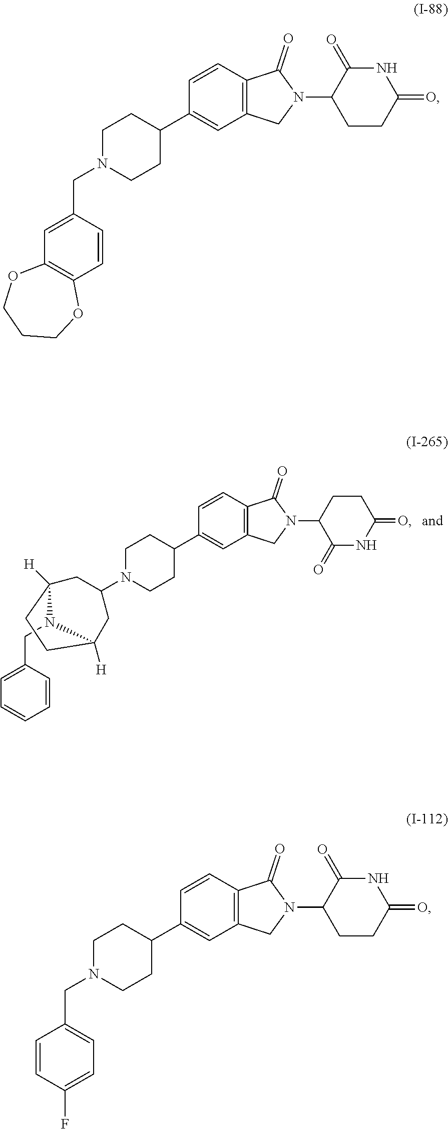 [SMILES: O=C1CCC(N2Cc3cc(C4CCN(Cc5ccc6c(c5)OCCCO6)CC4)ccc3C2=O)C(=O)N1 ; O=C1CCC(N2Cc3cc(C4CCN(C5C[C@H]6CC[C@@H](C5)N6Cc5ccccc5)CC4)ccc3C2=O)C(=O)N1 ; O=C1CCC(N2Cc3cc(C4CCN(Cc5ccc(F)cc5)CC4)ccc3C2=O)C(=O)N1]

or a pharmaceutically acceptable salt, hydrate, solvate, prodrug, stereoisomer, or tautomer thereof; and a second therapeutic agent.

In another aspect, the present disclosure relates to a pharmaceutical formulation comprising a therapeutically effective amount of (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s).

Another aspect of the present disclosure relates to a combination comprising a therapeutically effective amount of (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent.

In another aspect, the present disclosure relates to a combination comprising a therapeutically effective amount of (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s).

Another aspect of the present disclosure relates to a combination comprising a therapeutically effective amount of (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; (b) a second therapeutic agent; and (c) a pharmaceutically acceptable carrier or excipient.

In another aspect, the present disclosure relates to a combination comprising a therapeutically effective amount of (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; (b) one or more therapeutic agent(s); and (c) a pharmaceutically acceptable carrier or excipient.

Another aspect of the present disclosure relates to a pharmaceutical formulation comprising a therapeutically effective amount of (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; (b) a second therapeutic agent; and (c) a pharmaceutically acceptable carrier or excipient.

In another aspect, the present disclosure relates to a pharmaceutical formulation comprising a therapeutically effective amount of (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; (b) one or more therapeutic agent(s); and (c) a pharmaceutically acceptable carrier or excipient.

Another aspect of the present disclosure relates to a method of treating or preventing cancer comprising administering to a patient in need thereof a combination comprising (a) a compound of Formula (Ic), or a pharmaceutically acceptable salt, hydrate, solvate, prodrug, stereoisomer, or tautomer thereof; and (b) a second therapeutic agent.

In another aspect, the present disclosure relates to a method of treating or preventing cancer comprising administering to a patient in need thereof a combination comprising (a) a compound of Formula (Ic), or a pharmaceutically acceptable salt, hydrate, solvate, prodrug, stereoisomer, or tautomer thereof; and (b) one or more therapeutic agent(s).

Another aspect of the present disclosure relates to a method of treating or preventing cancer comprising administering to a patient in need thereof a combination comprising (a) a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent.

In another aspect, the present disclosure relates to a method of treating or preventing cancer comprising administering to a patient in need thereof a combination comprising (a) a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s).

Another aspect of the present disclosure relates to a method of treating or preventing cancer comprising administering to a patient in need thereof a pharmaceutical formulation comprising (a) a therapeutically effective amount of a compound of Formula (Ic), or a pharmaceutically acceptable salt, hydrate, solvate, prodrug, stereoisomer, or tautomer thereof; and (b) a second therapeutic agent.

In another aspect, the present disclosure relates to a method of treating or preventing cancer comprising administering to a patient in need thereof a pharmaceutical formulation comprising (a) a therapeutically effective amount of a compound of Formula (Ic), or a pharmaceutically acceptable salt, hydrate, solvate, prodrug, stereoisomer, or tautomer thereof; and (b) one or more therapeutic agent(s).

Another aspect of the present disclosure relates to a method of treating or preventing cancer comprising administering to the patient in need thereof a combination comprising (a) a pharmaceutical formulation comprising a therapeutically effective amount of a compound of Formula (Ic), or a pharmaceutically acceptable salt, hydrate, solvate, prodrug, stereoisomer, or tautomer thereof; and (b) a second therapeutic agent.

In another aspect, the present disclosure relates to a method of treating or preventing cancer comprising administering to the patient in need thereof a combination comprising (a) a pharmaceutical formulation comprising a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent.

Another aspect of the present disclosure relates to a method of treating or preventing cancer comprising administering to the patient in need thereof a combination comprising (a) a pharmaceutical formulation comprising a therapeutically effective amount of a compound of Formula (Ic), or a pharmaceutically acceptable salt, hydrate, solvate, prodrug, stereoisomer, or tautomer thereof; and (b) one or more therapeutic agent(s).

In another aspect, the present disclosure relates to a method of treating or preventing cancer comprising administering to the patient in need thereof a combination comprising (a) a pharmaceutical formulation comprising a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s).

Another aspect of the present disclosure relates to a method of treating or preventing cancer comprising administering to a patient in need thereof a pharmaceutical formulation comprising (a) a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent.

In another aspect, the present disclosure relates to a method of treating or preventing cancer comprising administering to a patient in need thereof a pharmaceutical formulation comprising (a) a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s).

Another aspect of the present disclosure relates to a combination comprising (a) a compound of Formula (Ic), or a pharmaceutically acceptable salt, hydrate, solvate, prodrug, stereoisomer, or tautomer thereof, or a pharmaceutical formulation comprising a therapeutically effective amount of a compound of Formula (Ic), or a pharmaceutically acceptable salt, hydrate, solvate, prodrug, stereoisomer, or tautomer thereof, and a pharmaceutically acceptable carrier or excipient; and (b) a second therapeutic agent for use in the treatment or prevention of cancer.

In another aspect, the present disclosure relates to a combination comprising (a) a compound of Formula (Ic), or a pharmaceutically acceptable salt, hydrate, solvate, prodrug, stereoisomer, or tautomer thereof, or a pharmaceutical formulation comprising a therapeutically effective amount of a compound of Formula (Ic), or a pharmaceutically acceptable salt, hydrate, solvate, prodrug, stereoisomer, or tautomer thereof, and a pharmaceutically acceptable carrier or excipient; and (b) one or more therapeutic agent(s) for use in the treatment or prevention of cancer.

Another aspect of the present disclosure relates to a combination comprising (a) a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) a second therapeutic agent for use in the treatment or prevention of cancer.

In another aspect, the present disclosure relates to a combination comprising (a) a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) one or more therapeutic agent(s) for use in the treatment or prevention of cancer.

Another aspect of the present disclosure relates to the use of a combination comprising (a) a compound of Formula (Ic), or a pharmaceutically acceptable salt, hydrate, solvate, prodrug, stereoisomer, or tautomer thereof, or a pharmaceutical formulation comprising a therapeutically effective amount of a compound of Formula (Ic), or a pharmaceutically acceptable salt, hydrate, solvate, prodrug, stereoisomer, or tautomer thereof; and (b) a second therapeutic agent for the treatment or prevention of cancer.

In another aspect, the present disclosure relates to the use of a combination comprising (a) a compound of Formula (Ic), or a pharmaceutically acceptable salt, hydrate, solvate, prodrug, stereoisomer, or tautomer thereof, or a pharmaceutical formulation comprising a therapeutically effective amount of a compound of Formula (Ic), or a pharmaceutically acceptable salt, hydrate, solvate, prodrug, stereoisomer, or tautomer thereof; and (b) one or more therapeutic agent(s) for the treatment or prevention of cancer.

Another aspect of the present disclosure relates to the use of a combination comprising (a) a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the treatment or prevention of cancer.

In another aspect, the present disclosure relates to the use of a combination comprising (a) a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the treatment or prevention of cancer.

Another aspect of the present disclosure relates to the use of a combination comprising (a) a compound of Formula (Ic), or a pharmaceutically acceptable salt, hydrate, solvate, prodrug, stereoisomer, or tautomer thereof, or a pharmaceutical formulation comprising a therapeutically effective amount of a compound of Formula (Ic), or a pharmaceutically acceptable salt, hydrate, solvate, prodrug, stereoisomer, or tautomer thereof; and (b) a second therapeutic agent for the manufacture of a medicament for treating or preventing of cancer.

In another aspect, the present disclosure relates to the use of a combination comprising (a) a compound of Formula (Ic), or a pharmaceutically acceptable salt, hydrate, solvate, prodrug, stereoisomer, or tautomer thereof, or a pharmaceutical formulation comprising a therapeutically effective amount of a compound of Formula (Ic), or a pharmaceutically acceptable salt, hydrate, solvate, prodrug, stereoisomer, or tautomer thereof; and (b) one or more therapeutic agent(s) for the manufacture of a medicament for treating or preventing of cancer.

Another aspect of the present disclosure relates to the use of a combination comprising (a) a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the manufacture of a medicament for treating or preventing of cancer.

In another aspect, the present disclosure relates to the use of a combination comprising (a) a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a therapeutically effective amount of a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the manufacture of a medicament for treating or preventing of cancer.

Another aspect of the present disclosure relates to a method of treating or preventing cancer comprising administering to a patient in need thereof, a compound of Formula (Ic), or a pharmaceutically acceptable salt, hydrate, solvate, prodrug, stereoisomer, or tautomer thereof, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day.

In another aspect, the present disclosure relates to a method of treating or preventing cancer comprising administering to a patient in need thereof, a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day.

Another aspect of the present disclosure relates to a method of treating or preventing cancer comprising administering to a patient in need thereof a pharmaceutical formulation comprising a compound of Formula (I′), or a pharmaceutically acceptable salt, hydrate, solvate, prodrug, stereoisomer, or tautomer thereof, and a pharmaceutically acceptable carrier or excipient, wherein the pharmaceutical formulation comprises about 2 mg, or about 4 mg, or about 10 mg, or about 20 mg, or about 40 mg, or about 80 mg, or about 160 mg, or about 320 mg of the compound.

Another aspect of the present disclosure relates to a method of treating or preventing cancer comprising administering to a patient in need thereof a pharmaceutical formulation comprising a compound of Formula (Ic), or a pharmaceutically acceptable salt, hydrate, solvate, prodrug, stereoisomer, or tautomer thereof, and a pharmaceutically acceptable carrier or excipient, wherein the pharmaceutical formulation comprises about 2 mg, or about 4 mg, or about 10 mg, or about 20 mg, or about 40 mg, or about 80 mg, or about 160 mg, or about 320 mg of the compound.

In another aspect, the present disclosure relates to a method of treating or preventing cancer comprising administering to a patient in need thereof a pharmaceutical formulation comprising a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient, wherein the pharmaceutical formulation comprises about 2 mg, or about 4 mg, or about 10 mg, or about 20 mg, or about 40 mg, or about 80 mg, or about 160 mg, or about 320 mg of the compound.

Another aspect of the present disclosure relates to a method of treating or preventing an IKZF2-dependent disease by reducing or decreasing IKZF2 protein levels in a patient comprising administering to the patient in need thereof a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent, wherein reduction or decrease of IKZF2 protein levels treats or prevents the IKZF2-dependent disease.

In another aspect, the present disclosure relates to a method of treating or preventing an IKZF2-dependent disease by reducing or decreasing IKZF2 protein levels in a patient comprising administering to the patient in need thereof a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s), wherein reduction or decrease of IKZF2 protein levels treats or prevents the IKZF2-dependent disease.

Another aspect of the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for use in the treatment or prevention of an IKZF2-dependent disease by reducing or decreasing IKZF2 protein levels, wherein reduction or decrease of IKZF2 protein levels treats or prevents the IKZF2-dependent disease.

In another aspect, the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for use in the treatment or prevention of an IKZF2-dependent disease by reducing or decreasing IKZF2 protein levels, wherein reduction or decrease of IKZF2 protein levels treats or prevents the IKZF2-dependent disease.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the treatment or prevention of an IKZF2-dependent disease by reducing or decreasing IKZF2 protein levels, wherein reduction or decrease of IKZF2 protein levels treats or prevents the IKZF2-dependent disease.

In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the treatment or prevention of an IKZF2-dependent disease by reducing or decreasing IKZF2 protein levels, wherein reduction or decrease of IKZF2 protein levels treats or prevents the IKZF2-dependent disease.

Another aspect of the present disclosure relates to the use of a combination comprising (a) a compound of Formula (I′), a compound of Formula (Ic) or a compound of Formula (Ic), selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the manufacture of a medicament for treating or preventing an IKZF2-dependent disease by reducing or decreasing IKZF2 protein levels, wherein reduction or decrease of IKZF2 protein levels treats or prevents the IKZF2-dependent disease.

In another aspect, the present disclosure relates to the use of a combination comprising (a) a compound of Formula (I′), a compound of Formula (Ic) or a compound of Formula (Ic), selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the manufacture of a medicament for treating or preventing an IKZF2-dependent disease by reducing or decreasing IKZF2 protein levels, wherein reduction or decrease of IKZF2 protein levels treats or prevents the IKZF2-dependent disease.

Another aspect of the present disclosure relates to a method of treating or preventing an IKZF2-dependent disease by degrading IKZF2 in a patient comprising administering to the patient in need thereof a combination comprising (a)) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent, wherein degradation of IKZF2 treats or prevents the IKZF2-dependent disease.

In another aspect, the present disclosure relates to a method of treating or preventing an IKZF2-dependent disease by degrading IKZF2 in a patient comprising administering to the patient in need thereof a combination comprising (a)) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s), wherein degradation of IKZF2 treats or prevents the IKZF2-dependent disease.

Another aspect of the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for use in the treatment or prevention of an IKZF2-dependent disease by degrading IKZF2, wherein degradation of IKZF2 treats or prevents the IKZF2-dependent disease.

In another aspect, the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for use in the treatment or prevention of an IKZF2-dependent disease by degrading IKZF2, wherein degradation of IKZF2 treats or prevents the IKZF2-dependent disease.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the treatment or prevention of an IKZF2-dependent disease by degrading IKZF2, wherein degradation of IKZF2 treats or prevents the IKZF2-dependent disease.

In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the treatment or prevention of an IKZF2-dependent disease by degrading IKZF2, wherein degradation of IKZF2 treats or prevents the IKZF2-dependent disease.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the manufacture of a medicament for treating or preventing an IKZF2-dependent disease by degrading IKZF2, wherein degradation of IKZF2 treats or prevents the IKZF2-dependent disease.

In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the manufacture of a medicament for treating or preventing an IKZF2-dependent disease by degrading IKZF2, wherein degradation of IKZF2 treats or prevents the IKZF2-dependent disease.

Another aspect of the present disclosure relates to a method for treating a disease that is affected by the modulation of IKZF2 protein levels in a patient comprising administering to the patient in need thereof a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent.

In another aspect, the present disclosure relates to a method for treating a disease that is affected by the modulation of IKZF2 protein levels in a patient comprising administering to the patient in need thereof a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s).

Another aspect of the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for use in the treatment or prevention of a disease that is affected by the modulation of IKZF2 protein levels, wherein modulation of IKZF2 protein levels treats or prevents the disease.

In another aspect, the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for use in the treatment or prevention of a disease that is affected by the modulation of IKZF2 protein levels, wherein modulation of IKZF2 protein levels treats or prevents the disease.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the treatment or prevention of a disease that is affected by the modulation of IKZF2 protein levels, wherein modulation of IKZF2 protein levels treats or prevents the disease.

In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the treatment or prevention of a disease that is affected by the modulation of IKZF2 protein levels, wherein modulation of IKZF2 protein levels treats or prevents the disease.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the manufacture of a medicament for treating or preventing a disease that is affected by the modulation of IKZF2 protein levels, wherein modulation of IKZF2 protein levels treats or prevents the disease.

In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the manufacture of a medicament for treating or preventing a disease that is affected by the modulation of IKZF2 protein levels, wherein modulation of IKZF2 protein levels treats or prevents the disease.

Another aspect of the present disclosure relates to a method for treating or preventing a disease that is affected by a decrease or a reduction in IKZF2 protein levels in a patient comprising administering to the patient in need thereof a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent, wherein reduction or decrease of IKZF2 protein levels treats or prevents the disease.

In another aspect, the present disclosure relates to a method for treating or preventing a disease that is affected by a decrease or a reduction in IKZF2 protein levels in a patient comprising administering to the patient in need thereof a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s), wherein reduction or decrease of IKZF2 protein levels treats or prevents the disease.

Another aspect of the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for use in the treatment or prevention of a disease that is affected by a decrease or a reduction in IKZF2 protein levels wherein reduction or decrease of IKZF2 protein levels treats or prevents the disease.

In another aspect, the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for use in the treatment or prevention of a disease that is affected by a decrease or a reduction in IKZF2 protein levels wherein reduction or decrease of IKZF2 protein levels treats or prevents the disease.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the treatment or prevention of a disease that is affected by a decrease or a reduction in IKZF2 protein levels wherein reduction or decrease of IKZF2 protein levels treats or prevents the disease.

In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the treatment or prevention of a disease that is affected by a decrease or a reduction in IKZF2 protein levels wherein reduction or decrease of IKZF2 protein levels treats or prevents the disease.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent for the manufacture of a medicament for treating or preventing a disease that is affected by a decrease or a reduction in IKZF2 protein levels wherein reduction or decrease of IKZF2 protein levels treats or prevents the disease. In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) one or more therapeutic agent(s) for the manufacture of a medicament for treating or preventing a disease that is affected by a decrease or a reduction in IKZF2 protein levels wherein reduction or decrease of IKZF2 protein levels treats or prevents the disease.

Another aspect of the present disclosure relates to a method of treating cancer comprising administering to a patient in need thereof a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) a second therapeutic agent, wherein the cancer is a cancer for which the immune response is deficient or an immunogenic cancer.

In another aspect, the present disclosure relates to a method of treating cancer comprising administering to a patient in need thereof a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) one or more therapeutic agent(s), wherein the cancer is a cancer for which the immune response is deficient or an immunogenic cancer.

Another aspect of the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) a second therapeutic agent, for use in the treatment or prevention of cancer, wherein the cancer is a cancer for which the immune response is deficient or an immunogenic cancer.

In another aspect, the present disclosure relates to a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) one or more therapeutic agent(s), for use in the treatment or prevention of cancer, wherein the cancer is a cancer for which the immune response is deficient or an immunogenic cancer.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) a second therapeutic agent, for the treatment or prevention of cancer, wherein the cancer is a cancer for which the immune response is deficient or an immunogenic cancer.

In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) one or more therapeutic agent(s), for the treatment or prevention of cancer, wherein the cancer is a cancer for which the immune response is deficient or an immunogenic cancer.

Another aspect of the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) a second therapeutic agent for the manufacture of a medicament for treating or preventing of cancer, wherein the cancer is a cancer for which the immune response is deficient or an immunogenic cancer.

In another aspect, the present disclosure relates to the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, or a pharmaceutical formulation comprising a compound of Formula (I′), a compound of Formula (Ic), or a compound of Formula (Ic) selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and a pharmaceutically acceptable carrier or excipient; and (b) one or more therapeutic agent(s) for the manufacture of a medicament for treating or preventing of cancer, wherein the cancer is a cancer for which the immune response is deficient or an immunogenic cancer.

In another aspect, the present disclosure relates to a pharmaceutical combination comprising, a compound that has degrader activity for IKZF2 and one or more therapeutic agent(s), wherein the therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof.

Another aspect of the present disclosure relates to pharmaceutical combination comprising, a compound that has degrader activity for IKZF2 and one or more therapeutic agent(s), wherein the therapeutic agent is selected from a PD-1 inhibitor, a LAG-3 inhibitor, a cytokine, an A2A antagonist, a GITR agonist, a TIM-3 inhibitor, a STING agonist, and a TLR7 agonist, or a combination thereof.

In another aspect, the present disclosure relates to a pharmaceutical combination comprising, a compound that decreases IKZF2 levels in a patient and one or more therapeutic agent(s), wherein the therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof.

Another aspect of the present disclosure relates to a pharmaceutical combination comprising, a compound that decreases IKZF2 levels in a patient and one or more therapeutic agent(s), wherein the therapeutic agent is selected from a PD-1 inhibitor, a LAG-3 inhibitor, a cytokine, an A2A antagonist, a GITR agonist, a TIM-3 inhibitor, a STING agonist, and a TLR7 agonist, or a combination thereof.

In another aspect, the present disclosure relates to a method of treating or preventing cancer comprising administering to a patient in need thereof a compound that has degrader activity for IKZF2 in combination with one or more therapeutic agents, wherein the therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof. In one embodiment, the therapeutic agent is selected from a PD-1 inhibitor, a LAG-3 inhibitor, a cytokine, an A2A antagonist, a GITR agonist, a TIM-3 inhibitor, a STING agonist, and a TLR7 agonist.

Another aspect of the present disclosure relates to a method of treating or preventing cancer comprising administering to a patient in need thereof a compound that decreases IKZF2 levels in a patient in combination with one or more therapeutic agents, wherein the therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof. In one embodiment, the therapeutic agent is selected from a PD-1 inhibitor, a LAG-3 inhibitor, a cytokine, an A2A antagonist, a GITR agonist, a TIM-3 inhibitor, a STING agonist, and a TLR7 agonist.

In another aspect, the present disclosure relates to a method of treating or preventing cancer comprising administering to a patient in need thereof a compound that decreases IKZF2 levels in a patient in combination with one or more therapeutic agents, wherein the therapeutic agent is selected from a PD-1 inhibitor, a LAG-3 inhibitor, a cytokine, an A2A antagonist, a GITR agonist, a TIM-3 inhibitor, a STING agonist, and a TLR7 agonist, or a combination thereof.

In all aspects of the present disclosure above, the pharmaceutical formulation comprising (a) a compound of Formula (I′), a compound of Formula (Ic), or a compound of Formula (Ic) selected from Compound I-156, Compound I-57, Compound I-87, Compound I-88, Compound I-265, and Compound I-112, and (b) a second therapeutic agent, optionally further comprises a pharmaceutically acceptable carrier or excipient.

In all aspects of the present disclosure above, the pharmaceutical formulation comprising (a) a compound of Formula (I′), a compound of Formula (Ic), or a compound of Formula (Ic) selected from Compound I-156, Compound I-57, Compound I-87, Compound I-88, Compound I-265, and Compound I-112, and (b) one or more therapeutic agent (s), optionally further comprises a pharmaceutically acceptable carrier or excipient.

In all aspects of the present disclosure above, the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, and (b) a second therapeutic agent, optionally further comprises a pharmaceutically acceptable carrier or excipient for (a), (b), or both (a) and (b).

In all aspects of the present disclosure above, the use of a combination comprising (a) a compound of Formula (I′), a compound of Formula (Ic), or a compound of Formula (Ic) selected from Compound I-156, Compound I-57, Compound I-87, Compound I-88, Compound I-265, and Compound I-112, and (b) one or more therapeutic agent(s), optionally further comprises a pharmaceutically acceptable carrier or excipient for (a), (b), or both (a) and (b).

### BRIEF DESCRIPTION OF THE DRAWINGS

FIG. 1. is a graph showing the selectivity of Compound I-57 for the degradation of IKZF2 over the other IKAROS family members, IKZF1, IKZF4, and GSPT1 at various concentrations in HEK293T cells overexpressing prolabel-tagged target proteins. The results in FIG. 1 shows that Compound I-57 is a potent and specific degrader of IKZF2.

FIG. 2A is a graph showing IKZF2 degradation in primary Treg cells treated with DMSO as a control and various concentrations of Compound I-57.

FIG. 2B is a graph showing the change upregulation of IL2 mRNA in TCR-stimulated Jurkat cells after IKZF2 degradation when cells were treated with increasing concentrations of Compound I-57. As FIG. 2B shows, upon TCR stimulation, Jurkat cells expressed more IL-2 mRNA in a dose-dependent manner.

FIG. 2C is a bar graph showing the suppressive potency of Treg cells expanded in the presence of Compound I-57. As FIG. 2C shows, IKZF2 degradation with Compound I-57 has downstream biologic consequences in vitro with Treg cells showing reduced capacity to suppress Teff proliferation FIG. 2D is a graph showing the effect on IFNγ production in Teff cells treated with DMSO as a control, and 2.5 nM, 25 nM, and 2.5 μM of Compound I-57. The results show a concomitant increase in IFNγ production by IKZF2+ cells supporting the hypothesis that Compound I-57 could promote Teff function.

FIG. 3. is a bar graph showing the degradation of IKZF2 in primary PBMCs obtained from rabbit, dog, pig, cynomolgus monkey and human, and in primary splenocytes of mouse and rat and treated with Compound I-57. As FIG. 3 shows, degradation was observed in human, monkey and rabbit PBMCs, but not in PBMCs or splenocytes from mouse, rat, dog or pig, at concentrations up to 10 μM (˜4.2 ng/mL).

FIG. 4 is a graph showing the PK/PD relationship in the cynomolgus monkey after a single oral of 0.01, 0.1 or 1 mg/kg of Compound I-57.

FIG. 5. is a graph showing plasma concentration in the cynomolgus monkey of Compound I-57 and IKZF2 expression (as determined by flow cytometry) in FOXP3+ T cells from PBMCs after a single oral of 0.01, 0.1 or 1 mg/kg of Compound I-57.

FIG. 6 is a pictorial representation of the multi-dose PK/PD study design in the human PBMC adoptive transfer mouse model harboring MDA-MB231 xenografts. Fourteen consecutive daily doses of Compound I-57 was administered at 0.3 mg/kg, 1 mg/kg, 3 mg/kg or 30 mg/kg.

FIG. 7 is a graph showing the change in the IKZF2 expression in human CD4+FOXP3+ regulatory T cells isolated from MDA-MB231 tumor xenografts (Tumor) or blood (Periphery) following 14 daily oral doses of 0.3, 1, 3 and 30 mg/kg Compound I-57 administered to the hPBMC AdT model. Treatment with Compound I-57 resulted in robust dose and exposure-dependent IKZF2 degradation, i.e., reduction of the percentage of IKZF2 positive Tregs, in tumor and peripheral blood.

FIG. 8A is a bar graph showing the change in the IKZF2 protein levels in total tumor-infiltrating lymphocytes by immunohistochemistry (IHC) at 24 h post the 14th daily dose of 1, 3 or 30 mg/kg Compound I-57. Robust reduction in IKZF2 levels was detected at 1, 3 and 30 mg/kg doses with the maximal level of degradation (approximately 85%) observed at 30 mg/kg. FIG. 8B. shows representative images of IHC staining for IKZF2 from each treatment group.

FIG. 9A. is a graph showing the degradation of IKZF2 measured in FOXP3+ T cells upon repeated daily dosing in immunized cynomolgus monkeys treated daily with Compound I-57. Compound treatment was initiated at day 5.

FIG. 9B. is a graph showing proliferation of peripheral T cells (Mean+/−SEM, % of predose) upon treatment with 0.1 and 3 mg/kg of Compound I-57 in cynomolgus monkeys. As shown in FIG. 9B, the proportion of proliferative peripheral T cells (denoted by Ki67 staining) was increased in the highest dose group (3 mg/kg) in the recall response phase, compared to immunization alone. Levels of Ki67 remained elevated in this group until the end of the study, suggesting Compound I-57 treatment led to a sustained increase in immune responsiveness in these animals.

FIG. 10 is a pictorial representation of the study design for the FIH, open-label, phase I/Ib, multi-center study which consists of two dose escalation parts (Arms A and B), each followed by an expansion part.

### DETAILED DESCRIPTION OF THE DISCLOSURE

The present disclosure provides methods of treating and/or preventing a disease (e.g., cancer) comprising administering to a subject in need thereof a pharmaceutical formulation comprising a compound that has degrader activity for IKZF2, e.g., a 3-(1-oxoisoindolin-2-yl)piperidine-2,6-dione compound. In some aspects, the methods further comprise administering one or more agents, e.g., one or more anti-tumor agents; or one or more agents that are capable of modulating IKZF2 protein level. The disclosure further provides formulations, dosing, dosing regimens and schedules, biomarkers, pharmaceutical combinations, and other relevant clinical features.

According to the present disclosure, agents that can be used in combination with a compound that has degrader activity for IKZF2, e.g., a 3-(1-oxoisoindolin-2-yl)piperidine-2,6-dione compound can be, but are not limited to, an inhibitor of an inhibitory molecule (e.g., a checkpoint inhibitor), an activator of a costimulatory molecule, a chemotherapeutic agent, a targeted anti-cancer therapy, an oncolytic drug, a cytotoxic agent, or any of the therapeutic agents disclosed herein. In some embodiments, a compound that has degrader activity for IKZF2, e.g., a 3-(1-oxoisoindolin-2-yl)piperidine-2,6-dione compound is used in combination with one or more therapeutic agents chosen from: a PD-1 inhibitor, a LAG-3 inhibitor, a cytokine, an A2A antagonist, a GITR agonist, a TIM-3 inhibitor, a STING agonist, and a TLR7 agonist, for treating and/or preventing a patient with cancer.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

### A. Chemical Nomenclature, Terms, and Conventions

In the groups, radicals, or moieties defined below, the number of carbon atoms is often specified preceding the group, for example, (C₁-C₁₀)alkyl means an alkyl group or radical having 1 to 10 carbon atoms. In general, for groups comprising two or more subgroups, the last named group is the radical attachment point, for example, “alkylaryl” means a monovalent radical of the formula alkyl-aryl-, while “arylalkyl” means a monovalent radical of the formula aryl-alkyl-. Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” refer to one or more than one (e.g., to at least one) of the grammatical object of the article. By way of example, “an element” means one element or more than one element.

The term “and/or” means either “and” or “or” unless indicated otherwise.

The term “optionally substituted” means that a given chemical moiety (e.g., an alkyl group) can (but is not required to) be bonded other substituents (e.g., heteroatoms). For instance, an alkyl group that is optionally substituted can be a fully saturated alkyl chain (e.g., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, —OH, —CN, —COOH, —CH₂CN, —O—(C₁-C₆)alkyl, (C₁-C₆)alkyl, (C₁-C₆)alkoxy, (C₁-C₆)haloalkyl, (C₁-C₆)haloalkoxy, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O)(C₁-C₆)alkyl, —C(O)NH(C₁-C₆)alkyl, —S(O)₂(C₁-C₆)alkyl, —S(O)NH(C₁-C₆)alkyl, and S(O)N((C₁-C₆)alkyl)₂. The substituents can themselves be optionally substituted. “Optionally substituted” as used herein also refers to substituted or unsubstituted whose meaning is described below.

The term “substituted” means that the specified group or moiety bears one or more suitable substituents wherein the substituents may connect to the specified group or moiety at one or more positions.

For example, an aryl substituted with a cycloalkyl may indicate that the cycloalkyl connects to one atom of the aryl with a bond or by fusing with the aryl and sharing two or more common atoms.

The term “unsubstituted” means that the specified group bears no substituents.

Unless otherwise specifically defined, “aryl” means a cyclic, aromatic hydrocarbon group having 1 to 3 aromatic rings, including monocyclic or bicyclic groups such as phenyl, biphenyl, or naphthyl. When containing two aromatic rings (bicyclic, etc.), the aromatic rings of the aryl group are optionally joined at a single point (e.g., biphenyl), or fused (e.g., naphthyl). The aryl group is optionally substituted by one or more substituents, e.g., 1 to 5 substituents, at any point of attachment. Exemplary substituents include, but are not limited to, —H, -halogen, —CN, —O—(C₁-C₆)alkyl, (C₁-C₆)alkyl, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆) alkyl, NH₂, NH((C₁-C₆)alkyl), N((C₁-C₆)alkyl)₂, —S(O)₂—(C₁-C₆)alkyl, —S(O)NH(C₁-C₆)alkyl, and S(O)N((C₁-C₆)alkyl)₂. The substituents are themselves optionally substituted. Furthermore, when containing two fused rings, the aryl groups optionally have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic aromatic radical of 5 to 24 ring atoms or a polycyclic aromatic radical, containing one or more ring heteroatoms selected from N, O, or S, the remaining ring atoms being C. Heteroaryl as herein defined also means a bicyclic heteroaromatic group wherein the heteroatom is selected from N, O, or S. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1Δ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4 d]thiazolyl, imidazo[2,1-b][1,3,4]thiadiazolyl, thieno[2,3-b]pyrrolyl, 3H-indolyl, and derivatives thereof. Furthermore, when containing two fused rings the aryl groups herein defined may have an unsaturated or partially saturated ring fused with a fully saturated ring. Exemplary ring systems of these heteroaryl groups include indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuran, indolinyl, indolyl, and dihydrobenzoxanyl.

Halogen or “halo” mean fluorine, chlorine, bromine, or iodine.

“Alkyl” means a straight or branched chain saturated hydrocarbon containing 1-12 carbon atoms. Examples of a (C₁-C₆)alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

“Alkenyl” means a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted and may be straight or branched.

“Alkynyl” means a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

“Alkylene” or “alkylenyl” means a divalent alkyl radical. Any of the above mentioned monovalent alkyl groups may be an alkylene by abstraction of a second hydrogen atom from the alkyl. As herein defined, alkylene may also be a (C₁-C₆)alkylene. An alkylene may further be a (C₁-C₄)alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH—, and the like.

“Cycloalkyl” or “carbocyclyl” means a monocyclic or polycyclic saturated carbon ring containing 3-18 carbon atoms. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl and derivatives thereof. A (C₃-C₈)cycloalkyl is a cycloalkyl group containing between 3 and 8 carbon atoms. A cycloalkyl group can be fused (e.g., decalin) or bridged (e.g., norbomane).

“Heterocyclyl” or “heterocycloalkyl” means a saturated or partially saturated monocyclic or polycyclic ring containing carbon and at least one heteroatom selected from oxygen, nitrogen, or sulfur (O, N, or S) and wherein there is not delocalized n electrons (aromaticity) shared among the ring carbon or heteroatoms. The heterocycloalkyl ring structure may be substituted by one or more substituents. The substituents can themselves be optionally substituted. Examples of heterocyclyl rings include, but are not limited to, oxetanyl, azetadinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, imidazolinyl, dithiolanyl, and homotropanyl.

“Hydroxyalkyl” means an alkyl group substituted with one or more —OH groups. Examples of hydroxyalkyl groups include HO—CH₂—, HO—CH₂CH₂—, and CH₂—CH(OH)—.

“Haloalkyl” means an alkyl group substituted with one or more halogens. Examples of haloalkyl groups include, but are not limited to, trifluoromethyl, difluoromethyl, pentafluoroethyl, trichloromethyl, etc.

“Haloalkoxy” means an alkoxy group substituted with one or more halogens. Examples of haloalkyl groups include, but are not limited to, trifluoromethoxy, difluoromethoxy, pentafluoroethoxy, trichloromethoxy, etc.

“Cyano” means a substituent having a carbon atom joined to a nitrogen atom by a triple bond, e.g., C≡N.

“Amino” means a substituent containing at least one nitrogen atom (e.g., NH₂).

“Alkylamino” means an amino or NH₂ group where one of the hydrogens is replaced with an alkyl group, e.g., —NH(alkyl). Examples of alkylamino groups include, but are not limited to, methylamino (e.g., —NH(CH₃)), ethylamino, propylamino, iso-propylamino, n-butylamino, sec-butylamino, tert-butylamino, etc.

“Dialkylamino” means an amino or NH₂ group where both of the hydrogens are replaced with alkyl groups, e.g., —N(alkyl)₂. The alkyl groups on the amino group are the same or different alkyl groups. Examples of dialkylamino groups include, but are not limited to, dimethylamino (e.g., —N(CH₃)₂), diethylamino, dipropylamino, diiso-propylamino, di-n-butylamino, di-sec-butylamino, di-tert-butylamino, methyl(ethyl)amino, methyl(butylamino), etc.

“Spirocycloalkyl” or “spirocyclyl” means carbogenic bicyclic ring systems with both rings connected through a single atom. The rings can be different in size and nature, or identical in size and nature. Examples include spiropentane, spirohexane, spiroheptane, spirooctane, spirononane, or spirodecane. One or both of the rings in a spirocycle can be fused to another ring carbocyclic, heterocyclic, aromatic, or heteroaromatic ring. A (C₃-C₁₂)spirocycloalkyl is a spirocycle containing between 3 and 12 carbon atoms.

“Spiroheterocycloalkyl” or “spiroheterocyclyl” means a spirocycle wherein at least one of the rings is a heterocycle one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

### B. Salt, Prodrug, Derivative, and Solvate Terms and Conventions

“Prodrug” or “prodrug derivative” mean a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; Bioreversible Carriers in Drug Design, E. B. Roche (ed.), Elsevier, 1987, each of which is incorporated herein by reference in their entireties.

“Pharmaceutically acceptable prodrug” as used herein means a prodrug of a compound of the disclosure which is, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible.

“Salt” means an ionic form of the parent compound or the product of the reaction between the parent compound with a suitable acid or base to make the acid salt or base salt of the parent compound. Salts of the compounds of the present disclosure can be synthesized from the parent compounds which contain a basic or acidic moiety by conventional chemical methods. Generally, the salts are prepared by reacting the free base or acid parent compound with stoichiometric amounts or with an excess of the desired salt-forming inorganic or organic acid or base in a suitable solvent or various combinations of solvents.

“Pharmaceutically acceptable salt” means a salt of a compound of the disclosure which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. As the compounds of the present disclosure are useful in both free base and salt form, in practice, the use of the salt form amounts to use of the base form. Lists of suitable salts are found in, e.g., S. M. Birge et al., J. Pharm. Sci., 1977, 66, pp. 1-19, which is hereby incorporated by reference in its entirety.

“Pharmaceutically-acceptable acid addition salt” means those salts which retain the biological effectiveness and properties of the free bases and which are not biologically or otherwise undesirable, formed with inorganic acids such as hydrochloric acid, hydrobromic acid, hydroiodic acid, sulfuric acid, sulfamic acid, nitric acid, phosphoric acid, and the like, and organic acids such as acetic acid, trichloroacetic acid, trifluoroacetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, heptanoic acid, hexanoic acid, formic acid, fumaric acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, undecanoic acid, and the like.

“Pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

“Solvate” means a complex of variable stoichiometry formed by a solute, for example, a compound of Formula (I′) or Formula (I), or any compound disclosed herein) and solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, such solvents selected for the purpose of the disclosure do not interfere with the biological activity of the solute. Solvates encompasses both solution-phase and isolatable solvates. Representative solvates include hydrates, ethanolates, methanolates, and the like.

“Hydrate” means a solvate wherein the solvent molecule(s) is/are water.

The compounds of the present disclosure as discussed below include the free base or acid thereof, their salts, solvates, and prodrugs and may include oxidized sulfur atoms or quaternized nitrogen atoms in their structure, although not explicitly stated or shown, particularly the pharmaceutically acceptable forms thereof. Such forms, particularly the pharmaceutically acceptable forms, are intended to be embraced by the appended claims.

### C. Isomer Terms and Conventions

“Isomers” means compounds having the same number and kind of atoms, and hence the same molecular weight, but differing with respect to the arrangement or configuration of the atoms in space. The term includes stereoisomers and geometric isomers.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure, which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

“Enantiomers” means a pair of stereoisomers that are non-superimposable mirror images of each other.

“Diastereoisomers” or “diastereomers” mean optical isomers, which are not mirror images of each other.

“Racemic mixture” or “racemate” mean a mixture containing equal parts of individual enantiomers.

“Non-racemic mixture” means a mixture containing unequal parts of individual enantiomers.

“Geometrical isomer” means a stable isomer, which results from restricted freedom of rotation about double bonds (e.g., cis-2-butene and trans-2-butene) or in a cyclic structure (e.g., cis-1,3-dichlorocyclobutane and trans-1,3-dichlorocyclobutane). Because carbon-carbon double (olefinic) bonds, C═N double bonds, cyclic structures, and the like may be present in the compounds of the disclosure, the disclosure contemplates each of the various stable geometric isomers and mixtures thereof resulting from the arrangement of substituents around these double bonds and in these cyclic structures. The substituents and the isomers are designated using the cis/trans convention or using the E or Z system, wherein the term “E” means higher order substituents on opposite sides of the double bond, and the term “Z” means higher order substituents on the same side of the double bond. A thorough discussion of E and Z isomerism is provided in J. March, Advanced Organic Chemistry: Reactions, Mechanisms, and Structure, 4th ed., John Wiley & Sons, 1992, which is hereby incorporated by reference in its entirety. Several of the following examples represent single E isomers, single Z isomers, and mixtures of E/Z isomers. Determination of the E and Z isomers can be done by analytical methods such as x-ray crystallography, ¹H NMR, and ¹³C NMR.

Some of the compounds of the disclosure can exist in more than one tautomeric form. As mentioned above, the compounds of the disclosure include all such tautomers.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

Preparation of pure enantiomers or mixtures of desired enantiomeric excess (ee) or enantiomeric purity are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000. Furthermore, there are equally well-known methods for the quantitation of enantiomeric excess or purity, for example, GC, HPLC, CE, or NMR, and assignment of absolute configuration and conformation, for example, CD ORD, X-ray crystallography, or NMR.

In general, all tautomeric forms and isomeric forms and mixtures, whether individual geometric isomers or stereoisomers or racemic or non-racemic mixtures, of a chemical structure or compound is intended, unless the specific stereochemistry or isomeric form is specifically indicated in the compound name or structure.

### D. Pharmaceutical Administration and Treatment Terms and Conventions

A “patient” or “subject” is a mammal, e.g., a human, mouse, rat, guinea pig, dog, cat, horse, cow, pig, or nonhuman primate, such as a monkey, chimpanzee, baboon or, rhesus. In certain embodiments, the subject is a primate. In yet other embodiments, the subject is a human.

An “effective amount” or “therapeutically effective amount” when used in connection with a compound means an amount of a compound of the present disclosure in combination with the second therapeutic agent that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, the terms “pharmaceutical formulation” or “pharmaceutical composition” refers to a composition comprising one or more pharmaceutically active ingredients. In particular, a pharmaceutical formulation comprises (a) a compound of Formula (I′), or a pharmaceutically acceptable salt, hydrate, solvate, prodrug, stereoisomer, or tautomer thereof; and (b) a second therapeutic agent, preferably also including at least one pharmaceutically acceptable excipient or carrier, and more preferably where the pharmaceutically acceptable excipient or carrier does not react with the pharmaceutically active ingredients.

“Carrier” encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

A patient is “in need of” a treatment if such subject would benefit biologically, medically, or in quality of life from such treatment (preferably, a human).

As used herein, the term “inhibit”, “inhibition”, or “inhibiting” refers to the reduction or suppression of a given condition, symptom, or disorder, or disease, or a significant decrease in the baseline activity of a biological activity or process.

As used herein, the term “treat”, “treating”, or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing”, or “prevention” of any disease or disorder refers to the prophylactic treatment of the disease or disorder; or delaying the onset or progression of the disease or disorder.

“Pharmaceutically acceptable” means that the substance or composition must be compatible chemically and/or toxicologically, with the other ingredients comprising a formulation, and/or the mammal being treated therewith.

“Disorder” means, and is used interchangeably with, the terms disease, condition, or illness, unless otherwise indicated.

“Administer”, “administering”, or “administration” means to either directly administering a disclosed compound or pharmaceutically acceptable salt of the disclosed compound or a composition to a subject, or administering a prodrug derivative or analog of the compound or pharmaceutically acceptable salt of the compound, formulation, or combination comprising a compound or formulation to the subject, which can form an equivalent amount of active compound within the subject's body.

“Prodrug” means a compound which is convertible in vivo by metabolic means (e.g., by hydrolysis) to a disclosed compound.

“Compounds of the present disclosure”, “Compounds of Formula (I′)”, “compounds of the disclosure”, and equivalent expressions (unless specifically identified otherwise) refer to Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and the compounds of Formulae (I′), (I), (Ia), (Ib), (Ic), and (Id) as herein described including the tautomers, the prodrugs, salts particularly the pharmaceutically acceptable salts, and the solvates and hydrates thereof, where the context so permits thereof, as well as all stereoisomers (including diastereoisomers and enantiomers), rotamers, tautomers, and isotopically labelled compounds (including deuterium substitutions), as well as inherently formed moieties (e.g., polymorphs, solvates and/or hydrates). For purposes of this disclosure, solvates and hydrates are generally considered compositions. In general and preferably, the compounds of the disclosure and the formulas designating the compounds of the disclosure are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

“Stable compound” or “stable structure” means a compound that is sufficiently robust to survive isolation to a useful degree of purity from a reaction mixture, and formulation into an efficacious therapeutic or diagnostic agent. For example, a compound, which would have a “dangling valency” or is a carbanion is not a compound contemplated by the disclosure.

In a specific embodiment, the term “about” or “approximately” means within 20%, preferably within 10%, and more preferably within 5% of a given value or range.

The term “combination therapy” or “combination” or “in combination with” refers to the administration of two or more therapeutic agents to treat a condition or disorder described in the present disclosure (e.g., cancer). Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

A “therapeutic agent” as used herein refers to a therapy, e.g., a molecule, including but not limited to, a chemical compound, peptide, antibody, antibody fragment, antibody conjugate, or nucleic acid; a gene or cell therapy; or a radiation therapy, which is therapeutically active or enhances the therapeutic activity when administered to a patient in combination with a compound of the present disclosure or which reduces one or more side effects of the compound of the present disclosure when administered to a patient in combination with a compound of the present disclosure.

“Cancer” means any cancer caused by the uncontrolled proliferation of aberrant cells, such as tumors, neoplasms, carcinomas, sarcomas, leukemias, lymphomas, and the like. Cancer cells can spread locally or through the bloodstream and lymphatic system to other parts of the body. For example, cancers include, but are not limited to, mesothelioma, leukemias, and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, and nasopharyngeal), esophageal cancer, genitourinary cancers (e.g., prostate, bladder, renal, uterine, ovarian, testicular), lung cancer (e.g., small-cell and non-small cell), breast cancer, pancreatic cancer, melanoma, and other skin cancers, stomach cancer, brain tumors, tumors related to Gorlin's syndrome (e.g., medulloblastoma, meningioma, etc.), liver cancer, non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST). Additional exemplary forms of cancer which may be treated by the compounds and compositions described herein include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

The second agent can be an anti-cancer agent. The term “anti-cancer” or “anti-cancer agent” pertains to an agent which treats a cancer (i.e., a compound, antibody, etc. which is useful in the treatment of a cancer). The anti-cancer effect may arise through one or more mechanisms, including, but not limited to, the regulation of cell growth or proliferation, the inhibition of angiogenesis (the formation of new blood vessels), the inhibition of metastasis (the spread of a tumor from its origin), the inhibition of invasion (the spread of tumor cells into neighboring normal structures), the inhibition of a checkpoint molecule, or the promotion of apoptosis.

The anti-cancer agent is can be an anti-proliferative agent or an immunomodulatory agent. In one embodiment, the second agent is an immunomodulatory agent.

The term “antiproliferative” or “antiproliferative agent” as used herein pertains to an agent, which inhibits cell growth or cell proliferation. The anti-proliferative agent can be a cytotoxic agent (e.g., alkylating agent, antimetabolites, etc.), a targeted agent (e.g., EGF inhibitor, Tyrosine protein kinase inhibitor, angiogenesis inhibitor, etc.), or a hormonal agent (e.g., estrogens selective estrogen receptor modulators, etc.). Examples of antiproliferative agents include alkylating agents, anti-metabolites, an antibiotic, a detoxifying agent, an EGFR inhibitor, a HER2 inhibitor, a histone deacetylase inhibitor, a hormone, a mitotic inhibitor, an MTOR inhibitor, a multi-kinase inhibitor, a serine/threonine inhibitor, a tyrosine kinase inhibitor, a VEGF/VEGFR inhibitor; a taxane or taxane derivative, an aromatase inhibitor, an anthracycline, a microtubule targeting drug, a topoisomerase poison drug, an inhibitor of a molecular target or enzyme.

The term “immunomodulatory agent” is agent that modifies the immune response or the functioning of the immune system (as by the stimulation of antibody formation or the inhibition of white blood cell activity). The immunomodulatory agents can be an immunomodulator, a cytokine, a vaccine, or an anti-body.

The term “immunomodulator” is an inhibitor of an immune checkpoint molecule.

Additional cancers that the compounds and compositions described herein may be useful in preventing, treating, and studying are, for example, colon carcinoma, familiary adenomatous polyposis carcinoma, and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing's sarcoma, and plasmocytoma.

“Simultaneously” or “simultaneous” when referring to a method of treating or a therapeutic use means with a combination of a compound of Formula (I′) and one or more second agent(s) means administration of the compound and the one or more second agent(s) by the same route and at the same time.

“Separately” or “separate” when referring to a method of treating or a therapeutic use means with a combination of a compound of Formula (I′) and one or more second agent(s) means administration of the compound and the one or more second agent(s) by different routes and at approximately the same time.

By therapeutic administration “over a period of time” means, when referring to a method of treating or a therapeutic use with a combination of a compound of Formula (I′) and one or more second agent(s), administration of the compound and the one or more second agent(s) by the same or different routes and at different times. In some embodiments, the administration of the compound or the one or more second agent(s) occurs before the administration of the other begins. In this way, it is possible to administer a one of the active ingredients (i.e., a compound of the Formula (I′) or one or more second agent(s)) for several months before administering the other active ingredient or ingredients. In this case, no simultaneous administration occurs. Another therapeutic administration over a period of time consists of the administration over time of the two or more active ingredients of the combination using different frequencies of administration for each of the active ingredients, whereby at certain time points in time simultaneous administration of all of the active ingredients takes place whereas at other time points in time only a part of the active ingredients of the combination may be administered (e.g., for example. a compound of formula (I′) and the one or more second agents the therapeutic administration over a period of time could be such that a compound of Formula (I′) is administered once a day and the one or more second agent(s) is administered once every four weeks.)

“IKZF2-dependent disease or disorder” means any disease or disorder, which is directly or indirectly affected by the modulation of IKZF2 protein levels.

“IKZF4-dependent disease or disorder” means any disease or disorder, which is directly or indirectly affected by the modulation of IKZF4 protein levels.

### Specific Embodiments of the Compounds and Combinations

Embodiment 1a: A combination comprising, (a) a compound according to any one of Embodiments 1-70, or a pharmaceutically acceptable salt, hydrate, solvate, prodrug, stereoisomer, or tautomer thereof, and a second therapeutic agent.

Embodiment 1b: A pharmaceutical formulation comprising, (a) a compound according to any one of Embodiments 1-70, or a pharmaceutically acceptable salt, hydrate, solvate, prodrug, stereoisomer, or tautomer thereof, and a second therapeutic agent.

Embodiment 1: A compound of Formula (I′):

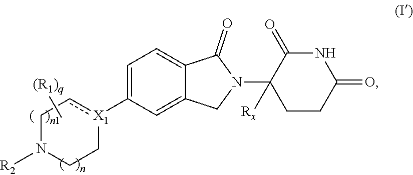

wherein:
X₁ is CR₃;
[symbol] is optionally a double bond when X₁ is CR₃ and R₃ is absent;
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
R₃ is H or R₃ is absent when [symbol] is a double bond;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R_{6′} are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S;
Rₓ is H or D;
p is 0, 1, or 2;
n is 0, 1, or 2;
n1 is 1 or 2, wherein n+n1≤3; and
q is 0, 1, 2, 3, or 4;
or pharmaceutically acceptable salts, hydrates, solvates, prodrugs, stereoisomers, and tautomers thereof.

Embodiment 2: The compound according to Embodiment 1, wherein the compound of Formula (I′) has a Formula (I), Formula (Ia), Formula (Tb), Formula (Ic), or Formula (Id):

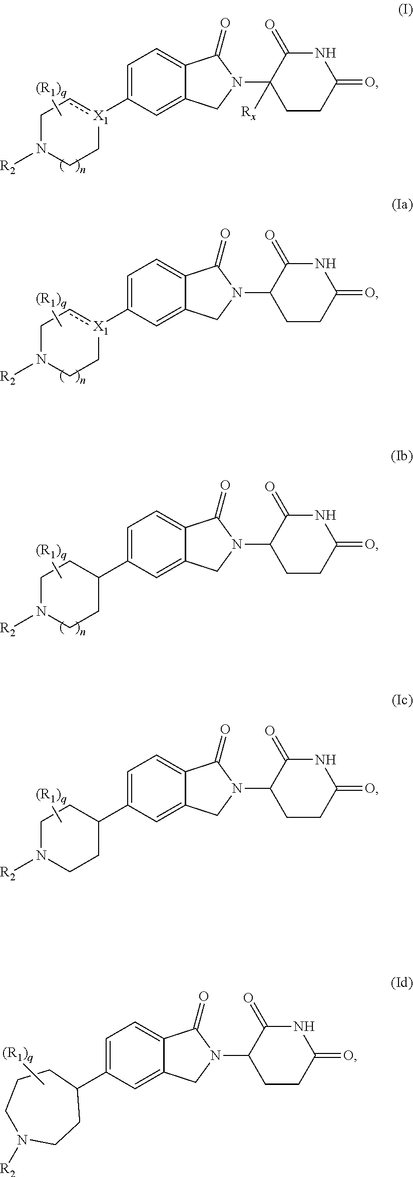

or pharmaceutically acceptable salts, hydrates, solvates, prodrugs, stereoisomers, and tautomers thereof.

Embodiment 3: The compound according to Embodiment 1 or 2, wherein X₁ is CH and n is 1.

Embodiment 4: The compound according to any one of Embodiments 1-3, wherein X₁ is CH, n is 1, and q is 0.

Embodiment 5: The compound according to any one of Embodiments 1-3, wherein X₁ is CH, n is 1, and q is 0 or 1.

Embodiment 6: The compound according to any one of Embodiments 1-3 or 5, wherein X₁ is CH, n is 1, q is 0 or 1, and R₁ is (C₁-C₆)alkyl.

Embodiment 7: The compound according to any one of Embodiments 1-3 or 5, wherein X₁ is CH, n is 1, q is 0 or 1, R₁ is (C₁-C₆)alkyl, and R₂ is (C₁-C₆)alkyl optionally substituted with one to three R₄.

Embodiment 8: The compound according to any one of Embodiments 1-3 or 5, wherein X₁ is CH, n is 1, q is 0 or 1, R₁ is (C₁-C₆)alkyl, and R₂ is (C₁-C₆)alkyl substituted with one to three R₄.

Embodiment 9: The compound according to any one of Embodiments 1-4, wherein X₁ is CH, n is 1, q is 0, and R₂ is (C₁-C₆)alkyl optionally substituted with one to three R₄.

Embodiment 10: The compound according to any one of Embodiments 1-4, wherein X₁ is CH, n is 1, q is 0, and R₂ is (C₁-C₆)alkyl substituted with one to three R₄.

Embodiment 11: The compound according to any one of Embodiments 1-3 or 5, wherein X₁ is CH, n is 1, q is 0 or 1, R₁ is (C₁-C₆)alkyl, R₂ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 12: The compound according to any one of Embodiments 1-3 or 5, wherein X₁ is CH, n is 1, q is 0 or 1, R₁ is (C₁-C₆)alkyl, R₂ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 13: The compound according to any one of Embodiments 1-3 or 5, wherein X₁ is CH, n is 1, q is 0 or 1, R₁ is (C₁-C₆)alkyl, R₂ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 14: The compound according to any one of Embodiments 1-3 or 5, wherein X₁ is CH, n is 1, q is 0 or 1, R₁ is (C₁-C₆)alkyl, R₂ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 15: The compound according to any one of Embodiments 1-5, wherein X₁ is CH, n is 1, q is 0, and R₂ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅. In yet another embodiment, X₁ is CH, n is 1, q is 0, and R₂ is (C₆-C₁₀)aryl, (C₃-C₈)cycloalkyl, or 5- to 7-membered heterocycloalkyl comprising 1 to 3 heteroatoms selected from O, N, and S.

Embodiment 16: The compound according to any one of Embodiments 1-5, wherein X₁ is CH, n is 1, q is 0, and R₂ is (C₆-C₁₀)aryl optionally substituted with one to three R₅.

Embodiment 17: The compound according to any one of Embodiments 1-5, wherein X₁ is CH, n is 1, q is 0, and R₂ is 5- or 6-membered heteroaryl comprising 1 to 3 heteroatoms selected from O, N, and S optionally substituted with one to three R₅.

Embodiment 18: The compound according to any one of Embodiments 1-5, wherein X₁ is CH, n is 1, q is 0, and R₂ is (C₃-C₅)cycloalkyl optionally substituted with one to three R₅. In another embodiment, X₁ is CH, n is 1, q is 0, and R₂ is 5- to 7-membered heterocycloalkyl comprising 1 to 3 heteroatoms selected from O, N, and S, optionally substituted with one to three R₅.

Embodiment 19: The compound according to any one of Embodiments 1-3 or 5, wherein X₁ is CH, n is 1, q is 0 or 1, R₁ is (C₁-C₆)alkyl, and R₂ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

Embodiment 20: The compound according to any one of Embodiments 1-3 or 5, wherein X₁ is CH, n is 1, q is 0 or 1, R₁ is (C₁-C₆)alkyl, and R₂ is (C₆-C₁₀)aryl, (C₃-C₈)cycloalkyl, or 5- to 7-membered heterocycloalkyl comprising 1 to 3 heteroatoms selected from O, N, and S.

Embodiment 21: The compound according to any one of Embodiments 1-3 or 5, wherein X₁ is CH, n is 1, q is 0 or 1, R₁ is (C₁-C₆)alkyl, and R₂ is (C₆-C₁₀)aryl optionally substituted with one to three R₅.

Embodiment 22: The compound according to any one of Embodiments 1-5, wherein X₁ is CH, n is 1, q is 0, and R₂ is 5- or 6-membered heteroaryl comprising 1 to 3 heteroatoms selected from O, N, and S optionally substituted with one to three R₅.

Embodiment 23: The compound according to any one of Embodiments 1-3 or 5, wherein X₁ is CH, n is 1, q is 0 or 1, R₁ is (C₁-C₆)alkyl, and R₂ is (C₃-C₈)cycloalkyl optionally substituted with one to three R₅. In another embodiment, X₁ is CH, n is 1, q is 0 or 1, R₁ is (C₁-C₆)alkyl, and R₂ is 5- to 7-membered heterocycloalkyl comprising 1 to 3 heteroatoms selected from O, N, and S, optionally substituted with one to three R₅.

Embodiment 24: The compound according to any one of Embodiments 1-5, wherein X₁ is CH, n is 1, q is 0, and R₂ is (C₁-C₆)alkyl optionally substituted with one to three R₄.

Embodiment 25: The compound according to any one of Embodiments 1-5, wherein X₁ is CH, n is 1, q is 0, and R₂ is (C₁-C₆)alkyl substituted with one to three R₄.

Embodiment 26: The compound according to any one of Embodiments 1-5, wherein X₁ is CH, n is 1, q is 0, R₂ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 27: The compound according to any one of Embodiments 1-5, wherein X₁ is CH, n is 1, q is 0, R₂ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 28: The compound according to any one of Embodiments 1-5, wherein X₁ is CH, n is 1, q is 0, R₂ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 29: The compound according to any one of Embodiments 1-5, wherein X₁ is CH, n is 1, q is 0, R₂ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 30: The compound according to any one of Embodiments 1-3, wherein X₁ is CH, n is 1, n1 is 1, q is 0, R₂ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 31: The compound according to any one of Embodiments 1-3, wherein X₁ is CH, n is 1, n1 is 1, q is 0, R₂ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 32: The compound according to any one of Embodiments 1-5, wherein X₁ is CH, n is 1, q is 0, R₂ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 33: The compound according to any one of Embodiments 1-5, wherein X₁ is CH, n is 1, q is 0, R₂ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 34: The compound according to any one of Embodiments 1-5, wherein X₁ is CH, n is 1, q is 0, R₂ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 35: The compound according to any one of Embodiments 1-5, wherein X₁ is CH, n is 1, q is 0, R₂ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 36: The compound according to any one of Embodiments 1-3, wherein X₁ is CH, n is 1, n1 is 1, q is 0, R₂ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 37: The compound according to any one of Embodiments 1-3, wherein X₁ is CH, n is 1, n1 is 1, q is 0, R₂ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 38: The compound according to any one of Embodiments 1-5, wherein X₁ is CH, n is 1, q is 0, R₂ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 39: The compound according to any one of Embodiments 1-5, wherein X₁ is CH, n is 1, q is 0, R₂ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 40: The compound according to any one of Embodiments 1-3, wherein X₁ is CH, n is 1, n1 is 1, q is 0, R₂ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 41: The compound according to any one of Embodiments 1-3, wherein X₁ is CH, n is 1, n1 is 1, q is 0, R₂ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 42: The compound according to any one of Embodiments 1-5, wherein X₁ is CH, n is 1, q is 0, R₂ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the aryl and heteroaryl groups are optionally substituted with one to three R₇.

Embodiment 43: The compound according to any one of Embodiments 1-5, wherein X₁ is CH, n is 1, q is 0, R₂ is (C₁-C₆)alkyl substituted with one to three R₄, and each R₄ is independently selected from phenyl and 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl and heteroaryl groups are optionally substituted with one to three R₇.

Embodiment 44: The compound according to any one of Embodiments 1-3, wherein X₁ is CH, n is 1, n1 is 1, q is 0, R₂ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the aryl and heteroaryl groups are optionally substituted with one to three R₇.

Embodiment 45: The compound according to any one of Embodiments 1-3, wherein X₁ is CH, n is 1, n1 is 1, q is 0, R₂ is (C₁-C₆)alkyl substituted with one to three R₄, and each R₄ is independently selected from phenyl and 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl and heteroaryl groups are optionally substituted with one to three R₇.

Embodiment 46: The compound according to any one of Embodiments 1-5, wherein X₁ is CH, n is 1, q is 0, R₂ is (C₁-C₆)alkyl optionally substituted with one to three R₄, and each R₄ is phenyl optionally substituted with one to three R₇.

Embodiment 47: The compound according to any one of Embodiments 1-5, wherein X₁ is CH, n is 1, q is 0, R₂ is (C₁-C₆)alkyl substituted with one to three R₄, and each R₄ is phenyl optionally substituted with one to three R₇.

Embodiment 48: The compound according to any one of Embodiments 1-3, wherein X₁ is CH, n is 1, n1 is 1, q is 0, R₂ is (C₁-C₆)alkyl optionally substituted with one to three R₄, and each R₄ is phenyl optionally substituted with one to three R₇.

Embodiment 49: The compound according to any one of Embodiments 1-3, wherein X₁ is CH, n is 1, n1 is 1, q is 0, R₂ is (C₁-C₆)alkyl substituted with one to three R₄, and each R₄ is phenyl optionally substituted with one to three R₇.

Embodiment 50: The compound according to Embodiment 1 or 2 wherein X₁ is CH and n is 2.

Embodiment 51: The compound according to Embodiment 50, wherein X₁ is CH, n is 2, and q is 0.

Embodiment 52: The compound according to Embodiment 50, wherein X₁ is CH, n is 2, and q is 0 or 1.

Embodiment 53: The compound according to Embodiment 50 or 52, wherein X₁ is CH, n is 2, q is 0 or 1, and R₁ is (C₁-C₆)alkyl.

Embodiment 54: The compound according to Embodiment 50 or 52, wherein X₁ is CH, n is 2, q is 0 or 1, R₁ is (C₁-C₆)alkyl, and R₂ is (C₁-C₆)alkyl optionally substituted with one to three R₄. In another embodiment, X₁ is CH, n is 2, q is 0 or 1, R₁ is (C₁-C₆)alkyl, and R₂ is (C₁-C₆)alkyl substituted with one to three R₄.

Embodiment 55: The compound according to any one of Embodiments 50-52, wherein X₁ is CH, n is 2, q is 0, and R₂ is (C₁-C₆)alkyl optionally substituted with one to three R₄. In another embodiment, X₁ is CH, n is 2, q is 0, and R₂ is (C₁-C₆)alkyl substituted with one to three R₄.

Embodiment 56: The compound according to Embodiment 50 or 52, wherein X₁ is CH, n is 2, q is 0 or 1, R₁ is (C₁-C₆)alkyl, R₂ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 57: The compound according to Embodiment 50 or 52, wherein X₁ is CH, n is 2, q is 0 or 1, R₁ is (C₁-C₆)alkyl, R₂ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 58: The compound according to Embodiment 50 or 52, wherein X₁ is CH, n is 2, q is 0 or 1, R₁ is (C₁-C₆)alkyl, R₂ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 59: The compound according to Embodiment 50 or 52, wherein X₁ is CH, n is 2, q is 0 or 1, R₁ is (C₁-C₆)alkyl, R₂ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

Embodiment 60: The compound according to any one of Embodiments 50-52, wherein X₁ is CH, n is 2, q is 0, and R₂ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

Embodiment 61: The compound according to any one of Embodiments 50-52, wherein X₁ is CH, n is 2, q is 0, and R₂ is (C₆-C₁₀)aryl, (C₃-C₈)cycloalkyl, or 5- to 7-membered heterocycloalkyl comprising 1 to 3 heteroatoms selected from O, N, and S.

Embodiment 62: The compound according to any one of Embodiment 50-52, wherein X₁ is CH, n is 2, q is 0, and R₂ is (C₆-C₁₀)aryl optionally substituted with one to three R₅. In another embodiment, X₁ is CH, n is 2, q is 0, and R₂ is 5- or 6-membered heteroaryl comprising 1 to 3 heteroatoms selected from O, N, and S optionally substituted with one to three R₅.

Embodiment 63: The compound according to any one of Embodiment 50-52, wherein X₁ is CH, n is 2, q is 0, and R₂ is (C₃-C₈)cycloalkyl optionally substituted with one to three R₅. In another embodiment, X₁ is CH, n is 2, q is 0, and R₂ is 5- to 7-membered heterocycloalkyl comprising 1 to 3 heteroatoms selected from O, N, and S, optionally substituted with one to three R₅.

Embodiment 64: The compound according to Embodiment 50 or 52, wherein X₁ is CH, n is 2, q is 0 or 1, R₁ is (C₁-C₆)alkyl, and R₂ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

Embodiment 65: The compound according to Embodiment 50 or 52, wherein X₁ is CH, n is 2, q is 0 or 1, R₁ is (C₁-C₆)alkyl, and R₂ is (C₆-C₁₀)aryl, (C₃-C₈)cycloalkyl, or 5- to 7-membered heterocycloalkyl comprising 1 to 3 heteroatoms selected from O, N, and S.

Embodiment 66: The compound according to Embodiment 50 or 52, wherein X₁ is CH, n is 2, q is 0 or 1, R₁ is (C₁-C₆)alkyl, and R₂ is (C₆-C₁₀)aryl optionally substituted with one to three R₅. In another embodiment, X₁ is CH, n is 2, q is 0, and R₂ is 5- or 6-membered heteroaryl comprising 1 to 3 heteroatoms selected from O, N, and S optionally substituted with one to three R₅.

Embodiment 67: The compound according to Embodiment 50 or 52, wherein X₁ is CH, n is 2, q is 0 or 1, R₁ is (C₁-C₆)alkyl, and R₂ is (C₃-C₈)cycloalkyl optionally substituted with one to three R₅.

Embodiment 68: The compound according to Embodiment 50 or 52, wherein X₁ is CH, n is 2, q is 0 or 1, R₁ is (C₁-C₆)alkyl, and R₂ is 5- to 7-membered heterocycloalkyl comprising 1 to 3 heteroatoms selected from O, N, and S, optionally substituted with one to three R₅.

Embodiment 69: The compound according to Embodiment 1, wherein the compound of Formula (I′) is selected from Compound I-156, Compound I-57, Compound I-87, Compound I-88, Compound I-265, and Compound I-112.

Embodiment 70: The compound according to Embodiment 1, wherein the compound of Formula (I′) is selected from:

| Cmpd | |
|---|---|
| No. | Compound Name |
| I-1 | 3-(5-(1-ethylpiperidin-4-yl)-1- |
| | oxoisoindolin-2-yl)piperidine-2,6-dione; |
| I-2 | 3-(1-oxo-5-(1-propylpiperidin-4- |
| | yl)isoindolin-2-yl)piperidine-2,6-dione; |
| I-3 | 3-(5-(1-(cyclopropylmethyl)piperidin-4-yl)- |
| | 1-oxoisoindolin-2-yl)piperidine-2,6-dione; |
| I-4 | 3-(5-(1-isobutylpiperidin-4-yl)-1- |
| | oxoisoindolin-2-yl)piperidine-2,6-dione; |
| I-5 | 3-(5-(1-(cyclobutylmethyl)piperidin-4-yl)- |
| | 1-oxoisoindolin-2-yl)piperidine-2,6-dione; |
| I-6 | 3-(5-(1-(oxazol-2-ylmethyl)piperidin-4-yl)- |
| | 1-oxoisoindolin-2-yl)piperidine-2,6-dione; |
| I-7 | 3-(1-oxo-5-(1-(thiazol-2-ylmethyl)piperidin- |
| | 4-yl)isoindolin-2-yl)piperidine-2,6-dione; |
| I-8 | 3-(5-(1-(cyclopentylmethyl)piperidin-4-yl)- |
| | 1-oxoisoindolin-2-yl)piperidine-2,6-dione; |
| I-9 | 3-(5-(1-((5-chlorothiophen-2-yl)methyl)piperidin- |
| | 4-yl)-1-oxoisoindolin-2-yl)piperidine-2,6-dione; |
| I-10 | 3-(5-(1-((2-chlorothiazol-5-yl)methyl)piperidin- |
| | 4-yl)-1-oxoisoindolin-2-yl)piperidine-2,6-dione; |
| I-11 | 3-(5-(1-(cyclohexylmethyl)piperidin-4-yl)- |
| | 1-oxoisoindolin-2-yl)piperidine-2,6-dione; |
| I-12 | 3-(1-oxo-5-(1-(2-(pyrrolidin-1-yl)ethyl)piperidin- |
| | 4-yl)isoindolin-2-yl)piperidine-2,6-dione; |
| I-13 | 3-(1-oxo-5-(1-((tetrahydro-2H-pyran-4-yl)methyl)piperidin- |
| | 4-yl)isoindolin-2-yl)piperidine-2,6-dione; |
| I-14 | 3-(1-oxo-5-(1-phenethylpiperidin-4- |
| | yl)isoindolin-2-yl)piperidine-2,6-dione; |
| I-15 | 3-(5-(1-(3-fluorobenzyl)piperidin-4-yl)- |
| | 1-oxoisoindolin-2-yl)piperidine-2,6-dione; |
| I-16 | 3-(5-(1-(3-chlorobenzyl)piperidin-4-yl)- |
| | 1-oxoisoindolin-2-yl)piperidine-2,6-dione; |
| I-17 | 3-(5-(1-(2-fluorobenzyl)piperidin-4-yl)- |
| | 1-oxoisoindolin-2-yl)piperidine-2,6-dione; |
| I-18 | 3-(5-(1-(2-chlorobenzyl)piperidin-4-yl)- |
| | 1-oxoisoindolin-2-yl)piperidine-2,6-dione; |
| I-19 | 3-(1-oxo-5-(1-(2-(piperidin-1-yl)ethyl)piperidin- |
| | 4-yl)isoindolin-2-yl)piperidine-2,6-dione; |
| I-20 | 3-(5-(1-((3,5-dimethylisoxazol-4-yl)methyl)piperidin- |
| | 4-yl)-1-oxoisoindolin-2-yl)piperidine-2,6-dione; |
| I-21 | 3-(5-(1-((1,3-dimethyl-1H-pyrazol-5-yl)methyl)piperidin- |
| | 4-yl)-1-oxoisoindolin-2-yl)piperidine-2,6-dione; |
| I-22 | 3-(5-(1-((6-methylpyridin-2-yl)methyl)piperidin- |
| | 4-yl)-1-oxoisoindolin-2-yl)piperidine-2,6-dione; |
| I-23 | 3-(5-(1-(3-morpholinopropyl)piperidin-4-yl)- |
| | 1-oxoisoindolin-2-yl)piperidine-2,6-dione; |
| I-24 | 3-(5-(1-(2,6-difluorobenzyl)piperidin-4-yl)- |
| | 1-oxoisoindolin-2-yl)piperidine-2,6-dione; |
| I-25 | 3-(5-(1-(2,6-dichlorobenzyl)piperidin-4-yl)- |
| | 1-oxoisoindolin-2-yl)piperidine-2,6-dione; |
| I-26 | 3-(5-(1-(3,5-difluorobenzyl)piperidin-4-yl)- |
| | 1-oxoisoindolin-2-yl)piperidine-2,6-dione; |
| I-27 | 3-(5-(1-(3,5-dibromobenzyl)piperidin-4-yl)- |
| | 1-oxoisoindolin-2-yl)piperidine-2,6-dione; |
| I-28 | 3-(5-(1-(3-chloro-5-fluorobenzyl)piperidin-4-yl)- |
| | 1-oxoisoindolin-2-yl)piperidine-2,6-dione; |
| I-29 | 3-(5-(1-(2,5-difluorobenzyl)piperidin-4-yl)- |
| | 1-oxoisoindolin-2-yl)piperidine-2,6-dione; |
| I-30 | 3-(5-(1-(2,5-dichlorobenzyl)piperidin-4-yl)- |
| | 1-oxoisoindolin-2-yl)piperidine-2,6-dione; |
| I-31 | 4-((4-(2-(2,6-dioxopiperidin-3-yl)-1-oxoisoindolin- |
| | 5-yl)piperidin-1-yl)methyl)benzonitrile |
| | (or 3-(5-(1-(4-nitrilebenzyl)piperidin-4-yl)-1- |
| | oxoisoindolin-2-yl)piperidine-2,6-dione); |
| I-32 | 3-(5-(1-(4-(hydroxymethyl)benzyl)piperidin-4- |
| | yl)-1-oxoisoindolin-2-yl)piperidine-2,6-dione; |
| I-33 | 3-(5-(1-(3,4-dichlorobenzyl)piperidin-4-yl)- |
| | 1-oxoisoindolin-2-yl)piperidine-2,6-dione; |
| I-34 | 3-(5-(1-(4-chloro-2-fluorobenzyl)piperidin-4- |
| | yl)-1-oxoisoindolin-2-yl)piperidine-2,6-dione; |
| I-35 | 3-(5-(1-(2-chloro-4-fluorobenzyl)piperidin-4- |
| | yl)-1-oxoisoindolin-2-yl)piperidine-2,6-dione; |
| I-36 | 3-((4-(2-(2,6-dioxopiperidin-3-yl)-1- |
| | oxoisoindolin-5-yl)piperidin-1-yl)methyl)benzonitrile |
| I-37 | 3-(5-(1-(2,3-difluorobenzyl)piperidin-4-yl)- |
| | 1-oxoisoindolin-2-yl)piperidine-2,6-dione; |
| I-38 | 2-((4-(2-(2,6-dioxopiperidin-3-yl)-1- |
| | oxoisoindolin-5-yl)piperidin-1-yl)methyl)benzonitrile; |
| I-39 | 3-(5-(1-(4-methoxybenzyl)piperidin-4-yl)- |
| | 1-oxoisoindolin-2-yl)piperidine-2,6-dione; |
| I-40 | 3-(5-(1-(2,5-dimethylbenzyl)piperidin-4-yl)- |
| | 1-oxoisoindolin-2-yl)piperidine-2,6-dione; |
| I-41 | 3-(5-(1-(3,4-dimethylbenzyl)piperidin-4-yl)- |
| | 1-oxoisoindolin-2-yl)piperidine-2,6-dione; |
| I-42 | 3-(5-(1-(2,4-dimethylbenzyl)piperidin-4-yl)- |
| | 1-oxoisoindolin-2-yl)piperidine-2,6-dione; |
| I-43 | 3-(5-(1-((1H-indazol-4-yl)methyl)piperidin-4- |
| | yl)-1-oxoisoindolin-2-yl)piperidine-2,6-dione; |
| I-44 | 3-(5-(1-((1H-benzo[d]imidazol-2-yl)methyl)piperidin- |
| | 4-yl)-1-oxoisoindolin-2-yl)piperidine-2,6-dione; |
| I-45 | 3-(5-(1-(4-isopropylbenzyl)piperidin-4-yl)- |
| | 1-oxoisoindolin-2-yl)piperidine-2,6-dione; |
| I-46 | methyl 5-((4-(2-(2,6-dioxopiperidin-3-yl)-1-oxoisoindolin- |
| | 5-yl)piperidin-1-yl)methyl)furan-2-carboxylate; |
| I-47 | 3-(5-(1-(naphthalen-2-ylmethyl)piperidin-4- |
| | yl)-1-oxoisoindolin-2-yl)piperidine-2,6-dione; |
| I-48 | 3-(1-oxo-5-(1-(quinolin-2-ylmethyl)piperidin- |
| | 4-yl)isoindolin-2-yl)piperidine-2,6-dione; |
| I-49 | 3-(5-(1-(naphthalen-1-ylmethyl)piperidin-4- |
| | yl)-1-oxoisoindolin-2-yl)piperidine-2,6-dione; |
| I-50 | 3-(5-(1-((1-methyl-1H-benzo[d]imidazol-2-yl)methyl)piperidin- |
| | 4-yl)-1-oxoisoindolin-2-yl)piperidine-2,6-dione; |
| I-51 | 3-(1-oxo-5-(1-(4-(trifluoromethoxy)benzyl)piperidin- |
| | 4-yl)isoindolin-2-yl)piperidine-2,6-dione; |
| I-52 | 3-(5-(1-(4-(1H-pyrrol-1-yl)benzyl)piperidin-4- |
| | yl)-1-oxoisoindolin-2-yl)piperidine-2,6-dione; |
| I-53 | 3-(5-(1-(4-(1H-1,2,4-triazol-1-yl)benzyl)piperidin- |
| | 4-yl)-1-oxoisoindolin-2-yl)piperidine-2,6-dione; |
| I-54 | 3-(1-oxo-5-(1-(3-(trifluoromethoxy)benzyl)piperidin- |
| | 4-yl)isoindolin-2-yl)piperidine-2,6-dione; |
| I-55 | 3-(1-oxo-5-(1-(2-(trifluoromethoxy)benzyl)piperidin- |
| | 4-yl)isoindolin-2-yl)piperidine-2,6-dione; |
| I-56 | 3-(1-oxo-5-(1-((3-phenyl-1,2,4-oxadiazol-5- |
| | yl)methyl)piperidin-4-yl)isoindolin-2-yl)piperidine-2,6-dione; |
| I-57 | 3-(5-(1-benzylpiperidin-4-yl)-1- |
| | oxoisoindolin-2-yl)piperidine-2,6-dione; |
| I-58 | 3-(1-oxo-5-(1-(pyridin-2-ylmethyl)piperidin- |
| | 4-yl)isoindolin-2-yl)piperidine-2,6-dione; |
| I-59 | 3-(1-oxo-5-(1-(pyridin-3-ylmethyl)piperidin- |
| | 4-yl)isoindolin-2-yl)piperidine-2,6-dione; |
| I-60 | 3-(1-oxo-5-(1-(pyridin-4-ylmethyl)piperidin- |
| | 4-yl)isoindolin-2-yl)piperidine-2,6-dione; |
| I-61 | 3-(1-oxo-5-(1-(pyrimidin-5-ylmethyl)piperidin- |
| | 4-yl)isoindolin-2-yl)piperidine-2,6-dione; |
| I-62 | 3-(1-oxo-5-(1-(1-phenylethyl)piperidin-4- |
| | yl)isoindolin-2-yl)piperidine-2,6-dione; |
| I-63 | 3-(5-(1-(4-(fluoromethyl)benzyl)piperidin-4- |
| | yl)-1-oxoisoindolin-2-yl)piperidine-2,6-dione; |
| I-64 | 3-(5-(1-(3,4-difluorobenzyl)piperidin-4-yl)- |
| | 1-oxoisoindolin-2-yl)piperidine-2,6-dione; |
| I-65 | 2-((4-(2-(2,6-dioxopiperidin-3-yl)-1-oxoisoindolin-5- |
| | yl)piperidin-1-yl)methyl)pyrimidine-5-carbonitrile |
| I-66 | 3-(5-(1-(4-ethylbenzyl)piperidin-4-yl)-1- |
| | oxoisoindolin-2-yl)piperidine-2,6-dione; |
| I-67 | 3-(5-(1-(2-methoxybenzyl)piperidin-4-yl)- |
| | 1-oxoisoindolin-2-yl)piperidine-2,6-dione; |
| I-68 | 3-(5-(1-((2-methoxypyrimidin-5-yl)methyl)piperidin- |
| | 4-yl)-1-oxoisoindolin-2-yl)piperidine-2,6-dione; |
| I-69 | 3-(5-(1-(3-fluoro-4-methylbenzyl)piperidin-4- |
| | yl)-1-oxoisoindolin-2-yl)piperidine-2,6-dione; |
| I-70 | 3-(5-(1-(4-(difluoromethyl)benzyl)piperidin-4- |
| | yl)-1-oxoisoindolin-2-yl)piperidine-2,6-dione; |
| I-71 | 4-((4-(2-(2,6-dioxopiperidin-3-yl)-1- |
| | oxoisoindolin-5-yl)piperidin-1-yl)methyl)benzamide; |
| I-72 | 4-((4-(2-(2,6-dioxopiperidin-3-yl)-1- |
| | oxoisoindolin-5-yl)piperidin-1-yl)methyl)benzoic acid; |
| I-73 | 3-(5-(1-(3-(difluoromethyl)benzyl)piperidin-4- |
| | yl)-1-oxoisoindolin-2-yl)piperidine-2,6-dione; |
| I-74 | 3-((4-(2-(2,6-dioxopiperidin-3-yl)-1- |
| | oxoisoindolin-5-yl)piperidin-1-yl)methyl)benzoic acid; |
| I-75 | 3-(1-oxo-5-(1-(4-propylbenzyl)piperidin-4- |
| | yl)isoindolin-2-yl)piperidine-2,6-dione; |
| I-76 | 3-(1-oxo-5-(1-(4-(trifluoromethyl)benzyl)piperidin- |
| | 4-yl)isoindolin-2-yl)piperidine-2,6-dione; |
| I-77 | 3-(5-(1-(4-(difluoromethoxy)benzyl)piperidin- |
| | 4-yl)-1-oxoisoindolin-2-yl)piperidine-2,6-dione; |
| I-78 | 3-(1-oxo-5-(1-((5-(trifluoromethyl)pyridin-2- |
| | yl)methyl)piperidin-4-yl)isoindolin-2- |
| | yl)piperidine-2,6-dione; |
| I-79 | 3-(5-(1-(3-(difluoromethoxy)benzyl)piperidin- |
| | 4-yl)-1-oxoisoindolin-2-yl)piperidine-2,6-dione; |
| I-80 | 3-(5-(1-(2-(difluoromethoxy)benzyl)piperidin- |
| | 4-yl)-1-oxoisoindolin-2-yl)piperidine-2,6-dione; |
| I-81 | 3-(5-(1-(4-cyclobutylbenzyl)piperidin-4-yl)- |
| | 1-oxoisoindolin-2-yl)piperidine-2,6-dione; |
| I-82 | 3-(5-(1-((2,3-dihydrobenzo[b][1,4]dioxin-5- |
| | yl)methyl)piperidin-4-yl)-1-oxoisoindolin-2- |
| | yl)piperidine-2,6-dione; |
| I-83 | 3-(5-(1-((2,3-dihydrobenzo[b][1,4]dioxin-6- |
| | yl)methyl)piperidin-4-yl)-1-oxoisoindolin-2- |
| | yl)piperidine-2,6-dione; |
| I-84 | 3-(5-(1-(4-(tert-butyl)benzyl)piperidin-4-yl)- |
| | 1-oxoisoindolin-2-yl)piperidine-2,6-dione; |
| I-85 | 3-(5-(1-(4-isobutylbenzyl)piperidin-4-yl)- |
| | 1-oxoisoindolin-2-yl)piperidine-2,6-dione; |
| I-86 | N-(4-((4-(2-(2,6-dioxopiperidin-3-yl)-1-oxoisoindolin- |
| | 5-yl)piperidin-1-yl)methyl)phenyl)acetamide; |
| I-87 | 3-(5-(1-((2,2-difluorobenzo[d][1,3]dioxol-5- |
| | yl)methyl)piperidin-4-yl)-1-oxoisoindolin-2- |
| | yl)piperidine-2,6-dione; |
| I-88 | 3-(5-(1-((3,4-dihydro-2H-benzo[b][1,4]dioxepin- |
| | 7-yl)methyl)piperidin-4-yl)-1-oxoisoindolin-2- |
| | yl)piperidine-2,6-dione; |
| I-89 | 3-(1-oxo-5-(1-(4-(tert-pentyl)benzyl)piperidin- |
| | 4-yl)isoindolin-2-yl)piperidine-2,6-dione; |
| I-90 | 3-(5-(1-([1,1′-biphenyl]-4-ylmethyl)piperidin-4- |
| | yl)-1-oxoisoindolin-2-yl)piperidine-2,6-dione; |
| I-91 | 3-(5-(1-(4-(1H-pyrazol-1-yl)benzyl)piperidin-4- |
| | yl)-1-oxoisoindolin-2-yl)piperidine-2,6-dione; |
| I-92 | 3-(5-(1-(4-(1H-imidazol-1-yl)benzyl)piperidin-4- |
| | yl)-1-oxoisoindolin-2-yl)piperidine-2,6-dione; |
| I-93 | 3-(5-(1-(3-(1H-pyrazol-1-yl)benzyl)piperidin-4- |
| | yl)-1-oxoisoindolin-2-yl)piperidine-2,6-dione; |
| I-94 | 3-(5-(1-(4-cyclohexylbenzyl)piperidin-4-yl)- |
| | 1-oxoisoindolin-2-yl)piperidine-2,6-dione; |
| I-95 | 3-(1-oxo-5-(1-(pyrimidin-2-ylmethyl)piperidin- |
| | 4-yl)isoindolin-2-yl)piperidine-2,6-dione; |
| I-96 | 3-(5-(1-(4-bromobenzyl)piperidin-4-yl)-1- |
| | oxoisoindolin-2-yl)piperidine-2,6-dione; |
| I-97 | 3-(5-(1-(4-chlorobenzyl)piperidin-4-yl)-1- |
| | oxoisoindolin-2-yl)piperidine-2,6-dione; |
| I-98 | 3-(5-(1-(3,5-dichlorobenzyl)piperidin-4-yl)- |
| | 1-oxoisoindolin-2-yl)piperidine-2,6-dione; |
| I-99 | 3-(5-(1-(4-chloro-3-fluorobenzyl)piperidin-4- |
| | yl)-1-oxoisoindolin-2-yl)piperidine-2,6-dione; |
| I-100 | 3-(5-(1-(3-chloro-4-fluorobenzyl)piperidin-4- |
| | yl)-1-oxoisoindolin-2-yl)piperidine-2,6-dione; |
| I-101 | 3-(5-(1-(2,4-difluorobenzyl)piperidin-4-yl)- |
| | 1-oxoisoindolin-2-yl)piperidine-2,6-dione; |
| I-102 | 3-(5-(1-(3-methoxybenzyl)piperidin-4-yl)- |
| | 1-oxoisoindolin-2-yl)piperidine-2,6-dione; |
| I-103 | 3-(5-(1-(benzo[c][1,2,5]oxadiazol-5-ylmethyl)piperidin- |
| | 4-yl)-1-oxoisoindolin-2-yl)piperidine-2,6-dione; |
| I-104 | 3-(5-(1-(2-cyclopropylbenzyl)piperidin-4-yl)- |
| | 1-oxoisoindolin-2-yl)piperidine-2,6-dione; |
| I-105 | 3-(5-(1-((1,3-dihydroisobenzofuran-5-yl)methyl)piperidin- |
| | 4-yl)-1-oxoisoindolin-2-yl)piperidine-2,6-dione; |
| I-106 | 3-(1-oxo-5-(1-(2-(trifluoromethyl)benzyl)piperidin- |
| | 4-yl)isoindolin-2-yl)piperidine-2,6-dione; |
| I-107 | 3-(5-(1-(3-(tert-butyl)benzyl)piperidin-4-yl)- |
| | 1-oxoisoindolin-2-yl)piperidine-2,6-dione; |
| I-108 | 3-(5-(1-(3-isopropoxybenzyl)piperidin-4-yl)- |
| | 1-oxoisoindolin-2-yl)piperidine-2,6-dione; |
| I-109 | 3-(1-oxo-5-(1-(4-(thiophen-3-yl)benzyl)piperidin- |
| | 4-yl)isoindolin-2-yl)piperidine-2,6-dione; |
| I-110 | 3-(5-(1-(4-cyclopentylbenzyl)piperidin-4-yl)- |
| | 1-oxoisoindolin-2-yl)piperidine-2,6-dione; |
| I-111 | 3-(1-oxo-5-(1-(4-(pyrrolidin-1-yl)benzyl)piperidin- |
| | 4-yl)isoindolin-2-yl)piperidine-2,6-dione; |
| I-112 | 3-(5-(1-(4-fluorobenzyl)piperidin-4-yl)- |
| | 1-oxoisoindolin-2-yl)piperidine-2,6-dione; |
| I-113 | 3-(5-(1-(2,4-dichlorobenzyl)piperidin-4-yl)- |
| | 1-oxoisoindolin-2-yl)piperidine-2,6-dione; |
| I-114 | 3-(1-oxo-5-(1-(quinolin-8-ylmethyl)piperidin- |
| | 4-yl)isoindolin-2-yl)piperidine-2,6-dione; |
| I-115 | 3-(5-(1-((1-methyl-1H-pyrazol-4-yl)methyl)piperidin- |
| | 4-yl)-1-oxoisoindolin-2-yl)piperidine-2,6-dione; |
| I-116 | 3-(5-(1-((1H-pyrazol-4-yl)methyl)piperidin-4- |
| | yl)-1-oxoisoindolin-2-yl)piperidine-2,6-dione; |
| I-117 | 3-(5-(1-((1-methyl-1H-pyrazol-3-yl)methyl)piperidin- |
| | 4-yl)-1-oxoisoindolin-2-yl)piperidine-2,6-dione; |
| I-118 | 3-(5-(1-((1H-pyrazol-3-yl)methyl)piperidin-4- |
| | yl)-1-oxoisoindolin-2-yl)piperidine-2,6-dione; |
| I-119 | 3-(5-(1-((1H-pyrrol-3-yl)methyl)piperidin-4- |
| | yl)-1-oxoisoindolin-2-yl)piperidine-2,6-dione; |
| I-120 | 3-(5-(1-((1H-imidazol-5-yl)methyl)piperidin-4- |
| | yl)-1-oxoisoindolin-2-yl)piperidine-2,6-dione; |
| I-121 | 3-(5-(1-((1-ethyl-1H-pyrazol-3-yl)methyl)piperidin- |
| | 4-yl)-1-oxoisoindolin-2-yl)piperidine-2,6-dione; |
| I-122 | 3-(5-(1-((2-aminopyrimidin-5-yl)methyl)piperidin- |
| | 4-yl)-1-oxoisoindolin-2-yl)piperidine-2,6-dione; |
| I-123 | 3-(5-(1-((6-aminopyridin-3-yl)methyl)piperidin- |
| | 4-yl)-1-oxoisoindolin-2-yl)piperidine-2,6-dione; |
| I-124 | 3-(5-(1-((5-amino-1-methyl-1H-pyrazol-4-yl)methyl)piperidin- |
| | 4-yl)-1-oxoisoindolin-2-yl)piperidine-2,6-dione; |
| I-125 | 3-(5-(1-((6-methylimidazo[2,1-b]thiazol-5-yl)methyl)piperidin- |
| | 4-yl)-1-oxoisoindolin-2-yl)piperidine-2,6-dione; |
| I-126 | 3-(5-(1-(imidazo[1,2-a]pyrazin-3-ylmethyl)piperidin- |
| | 4-yl)-1-oxoisoindolin-2-yl)piperidine-2,6-dione; |
| I-127 | 3-(5-(1-([1,2,4]triazolo[1,5-a]pyridin-5- |
| | ylmethyl)piperidin-4-yl)-1-oxoisoindolin-2- |
| | yl)piperidine-2,6-dione; |
| I-128 | 3-(1-oxo-5-(1-(pyrazolo[1,5-a]pyridin-4- |
| | ylmethyl)piperidin-4-yl)isoindolin-2-yl)piperidine-2,6-dione; |
| I-129 | 3-(5-(1-((1,4-dimethyl-1H-imidazol-2-yl)methyl)piperidin- |
| | 4-yl)-1-oxoisoindolin-2-yl)piperidine-2,6-dione; |
| I-130 | 3-(5-(1-(benzo[d]thiazol-5-ylmethyl)piperidin-4- |
| | yl)-1-oxoisoindolin-2-yl)piperidine-2,6-dione; |
| I-131 | 3-(1-oxo-5-(1-(pyrazolo[1,5-a]pyrimidin-6- |
| | ylmethyl)piperidin-4-yl)isoindolin-2-yl)piperidine-2,6-dione; |
| I-132 | 3-(5-(1-(imidazo[1,2-a]pyrimidin-3-ylmethyl)piperidin- |
| | 4-yl)-1-oxoisoindolin-2-yl)piperidine-2,6-dione; |
| I-133 | 3-(5-(1-(imidazo[1,2-a]pyrimidin-2-ylmethyl)piperidin- |
| | 4-yl)-1-oxoisoindolin-2-yl)piperidine-2,6-dione; |
| I-134 | 3-(5-(1-((1-cyclobutyl-1H-1,2,3-triazol-4- |
| | yl)methyl)piperidin-4-yl)-1-oxoisoindolin- |
| | 2-yl)piperidine-2,6-dione; |
| I-135 | 3-(1-oxo-5-(1-((4,5,6,7-tetrahydropyrazolo[1,5-a]pyridin-2- |
| | yl)methyl)piperidin-4-yl)isoindolin-2-yl)piperidine-2,6-dione; |
| I-136 | 3-(5-(1-((1H-indol-2-yl)methyl)piperidin-4- |
| | yl)-1-oxoisoindolin-2-yl)piperidine-2,6-dione; |
| I-137 | 3-(5-(1-((1H-indazol-6-yl)methyl)piperidin-4- |
| | yl)-1-oxoisoindolin-2-yl)piperidine-2,6-dione; |
| I-138 | 3-(5-(1-((1H-pyrrolo[2,3-b]pyridin-3-yl)methyl)piperidin- |
| | 4-yl)-1-oxoisoindolin-2-yl)piperidine-2,6-dione; |
| I-139 | 3-((4-(2-(2,6-dioxopiperidin-3-yl)-1- |
| | oxoisoindolin-5-yl)piperidin-1-yl)methyl)benzamide |
| I-140 | 3-(5-(1-((1H-pyrrolo[2,3-b]pyridin-6-yl)methyl)piperidin- |
| | 4-yl)-1-oxoisoindolin-2-yl)piperidine-2,6-dione; |
| I-141 | 3-(5-(1-((3,4-dihydro-2H-benzo[b][1,4]thiazin- |
| | 6-yl)methyl)piperidin-4-yl)-1-oxoisoindolin-2- |
| | yl)piperidine-2,6-dione; |
| I-142 | 3-(1-oxo-5-(1-((2-(pyrrolidin-1-yl)pyrimidin-5- |
| | yl)methyl)piperidin-4-yl)isoindolin-2-yl)piperidine-2,6-dione; |
| I-143 | 3-(5-(1-((2-(tert-butyl)thiazol-4-yl)methyl)piperidin- |
| | 4-yl)-1-oxoisoindolin-2-yl)piperidine-2,6-dione; |
| I-144 | 3-(1-oxo-5-(1-((2-(thiophen-2-yl)thiazol-5- |
| | yl)methyl)piperidin-4-yl)isoindolin-2-yl)piperidine-2,6-dione; |
| I-145 | 3-(5-(1-((2-cyclohexylthiazol-5-yl)methyl)piperidin- |
| | 4-yl)-1-oxoisoindolin-2-yl)piperidine-2,6-dione; |
| I-146 | 3-(5-(1-((5-cyclopropyl-1H-pyrazol-3-yl)methyl)piperidin- |
| | 4-yl)-1-oxoisoindolin-2-yl)piperidine-2,6-dione; |
| I-147 | 3-(5-(1-((2-morpholinopyrimidin-5-yl)methyl)piperidin- |
| | 4-yl)-1-oxoisoindolin-2-yl)piperidine-2,6-dione; |
| I-148 | 3-(1-oxo-5-(1-((3-phenyl-1H-pyrazol-4-yl)methyl)piperidin- |
| | 4-yl)isoindolin-2-yl)piperidine-2,6-dione; |
| I-149 | 3-(5-(1-((6-methyl-1H-indol-3-yl)methyl)piperidin- |
| | 4-yl)-1-oxoisoindolin-2-yl)piperidine-2,6-dione; |
| I-150 | methyl 4-((4-(2-(2,6-dioxopiperidin-3-yl)-1-oxoisoindolin- |
| | 5-yl)piperidin-1-yl)methyl)-1H-pyrrole-2-carboxylate |
| I-151 | 3-(1-oxo-5-(1-((3-(pyridin-3-yl)-1H-pyrazol-4- |
| | yl)methyl)piperidin-4-yl)isoindolin-2-yl)piperidine-2,6-dione; |
| I-152 | 3-(1-oxo-5-(1-((2-phenyl-1H-imidazol-4-yl)methyl)piperidin- |
| | 4-yl)isoindolin-2-yl)piperidine-2,6-dione; |
| I-153 | 3-(1-oxo-5-(1-((5-(pyridin-2-yl)-1H-pyrazol-3- |
| | yl)methyl)piperidin-4-yl)isoindolin-2-yl)piperidine-2,6-dione; |
| I-154 | 3-(1-oxo-5-(1-((4-phenyl-1H-imidazol-2-yl)methyl)piperidin- |
| | 4-yl)isoindolin-2-yl)piperidine-2,6-dione; |
| I-155 | 3-(1-oxo-5-(piperidin-4-yl)isoindolin- |
| | 2-yl)piperidine-2,6-dione; |
| I-156 | 3-(5-(1-(3,5-difluoro-4-hydroxybenzyl)piperidin- |
| | 4-yl)-1-oxoisoindolin-2-yl)piperidine-2,6-dione; |
| I-157 | 3-(5-(1-(2-methylbenzyl)piperidin-4-yl)- |
| | 1-oxoisoindolin-2-yl)piperidine-2,6-dione; |
| I-158 | 3-(5-(1-(4-methylbenzyl)piperidin-4-yl)- |
| | 1-oxoisoindolin-2-yl)piperidine-2,6-dione; |
| I-159 | 3-(5-(1-(3,5-dimethylbenzyl)piperidin-4-yl)- |
| | 1-oxoisoindolin-2-yl)piperidine-2,6-dione; |
| I-160 | 3-(5-((2S)-1-benzyl-2-methylpiperidin-4-yl)- |
| | 1-oxoisoindolin-2-yl)piperidine-2,6-dione; |
| I-161 | 3-(5-((2R)-1-benzyl-2-methylpiperidin-4-yl)- |
| | 1-oxoisoindolin-2-yl)piperidine-2,6-dione; |
| I-162 | 3-(5-(1-benzyl-2-methylpiperidin-4-yl)- |
| | 1-oxoisoindolin-2-yl)piperidine-2,6-dione; |
| I-163 | 3-(5-(1-methyl-1,2,3,6-tetrahydropyridin-4- |
| | yl)-1-oxoisoindolin-2-yl)piperidine-2,6-dione; |
| I-164 | 3-(1-oxo-5-(1-((5,6,7,8-tetrahydronaphthalen-1- |
| | yl)methyl)piperidin-4-yl)isoindolin-2-yl)piperidine-2,6-dione; |
| I-165 | 3-(5-(azepan-4-yl)-1-oxoisoindolin-2-yl)piperidine-2,6-dione; |
| I-166 | 3-(5-((R)-azepan-4-yl)-1-oxoisoindolin- |
| | 2-yl)piperidine-2,6-dione; |
| I-167 | 3-(5-((S)-azepan-4-yl)-1-oxoisoindolin- |
| | 2-yl)piperidine-2,6-dione; |
| I-168 | 3-(1-oxo-5-(1-((1,2,3,4-tetrahydronaphthalen-1- |
| | yl)methyl)piperidin-4-yl)isoindolin-2-yl)piperidine-2,6-dione; |
| I-169 | methyl 2-(4-(2-(2,6-dioxopiperidin-3-yl)- |
| | 1-oxoisoindolin-5-yl)piperidin-1-yl)acetate |
| I-170 | 3-(1-oxo-5-(1-phenylpiperidin-4- |
| | yl)isoindolin-2-yl)piperidine-2,6-dione; |
| I-171 | 3-(1-oxo-5-(2,2,6,6-tetramethylpiperidin- |
| | 4-yl)isoindolin-2-yl)piperidine-2,6-dione; |
| I-172 | 3-(5-(1-benzyl-1,2,3,6-tetrahydropyridin-4- |
| | yl)-1-oxoisoindolin-2-yl)piperidine-2,6-dione; |
| I-173 | 3-(5-(1-(3-methylbenzyl)piperidin-4-yl)- |
| | 1-oxoisoindolin-2-yl)piperidine-2,6-dione; |
| I-174 | 3-(5-(1-(2,6-dimethylbenzyl)piperidin-4-yl)- |
| | 1-oxoisoindolin-2-yl)piperidine-2,6-dione; |
| I-175 | 3-(1-oxo-5-(1-((5,6,7,8-tetrahydronaphthalen-2- |
| | yl)methyl)piperidin-4-yl)isoindolin-2-yl)piperidine-2,6-dione; |
| I-176 | ethyl 2-(4-(2-(2,6-dioxopiperidin-3-yl)-1- |
| | oxoisoindolin-5-yl)piperidin-1-yl)acetate |
| I-177 | tert-butyl 2-(4-(2-(2,6-dioxopiperidin-3-yl)- |
| | 1-oxoisoindolin-5-yl)piperidin-1-yl)acetate |
| I-178 | 2-(4-(2-(2,6-dioxopiperidin-3-yl)-1- |
| | oxoisoindolin-5-yl)piperidin-1-yl)acetic acid |
| I-179 | 3-(1-oxo-5-(1-(3,3,3-trifluoropropyl)piperidin- |
| | 4-yl)isoindolin-2-yl)piperidine-2,6-dione; |
| I-180 | 2-(4-(2-(2,6-dioxopiperidin-3-yl)-1-oxoisoindolin- |
| | 5-yl)piperidin-1-yl)-N-phenylacetamide |
| I-181 | 3-(5-(1-(3-fluoropropyl)piperidin-4-yl)- |
| | 1-oxoisoindolin-2-yl)piperidine-2,6-dione; |
| I-182 | tert-butyl 4-((4-(2-(2,6-dioxopiperidin-3-yl)-1- |
| | oxoisoindolin-5-yl)piperidin-1-yl)methyl)benzoate; |
| I-183 | 3-(5-(2-methylpiperidin-4-yl)-1- |
| | oxoisoindolin-2-yl)piperidine-2,6-dione; |
| I-184 | 3-(5-(3,3-dimethylpiperidin-4-yl)-1- |
| | oxoisoindolin-2-yl)piperidine-2,6-dione; |
| I-185 | 3-(5-(1-benzyl-3,3-dimethylpiperidin-4-yl)- |
| | 1-oxoisoindolin-2-yl)piperidine-2,6-dione; |
| I-186 | 5-(3-methylpiperidin-4-yl)-2-(2- |
| | oxopiperidin-3-yl)isoindolin-1-one; |
| I-187 | 3-(5-(1-benzyl-3-methylpiperidin-4-yl)- |
| | 1-oxoisoindolin-2-yl)piperidine-2,6-dione; |
| I-188 | 3-(5-(8-azabicyclo[3.2.1]octan-3-yl)-1- |
| | oxoisoindolin-2-yl)piperidine-2,6-dione; |
| I-189 | 3-(5-(1-(2-hydroxy-1-phenylethyl)piperidin-4- |
| | yl)-1-oxoisoindolin-2-yl)piperidine-2,6-dione; |
| I-190 | 3-(5-((S)-1-benzylazepan-4-yl)-1-oxoisoindolin- |
| | 2-yl)piperidine-2,6-dione; |
| I-191 | 3-(5-(1-benzyl-2,5-dihydro-1H-pyrrol-3-yl)- |
| | 1-oxoisoindolin-2-yl)piperidine-2,6-dione; |
| I-192 | 3-(5-(1-benzyl-2-oxo-1,2-dihydropyridin-4- |
| | yl)-1-oxoisoindolin-2-yl)piperidine-2,6-dione; |
| I-193 | 3-(5-(1-benzyl-2-oxopiperidin-4-yl)-1- |
| | oxoisoindolin-2-yl)piperidine-2,6-dione; |
| I-194 | 3-(1-oxo-5-(2-oxopiperidin-4- |
| | yl)isoindolin-2-yl)piperidine-2,6-dione; |
| I-195 | 3-(1-oxo-5-(2-oxo-1,2-dihydropyridin-4- |
| | yl)isoindolin-2-yl)piperidine-2,6-dione; |
| I-196 | 3-(1-oxo-5-(1,2,3,4-tetrahydroquinolin-4- |
| | yl)isoindolin-2-yl)piperidine-2,6-dione; |
| I-197 | 3-(5-(1-benzyl-1,2,3,4-tetrahydroquinolin-4- |
| | yl)-1-oxoisoindolin-2-yl)piperidine-2,6-dione; |
| I-198 | 3-(5-(1-((1-benzyl-1H-tetrazol-5-yl)methyl)piperidin- |
| | 4-yl)-1-oxoisoindolin-2-yl)piperidine-2,6-dione; |
| I-199 | 3-(1-oxo-5-(1-((5-phenyl-1,3,4-oxadiazol-2- |
| | yl)methyl)piperidin-4-yl)isoindolin-2-yl)piperidine-2,6-dione; |
| I-200 | 3-(5-(1-(benzo[d]thiazol-2-ylmethyl)piperidin-4- |
| | yl)-1-oxoisoindolin-2-yl)piperidine-2,6-dione; |
| I-201 | 3-(1-oxo-5-(1-((3-(pyridin-2-yl)-1H-pyrazol-5- |
| | yl)methyl)piperidin-4-yl)isoindolin-2-yl)piperidine-2,6-dione; |
| I-202 | 3-(5-(1-((R)-2-hydroxy-1-phenylethyl)piperidin- |
| | 4-yl)-1-oxoisoindolin-2-yl)piperidine-2,6-dione; |
| I-203 | 3-(5-(1-((1-methyl-1H-indazol-3-yl)methyl)piperidin- |
| | 4-yl)-1-oxoisoindolin-2-yl)piperidine-2,6-dione; |
| I-204 | 3-(5-(1-((1,2,4-oxadiazol-3-yl)methyl)piperidin- |
| | 4-yl)-1-oxoisoindolin-2-yl)piperidine-2,6-dione; |
| I-205 | 3-(5-(1-(4-hydroxy-3-((4-methylpiperazin-1- |
| | yl)methyl)benzyl)piperidin-4-yl)-1-oxoisoindolin- |
| | 2-yl)piperidine-2,6-dione; |
| I-206 | 2-(4-((4-(2-(2,6-dioxopiperidin-3-yl)-1-oxoisoindolin- |
| | 5-yl)piperidin-1-yl)methyl)phenyl)acetonitrile; |
| I-207 | 3-(5-(1-((2-(4-chlorophenyl)-5-methyloxazol- |
| | 4-yl)methyl)piperidin-4-yl)-1-oxoisoindolin- |
| | 2-yl)piperidine-2,6-dione; |
| I-208 | 3-(5-(1-((7-hydroxy-2-methylpyrazolo[1,5-a]pyrimidin-5- |
| | yl)methyl)piperidin-4-yl)-1-oxoisoindolin-2-yl)piperidine- |
| | 2,6-dione; |
| I-209 | 3-(5-(1-(2,2-difluoro-1-phenylethyl)piperidin-4- |
| | yl)-1-oxoisoindolin-2-yl)piperidine-2,6-dione; |
| I-210 | 3-(5-(1-((3-fluorobicyclo[1.1.1]pentan-1-yl)methyl)piperidin- |
| | 4-yl)-1-oxoisoindolin-2-yl)piperidine-2,6-dione; |
| I-211 | 3-(1-oxo-5-(1-((2-phenylthiazol-4-yl)methyl)piperidin- |
| | 4-yl)isoindolin-2-yl)piperidine-2,6-dione; |
| I-212 | 3-(5-(1-(2-fluoro-1-phenylethyl)piperidin-4- |
| | yl)-1-oxoisoindolin-2-yl)piperidine-2,6-dione; |
| I-213 | 3-(1-oxo-5-(1-((4-oxo-3,4-dihydrothieno[3,2-d]pyrimidin-2- |
| | yl)methyl)piperidin-4-yl)isoindolin-2-yl)piperidine-2,6-dione; |
| I-214 | 3-(1-oxo-5-(1-(quinolin-4-ylmethyl)piperidin- |
| | 4-yl)isoindolin-2-yl)piperidine-2,6-dione; |
| I-215 | 3-(5-(1-(3,5-bis(trifluoromethyl)benzyl)piperidin- |
| | 4-yl)-1-oxoisoindolin-2-yl)piperidine-2,6-dione; |
| I-216 | 3-((4-(2-(2,6-dioxopiperidin-3-yl)-1-oxoisoindolin-5- |
| | yl)piperidin-1-yl)methyl)-N,N-dimethylbenzenesulfonamide; |
| I-217 | 6-((4-(2-(2,6-dioxopiperidin-3-yl)-1-oxoisoindolin- |
| | 5-yl)piperidin-1-yl)methyl)picolinonitrile; |
| I-218 | 2-(4-((4-(2-(2,6-dioxopiperidin-3-yl)-1-oxoisoindolin- |
| | 5-yl)piperidin-1-yl)methyl)phenoxy)acetonitrile; |
| I-219 | 3-(5-(1-((1H-indazol-5-yl)methyl)piperidin-4- |
| | yl)-1-oxoisoindolin-2-yl)piperidine-2,6-dione; |
| I-220 | 3-(5-(1-(2,2-difluoroethyl)piperidin-4-yl)- |
| | 1-oxoisoindolin-2-yl)piperidine-2,6-dione; |
| I-221 | 3-(5-(1-((7-methyl-4-oxo-4H-pyrido[1,2-a]pyrimidin- |
| | 2-yl)methyl)piperidin-4-yl)-1-oxoisoindolin-2- |
| | yl)piperidine-2,6-dione; |
| I-222 | benzyl 4-(2-(2,6-dioxopiperidin-3-yl)-1- |
| | oxoisoindolin-5-yl)piperidine-1-carboxylate; |
| I-223 | 3-(1-oxo-5-(1-(2-phenylacetyl)piperidin-4- |
| | yl)isoindolin-2-yl)piperidine-2,6-dione; |
| I-224 | 3-(1-oxo-5-(1-(2,2,2-trifluoro-1-phenylethyl)piperidin- |
| | 4-yl)isoindolin-2-yl)piperidine-2,6-dione; |
| I-225 | 3-(5-(1-(4-(5-methylbenzo[d]thiazol-2-yl)benzyl)piperidin- |
| | 4-yl)-1-oxoisoindolin-2-yl)piperidine-2,6-dione; |
| I-226 | 3-(5-(1-(isoquinolin-1-ylmethyl)piperidin-4- |
| | yl)-1-oxoisoindolin-2-yl)piperidine-2,6-dione; |
| I-227 | 3-(5-(1-(4-(4-methoxypiperidin-1-yl)benzyl)piperidin- |
| | 4-yl)-1-oxoisoindolin-2-yl)piperidine-2,6-dione; |
| I-228 | 3-(5-(1-(4-(isopropylthio)benzyl)piperidin-4- |
| | yl)-1-oxoisoindolin-2-yl)piperidine-2,6-dione; |
| I-229 | tert-butyl (5-((4-(2-(2,6-dioxopiperidin-3-yl)- |
| | 1-oxoisoindolin-5-yl)piperidin-1-yl)methyl)-4- |
| | (trifluoromethyl)thiazol-2-yl)carbamate; |
| I-230 | 3-(1-oxo-5-(1-((S)-1-phenylethyl)piperidin- |
| | 4-yl)isoindolin-2-yl)piperidine-2,6-dione; |
| I-231 | 2-(4-((4-(2-(2,6-dioxopiperidin-3-yl)-1-oxoisoindolin- |
| | 5-yl)piperidin-1-yl)methyl)phenyl)acetic acid; |
| I-232 | 3-(5-(1-((7-fluoroquinolin-2-yl)methyl)piperidin- |
| | 4-yl)-1-oxoisoindolin-2-yl)piperidine-2,6-dione; |
| I-233 | 3-(5-(1-((5-methyl-2-(4-(trifluoromethyl)phenyl)oxazol- |
| | 4-yl)methyl)piperidin-4-yl)-1-oxoisoindolin-2-yl)piperidine- |
| | 2,6-dione; |
| I-234 | 3-(5-(1-((2-amino-4-(trifluoromethyl)thiazol- |
| | 5-yl)methyl)piperidin-4-yl)-1-oxoisoindolin- |
| | 2-yl)piperidine-2,6-dione; |
| I-235 | 3-((4-(2-(2,6-dioxopiperidin-3-yl)-1-oxoisoindolin-5- |
| | yl)piperidin-1-yl)methyl)-1,2,4-oxadiazole-5-carboxamide; |
| I-236 | 3-(5-(1-(3-(morpholinosulfonyl)benzyl)piperidin- |
| | 4-yl)-1-oxoisoindolin-2-yl)piperidine-2,6-dione; |
| I-237 | 4-((4-(2-(2,6-dioxopiperidin-3-yl)-1-oxoisoindolin-5- |
| | yl)piperidin-1-yl)methyl)-N,N-dimethylbenzenesulfonamide; |
| I-238 | 3-(1-oxo-5-(1-(thiazol-4-ylmethyl)piperidin- |
| | 4-yl)isoindolin-2-yl)piperidine-2,6-dione; |
| I-239 | 3-(1-oxo-5-(1-(quinoxalin-6-ylmethyl)piperidin- |
| | 4-yl)isoindolin-2-yl)piperidine-2,6-dione; |
| I-240 | 3-(5-(1-((2-(4-fluorophenyl)-5-methyloxazol- |
| | 4-yl)methyl)piperidin-4-yl)-1-oxoisoindolin- |
| | 2-yl)piperidine-2,6-dione; |
| I-241 | 3-(1-oxo-5-(1-((3-(m-tolyl)-1,2,4-oxadiazol-5- |
| | yl)methyl)piperidin-4-yl)isoindolin-2-yl)piperidine-2,6-dione; |
| I-242 | 3-(5-(1-(4-(tert-butyl)benzoyl)piperidin-4- |
| | yl)-1-oxoisoindolin-2-yl)piperidine-2,6-dione; |
| I-243 | 3-(1-oxo-5-(1-((5-(4-(trifluoromethyl)phenyl)-1,2,4- |
| | oxadiazol-3-yl)methyl)piperidin-4-yl)isoindolin-2- |
| | yl)piperidine-2,6-dione; |
| I-244 | 3-(5-(1-(4-((4-fluorobenzyl)oxy)benzyl)piperidin- |
| | 4-yl)-1-oxoisoindolin-2-yl)piperidine-2,6-dione; |
| I-245 | 3-(5-(1-((3-methylisoxazol-5-yl)methyl)piperidin- |
| | 4-yl)-1-oxoisoindolin-2-yl)piperidine-2,6-dione; |
| I-246 | 3-(5-(1-(isoxazol-3-ylmethyl)piperidin-4-yl)- |
| | 1-oxoisoindolin-2-yl)piperidine-2,6-dione; |
| I-247 | 3-(1-oxo-5-(1-((R)-1-phenylethyl)piperidin- |
| | 4-yl)isoindolin-2-yl)piperidine-2,6-dione; |
| I-248 | 3-(5-(1-(4-(methoxymethyl)benzyl)piperidin-4- |
| | yl)-1-oxoisoindolin-2-yl)piperidine-2,6-dione; |
| I-249 | 3-(5-(1-((S)-2-hydroxy-1-phenylethyl)piperidin- |
| | 4-yl)-1-oxoisoindolin-2-yl)piperidine-2,6-dione; |
| I-250 | 3-(1-oxo-5-(1-(phenylsulfonyl)piperidin- |
| | 4-yl)isoindolin-2-yl)piperidine-2,6-dione; |
| I-251 | 3-(5-(1-((5-methyl-3-phenylisoxazol-4-yl)methyl)piperidin- |
| | 4-yl)-1-oxoisoindolin-2-yl)piperidine-2,6-dione; |
| I-252 | 3-(5-(1-(4-((difluoromethyl)sulfonyl)benzyl)piperidin- |
| | 4-yl)-1-oxoisoindolin-2-yl)piperidine-2,6-dione; |
| I-253 | 3-(1-oxo-5-(1-(2,2,2-trifluoroethyl)piperidin- |
| | 4-yl)isoindolin-2-yl)piperidine-2,6-dione; |
| I-254 | methyl 2-((4-(2-(2,6-dioxopiperidin-3-yl)-1-oxoisoindolin- |
| | 5-yl)piperidin-1-yl)methyl)oxazole-4-carboxylate; |
| I-255 | 3-(1-oxo-5-(1-(4-(pyridin-2-ylmethoxy)benzyl)piperidin- |
| | 4-yl)isoindolin-2-yl)piperidine-2,6-dione; |
| I-256 | 3-(5-(1-acetylpiperidin-4-yl)-1- |
| | oxoisoindolin-2-yl)piperidine-2,6-dione; |
| I-257 | 3-(5-(1-((5-methyl-2-phenyloxazol-4-yl)methyl)piperidin- |
| | 4-yl)-1-oxoisoindolin-2-yl)piperidine-2,6-dione; |
| I-258 | 3-(5-(1-((3-cyclohexylisoxazol-5-yl)methyl)piperidin- |
| | 4-yl)-1-oxoisoindolin-2-yl)piperidine-2,6-dione; |
| I-259 | 3-(1-oxo-5-(1-((2-oxo-2,3-dihydro-1H-benzo[d]imidazol-5- |
| | yl)methyl)piperidin-4-yl)isoindolin-2-yl)piperidine-2,6-dione; |
| I-260 | 3-(5-(1-benzylpyrrolidin-3-yl)-1- |
| | oxoisoindolin-2-yl)piperidine-2,6-dione; |
| I-261 | (R)-3-(5-((R)-1-benzylazepan-4-yl)- |
| | 1-oxoisoindolin-2-yl)piperidine-2,6-dione; |
| I-262 | (S)-3-(5-((S)-1-benzylazepan-4-yl)- |
| | 1-oxoisoindolin-2-yl)piperidine-2,6-dione; |
| I-263 | 3-(5-(1-benzylazepan-4-yl)-1- |
| | oxoisoindolin-2-yl)piperidine-2,6-dione; |
| I-264 | 3-(5-(1-methyl-2,3,6,7-tetrahydro-1H-azepin-4- |
| | yl)-1-oxoisoindolin-2-yl)piperidine-2,6-dione; |
| I-265 | 3-(5-(8-benzyl-8-azabicyclo[3.2.1]octan-3-yl)- |
| | 1-oxoisoindolin-2-yl)piperidine-2,6-dione; |
| I-266 | trans-3-(1-oxo-5-(1-((4- |
| | (trifluoromethyl)cyclohexyl)methyl)piperidin- |
| | 4-yl)isoindolin-2-yl)piperidine-2,6-dione; |
| I-267 | (S)-3-(1-oxo-5-((S)-piperidin-3- |
| | yl)isoindolin-2-yl)piperidine-2,6-dione; |
| I-268 | 3-(5-(1-acetyl-1,2,5,6-tetrahydropyridin-3-yl)- |
| | 1-oxoisoindolin-2-yl)piperodome-2,6-dione; |
| I-269 | (R)-3-(5-((R)-1-acetylpyrrolidin-3-yl)- |
| | 1-oxoisoindolin-2-yl)piperidine-2,6-dione; |
| I-270 | 3-(5-(1-acetyl-1,2,3,6-tetrahydropyridin-4- |
| | yl)-1-oxoisoindolin-2-yl)piperidine-2,6-dione; |
| I-271 | 3-(5-(octahydroindolizin-7-yl)-1- |
| | oxoisoindolin-2-yl)piperidine-2,6-dione; |
| I-272 | (R)-3-(5-((S)-1-benzylazepan-4-yl)-1- |
| | oxoisoindolin-2-yl)piperidine-2,6-dione; |
| I-273 | 3-(5-((R)-1-benzylazepan-4-yl)-1- |
| | oxoisoindolin-2-yl)piperidine-2,6-dione; |
| I-274 | 3-(5-(2,5-dihydro-1H-pyrrol-3-yl)-1- |
| | oxoisoindolin-2-yl)piperidine-2,6-dione; |
| I-275 | 3-(5-(1-acetyl-2,5-dihydro-1H-pyrrol-3-yl)- |
| | 1-oxoisoindolin-2-yl)piperidine-2,6-dione; |
| I-276 | cis-3-(1-oxo-5-(1-((4- |
| | (trifluoromethyl)cyclohexyl)methyl)piperidin- |
| | 4-yl)isoindolin-2-yl)piperidine-2,6-dione; |
| I-277 | 3-(1-oxo-5-(2,3,6,7-tetrahydro-1H-azepin- |
| | 4-yl)isoindolin-2-yl)piperidine-2,6-dione; |
| I-278 | 3-(5-(1-methylazepan-4-yl)-1- |
| | oxoisoindolin-2-yl)piperidine-2,6-dione; |
| I-279 | (R)-3-(1-oxo-5-((S)-piperidin-3- |
| | yl)isoindolin-2-yl)piperidine-2,6-dione; |
| I-280 | 3-(1-oxo-5-(1,2,3,6-tetrahydropyridin- |
| | 4-yl)isoindolin-2-yl)piperidine-2,6-dione; |
| I-281 | (S)-3-(5-((R)-1-benzylazepan-4-yl)-1- |
| | oxoisoindolin-2-yl)piperidine-2,6-dione; |
| I-282 | 3-(1-oxo-5-(1,2,5,6-tetrahydropyridin-3- |
| | yl)isoindolin-2-yl)piperidine-2,6-dione; |
| I-283 | 3-(1-oxo-5-(2,2,6,6-tetramethyl-1,2,3,6- |
| | tetrahydropyridin-4-yl)isoindolin-2-yl)piperidine-2,6-dione; |
| I-284 | (S)-3-(5-((R)-1-acetylpyrrolidin-3-yl)- |
| | 1-oxoisoindolin-2-yl)piperidine-2,6-dione; |
| I-285 | 3-(5-(1-((6-isopropoxypyridin-3-yl)methyl)piperidin- |
| | 4-yl)-1-oxoisoindolin-2-yl)piperidine-2,6-dione; |
| I-286 | 3-(1-oxo-5-(1-((1-phenyl-1H-pyrazol-5-yl)methyl)piperidin- |
| | 4-yl)isoindolin-2-yl)piperidine-2,6-dione; |
| I-287 | 3-(5-(1-(4-ethoxybenzyl)piperidin-4-yl)- |
| | 1-oxoisoindolin-2-yl)piperidine-2,6-dione; |
| I-288 | 3-(1-oxo-5-(1-((1-phenyl-1H-pyrazol-4-yl)methyl)piperidin- |
| | 4-yl)isoindolin-2-yl)piperidine-2,6-dione; |
| I-289 | 3-(5-(1-((1-isopropyl-1H-pyrazol-5-yl)methyl)piperidin- |
| | 4-yl)-1-oxoisoindolin-2-yl)piperidine-2,6-dione; |
| I-290 | 3-(5-(1-(isothiazol-5-ylmethyl)piperidin-4- |
| | yl)-1-oxoisoindolin-2-yl)piperidine-2,6-dione; |
| I-291 | 3-(5-(1-((1-isopropyl-1H-pyrazol-4-yl)methyl)piperidin- |
| | 4-yl)-1-oxoisoindolin-2-yl)piperidine-2,6-dione; |
| I-292 | 3-(5-(1-((1H-pyrazol-5-yl)methyl)piperidin-4- |
| | yl)-1-oxoisoindolin-2-yl)piperidine-2,6-dione; |
| I-293 | 3-(5-(1-((5-isopropoxypyridin-2-yl)methyl)piperidin- |
| | 4-yl)-1-oxoisoindolin-2-yl)piperidine-2,6-dione; |
| I-294 | 3-(1-oxo-5-(1-((1-(pyridin-3-yl)-1H-pyrazol-5- |
| | yl)methyl)piperidin-4-yl)isoindolin-2-yl)piperidine-2,6-dione; |
| I-295 | 3-(1-oxo-5-(1-((1-(pyridin-3-yl)-1H-pyrazol-4- |
| | yl)methyl)piperidin-4-yl)isoindolin-2-yl)piperidine-2,6-dione; |
| I-296 | 5-((4-(2-(2,6-dioxopiperidin-3-yl)-1-oxoisoindolin- |
| | 5-yl)piperidin-1-yl)methyl)-2-fluorobenzonitrile |
| I-297 | 3-(5-(1-((5-fluoropyridin-2-yl)methyl)piperidin- |
| | 4-yl)-1-oxoisoindolin-2-yl)piperidine-2,6-dione; |
| I-298 | 3-(5-(1-((1-ethyl-3-(pyridin-3-yl)-1H-pyrazol- |
| | 4-yl)methyl)piperidin-4-yl)-1-oxoisoindolin-2- |
| | yl)piperidine-2,6-dione; |
| I-299 | 3-(5-(1-((6-methoxypyridin-2-yl)methyl)piperidin- |
| | 4-yl)-1-oxoisoindolin-2-yl)piperidine-2,6-dione; |
| I-300 | 3-(5-(1-((3-((3S,5S)-adamantan-1-yl)-1H-pyrazol- |
| | 5-yl)methyl)piperidin-4-yl)-1-oxoisoindolin-2- |
| | yl)piperidine-2,6-dione; |
| I-301 | 3-(5-(1-((6-isopropoxypyridin-2-yl)methyl)piperidin- |
| | 4-yl)-1-oxoisoindolin-2-yl)piperidine-2,6-dione; |
| I-302 | 3-(5-(1-((1-benzyl-5-(pyridin-2-yl)-1H-pyrazol- |
| | 3-yl)methyl)piperidin-4-yl)-1-oxoisoindolin-2- |
| | yl)piperidine-2,6-dione; and |
| I-303 | trans-3-(5-(1-((4-methoxycyclohexyl)methyl)piperidin- |
| | 4-yl)-1-oxoisoindolin-2-yl)piperidine-2,6-dione. |

Embodiment 71: A combination comprising, a compound selected from Compound 1-156, Compound 1-57, Compound 1-87, Compound 1-88, Compound 1-265, and Compound 1-112, and a second agent.

Embodiment 72: The combination according to Embodiment 71, wherein the compound is Compound I-156.

Embodiment 73: The combination according to Embodiment 71, wherein the compound is Compound I-57.

Embodiment 74: The combination according to Embodiment 71, wherein the compound is Compound I-87.

Embodiment 75: The combination according to Embodiment 71, wherein the compound is Compound I-88.

Embodiment 76: The combination according to Embodiment 71, wherein the compound is Compound I-265.

Embodiment 77: The combination according to Embodiment 71, wherein the compound is Compound I-112.

Embodiment 78: The combination according to any one of Embodiments 71-77, wherein the combination comprises about 2 mg, or about 10 mg, or about 20 mg, or about 40 mg, or about 80 mg, or about 160 mg, or about 320 mg of the compound.

Embodiment 79: The combination according to any one of Embodiments 71-78, wherein the combination comprises about 100 mg, or about 200 mg, or about 300 mg, or about 400 mg, or about 500 mg of the second therapeutic agent.

Embodiment 80: The combination according to any one of Embodiments 71-79, wherein the combination comprises about 2 mg, or about 10 mg, or about 20 mg, or about 40 mg, or about 80 mg, or about 160 mg, or about 320 mg of the compound; and about 100 mg, or about 200 mg, or about 300 mg, or about 400 mg, or about 500 mg of the second therapeutic agent.

Embodiment 81: The combination according to any one of Embodiments 71-80, wherein the combination comprises about 400 mg of the second therapeutic agent.

Embodiment 82: The combination according to any one of Embodiments 71-81, wherein the second therapeutic agent is an immunomodulator.

Embodiment 83: The combination according to Embodiment 82, wherein the immunomodulator is an immune checkpoint inhibitor.

Embodiment 84: The combination according to Embodiment 83, wherein the immune checkpoint inhibitor is a PD-1 inhibitor.

Embodiment 85: The combination according to Embodiment 84, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 86: The combination according to Embodiment 85, wherein the PD-1 inhibitor is PDR001.

Embodiment 87: A method of treating or preventing cancer comprising administering to a patient in need thereof a combination according to any one of Embodiments 71-86.

Embodiment 88: A combination according to any one of Embodiments 71-86 for use in the treatment or prevention of cancer.

Embodiment 89: Use of a combination according to any one of Embodiments 71-86 for the treatment or prevention of cancer.

Embodiment 90: Use of a combination according to any one of Embodiments 71-86 for the manufacture of a medicament for treating or preventing of cancer.

Embodiment 91: A method of treating or preventing cancer comprising administering to a patient in need thereof, a compound according to any one of Embodiments 1-70, or a pharmaceutically acceptable salt, hydrate, solvate, prodrug, stereoisomer, or tautomer thereof, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 92: A method of treating or preventing cancer comprising administering to a patient in need thereof a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient, wherein the pharmaceutical formulation comprises about 2 mg, or about 4 mg, or about 10 mg, or about 20 mg, or about 40 mg, or about 80 mg, or about 160 mg, or about 320 mg of the compound.

Embodiment 93: A method of treating or preventing an IKZF2-dependent disease by reducing or decreasing IKZF2 protein levels in a patient comprising administering to the patient in need thereof a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent, wherein reduction or decrease of IKZF2 protein levels treats or prevents the IKZF2-dependent disease.

Embodiment 94: A combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for use in the treatment or prevention of an IKZF2-dependent disease by reducing or decreasing IKZF2 protein levels, wherein reduction or decrease of IKZF2 protein levels treats or prevents the IKZF2-dependent disease.

Embodiment 95: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for the treatment or prevention of an IKZF2-dependent disease by reducing or decreasing IKZF2 protein levels, wherein reduction or decrease of IKZF2 protein levels treats or prevents the IKZF2-dependent disease.

Embodiment 96: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for the manufacture of a medicament for treating or preventing an IKZF2-dependent disease by reducing or decreasing IKZF2 protein levels, wherein reduction or decrease of IKZF2 protein levels treats or prevents the IKZF2-dependent disease.

Embodiment 97: A method of treating or preventing an IKZF2-dependent disease by degrading IKZF2 in a patient comprising administering to the patient in need thereof a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent, wherein degradation of IKZF2 treats or prevents the IKZF2-dependent disease.

Embodiment 98: A combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for use in the treatment or prevention of an IKZF2-dependent disease by degrading IKZF2, wherein degradation of IKZF2 treats or prevents the IKZF2-dependent disease.

Embodiment 99: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for the treatment or prevention of an IKZF2-dependent disease by degrading IKZF2, wherein degradation of IKZF2 treats or prevents the IKZF2-dependent disease.

Embodiment 100: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for the manufacture of a medicament for treating or preventing an IKZF2-dependent disease by degrading IKZF2, wherein degradation of IKZF2 treats or prevents the IKZF2-dependent disease.

Embodiment 101: A method for treating a disease that is affected by the modulation of IKZF2 protein levels in a patient comprising administering to the patient in need thereof a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent.

Embodiment 102: A combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for use in the treatment or prevention of a disease that is affected by the modulation of IKZF2 protein levels, wherein modulation of IKZF2 protein levels treats or prevents the disease.

Embodiment 103: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for the treatment or prevention of a disease that is affected by the modulation of IKZF2 protein levels, wherein modulation of IKZF2 protein levels treats or prevents the disease.

Embodiment 104: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for the manufacture of a medicament for treating or preventing a disease that is affected by the modulation of IKZF2 protein levels, wherein modulation of IKZF2 protein levels treats or prevents the disease.

Embodiment 105: A method for treating or preventing a disease that is affected by a decrease or a reduction in IKZF2 protein levels in a patient comprising administering to the patient in need thereof a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent, wherein reduction or decrease of IKZF2 protein levels treats or prevents the disease.

Embodiment 106: A combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for use in the treatment or prevention of a disease that is affected by a decrease or a reduction in IKZF2 protein levels wherein reduction or decrease of IKZF2 protein levels treats or prevents the disease.

Embodiment 107: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for the treatment or prevention of a disease that is affected by a decrease or a reduction in IKZF2 protein levels wherein reduction or decrease of IKZF2 protein levels treats or prevents the disease.

Embodiment 108: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent for the manufacture of a medicament for treating or preventing a disease that is affected by a decrease or a reduction in IKZF2 protein levels wherein reduction or decrease of IKZF2 protein levels treats or prevents the disease.

Embodiment 109: A method of treating or preventing cancer comprising administering to a patient in need thereof a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent, wherein the cancer is a cancer for which the immune response is deficient or an immunogenic cancer.

Embodiment 110: A combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent, for use in the treatment or prevention of cancer, wherein the cancer is a cancer for which the immune response is deficient or an immunogenic cancer.

Embodiment 111: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent, for the treatment or prevention of cancer, wherein the cancer is a cancer for which the immune response is deficient or an immunogenic cancer.

Embodiment 112: Use of a combination comprising (a) a compound according to any one of Embodiments 1-70, or a pharmaceutically acceptable salt, hydrate, solvate, prodrug, stereoisomer, or tautomer thereof, or a pharmaceutical formulation comprising a compound according to any one of Embodiments 1-70, or a pharmaceutically acceptable salt, hydrate, solvate, prodrug, stereoisomer, or tautomer thereof, and a pharmaceutically acceptable carrier or excipient; and (b) a second therapeutic agent, for the manufacture of a medicament for treating or preventing of cancer, wherein the cancer is a cancer for which the immune response is deficient or an immunogenic cancer.

Embodiment 113: A method of treating cancer comprising administering to a patient in need thereof a combination according to any one of Embodiments 71-86.

Embodiment 114: A combination according to any one of Embodiments 71-86 for use in the treatment or prevention of cancer.

Embodiment 115: Use of a combination according to any one of Embodiments 71-86 for the treatment or prevention of cancer.

Embodiment 116: Use of a combination according to any one of Embodiments 71-86 for the manufacture of a medicament for treating or preventing of cancer.

Embodiment 117: A method of treating or preventing cancer comprising administering to a patient in need thereof a combination according to any one of Embodiments 71-86.

Embodiment 118: A method of treating or preventing an IKZF2-dependent disease by reducing or decreasing IKZF2 protein levels in a patient comprising administering to the patient in need thereof a combination according to any one of Embodiments 71-86, wherein reduction or decrease of IKZF2 protein levels treats or prevents the IKZF2-dependent disease.

Embodiment 119: A combination according to any one of Embodiments 71-86 for use in the treatment or prevention of an IKZF2-dependent disease by reducing or decreasing IKZF2 protein levels, wherein reduction or decrease of IKZF2 protein levels treats or prevents the IKZF2-dependent disease.

Embodiment 120: Use of a combination according to any one of Embodiments 71-86 for the treatment or prevention of an IKZF2-dependent disease by reducing or decreasing IKZF2 protein levels, wherein reduction or decrease of IKZF2 protein levels treats or prevents the IKZF2-dependent disease.

Embodiment 121: Use of a combination according to any one of Embodiments 71-86 for the manufacture of a medicament for treating or preventing an IKZF2-dependent disease by reducing or decreasing IKZF2 protein levels, wherein reduction or decrease of IKZF2 protein levels treats or prevents the IKZF2-dependent disease.

Embodiment 122: A method of treating or preventing an IKZF2-dependent disease by degrading IKZF2 in a patient comprising administering to the patient in need thereof a combination according to any one of Embodiments 71-86, wherein degradation of IKZF2 treats or prevents the IKZF2-dependent disease.

Embodiment 123: A combination according to any one of Embodiments 71-86 for use in the treatment or prevention of an IKZF2-dependent disease by degrading IKZF2, wherein degradation of IKZF2 treats or prevents the IKZF2-dependent disease.

Embodiment 124: Use of a combination according to any one of Embodiments 71-86 for the treatment or prevention of an IKZF2-dependent disease by degrading IKZF2, wherein degradation of IKZF2 treats or prevents the IKZF2-dependent disease.

Embodiment 125: Use of a combination according to any one of Embodiments 71-86 for the manufacture of a medicament for treating or preventing an IKZF2-dependent disease by degrading IKZF2, wherein degradation of IKZF2 treats or prevents the IKZF2-dependent disease.

Embodiment 126: A method for treating a disease that is affected by the modulation of IKZF2 protein levels in a patient comprising administering to the patient in need thereof a combination according to any one of Embodiments 71-86.

Embodiment 127: A combination according to any one of Embodiments 71-86 for use in the treatment or prevention of a disease that is affected by the modulation of IKZF2 protein levels, wherein modulation of IKZF2 protein levels treats or prevents the disease.

Embodiment 128: Use of a combination according to any one of Embodiments 71-86 for the treatment or prevention of a disease that is affected by the modulation of IKZF2 protein levels, wherein modulation of IKZF2 protein levels treats or prevents the disease.

Embodiment 129: Use of a combination according to any one of Embodiments 71-86 for the manufacture of a medicament for treating or preventing a disease that is affected by the modulation of IKZF2 protein levels, wherein modulation of IKZF2 protein levels treats or prevents the disease.

Embodiment 130: A method for treating or preventing a disease that is affected by a decrease or a reduction in IKZF2 protein levels in a patient comprising administering to the patient in need thereof a combination according to any one of Embodiments 71-86, wherein reduction or decrease of IKZF2 protein levels treats or prevents the disease.

Embodiment 131: A combination according to any one of Embodiments 71-86 for use in the treatment or prevention of a disease that is affected by a decrease or a reduction in IKZF2 protein levels wherein reduction or decrease of IKZF2 protein levels treats or prevents the disease.

Embodiment 132: Use of a combination according to any one of Embodiments 71-86 for the treatment or prevention of a disease that is affected by a decrease or a reduction in IKZF2 protein levels wherein reduction or decrease of IKZF2 protein levels treats or prevents the disease.

Embodiment 133: Use of a combination according to any one of Embodiments 71-86 for the manufacture of a medicament for treating or preventing a disease that is affected by a decrease or a reduction in IKZF2 protein levels wherein reduction or decrease of IKZF2 protein levels treats or prevents the disease.

Embodiment 134: A method of treating cancer comprising administering to a patient in need thereof a combination according to any one of Embodiments 71-86, wherein the cancer is a cancer for which the immune response is deficient or an immunogenic cancer.

Embodiment 135: A combination according to any one of Embodiments 71-86 for use in the treatment or prevention of cancer, wherein the cancer is a cancer for which the immune response is deficient or an immunogenic cancer.

Embodiment 136: Use of a combination according to any one of Embodiments 71-86 for the treatment or prevention of cancer, wherein the cancer is a cancer for which the immune response is deficient or an immunogenic cancer.

Embodiment 137: Use of a combination according to any one of Embodiments 71-86 for the manufacture of a medicament for treating or preventing of cancer, wherein the cancer is a cancer for which the immune response is deficient or an immunogenic cancer.

Embodiment 138: A method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a second therapeutic agent.

Embodiment 139: The method according to Embodiment 138, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 140: The method according to Embodiment 138 or 139, wherein the cancer is selected from non-small cell lung cancer (NSCLC), melanoma, triple-negative breast cancer (TNBC), nasopharyngeal cancer (NPC), and microsatellite stable colorectal cancer (mssCRC).

Embodiment 141: The method according to any one of Embodiments 138-140, wherein the amount of Compound I-156, Compound I-57, Compound I-87, Compound I-88, Compound I-265, or Compound I-112, or a pharmaceutically acceptable salt, hydrate, solvate, prodrug, stereoisomer, or tautomer thereof, is effective to treat or prevent the cancer.

Embodiment 142: The method according to any one of Embodiments 138-141, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

Embodiment 143: The method according to any one of Embodiments 138-142, wherein the compound is Compound I-156.

Embodiment 144: The method according to any one of Embodiments 138-142, wherein the compound is Compound I-57.

Embodiment 145: The method according to any one of Embodiments 138-142, wherein the compound is Compound I-87.

Embodiment 146: The method according to any one of Embodiments 138-142, wherein the compound is Compound I-88.

Embodiment 147: The method according to any one of Embodiments 138-142, wherein the compound is Compound I-265.

Embodiment 148: The method according to any one of Embodiments 138-142, wherein the compound is Compound I-112.

Embodiment 149: The method according to any one of Embodiments 138-148, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 150: The method according to any one of Embodiments 138-149, wherein the compound is administered orally.

Embodiment 151: The method according to any one of Embodiments 138-150, wherein the second therapeutic agent is administered at a dose of about 100 mg once every four weeks, or about 200 mg once every four weeks, or about 300 mg once every four weeks, or about 400 mg once every four weeks, or about 500 mg once every four weeks.

Embodiment 152: The method according to any one of Embodiments 138-151, wherein the second therapeutic agent is administered at a dose of about 400 mg once every four weeks.

Embodiment 153: The method according to any one of Embodiments 138-152, wherein the second therapeutic agent is administered intravenously.

Embodiment 154: The method according to any one of Embodiments 138-153, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 155: The method according to any one of Embodiments 138-154, wherein the second therapeutic agent is an immunomodulator.

Embodiment 156: The method according to Embodiment 155, wherein the immunomodulator is an immune checkpoint inhibitor.

Embodiment 157: The method according to Embodiment 156, wherein the immune checkpoint inhibitor is a PD-1 inhibitor.

Embodiment 158: The method according to Embodiment 157, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 159: The method according to Embodiment 158, wherein the PD-1 inhibitor is PDR001.

Embodiment 160: A method of treating or preventing cancer comprising administering to a patient in need thereof a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 161: The method according to Embodiment 160, wherein the amount of Compound I-156, Compound I-57, Compound I-87, Compound I-88, Compound I-265, or Compound I-112, or a pharmaceutically acceptable salt, hydrate, solvate, prodrug, stereoisomer, or tautomer thereof, is effective to treat or prevent the cancer.

Embodiment 162: The method according to Embodiment 160 or 161, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 163: The method according to any one of Embodiments 160-162, wherein the cancer is selected from non-small cell lung cancer (NSCLC), melanoma, triple-negative breast cancer (TNBC), nasopharyngeal cancer (NPC), and microsatellite stable colorectal cancer (mssCRC).

Embodiment 164: The method according to any one of Embodiments 160-163, wherein the compound is Compound I-156.

Embodiment 165: The method according to any one of Embodiments 160-163, wherein the compound is Compound I-57.

Embodiment 166: The method according to any one of Embodiments 160-163, wherein the compound is Compound I-87.

Embodiment 167: The method according to any one of Embodiments 160-163, wherein the compound is Compound I-88.

Embodiment 168: The method according to any one of Embodiments 160-163, wherein the compound is Compound I-265.

Embodiment 169: The method according to any one of Embodiments 160-163, wherein the compound is Compound I-112.

Embodiment 170: The method according to any one of Embodiments 160-169, further comprising a second therapeutic agent.

Embodiment 171: The method according to Embodiment 170, wherein the second therapeutic agent is administered at a dose of about 100 mg once every four weeks, or about 200 mg once every four weeks, or about 300 mg once every four weeks, or about 400 mg once every four weeks, or about 500 mg once every four weeks.

Embodiment 172: The method according to Embodiment 170 or 171, wherein the second therapeutic agent is administered at a dose of about 400 mg once every four weeks.

Embodiment 173: The method according to any one of Embodiments 170-172, wherein the second therapeutic agent is administered intravenously.

Embodiment 174: The method according to any one of Embodiments 170-173, wherein the second therapeutic agent is an immunomodulator.

Embodiment 175: The method according to Embodiment 174, wherein the immunomodulator is an immune checkpoint inhibitor.

Embodiment 176: The method according to Embodiment 175, wherein the immune checkpoint inhibitor is a PD-1 inhibitor.

Embodiment 176: The method according to Embodiment 175, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 177: The method according to Embodiment 176, wherein the PD-1 inhibitor is PDR001.

Embodiment 178: The method according to any one of Embodiments 170-177, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

Embodiment 179: The method, compound for use, or the use according to any one of Embodiments 87-178, wherein the method further comprises measuring the level of at least one biomarker selected from IKZF2, PD-L1, CD8, and FOXP3.

Embodiment 180: The method, compound for use, or the use according to Embodiment 179, wherein the level of IKZF2 is reduced.

Embodiment 181: The method, compound for use, or the use according to any one of Embodiments 87-180, wherein the patient was previously treated with an anti-PD-1/PD-L1 therapy.

Embodiment 182: The method, compound for use, or the use according to any one of Embodiments 87-181, wherein the patient being treated for NSCLC or melanoma, or a combination thereof, was primarily refractory to anti-PD-1/PD-L1 therapy agent showing no significant radiologic response during treatment with an anti-PD-1/PD-L1 agent <6 months prior to disease progression.

Embodiment 183: The method, compound for use, or the use according to any one of Embodiments 87-182, wherein the patient being treated for NPC, mssCRC, or TNBC, or a combination thereof, was naive to anti-PD-1/PD-L1 therapy.

Embodiment 184: The method, compound for use, or the use according to any one of Embodiments 87-183, wherein the patient has not been treated with an IKZF2 targeting agent.

Embodiment 185: The method according to any one of Embodiments 87-184, wherein the patient does not show the presence of symptomatic central nervous system (CNS) metastases, or CNS metastases requiring local CNS-directed therapy (such as radiotherapy or surgery), or increasing doses of corticosteroids within 2 weeks prior to the time of the first administration of the compound or the combination comprising the compound and a second agent.

Embodiment 186: The method, compound for use, or the use according to any one of Embodiments 87-185, wherein the patient does not have a history of severe hypersensitivity reactions to any ingredient of study drug(s) and other mAbs and/or their excipients.

Embodiment 187: The method, compound for use, or the use according to any one of Embodiments 87-186, wherein the patient does not have clinically significant cardiac disease or impaired cardiac function.

Embodiment 188: The method, compound for use, or the use according to any one of Embodiments 87-187, wherein the patient does not have any one of the following clinically significant cardiac disease or impaired cardiac function:
(i) clinically significant and/or uncontrolled heart disease such as congestive heart failure requiring treatment with NYHA grade >2;
(ii) uncontrolled hypertension or clinically significant arrhythmia;
(iii) QT interval corrected by Fridericia's formula (QTcF) >450 msec in male patients, or >460 msec female patients;
(iv) QTc that is not assessable;
(v) congenital long QT syndrome;
(vi) history of familial long QT syndrome or known family history of as Torsades de Pointes; and (vii) acute myocardial infarction or unstable angina pectoris <3 months prior to the time of the first administration of the compound or the combination comprising the compound and a second agent.

Embodiment 189: The method, compound for use, or the use according to any one of Embodiments 87-188, wherein the patient does not have HIV infection.

Embodiment 190: The method, compound for use, or the use according any one of Embodiments 87-189, wherein the patient does not have hepatitis B virus (HBV) infection.

Embodiment 191: The method, compound for use, or the use according to any one of Embodiments 87-190, wherein the patient does not have hepatitis C virus (HCV) infection.

Embodiment 192: The method, compound for use, or the use according to any one of Embodiments 87-191, wherein the patient does not have active, known, or suspected autoimmune disease.

Embodiment 193: The method, compound for use, or the use according to any one of Embodiments 87-192, wherein the patient does not have the presence or history of interstitial lung disease or interstitial pneumonitis, including clinically significant radiation or drug-induced pneumonitis.

Embodiment 194: The method, compound for use, or the use according to any one of Embodiments 87-193, wherein the patient has not been treated with
(i) a cytotoxic or targeted antineoplastics within 3 weeks prior to the time of the first administration of the compound or the combination comprising the compound and a second agent;
(ii) systemic chronic steroid therapy (>10 mg/day prednisone or equivalent) or any other immunosuppressive therapy within 7 days prior to the time of the first administration of the compound or the combination comprising the compound and a second agent;
(iii) radiotherapy within 2 weeks prior to the time of the first administration of the compound or the combination comprising the compound and a second agent; or
(iv) any immunosuppressive medication that would interfere with the action of the compound or the combination comprising the compound and a second agent;
or a combination thereof.

Embodiment 195: The method, compound for use, or the use according to any one of Embodiments 87-194, wherein the patient has not been using any live vaccines against infectious diseases within 4 weeks prior to the time of the first administration of the compound or the combination comprising the compound and a second agent; or using hematopoietic colony-stimulating growth factors thrombopoietin mimetics or erythroid stimulating agents within <2 weeks prior to the time of the first administration of the compound or the combination comprising the compound and a second agent.

Embodiment 196: The combination according to Embodiment 88 or 114, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 197: The use according to any one of Embodiments 89, 90, 115, or 116, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 198: The method according to any one of Embodiments 87, 91, 92, 113, or 117, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 199: The combination according to any one of Embodiments 94, 98, 102, 106, 119, 123, 127, or 131, wherein the disease is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 200: The use according to any one of Embodiments 95, 96, 99, 100, 103, 104, 107, 108, 120, 121, 124, 125, 128, 129, 132, or 133, wherein the disease is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 201: The method according to any one of Embodiments 93, 97, 101, 105, 118, 122, 126, or 130, wherein the disease is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 202: A pharmaceutical formulation comprising, a selected from Compound 1-156, Compound I-57, Compound I-87, Compound I-88, Compound I-265, and Compound I-112, and a second agent.

Embodiment 203: The formulation according to Embodiment 202, wherein the compound is Compound I-156.

Embodiment 204: The formulation according to Embodiment 202, wherein the compound is Compound I-57.

Embodiment 205: The formulation according to Embodiment 202, wherein the compound is Compound I-87.

Embodiment 206: The formulation according to Embodiment 202, wherein the compound is Compound I-88.

Embodiment 207: The formulation according to Embodiment 202, wherein the compound is Compound 1-265.

Embodiment 208: The formulation according to Embodiment 202, wherein the compound is Compound I-112.

Embodiment 209: The formulation according to any one of Embodiments 202-208, wherein the formulation comprises about 2 mg, or about 10 mg, or about 20 mg, or about 40 mg, or about 80 mg, or about 160 mg, or about 320 mg of the compound.

Embodiment 210: The formulation according to any one of Embodiments 202-209, wherein the formulation comprises about 100 mg, or about 200 mg, or about 300 mg, or about 400 mg, or about 500 mg of the second therapeutic agent.

Embodiment 211: The formulation according to any one of Embodiments 202-210, wherein the formulation comprises about 2 mg, or about 10 mg, or about 20 mg, or about 40 mg, or about 80 mg, or about 160 mg, or about 320 mg of the compound; and about 100 mg, or about 200 mg, or about 300 mg, or about 400 mg, or about 500 mg of the second therapeutic agent.

Embodiment 212: The formulation according to any one of Embodiments 202-211, wherein the formulation comprises about 400 mg of the second therapeutic agent.

Embodiment 213: The formulation according to any one of Embodiments 202-212, wherein the second therapeutic agent is an immunomodulator.

Embodiment 214: The formulation according to Embodiment 213, wherein the immunomodulator is an immune checkpoint inhibitor.

Embodiment 215: The formulation according to Embodiment 214, wherein the immune checkpoint inhibitor is a PD-1 inhibitor.

Embodiment 216: The formulation according to Embodiment 215, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 217: The formulation according to Embodiment 216, wherein the PD-1 inhibitor is PDR001.

Embodiment 218: The combination according to Embodiment 1a or formulation according to Embodiment 1b, wherein the combination comprises about 2 mg, or about 10 mg, or about 20 mg, or about 40 mg, or about 80 mg, or about 160 mg, or about 320 mg of the compound.

Embodiment 219: The combination according to Embodiment 1a or formulation according to Embodiment 1b, wherein the combination comprises between about 1 to about 10 mg, or between about 10 mg to about 20 mg, or between about 20 to about 30 mg, or between about 30 mg to about 40 mg, or between about 40 mg to about 50 mg, or between about 50 mg to about 60 mg, or between about 60 mg to about 70 mg, or between about 70 mg to about 80 mg, or between about 80 mg to about 90 mg, or between about 90 mg to about 100 mg, or between about 100 mg to about 110 mg, or between about 110 mg to about 120 mg, or between about 120 mg to about 130 mg, or between about 130 mg to about 140 mg, or between about 140 mg to about 150 mg, or between about 150 mg to about 160 mg, or between about 160 mg to about 170 mg, or between about 170 mg to about 180 mg, or between about 180 mg to about 190 mg, or between about 190 mg to about 200 mg, or between about 200 mg to about 210 mg, or between about 210 mg to about 220 mg, or between about 220 mg to about 230 mg, or between about 230 mg to about 240 mg, or between about 240 mg to about 250 mg, or between about 250 mg to about 260 mg, or between about 260 mg to about 270 mg, or between about 270 mg to about 280 mg, or between about 280 mg to about 290 mg, or between about 290 mg to about 300 mg, or between about 300 mg to about 310 mg, or between about 310 mg to about 320 mg, or between about 320 mg to about 330 mg, or between about 330 mg to about 340 mg, or between about 340 mg to about 350 mg, or between about 350 mg to about 360 mg, or between about 360 mg to about 370 mg, or between about 370 mg to about 380 mg, or between about 380 mg to about 390 mg, or between about 390 mg to about 400 mg, or between about 400 mg to about 420 mg, or between about 420 mg to about 430 mg, or between about 430 mg to about 440 mg, or between about 440 mg to about 450 mg, or between about 450 mg to about 460 mg, or between about 460 mg to about 470 mg, or between about 470 mg to about 480 mg, or between about 480 mg to about 490 mg, or between about 490 mg to about 500 mg of the compound.

Embodiment 220: The combination according to Embodiment 1a or formulation according to Embodiment 1b, wherein the combination comprises about 0.1 mg, or about 0.5 mg, or about 1 mg, or about 2 mg, or about 3 mg, or about 4 mg, or about 5 mg, or about 10 mg, or about 15 mg, or about 20 mg, or about 25 mg, or about 30 mg, or about 35 mg, or about 40 mg, or about 45 mg, or about 50 mg, or about 55 mg, or about 60 mg, or about 65 mg, or about 70 mg, or about 75 mg, or about 80 mg, or about 85 mg, or about 90 mg, or about 95 mg, or about 100 mg, or about 110 mg, or about 120 mg, or about 130 mg, or about 140 mg, or about 150 mg, or about 160 mg, or about 170 mg, or about 180 mg, or about 190 mg, or about 200 mg, or about 210 mg, or about 220 mg, or about 230 mg, or about 240 mg, or about 250 mg, or about 260 mg, or about 270 mg, or about 280 mg, or about 290 mg, or about 300 mg, or about 310 mg, or about 320 mg, or about 330 mg, or about 340 mg, or about 350 mg, or about 360 mg, or about 370 mg, or about 380 mg, or about 390 mg, or about 400 mg, or about 410 mg, or about 420 mg, or about 430 mg, or about 440 mg, or about 450 mg, or about 460 mg, or about 470 mg, or about 480 mg, or about 500 mg of the compound.

Embodiment 221: The combination according to Embodiment 1a or formulation according to Embodiment 1b, wherein the combination comprises about 100 mg, or about 200 mg, or about 300 mg, or about 400 mg, or about 500 mg of the second therapeutic agent.

Embodiment 222: The combination according to Embodiment 1a or formulation according to Embodiment 1b, wherein the second therapeutic agent is an immunomodulator.

Embodiment 223: The combination according to Embodiment 1a or formulation according to Embodiment 1b, wherein the second therapeutic agent.is an immune checkpoint inhibitor.

Embodiment 224: The combination according to Embodiment 1a or formulation according to Embodiment 1b, wherein the second therapeutic agent.is a PD-1 inhibitor.

Embodiment 225: The combination according to Embodiment 1a or formulation according to Embodiment 1b, wherein the second therapeutic agent is a PD-1 inhibitor selected from PDR001, Nivolumab, Pembrolizumab, Pidilizumab, MEDI0680, REGN2810, TSR-042, PF-06801591, BGB-A317, BGB-108, INCSHR1210, and AMP-224.

Embodiment 226: The combination according to Embodiment 1a or formulation according to Embodiment 1b, wherein the second therapeutic agent is PDR001.

Embodiment 227: The combination according to Embodiment 1a or formulation according to Embodiment 1b, wherein the second agent is selected from a PD-1 inhibitor, a LAG-3 inhibitor, a cytokine, an A2A antagonist, a GITR agonist, a TIM-3 inhibitor, a STING agonist, and a TLR7 agonist.

Embodiment 228: The combination according to Embodiment 1a or formulation according to Embodiment 1b, wherein the second agent is a LAG-3 inhibitor.

Embodiment 229: The combination according to Embodiment 1a or formulation according to Embodiment 1b, wherein the second agent is a cytokine.

Embodiment 230: The combination according to Embodiment 1a or formulation according to Embodiment 1b, wherein the second agent is an A2A antagonist.

Embodiment 231: The combination according to Embodiment 1a or formulation according to Embodiment 1b, wherein the second agent is a GITR agonist.

Embodiment 232: The combination according to Embodiment 1a or formulation according to Embodiment 1b, wherein the second agent is a TIM-3 inhibitor.

Embodiment 233: The combination according to Embodiment 1a or formulation according to Embodiment 1b, wherein the second agent is a STING agonist.

Embodiment 234: The combination according to Embodiment 1a or formulation according to Embodiment 1b, wherein the second agent is a TLR7 agonist.

Embodiment 235: The combination according to Embodiment 1a or formulation according to Embodiment 1b, wherein the combination comprises between about 10 to about 50 mg, or between about 50 to about 100 mg, or between about 100 to about 200 mg, or between about 200 mg to about 300 mg, or between about 300 mg to about 400 mg, or between about 400 mg to about 500 mg or between about 500 mg to about 600 mg, or between about 600 mg to about 700 mg of the second therapeutic agent.

Embodiment 236: The combination according to Embodiment 1a or formulation according to Embodiment 1b, wherein the combination comprises between about 10 to about 50 mg, or between about 50 to about 100 mg, or between about 100 to about 150 mg, or between about 150 mg to about 200 mg, or between about 200 mg to about 250 mg, or between about 250 mg to about 300 mg or between about 350 mg to about 400 mg, or between about 400 mg to about 450 mg, or between about 450 mg to about 500 mg, or between about 500 mg to about 550 mg, or between about 550 mg to about 600 mg, or between about 600 mg to about 650 mg, or between about 650 mg to about 750 mg of the second therapeutic agent.

Embodiment 237: The combination according to Embodiment 1a or formulation according to Embodiment 1b, wherein the combination comprises 100 mg, or 200 mg, or 300 mg, or 400 mg, or 500 mg of the second therapeutic agent.

Embodiment 238: The combination according to Embodiment 1a or formulation according to Embodiment 1b, wherein the combination comprises between 10 to 50 mg, or between 50 to 100 mg, or between 100 to 200 mg, or between 200 mg to 300 mg, or between 300 mg to 400 mg, or between 400 mg to 500 mg or between 500 mg to 600 mg, or between 600 mg to 700 mg of the second therapeutic agent.

Embodiment 239: The combination according to Embodiment 1a or formulation according to Embodiment 1b, wherein the combination comprises between 10 to 50 mg, or between 50 to 100 mg, or between 100 to 150 mg, or between 150 mg to 200 mg, or between 200 mg to 250 mg, or between 250 mg to 300 mg or between 350 mg to 400 mg, or between 400 mg to 450 mg, or between 450 mg to 500 mg, or between 500 mg to 550 mg, or between 550 mg to 600 mg, or between 600 mg to 650 mg, or between 650 mg to 750 mg of the second therapeutic agent.

Embodiment 240: The combination according to Embodiment 1a or formulation according to Embodiment 1b, wherein the combination comprises 2 mg, or 10 mg, or 20 mg, or 40 mg, or 80 mg, or 160 mg, or 320 mg of the compound.

Embodiment 241: The combination according to Embodiment 1a or formulation according to Embodiment 1b, wherein the combination comprises between 1 to 10 mg, or between 10 mg to 20 mg, or between 20 to 30 mg, or between 30 mg to 40 mg, or between 40 mg to 50 mg, or between 50 mg to 60 mg, or between 60 mg to 70 mg, or between 70 mg to 80 mg, or between 80 mg to 90 mg, or between 90 mg to 100 mg, or between 100 mg to 110 mg, or between 110 mg to 120 mg, or between 120 mg to 130 mg, or between 130 mg to 140 mg, or between 140 mg to 150 mg, or between 150 mg to 160 mg, or between 160 mg to 170 mg, or between 170 mg to 180 mg, or between 180 mg to 190 mg, or between 190 mg to 200 mg, or between 200 mg to 210 mg, or between 210 mg to 220 mg, or between 220 mg to 230 mg, or between 230 mg to 240 mg, or between 240 mg to 250 mg, or between 250 mg to 260 mg, or between 260 mg to 270 mg, or between 270 mg to 280 mg, or between 280 mg to 290 mg, or between 290 mg to 300 mg, or between 300 mg to 310 mg, or between 310 mg to 320 mg, or between 320 mg to 330 mg, or between 330 mg to 340 mg, or between 340 mg to 350 mg, or between 350 mg to 360 mg, or between 360 mg to 370 mg, or between 370 mg to 380 mg, or between 380 mg to 390 mg, or between 390 mg to 400 mg, or between 400 mg to 420 mg, or between 420 mg to 430 mg, or between 430 mg to 440 mg, or between 440 mg to 450 mg, or between 450 mg to 460 mg, or between 460 mg to 470 mg, or between 470 mg to 480 mg, or between 480 mg to 490 mg, or between 490 mg to 500 mg of the compound.

Embodiment 242: The combination according to Embodiment 1a or formulation according to Embodiment 1b, wherein the combination comprises 0.1 mg, or 0.5 mg, or 1 mg, or 2 mg, or 3 mg, or 4 mg, or 5 mg, or 10 mg, or 15 mg, or 20 mg, or 25 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg, or 110 mg, or 120 mg, or 130 mg, or 140 mg, or 150 mg, or 160 mg, or 170 mg, or 180 mg, or 190 mg, or 200 mg, or 210 mg, or 220 mg, or 230 mg, or 240 mg, or 250 mg, or 260 mg, or 270 mg, or 280 mg, or 290 mg, or 300 mg, or 310 mg, or 320 mg, or 330 mg, or 340 mg, or 350 mg, or 360 mg, or 370 mg, or 380 mg, or 390 mg, or 400 mg, or 410 mg, or 420 mg, or 430 mg, or 440 mg, or 450 mg, or 460 mg, or 470 mg, or 480 mg, or 500 mg of the compound.

Embodiment 243: The method according to any one of Embodiments 87-190, wherein the patient has received prior treatment with an IKZF2 targeted agent; or the patient does not have the presence of symptomatic central nervous system (CNS) metastases, or CNS metastases that require local CNS-directed therapy (such as radiotherapy or surgery), or increasing doses of corticosteroids within the prior 2 weeks; or the patient does not have a history of severe hypersensitivity reactions to any ingredient of study drug(s) and other mAbs and/or their excipients; or the patient does not have impaired cardiac function or clinically significant cardiac disease; the patient does not have HIV infection; or the patient does not have hepatitis B virus (HBV) or hepatitis C virus (HCV) infection; or the patient does not have active, known or suspected autoimmune disease; and/or the patient does not have presence or history of interstitial lung disease or interstitial pneumonitis, including clinically significant radiation or drug-induced pneumonitis (i.e., affecting activities of daily living or requiring therapeutic intervention).

Embodiment 244: The method according to any one of Embodiments 87-190, wherein the patient have one or more of the following: (a) advanced/metastatic NSCLC, melanoma, NPC, mssCRC or TNBC; (b) have received standard therapy in the metastatic setting, are intolerant to standard therapy, or no effective therapy is available; (c) have a site of disease amenable to core needle biopsy, and be a candidate for tumor biopsy according to the treating institution's guidelines.

In some embodiments, the combination or formulation comprises about 2 mg, or about 10 mg, or about 20 mg, or about 40 mg, or about 80 mg, or about 160 mg, or about 320 mg of the compound.

In some embodiments, the combination or formulation comprises between about 1 to about 10 mg, or between about 10 mg to about 20 mg, or between about 20 to about 30 mg, or between about 30 mg to about 40 mg, or between about 40 mg to about 50 mg, or between about 50 mg to about 60 mg, or between about 60 mg to about 70 mg, or between about 70 mg to about 80 mg, or between about 80 mg to about 90 mg, or between about 90 mg to about 100 mg, or between about 100 mg to about 110 mg, or between about 110 mg to about 120 mg, or between about 120 mg to about 130 mg, or between about 130 mg to about 140 mg, or between about 140 mg to about 150 mg, or between about 150 mg to about 160 mg, or between about 160 mg to about 170 mg, or between about 170 mg to about 180 mg, or between about 180 mg to about 190 mg, or between about 190 mg to about 200 mg, or between about 200 mg to about 210 mg, or between about 210 mg to about 220 mg, or between about 220 mg to about 230 mg, or between about 230 mg to about 240 mg, or between about 240 mg to about 250 mg, or between about 250 mg to about 260 mg, or between about 260 mg to about 270 mg, or between about 270 mg to about 280 mg, or between about 280 mg to about 290 mg, or between about 290 mg to about 300 mg, or between about 300 mg to about 310 mg, or between about 310 mg to about 320 mg, or between about 320 mg to about 330 mg, or between about 330 mg to about 340 mg, or between about 340 mg to about 350 mg, or between about 350 mg to about 360 mg, or between about 360 mg to about 370 mg, or between about 370 mg to about 380 mg, or between about 380 mg to about 390 mg, or between about 390 mg to about 400 mg, or between about 400 mg to about 420 mg, or between about 420 mg to about 430 mg, or between about 430 mg to about 440 mg, or between about 440 mg to about 450 mg, or between about 450 mg to about 460 mg, or between about 460 mg to about 470 mg, or between about 470 mg to about 480 mg, or between about 480 mg to about 490 mg, or between about 490 mg to about 500 mg of the compound.

Embodiment 245: The method, compound for use, or the use according to any one of Embodiments 87-201, 243 or 244, wherein the combination is administered simultaneously, separately, or over a period of time.

Embodiment 246: A method of treating or preventing cancer comprising administering to a patient in need thereof a combination comprising, (a) a compound of Formula (Ic), or a pharmaceutically acceptable salt, hydrate, solvate, prodrug, stereoisomer, or tautomer thereof, wherein: each R₁ is independently (C₁-C₆)alkyl, (C₁-C₆)haloalkyl, (C₁-C₆)hydroxyalkyl, or halogen, or two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₅)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₃-C₇)cycloalkyl ring or a 5- to 7-membered heterocycloalkyl ring comprising 1 to 3 heteroatoms selected from O, N, and S optionally substituted with one or more R₁₀;
R₆ and R₆, are each independently H, (C₁-C₆)alkyl, or (C₆-C₁₀)aryl;
each R is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more Rn, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S; and
q is 0, 1, 2, 3, or 4; and
(b) a second therapeutic agent.

Embodiment 247: The method according to Embodiment 246, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

Embodiment 248: The method according to Embodiment 246 or 247, wherein the cancer is selected from non-small cell lung cancer (NSCLC), melanoma, triple-negative breast cancer (TNBC), nasopharyngeal cancer (NPC), and microsatellite stable colorectal cancer (mssCRC).

Embodiment 249: The method according to any one of Embodiments 246-248, wherein the compound and the second agent are administered simultaneously, separately, or over a period of time.

Embodiment 250: The method according to any one of Embodiments 246-249, wherein the amount of the compound of Formula (Ic), or a pharmaceutically acceptable salt, hydrate, solvate, prodrug, stereoisomer, or tautomer thereof, administered to the patient in need thereof is effective to treat or prevent the cancer.

Embodiment 251: The method according to any one of Embodiments 246-250, wherein the amounts of: (a) compound of Formula (Ic), or a pharmaceutically acceptable salt, hydrate, solvate, prodrug, stereoisomer, or tautomer thereof; and (b) the second therapeutic agent, administered to the patient in need thereof are effective to treat or prevent the cancer.

Embodiment 252: The method according to any one of Embodiments 246-251, wherein the compound of Formula (Ic) is selected from (I-156), (I-57), (I-87), (I-88), (I-265), and (I-112), or a pharmaceutically acceptable salt, hydrate, solvate, prodrug, stereoisomer, or tautomer thereof.

Embodiment 253: The method according to any one of Embodiments 246-252, wherein the compound of Formula (Ic) is Compound I-156.

Embodiment 254: The method according to any one of Embodiments 246-252, wherein the compound of Formula (Ic) is Compound I-57.

Embodiment 255: The method according to any one of Embodiments 246-252, wherein the compound of Formula (Ic) is Compound I-87.

Embodiment 256: The method according to any one of Embodiments 246-252, wherein the compound of Formula (Ic) is Compound I-88.

Embodiment 257: The method according to any one of Embodiments 246-252, wherein the compound of Formula (Ic) is Compound I-265.

Embodiment 258: The method according to any one of Embodiments 246-252, wherein the compound of Formula (Ic) is Compound I-112.

Embodiment 259: The method according to any one of Embodiments 246-258, wherein the second therapeutic agent is an immunomodulator.

Embodiment 260: The method according to Embodiment 259, wherein the second therapeutic agent is an immune checkpoint inhibitor.

Embodiment 261: The method according to Embodiment 260, wherein the second therapeutic agent is a PD-1 inhibitor.

Embodiment 262: The method according to Embodiment 261, wherein the PD-1 inhibitor is PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 263: The method according to Embodiment 262, wherein the PD-1 inhibitor is PDR001.

Embodiment 264: The method according to any one of Embodiments 246-263, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 265: The method according to any one of Embodiments 246-264, wherein the compound is administered orally.

Embodiment 266: The method according to any one of Embodiments 246-265, wherein the second therapeutic agent is administered at a dose of about 100 mg once every four weeks, or about 200 mg once every four weeks, or about 300 mg once every four weeks, or about 400 mg once every four weeks, or about 500 mg once every four weeks.

Embodiment 267: The method according to any one of Embodiments 246-266, wherein the second therapeutic agent is administered at a dose of about 400 mg once every four weeks.

Embodiment 268: The method according to any one of Embodiments 246-267, wherein the second therapeutic agent is administered intravenously.

Embodiment 269: The method according to any one of Embodiments 246-268, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 270: A method of treating or preventing cancer comprising administering to a patient in need thereof a compound of Formula (Ic),or a pharmaceutically acceptable salt, hydrate, solvate, prodrug, stereoisomer, or tautomer thereof, wherein:
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₅)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R₆, are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR8R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more Rn, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S; and
q is 0, 1, 2, 3, or 4;
wherein the compound of Formula (Ic) is administered orally at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 271: The method according to Embodiment 270, wherein the amount of the compound of Formula (Ic), or a pharmaceutically acceptable salt, hydrate, solvate, prodrug, stereoisomer, or tautomer thereof, is effective to treat or prevent the cancer.

Embodiment 272: The method according to Embodiment 270 or 271, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

Embodiment 273: The method according to any one of Embodiments 270-272, wherein the cancer is selected from non-small cell lung cancer (NSCLC), melanoma, triple-negative breast cancer (TNBC), nasopharyngeal cancer (NPC), and microsatellite stable colorectal cancer (mssCRC).

Embodiment 274: The method according to any one of Embodiments 270-273, wherein the compound of Formula (Ic) is selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof.

Embodiment 275: The method according to any one of Embodiments 270-274, wherein the compound of Formula (Ic) is Compound I-156.

Embodiment 276: The method according to any one of Embodiments 270-272, wherein the compound of Formula (Ic) is Compound I-57.

Embodiment 277: The method according to any one of Embodiments 270-272, wherein the compound of Formula (Ic) is Compound I-87.

Embodiment 278: The method according to any one of Embodiments 270-272, wherein the compound of Formula (Ic) is Compound I-88.

Embodiment 279: The method according to any one of Embodiments 270-272, wherein the compound of Formula (Ic) is Compound I-265.

Embodiment 280: The method according to any one of Embodiments 270-272, wherein the compound of Formula (Ic) is Compound I-112.

Embodiment 281: The method according to any one of Embodiments 270-272 further comprising a second therapeutic agent.

Embodiment 282: The method according to Embodiment 281, wherein the compound and the second agent are administered simultaneously, separately, or over a period of time.

Embodiment 283: The method according to Embodiment 281 or 282, wherein the second therapeutic agent is an immunomodulator.

Embodiment 284: The method according to Embodiment 283, wherein the immunomodulator is an immune checkpoint inhibitor.

Embodiment 285: The method according to Embodiment 284, wherein the immune checkpoint inhibitor is a PD-1 inhibitor.

Embodiment 286: The method according to Embodiment 285, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 287: The method according to Embodiment 286, wherein the PD-1 inhibitor is PDR001.

Embodiment 288: The method according to any one of Embodiments 270-287, wherein the second therapeutic agent is administered at a dose of about 100 mg once every four weeks, or about 200 mg once every four weeks, or about 300 mg once every four weeks, or about 400 mg once every four weeks, or about 500 mg once every four weeks.

Embodiment 289: The method according to any one of Embodiments 270-288, wherein the second therapeutic agent is administered at a dose of about 400 mg once every four weeks.

Embodiment 290: The method according to any one of Embodiments 270-289, wherein the second therapeutic agent is administered intravenously.

Embodiment 291: The method according to any one of Embodiments 270-290, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

Embodiment 292: The method according to any one of Embodiments 270-291, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

Embodiment 293: The method according to any one of Embodiments 246-292, wherein the method further comprises measuring the level of at least one biomarker selected from IKZF2, PD-L1, CD8, and FOXP3.

Embodiment 294: The method according to Embodiment 293, wherein the level of IKZF2 is reduced.

Embodiment 295: The method according to any one of Embodiments 246-294, wherein the patient was previously treated with an anti-PD-1/PD-L1 therapy.

Embodiment 296: The method according to any one of Embodiments 246-295, wherein the patient being treated for NSCLC or melanoma, or a combination thereof, was primarily refractory to anti-PD-1/PD-L1 therapy agent showing no significant radiologic response during treatment with an anti-PD-1/PD-L1 agent <6 months prior to disease progression.

Embodiment 297: The method according to any one of Embodiments 246-295, wherein the patient being treated for NPC, mssCRC, or TNBC, or a combination thereof, was naive to anti-PD-1/PD-L1 therapy.

Embodiment 298: The method according to any one of Embodiments 246-297, wherein the patient has not been treated with an IKZF2 targeting agent.

Embodiment 299: The method according to any one of Embodiments 246-298, wherein the patient does not show the presence of symptomatic central nervous system (CNS) metastases, or CNS metastases requiring local CNS-directed therapy (such as radiotherapy or surgery), or increasing doses of corticosteroids within 2 weeks prior to the time of the first administration of the compound or the combination comprising the compound and a second agent.

Embodiment 300: The method according to any one of Embodiments 246-299, wherein the patient does not have a history of severe hypersensitivity reactions to any ingredient of study drug(s) and other mAbs and/or their excipients.

Embodiment 301: The method according to any one of Embodiments 246-300, wherein the patient does not have clinically significant cardiac disease or impaired cardiac function.

Embodiment 302: The method according to any one of Embodiments 246-301, wherein the patient does not have any one of the following clinically significant cardiac disease or impaired cardiac function: (i) clinically significant and/or uncontrolled heart disease such as congestive heart failure requiring treatment with NYHA grade >2;
(ii) uncontrolled hypertension or clinically significant arrhythmia;
(iii) QT interval corrected by Fridericia's formula (QTcF) >450 msec in male patients, or >460 msec female patients;
(iv) QTc that is not assessable;
(v) congenital long QT syndrome;
(vi) history of familial long QT syndrome or known family history of as Torsades de Pointes; and
(vii) acute myocardial infarction or unstable angina pectoris <3 months prior to the time of the first administration of the compound or the combination comprising the compound and a second agent.

Embodiment 303: The method according to any one of Embodiments 246-302, wherein the patient does not have HIV infection.

Embodiment 304: The method according to any one of Embodiments 246-303, wherein the patient does not have hepatitis B virus (HBV) infection.

Embodiment 305: The method according to any one of Embodiments 246-304, wherein the patient does not have hepatitis C virus (HCV) infection.

Embodiment 306: The method according to any one of Embodiments 246-305, wherein the patient does not have active, known, or suspected autoimmune disease.

Embodiment 307: The method according to any one of Embodiments 246-306, wherein the patient does not have the presence or history of interstitial lung disease or interstitial pneumonitis, including clinically significant radiation or drug-induced pneumonitis.

Embodiment 308: The method according to any one of Embodiments 246-307, wherein the patient has not been treated with
(i) a cytotoxic or targeted antineoplastics within 3 weeks prior to the time of the first administration of the compound or the combination comprising the compound and a second agent;
(ii) systemic chronic steroid therapy (>10 mg/day prednisone or equivalent) or any other immunosuppressive therapy within 7 days prior to the time of the first administration of the compound or the combination comprising the compound and a second agent;
(iii) radiotherapy within 2 weeks prior to the time of the first administration of the compound or the combination comprising the compound and a second agent; or
(iv) any immunosuppressive medication that would interfere with the action of the compound or the combination comprising the compound and a second agent;
or a combination thereof.

Embodiment 309: The method according to any one of Embodiments 246-308, wherein the patient has not been using any live vaccines against infectious diseases within 4 weeks prior to the time of the first administration of the compound or the combination comprising the compound and a second agent; or using hematopoietic colony-stimulating growth factors thrombopoietin mimetics or erythroid stimulating agents within <2 weeks prior to the time of the first administration of the compound or the combination comprising the compound and a second agent.

Embodiment 310: The method according to any one of Embodiments 246-258, wherein the second therapeutic agent is a LAG-3 inhibitor.

Embodiment 311: The method according to any one of Embodiments 246-258 and 310, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 312: The method according to any one of Embodiments 246-258, 310, and 311, wherein the compound is administered orally.

Embodiment 313: The method according to any one of Embodiments 246-258 and 310-312, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 314: The method according to Embodiment 281 or 282, wherein the second therapeutic agent is a LAG-3 inhibitor.

Embodiment 315: The method according to any one of Embodiments 270-282 and 314, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

Embodiment 316: The method according to any one of Embodiments 270-282, 314, and 315, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

Embodiment 317: The method according to any one of Embodiments 246-258, wherein the second therapeutic agent is a cytokine.

Embodiment 318: The method according to any one of Embodiments 246-258 and 317, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 319: The method according to any one of Embodiments 246-258, 317, and 318, wherein the compound is administered orally.

Embodiment 320: The method according to any one of Embodiments 246-258 and 317-319, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 321: The method according to Embodiment 281 or 282, wherein the second therapeutic agent is a cytokine.

Embodiment 322: The method according to any one of Embodiments 270-282 and 321, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

Embodiment 323: The method according to any one of Embodiments 270-282, 321, and 322, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

Embodiment 324: The method according to any one of Embodiments 246-258, wherein the second therapeutic agent is an A2A antagonist.

Embodiment 325: The method according to any one of Embodiments 246-258 and 324, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 326: The method according to any one of Embodiments 246-258, 324, and 325, wherein the compound is administered orally.

Embodiment 327: The method according to any one of Embodiments 246-258 and 324-326, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 328: The method according to Embodiment 281 or 282, wherein the second therapeutic agent is an A2A antagonist.

Embodiment 329: The method according to any one of Embodiments 270-282 and 328, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

Embodiment 330: The method according to any one of Embodiments 270-282, 328, and 329, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

Embodiment 331: The method according to any one of Embodiments 246-258, wherein the second therapeutic agent is a GITR agonist.

Embodiment 332: The method according to any one of Embodiments 246-258 and 324, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 333: The method according to any one of Embodiments 246-258, 331, and 332, wherein the compound is administered orally.

Embodiment 334: The method according to any one of Embodiments 246-258 and 331-333, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 335: The method according to Embodiment 281 or 282, wherein the second therapeutic agent is a GITR agonist.

Embodiment 336: The method according to any one of Embodiments 270-282 and 335, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

Embodiment 337: The method according to any one of Embodiments 270-282, 335, and 336, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent Embodiment 338: The method according to any one of Embodiments 246-258, wherein the second therapeutic agent is a TIM-3 inhibitor.

Embodiment 339: The method according to any one of Embodiments 246-258 and 338, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 340: The method according to any one of Embodiments 246-258, 338, and 339, wherein the compound is administered orally.

Embodiment 341: The method according to any one of Embodiments 246-258 and 338-340, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 342: The method according to Embodiment 281 or 282, wherein the second therapeutic agent is a TIM-3 inhibitor.

Embodiment 343: The method according to any one of Embodiments 270-282 and 342, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

Embodiment 344: The method according to any one of Embodiments 270-282, 342, and 343, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent Embodiment 345: The method according to any one of Embodiments 246-258, wherein the second therapeutic agent is a STING agonist.

Embodiment 346: The method according to any one of Embodiments 246-258 and 345, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 347: The method according to any one of Embodiments 246-258, 345, and 346, wherein the compound is administered orally.

Embodiment 348: The method according to any one of Embodiments 246-258 and 345-347, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 349: The method according to Embodiment 281 or 282, wherein the second therapeutic agent is a STING agonist.

Embodiment 350: The method according to any one of Embodiments 270-282 and 349, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

Embodiment 351: The method according to any one of Embodiments 270-282, 349, and 350, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent Embodiment 352: The method according to any one of Embodiments 246-258, wherein the second therapeutic agent is a TLR7 agonist.

Embodiment 353: The method according to any one of Embodiments 246-258 and 352, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 354: The method according to any one of Embodiments 246-258, 352, and 353, wherein the compound is administered orally.

Embodiment 355: The method according to any one of Embodiments 246-258 and 352-354, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 356: The method according to Embodiment 281 or 282, wherein the second therapeutic agent is a TLR7 agonist.

Embodiment 357: The method according to any one of Embodiments 270-282 and 356, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

Embodiment 358: The method according to any one of Embodiments 270-282, 356, and 357, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

Embodiment 359: The method according to any one of Embodiments 246-258, 270-282, and 310-358, wherein the method further comprises measuring the level of at least one biomarker selected from IKZF2, PD-L1, CD8, and FOXP3.

Embodiment 360: The method according to Embodiment 359, wherein the level of IKZF2 is reduced.

Embodiment 361: The method according to any one of Embodiments 246-258, 270-282, and 310-360, wherein the patient was previously treated with an anti-PD-1/PD-L1 therapy.

Embodiment 362: The method according to any one of Embodiments 246-258, 270-282, and 310-361, wherein the patient being treated for NSCLC or melanoma, or a combination thereof, was primarily refractory to anti-PD-1/PD-L1 therapy agent showing no significant radiologic response during treatment with an anti-PD-1/PD-L1 agent <6 months prior to disease progression.

Embodiment 363: The method according to any one of Embodiments 246-258, 270-282, and 310-362, wherein the patient being treated for NPC, mssCRC, or TNBC, or a combination thereof, was naive to anti-PD-1/PD-L1 therapy.

Embodiment 364: The method according to any one of Embodiments 246-258, 270-282, and 310-363, wherein the patient has not been treated with an IKZF2 targeting agent.

Embodiment 365: The method according to any one of Embodiments 246-258, 270-282, and 310-364, wherein the patient does not show the presence of symptomatic central nervous system (CNS) metastases, or CNS metastases requiring local CNS-directed therapy (such as radiotherapy or surgery), or increasing doses of corticosteroids within 2 weeks prior to the time of the first administration of the compound or the combination comprising the compound and a second agent.

Embodiment 366: The method according to any one of Embodiments 246-258, 270-282, and 310-365, wherein the patient does not have a history of severe hypersensitivity reactions to any ingredient of study drug(s) and other mAbs and/or their excipients.

Embodiment 367: The method according to any one of Embodiments 246-258, 270-282, and 310-366, wherein the patient does not have clinically significant cardiac disease or impaired cardiac function.

Embodiment 368: The method according to any one of Embodiments 246-258, 270-282, and 310-367, wherein the patient does not have any one of the following clinically significant cardiac disease or impaired cardiac function:
(i) clinically significant and/or uncontrolled heart disease such as congestive heart failure requiring treatment with NYHA grade >2;
(ii) uncontrolled hypertension or clinically significant arrhythmia;
(iii) QT interval corrected by Fridericia's formula (QTcF) >450 msec in male patients, or >460 msec female patients;
(iv) QTc that is not assessable;
(v) congenital long QT syndrome;
(vi) history of familial long QT syndrome or known family history of as Torsades de Pointes; and
(vii) acute myocardial infarction or unstable angina pectoris <3 months prior to the time of the first administration of the compound or the combination comprising the compound and a second agent.

Embodiment 369: The method according to any one of Embodiments 246-258, 270-282, and 310-368, wherein the patient does not have HIV infection.

Embodiment 370: The method according to any one of Embodiments 246-258, 270-282, and 310-369, wherein the patient does not have hepatitis B virus (HBV) infection.

Embodiment 371: The method according to any one of Embodiments 246-258, 270-282, and 310-370, wherein the patient does not have hepatitis C virus (HCV) infection.

Embodiment 372: The method according to any one of Embodiments 246-258, 270-282, and 310-371, wherein the patient does not have active, known, or suspected autoimmune disease.

Embodiment 373: The method according to any one of Embodiments 246-258, 270-282, and 310-372, wherein the patient does not have the presence or history of interstitial lung disease or interstitial pneumonitis, including clinically significant radiation or drug-induced pneumonitis.

Embodiment 374: The method according to any one of Embodiments 246-258, 270-282, and 310-373, wherein the patient has not been treated with
(i) a cytotoxic or targeted antineoplastics within 3 weeks prior to the time of the first administration of the compound or the combination comprising the compound and a second agent;
(ii) systemic chronic steroid therapy (>10 mg/day prednisone or equivalent) or any other immunosuppressive therapy within 7 days prior to the time of the first administration of the compound or the combination comprising the compound and a second agent;
(iii) radiotherapy within 2 weeks prior to the time of the first administration of the compound or the combination comprising the compound and a second agent; or
(iv) any immunosuppressive medication that would interfere with the action of the compound or the combination comprising the compound and a second agent;
or a combination thereof.

Embodiment 375: The method according to any one of Embodiments 246-258, 270-282, and 310-374, wherein the patient has not been using any live vaccines against infectious diseases within 4 weeks prior to the time of the first administration of the compound or the combination comprising the compound and a second agent; or using hematopoietic colony-stimulating growth factors thrombopoietin mimetics or erythroid stimulating agents within <2 weeks prior to the time of the first administration of the compound or the combination comprising the compound and a second agent.

Embodiment 376: A pharmaceutical combination comprising, (a) a compound of Formula (Ic), or a pharmaceutically acceptable salt, hydrate, solvate, prodrug, stereoisomer, or tautomer thereof, wherein: each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₅)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₃-C₇)cycloalkyl ring or a 5- to 7-membered heterocycloalkyl ring comprising 1 to 3 heteroatoms selected from O, N, and S optionally substituted with one or more R₁₀;
R₆ and R₆, are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S; and
q is 0, 1, 2, 3, or 4; and
(b) a second therapeutic agent.

Embodiment 377: The combination according to Embodiment 376, wherein the compound of Formula (Ic) is selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof.

Embodiment 378: The combination according to Embodiment 376 or 377, wherein the compound of Formula (Ic) is Compound I-156.

Embodiment 379: The combination according to Embodiment 376 or 377 wherein the compound of Formula (Ic) is Compound I-57.

Embodiment 380: The combination according to Embodiment 376 or 377, wherein the compound of Formula (Ic) is Compound I-87.

Embodiment 381: The combination according to Embodiment 376 or 377, wherein the compound of Formula (Ic) is Compound I-88.

Embodiment 382: The combination according to Embodiment 376 or 377, wherein the compound of Formula (Ic) is Compound I-265.

Embodiment 383: The combination according to Embodiment 376 or 377, wherein the compound of Formula (Ic) is Compound I-112.

Embodiment 384: The combination according to any one of Embodiment 376-383, wherein the second therapeutic agent is an immunomodulator.

Embodiment 385: The combination according to Embodiment 384, wherein the immunomodulator is an immune checkpoint inhibitor.

Embodiment 386: The combination according to Embodiment 385, wherein the immune checkpoint inhibitor is a PD-1 inhibitor.

Embodiment 387: The combination according to Embodiment 386, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 388: The combination according to Embodiment 387, wherein the PD-1 inhibitor is PDR001.

Embodiment 389: The combination according to any one of c Embodiment 376-388, wherein the combination comprises about 2 mg, or about 10 mg, or about 20 mg, or about 40 mg, or about 80 mg, or about 160 mg, or about 320 mg of the compound.

Embodiment 390: The combination according to any one of Embodiment 376-389, wherein the combination comprises about 100 mg, or about 200 mg, or about 300 mg, or about 400 mg, or about 500 mg of the second therapeutic agent.

Embodiment 391: The combination according to any one of Embodiment 376-390, wherein the combination comprises about 2 mg, or about 10 mg, or about 20 mg, or about 40 mg, or about 80 mg, or about 160 mg, or about 320 mg of the compound; and about 100 mg, or about 200 mg, or about 300 mg, or about 400 mg, or about 500 mg of the second therapeutic agent.

Embodiment 392: A combination according to any one of Embodiment 376-391 for use in the treatment or prevention of cancer.

Embodiment 393: Use of the combination according to any one of Embodiment 376-391 for the manufacture of a medicament for treating or preventing cancer.

Embodiment 394: Use of the combination according to any one of Embodiment 376-391 for the treatment or prevention of cancer.

Embodiment 395: The combination according to Embodiment 392 or the use according to Embodiment 393 or 394, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 396: A pharmaceutical combination comprising, a compound that has degrader activity for IKZF2 and one or more therapeutic agent, wherein the one or more therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof.

Embodiment 397: A pharmaceutical combination comprising, a compound that has degrader activity for IKZF2 and one or more therapeutic agent(s), wherein the one or more therapeutic agent is selected from a PD-1 inhibitor, a LAG-3 inhibitor, a cytokine, an A2A antagonist, a GITR agonist, a TIM-3 inhibitor, a STING agonist, and a TLR7 agonist.

Embodiment 398: The combination of claim 397, wherein the one or more therapeutic agent is a PD-1 inhibitor.

Embodiment 399: The combination of claim 397, wherein the one or more therapeutic agent is a LAG-3 inhibitor.

Embodiment 400: The combination of claim 397, wherein the one or more therapeutic agent is a cytokine.

Embodiment 401: The combination of claim 397, wherein the one or more therapeutic agent is an A2A antagonist.

Embodiment 402: The combination of claim 397, wherein the one or more therapeutic agent is a GITR agonist.

Embodiment 403: The combination of claim 397, wherein the one or more therapeutic agent is a TIM-3 inhibitor.

Embodiment 404: The combination of claim 397, wherein the one or more therapeutic agent is a STING agonist.

Embodiment 405: The combination of claim 397, wherein the one or more therapeutic agent is a TLR7 agonist.

Embodiment 406: A pharmaceutical combination comprising, a compound that decreases IKZF2 levels in a patient and one or more therapeutic agent, wherein the one or more therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof.

Embodiment 407: A pharmaceutical combination comprising, a compound that decreases IKZF2 levels in a patient and one or more therapeutic agent(s), wherein the one or more therapeutic agent is selected from a PD-1 inhibitor, a LAG-3 inhibitor, a cytokine, an A2A antagonist, a GITR agonist, a TIM-3 inhibitor, a STING agonist, and a TLR7 agonist.

Embodiment 408: The combination of claim 407, wherein the one or more therapeutic agent is a PD-1 inhibitor.

Embodiment 409: The combination of claim 407, wherein the one or more therapeutic agent is a LAG-3 inhibitor.

Embodiment 410: The combination of claim 407, wherein the one or more therapeutic agent is a cytokine.

Embodiment 411: The combination of claim 407, wherein the one or more therapeutic agent is an A2A antagonist.

Embodiment 412: The combination of claim 407, wherein the one or more therapeutic agent is a GITR agonist.

Embodiment 413: The combination of claim 407, wherein the one or more therapeutic agent is a TIM-3 inhibitor.

Embodiment 414: The combination of claim 407, wherein the one or more therapeutic agent is a STING agonist.Embodiment 415: The combination of claim 407, wherein the one or more therapeutic agent is a TLR7 agonist.

Embodiment 415: A method of treating or preventing cancer comprising administering to a patient in need thereof a combination comprising,
(a) a compound of Formula (I′):

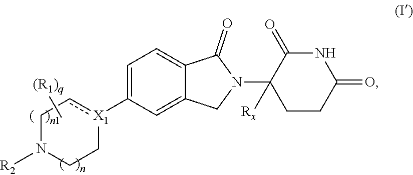

or pharmaceutically acceptable salts, hydrates, solvates, prodrugs, stereoisomers, and tautomers thereof,
wherein:
X₁ is CR₃;
[symbol] is optionally a double bond when X₁ is CR₃ and R₃ is absent;
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
R₃ is H or R₃ is absent when [symbol] is a double bond;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₅)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₃-C₇)cycloalkyl ring or a 5- to 7-membered heterocycloalkyl ring comprising 1 to 3 heteroatoms selected from O, N, and S optionally substituted with one or more R₁₀;
R₆ and R₆, are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S;
Rₓ is H or D;
p is 0, 1, or 2;
n is 0, 1, or 2;
n1 is 1 or 2, wherein n+n1≤3; and
q is 0, 1, 2, 3, or 4; and
(b) a second therapeutic agent.

Embodiment 416: The method according to Embodiment 415, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

Embodiment 417: The method according to Embodiment 415 or 416, wherein the cancer is selected from non-small cell lung cancer (NSCLC), melanoma, triple-negative breast cancer (TNBC), nasopharyngeal cancer (NPC), and microsatellite stable colorectal cancer (mssCRC).

Embodiment 418: The method according to any one of Embodiments 415-417, wherein the compound and the second agent are administered simultaneously, separately, or over a period of time.

Embodiment 419: The method according to any one of Embodiments 415-418, wherein the amount of the compound of Formula (I′), or a pharmaceutically acceptable salt, hydrate, solvate, prodrug, stereoisomer, or tautomer thereof, administered to the patient in need thereof is effective to treat or prevent the cancer.

Embodiment 420: The method according to any one of Embodiments 415-419, wherein the amounts of: (a) compound of Formula (I′), or a pharmaceutically acceptable salt, hydrate, solvate, prodrug, stereoisomer, or tautomer thereof; and (b) the second therapeutic agent, administered to the patient in need thereof are effective to treat or prevent the cancer.

Embodiment 421: The method according to any one of Embodiments 415-420, wherein the compound of Formula (I′) has a Formula (I), Formula (Ia), Formula (Ib), Formula (Ic), or Formula (Id):

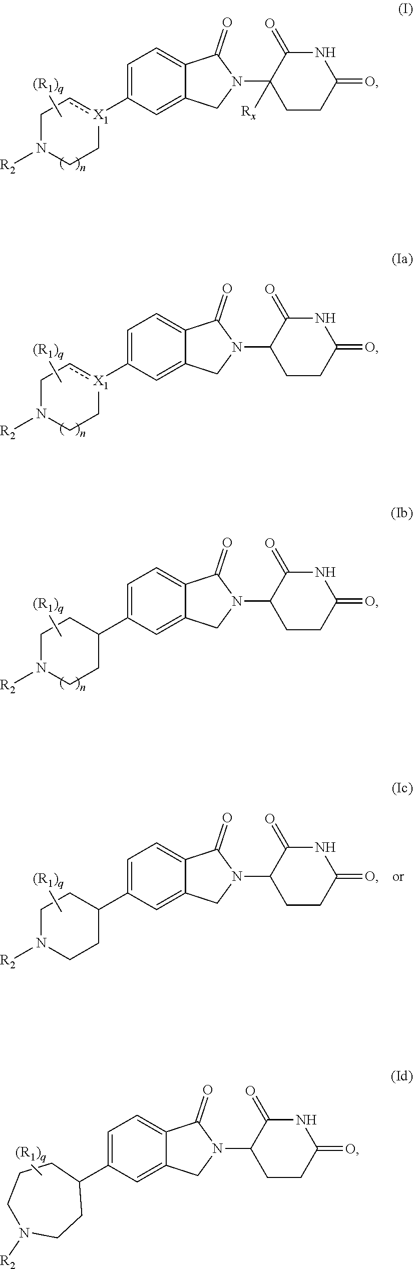

or pharmaceutically acceptable salts, hydrates, solvates, prodrugs, stereoisomers, and tautomers thereof,

Embodiment 422: The method according to any one of Embodiments 415-421, wherein the compound of Formula (F) is selected from

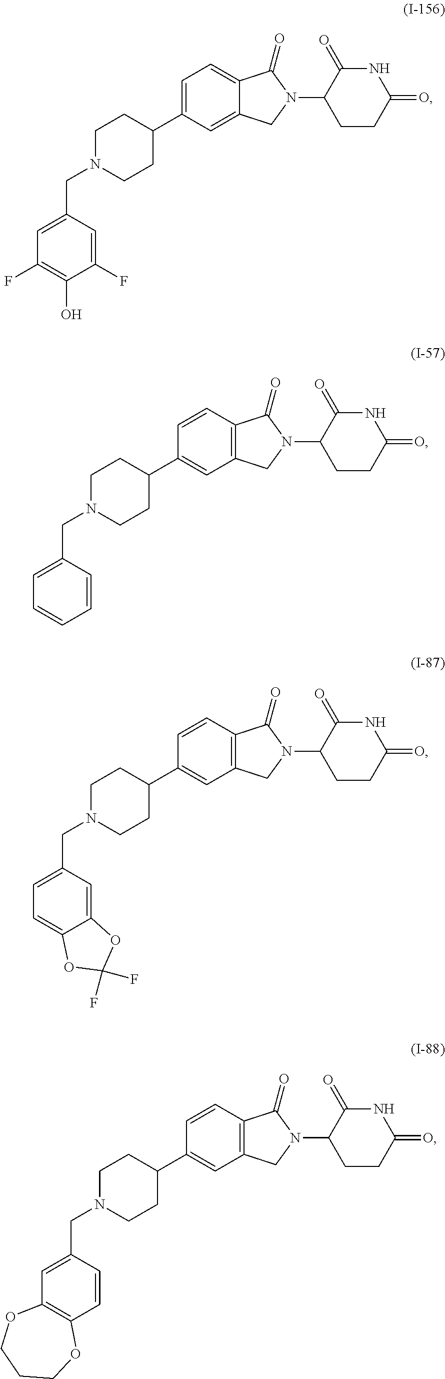 [SMILES: O=C1CCC(N2Cc3cc(C4CCN(Cc5cc(F)c(O)c(F)c5)CC4)ccc3C2=O)C(=O)N1 ; O=C1CCC(N2Cc3cc(C4CCN(Cc5ccccc5)CC4)ccc3C2=O)C(=O)N1 ; O=C1CCC(N2Cc3cc(C4CCN(Cc5ccc6c(c5)OC(F)(F)O6)CC4)ccc3C2=O)C(=O)N1 ; O=C1CCC(N2Cc3cc(C4CCN(Cc5ccc6c(c5)OCCCO6)CC4)ccc3C2=O)C(=O)N1]

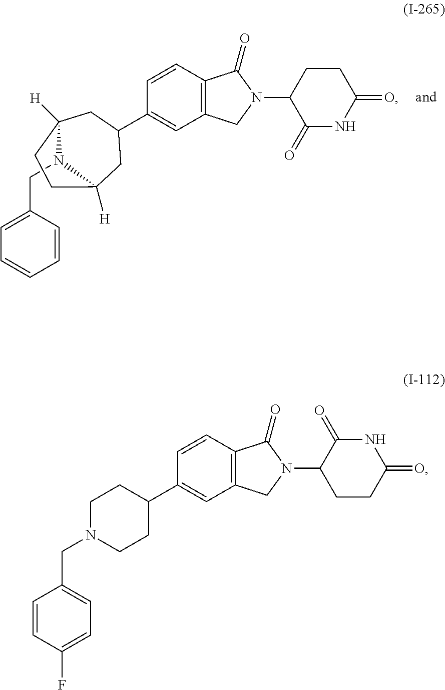 [SMILES: O=C1CCC(N2Cc3cc(C4C[C@H]5CC[C@@H](C4)N5Cc4ccccc4)ccc3C2=O)C(=O)N1 ; O=C1CCC(N2Cc3cc(C4CCN(Cc5ccc(F)cc5)CC4)ccc3C2=O)C(=O)N1]

or a pharmaceutically acceptable salt, hydrate, solvate, prodrug, stereoisomer, or tautomer thereof.

Embodiment 423: The method according to any one of Embodiments 415-422, wherein the compound of Formula (I′) is Compound I-156.

Embodiment 424: The method according to any one of Embodiments 415-422, wherein the compound of Formula (I′) is Compound I-57.

Embodiment 425: The method according to any one of Embodiments 415-422, wherein the compound of Formula (I′) is Compound I-87.

Embodiment 426: The method according to any one of Embodiments 415-422, wherein the compound of Formula (I′) is Compound I-88.

Embodiment 427: The method according to any one of Embodiments 415-422, wherein the compound of Formula (I′) is Compound I-265.

Embodiment 428: The method according to any one of Embodiments 415-422, wherein the compound of Formula (I′) is Compound I-112.

Embodiment 429: The method according to any one of Embodiments 415-428, wherein the second therapeutic agent is an immunomodulator.

Embodiment 430: The method according to Embodiment 429, wherein the second therapeutic agent is an immune checkpoint inhibitor.

Embodiment 431: The method according to Embodiment 430, wherein the second therapeutic agent is a PD-1 inhibitor.

Embodiment 432: The method according to Embodiment 431, wherein the PD-1 inhibitor is PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 433: The method according to Embodiment 432, wherein the PD-1 inhibitor is PDR001.

Embodiment 434: The method according to any one of Embodiments 415-433, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 435: The method according to any one of Embodiments 415-434, wherein the compound is administered orally.

Embodiment 436: The method according to any one of Embodiments 415-435, wherein the second therapeutic agent is administered at a dose of about 100 mg once every four weeks, or about 200 mg once every four weeks, or about 300 mg once every four weeks, or about 400 mg once every four weeks, or about 500 mg once every four weeks.

Embodiment 437: The method according to any one of Embodiments 415-436, wherein the second therapeutic agent is administered at a dose of about 400 mg once every four weeks.

Embodiment 438: The method according to any one of Embodiments 415-437, wherein the second therapeutic agent is administered intravenously.

Embodiment 439: The method according to any one of Embodiments 415-438, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

Embodiment 440: A method of treating or preventing cancer comprising administering to a patient in need thereof a compound of Formula (I′):

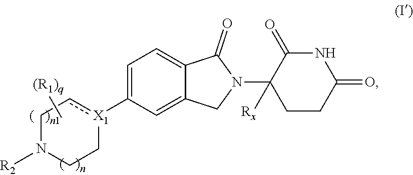

or a pharmaceutically acceptable salt, hydrate, solvate, prodrug, stereoisomer, or tautomer thereof,
wherein:
X₁ is CR₃;
[symbol] is optionally a double bond when X₁ is CR₃ and R₃ is absent;
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
R₃ is H or R₃ is absent when [symbol] is a double bond;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₅)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R₆, are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more Rn, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S;
Rₓ is H or D;
p is 0, 1, or 2;
n is 0, 1, or 2;
n1 is 1 or 2, wherein n+n1<3; and
q is 0, 1, 2, 3, or 4;
wherein the compound of Formula (I′) is administered orally at a dose of about 2 mg per day, or about 4 mg per day, or about 10 mg per day, or about 20 mg per day, or about 40 mg per day, or about 80 mg per day, or about 160 mg per day, or about 320 mg per day.

Embodiment 441: The method according to Embodiment 440, wherein the amount of the compound of Formula (I′), or a pharmaceutically acceptable salt, hydrate, solvate, prodrug, stereoisomer, or tautomer thereof, is effective to treat or prevent the cancer.

Embodiment 442: The method according to Embodiment 440 or 441, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

Embodiment 443: The method according to any one of Embodiments 440-442, wherein the cancer is selected from non-small cell lung cancer (NSCLC), melanoma, triple-negative breast cancer (TNBC), nasopharyngeal cancer (NPC), and microsatellite stable colorectal cancer (mssCRC).

Embodiment 444: The method according to any one of Embodiments 440-443, wherein the compound of Formula (I′) is selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof.

Embodiment 445: The method according to any one of Embodiments 440-444, wherein the compound of Formula (I′) is Compound I-156.

Embodiment 446: The method according to any one of Embodiments 440-444, wherein the compound of Formula (I′) is Compound I-57.

Embodiment 447: The method according to any one of Embodiments 440-444, wherein the compound of Formula (I′) is Compound I-87.

Embodiment 448: The method according to any one of Embodiments 440-444, wherein the compound of Formula (I′) is Compound I-88.

Embodiment 449: The method according to any one of Embodiments 440-444, wherein the compound of Formula (I′) is Compound I-265.

Embodiment 450: The method according to any one of claims 440-444, wherein the compound of Formula (I′) is Compound I-112.

Embodiment 451: The method according to any one of claims 440-450 further comprising a second therapeutic agent.

Embodiment 452: The method according to Embodiment 451, wherein the compound and the second agent are administered simultaneously, separately, or over a period of time.

Embodiment 453: The method according to Embodiment 451 or 452, wherein the second therapeutic agent is an immunomodulator.

Embodiment 454: The method according to Embodiment 453, wherein the immunomodulator is an immune checkpoint inhibitor.

Embodiment 455: The method according to Embodiment 454, wherein the immune checkpoint inhibitor is a PD-1 inhibitor.

Embodiment 456: The method according to Embodiment 455, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 457: The method according to Embodiment 456, wherein the PD-1 inhibitor is PDR001.

Embodiment 458: The method according to any one of Embodiments 451-42, wherein the second therapeutic agent is administered at a dose of about 100 mg once every four weeks, or about 200 mg once every four weeks, or about 300 mg once every four weeks, or about 400 mg once every four weeks, or about 500 mg once every four weeks.

Embodiment 459: The method according to any one of Embodiments 451-458, wherein the second therapeutic agent is administered at a dose of about 400 mg once every four weeks.

Embodiment 460: The method according to any one of Embodiments 451-459, wherein the second therapeutic agent is administered intravenously.

Embodiment 461: The method according to any one of Embodiments 451-460, wherein the amounts of: (a) the compound of Formula (I′), or a pharmaceutically acceptable salt, hydrate, solvate, prodrug, stereoisomer, or tautomer thereof; and (b) the second therapeutic agent, are effective to treat or prevent the cancer.

Embodiment 462: The method according to any one of Embodiments 451-461, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

Embodiment 463: The method according to any one of Embodiments 415-462, wherein the method further comprises measuring the level of at least one biomarker selected from IKZF2, PD-L1, CD8, and FOXP3.

Embodiment 464: The method according to Embodiment 463, wherein the level of IKZF2 is reduced.

Embodiment 465: The method according to any one of Embodiments 415-464, wherein the patient was previously treated with an anti-PD-1/PD-L1 therapy.

Embodiment 466: The method according to any one of Embodiments 415-465, wherein the patient being treated for NSCLC or melanoma, or a combination thereof, was primarily refractory to anti-PD-1/PD-L1 therapy agent showing no significant radiologic response during treatment with an anti-PD-1/PD-L1 agent <6 months prior to disease progression.

Embodiment 467: The method according to any one of Embodiments 415-465, wherein the patient being treated for NPC, mssCRC, or TNBC, or a combination thereof, was naive to anti-PD-1/PD-L1 therapy.

Embodiment 468: The method according to any one of Embodiments 415-467, wherein the patient has not been treated with an IKZF2 targeting agent.

Embodiment 469: The method according to any one of Embodiments 415-468, wherein the patient does not show the presence of symptomatic central nervous system (CNS) metastases, or CNS metastases requiring local CNS-directed therapy (such as radiotherapy or surgery), or increasing doses of corticosteroids within 2 weeks prior to the time of the first administration of the compound or the combination comprising the compound and a second agent.

Embodiment 470: The method according to any one of Embodiments 415-469, wherein the patient does not have a history of severe hypersensitivity reactions to any ingredient of study drug(s) and other mAbs and/or their excipients.

Embodiment 471: The method according to any one of Embodiments 415-470, wherein the patient does not have clinically significant cardiac disease or impaired cardiac function.

Embodiment 472: The method according to any one of Embodiments 415-471, wherein the patient does not have any one of the following clinically significant cardiac disease or impaired cardiac function:
(i) clinically significant and/or uncontrolled heart disease such as congestive heart failure requiring treatment with NYHA grade ≥2;
(ii) uncontrolled hypertension or clinically significant arrhythmia;
(iii) QT interval corrected by Fridericia's formula (QTcF) >450 msec in male patients, or >460 msec female patients;
(iv) QTc that is not assessable;
(v) congenital long QT syndrome;
(vi) history of familial long QT syndrome or known family history of as Torsades de Pointes; and
(vii) acute myocardial infarction or unstable angina pectoris ≤3 months prior to the time of the first administration of the compound or the combination comprising the compound and a second agent.

Embodiment 473: The method according to any one of claims 1-472, wherein the patient does not have HIV infection.

Embodiment 474: The method according to any one of Embodiments 415-473, wherein the patient does not have hepatitis B virus (HBV) infection.

Embodiment 475: The method according to any one of Embodiments 415-474, wherein the patient does not have hepatitis C virus (HCV) infection.

Embodiment 476: The method according to any one of Embodiments 415-475, wherein the patient does not have active, known, or suspected autoimmune disease.

Embodiment 477: The method according to any one of Embodiments 415-476, wherein the patient does not have the presence or history of interstitial lung disease or interstitial pneumonitis, including clinically significant radiation or drug-induced pneumonitis.

Embodiment 478: The method according to any one of Embodiments 415-477, wherein the patient has not been treated with
(i) a cytotoxic or targeted antineoplastics within 3 weeks prior to the time of the first administration of the compound or the combination comprising the compound and a second agent;
(ii) systemic chronic steroid therapy (>10 mg/day prednisone or equivalent) or any other immunosuppressive therapy within 7 days prior to the time of the first administration of the compound or the combination comprising the compound and a second agent;
(iii) radiotherapy within 2 weeks prior to the time of the first administration of the compound or the combination comprising the compound and a second agent; or
(iv) any immunosuppressive medication that would interfere with the action of the compound or the combination comprising the compound and a second agent;
or a combination thereof.

Embodiment 479: The method according to any one of Embodiments 415-478, wherein the patient has not been using any live vaccines against infectious diseases within 4 weeks prior to the time of the first administration of the compound or the combination comprising the compound and a second agent; or using hematopoietic colony-stimulating growth factors thrombopoietin mimetics or erythroid stimulating agents within <2 weeks prior to the time of the first administration of the compound or the combination comprising the compound and a second agent.

Embodiment 480: A pharmaceutical combination comprising,
(a) a compound of Formula (I′):

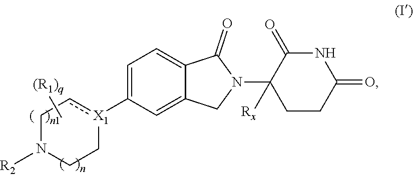

or a pharmaceutically acceptable salt, hydrate, solvate, prodrug, stereoisomer, or tautomer thereof,
wherein:
X₁ is CR₃;
[symbol] is optionally a double bond when X₁ is CR₃ and R₃ is absent;
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
R₃ is H or R₃ is absent when [symbol] is a double bond;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₅)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R₆, are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S;
Rₓ is H or D;
p is 0, 1, or 2;
n is 0, 1, or 2;
n1 is 1 or 2, wherein n+n1≤3; and
q is 0, 1, 2, 3, or 4; and
(b) a second therapeutic agent.

Embodiment 481: The combination according to Embodiment 480, wherein the compound of Formula (I′) is selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof.

Embodiment 482: The combination according to Embodiment 480 or 481, wherein the compound of Formula (I′) is Compound I-156.

Embodiment 483: The combination according to Embodiment 480 or 481, wherein the compound of Formula (I′) is Compound I-57.

Embodiment 484: The combination according to Embodiment 480 or 481, wherein the compound of Formula (I′) is Compound I-87.

Embodiment 485: The combination according to Embodiment 480 or 481, wherein the compound of Formula (I′) is Compound I-88.

Embodiment 486: The combination according to Embodiment 480 or 481, wherein the compound of Formula (I′) is Compound I-265.

Embodiment 487: The combination according to Embodiment 480 or 481, wherein the compound of Formula (I′) is Compound I-112.

Embodiment 488: The combination according to any one of claims 480-487, wherein the second therapeutic agent is an immunomodulator.

Embodiment 489: The combination according to Embodiment 488, wherein the immunomodulator is an immune checkpoint inhibitor.

Embodiment 490: The combination according to Embodiment 489, wherein the immune checkpoint inhibitor is a PD-1 inhibitor.

Embodiment 491: The combination according to claim Embodiment 490, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

Embodiment 492: The combination according to claim Embodiment 491, wherein the PD-1 inhibitor is PDR001.

Embodiment 493: The combination according to any one of Embodiments 480-492, wherein the combination comprises about 2 mg, or about 10 mg, or about 20 mg, or about 40 mg, or about 80 mg, or about 160 mg, or about 320 mg of the compound.

Embodiment 494: The combination according to any one of Embodiments 480-493, wherein the combination comprises about 100 mg, or about 200 mg, or about 300 mg, or about 400 mg, or about 500 mg of the second therapeutic agent.

Embodiment 495: The combination according to any one of Embodiments 480-494, wherein the combination comprises about 2 mg, or about 10 mg, or about 20 mg, or about 40 mg, or about 80 mg, or about 160 mg, or about 320 mg of the compound; and about 100 mg, or about 200 mg, or about 300 mg, or about 400 mg, or about 500 mg of the second therapeutic agent.

Embodiment 496: A combination according to any one of Embodiments 480-495 for use in the treatment or prevention of cancer.

Embodiment 497: Use of the combination according to any one of Embodiments 480-495 for the manufacture of a medicament for treating or preventing cancer.

Embodiment 498: Use of the combination according to any one of Embodiments 480-495 for the treatment or prevention of cancer.

Embodiment 499: The combination according to Embodiment 496 or the use according to Embodiments 497 or 498, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

Embodiment 500: A method of treating or preventing cancer comprising administering to a patient in need thereof a compound that has degrader activity for IKZF2 in combination with one or more therapeutic agents, wherein the therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof.

Embodiment 501: The method of Embodiment 500, wherein the one or more therapeutic agents is selected from a PD-1 inhibitor, a LAG-3 inhibitor, a cytokine, an A2A antagonist, a GITR agonist, a TIM-3 inhibitor, a STING agonist, and a TLR7 agonist.

Embodiment 502: The method of Embodiment 500, wherein the one or more therapeutic agents is a PD-1 inhibitor.

Embodiment 503: The method of Embodiment 500, wherein the one or more therapeutic agents is a LAG-3 inhibitor.

Embodiment 504: The method of Embodiment 500, wherein the one or more therapeutic agents is a cytokine.

Embodiment 505: The method of Embodiment 500, wherein the one or more therapeutic agents is an A2A antagonist.

Embodiment 506: The method of Embodiment 500, wherein the one or more therapeutic agents is a GITR agonist.

Embodiment 507: The method of Embodiment 500, wherein the one or more therapeutic agents is a TIM-3 inhibitor.

Embodiment 508: The method of Embodiment 500, wherein the one or more therapeutic agents is a STING agonist.

Embodiment 509: The method of Embodiment 500, wherein the one or more therapeutic agents is a TLR7 agonist.

In some embodiments, the combination or formulation comprises about 0.1 mg, or about 0.5 mg, or about 1 mg, or about 2 mg, or about 3 mg, or about 4 mg, or about 5 mg, or about 10 mg, or about 15 mg, or about 20 mg, or about 25 mg, or about 30 mg, or about 35 mg, or about 40 mg, or about 45 mg, or about 50 mg, or about 55 mg, or about 60 mg, or about 65 mg, or about 70 mg, or about 75 mg, or about 80 mg, or about 85 mg, or about 90 mg, or about 95 mg, or about 100 mg, or about 110 mg, or about 120 mg, or about 130 mg, or about 140 mg, or about 150 mg, or about 160 mg, or about 170 mg, or about 180 mg, or about 190 mg, or about 200 mg, or about 210 mg, or about 220 mg, or about 230 mg, or about 240 mg, or about 250 mg, or about 260 mg, or about 270 mg, or about 280 mg, or about 290 mg, or about 300 mg, or about 310 mg, or about 320 mg, or about 330 mg, or about 340 mg, or about 350 mg, or about 360 mg, or about 370 mg, or about 380 mg, or about 390 mg, or about 400 mg, or about 410 mg, or about 420 mg, or about 430 mg, or about 440 mg, or about 450 mg, or about 460 mg, or about 470 mg, or about 480 mg, or about 500 mg of the compound.

In some embodiments, the combination or formulation comprises between about 10 to about 50 mg, or between about 50 to about 100 mg, or between about 100 to about 200 mg, or between about 200 mg to about 300 mg, or between about 300 mg to about 400 mg, or between about 400 mg to about 500 mg or between about 500 mg to about 600 mg, or between about 600 mg to about 700 mg of the second therapeutic agent.

In some embodiments, the combination or formulation comprises between about 10 to about 50 mg, or between about 50 to about 100 mg, or between about 100 to about 150 mg, or between about 150 mg to about 200 mg, or between about 200 mg to about 250 mg, or between about 250 mg to about 300 mg or between about 350 mg to about 400 mg, or between about 400 mg to about 450 mg, or between about 450 mg to about 500 mg, or between about 500 mg to about 550 mg, or between about 550 mg to about 600 mg, or between about 600 mg to about 650 mg, or between about 650 mg to about 750 mg of the second therapeutic agent.

In some embodiments, the combination or formulation comprises 100 mg, or 200 mg, or 300 mg, or 400 mg, or 500 mg of the second therapeutic agent.

In some embodiments, the combination or formulation comprises between 10 to 50 mg, or between 50 to 100 mg, or between 100 to 200 mg, or between 200 mg to 300 mg, or between 300 mg to 400 mg, or between 400 mg to 500 mg or between 500 mg to 600 mg, or between 600 mg to 700 mg or between 600 mg to 800 mg of the second therapeutic agent.

In some embodiments, the combination or formulation comprises between 10 to 50 mg, or between 50 to 100 mg, or between 100 to 150 mg, or between 150 mg to 200 mg, or between 200 mg to 250 mg, or between 250 mg to 300 mg or between 350 mg to 400 mg, or between 400 mg to 450 mg, or between 450 mg to 500 mg, or between 500 mg to 550 mg, or between 550 mg to 600 mg, or between 600 mg to 650 mg, or between 650 mg to 750 mg of the second therapeutic agent.

In some embodiments, the combination or formulation comprises 2 mg, or 10 mg, or 20 mg, or 40 mg, or 80 mg, or 160 mg, or 320 mg of the compound.

In some embodiments, the combination or formulation comprises between 1 to 10 mg, or between 10 mg to 20 mg, or between 20 to 30 mg, or between 30 mg to 40 mg, or between 40 mg to 50 mg, or between 50 mg to 60 mg, or between 60 mg to 70 mg, or between 70 mg to 80 mg, or between 80 mg to 90 mg, or between 90 mg to 100 mg, or between 100 mg to 110 mg, or between 110 mg to 120 mg, or between 120 mg to 130 mg, or between 130 mg to 140 mg, or between 140 mg to 150 mg, or between 150 mg to 160 mg, or between 160 mg to 170 mg, or between 170 mg to 180 mg, or between 180 mg to 190 mg, or between 190 mg to 200 mg, or between 200 mg to 210 mg, or between 210 mg to 220 mg, or between 220 mg to 230 mg, or between 230 mg to 240 mg, or between 240 mg to 250 mg, or between 250 mg to 260 mg, or between 260 mg to 270 mg, or between 270 mg to 280 mg, or between 280 mg to 290 mg, or between 290 mg to 300 mg, or between 300 mg to 310 mg, or between 310 mg to 320 mg, or between 320 mg to 330 mg, or between 330 mg to 340 mg, or between 340 mg to 350 mg, or between 350 mg to 360 mg, or between 360 mg to 370 mg, or between 370 mg to 380 mg, or between 380 mg to 390 mg, or between 390 mg to 400 mg, or between 400 mg to 420 mg, or between 420 mg to 430 mg, or between 430 mg to 440 mg, or between 440 mg to 450 mg, or between 450 mg to 460 mg, or between 460 mg to 470 mg, or between 470 mg to 480 mg, or between 480 mg to 490 mg, or between 490 mg to 500 mg of the compound.

In some embodiments, the combination or formulation comprises 0.1 mg, or 0.5 mg, or 1 mg, or 2 mg, or 3 mg, or 4 mg, or 5 mg, or 10 mg, or 15 mg, or 20 mg, or 25 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg, or 110 mg, or 120 mg, or 130 mg, or 140 mg, or 150 mg, or 160 mg, or 170 mg, or 180 mg, or 190 mg, or 200 mg, or 210 mg, or 220 mg, or 230 mg, or 240 mg, or 250 mg, or 260 mg, or 270 mg, or 280 mg, or 290 mg, or 300 mg, or 310 mg, or 320 mg, or 330 mg, or 340 mg, or 350 mg, or 360 mg, or 370 mg, or 380 mg, or 390 mg, or 400 mg, or 410 mg, or 420 mg, or 430 mg, or 440 mg, or 450 mg, or 460 mg, or 470 mg, or 480 mg, or 500 mg of the compound.

In some embodiments, the second therapeutic agent is an immunomodulator.

In some embodiments, the second therapeutic agent is an immune checkpoint inhibitor.

In some embodiments, the second therapeutic agent is a PD-1 inhibitor.

In some embodiments, the second therapeutic agent is a PD-1 inhibitor selected from PDR001, Nivolumab, Pembrolizumab, Pidilizumab, MEDI0680, REGN2810, TSR-042, PF-06801591, BGB-A317, BGB-108, INCSHR1210, and AMP-224.

In some embodiments, the second therapeutic agent is PDR001.

In some embodiments, the second therapeutic agent is selected from a PD-1 inhibitor, a LAG-3 inhibitor, a cytokine, an A2A antagonist, a GITR agonist, a TIM-3 inhibitor, a STING agonist, and a TLR7 agonist.

In some embodiments, the second therapeutic agent is a LAG-3 inhibitor.

In some embodiments, the second therapeutic agent is a cytokine.

In some embodiments, the second therapeutic agent is an A2A antagonist.

In some embodiments, the second therapeutic agent is a GITR agonist.

In some embodiments, the second therapeutic agent is a TIM-3 inhibitor.

In some embodiments, the second therapeutic agent is a STING agonist.

In some embodiments, the second therapeutic agent is a TLR7 agonist.

In some embodiments, the method further comprises measuring the level of at least one biomarker selected from IKZF2, PD-L1, CD8, and FOXP3.

In some embodiments, the method further comprises measuring the level of at least two biomarker selected from IKZF2, PD-L1, CD8, and FOXP3.

In some embodiments, the method further comprises measuring the level of at least three biomarker selected from IKZF2, PD-L1, CD8, and FOXP3.

In some embodiments, the method further comprises measuring the level of IKZF2, PD-L1, CD8, and FOXP3.

In some embodiments, the method further comprises measuring the level of IKZF2 In some embodiments, the method further comprises measuring the level of PD-L1.

In some embodiments, the method further comprises measuring the level of CD8.

In some embodiments, the method further comprises measuring the level of FOXP3.

In some embodiments, the level of IKZF2 is reduced when the patient is treated with a combination according to 1a or a formulation according to 1b.

In some embodiments, the patient was previously treated with an anti-PD-1/PD-L1 therapy.

In some embodiments, the patient being treated for NSCLC or melanoma, or a combination thereof, was primarily refractory to anti-PD-1/PD-L1 therapy agent showing no significant radiologic response during treatment with an anti-PD-1/PD-L1 agent <6 months prior to disease progression.

In some embodiments, the patient being treated for NSCLC was primarily refractory to anti-PD-1/PD-L1 therapy agent showing no significant radiologic response during treatment with an anti-PD-1/PD-L1 agent <6 months prior to disease progression.

In some embodiments, the patient being treated for melanoma was primarily refractory to anti-PD-1/PD-L1 therapy agent showing no significant radiologic response during treatment with an anti-PD-1/PD-L1 agent <6 months prior to disease progression.

In some embodiments, the patient being treated for NPC was naive to anti-PD-1/PD-L1 therapy.

In some embodiments, the patient being treated for mssCRC was naive to anti-PD-1/PD-L1 therapy.

In some embodiments, the patient being treated for TNBC was naive to anti-PD-1/PD-L1 therapy.

In some embodiments, the patient has not been treated with an IKZF2 targeting agent.

In some embodiments, the patient does not show the presence of symptomatic central nervous system (CNS) metastases, or CNS metastases requiring local CNS-directed therapy (such as radiotherapy or surgery), or increasing doses of corticosteroids within 2 weeks prior to the time of the first administration of the compound or the combination comprising the compound and a second agent.

In some embodiments, the patient does not have a history of severe hypersensitivity reactions to any ingredient of study drug(s) and other mAbs and/or their excipients.

In some embodiments, the patient does not have clinically significant cardiac disease or impaired cardiac function.

In some embodiments, the patient does not have any one of the following clinically significant cardiac disease or impaired cardiac function, including any of the following: (i) clinically significant and/or uncontrolled heart disease such as congestive heart failure requiring treatment with NYHA grade >2; (ii) uncontrolled hypertension or clinically significant arrhythmia; (iii) QT interval corrected by Fridericia's formula (QTcF) >450 msec in male patients, or >460 msec female patients; (iv) QTc that is not assessable; (v) congenital long QT syndrome; (vi) history of familial long QT syndrome or known family history of as Torsades de Pointes; and (vii) acute myocardial infarction or unstable angina pectoris <3 months prior to the time of the first administration of the compound or the combination comprising the compound and a second agent.

In some embodiments, the patient does not have HIV infection.

In some embodiments, the patient does not have hepatitis B virus (HBV) infection.

In some embodiments, the patient does not have hepatitis C virus (HCV) infection.

In some embodiments, the patient does not have active, known, or suspected autoimmune disease.

In some embodiments, the patient does not have the presence or history of interstitial lung disease or interstitial pneumonitis, including clinically significant radiation or drug-induced pneumonitis.

In some embodiments, the patient has not been treated with cytotoxic or targeted antineoplastics within 3 weeks prior to the time of the first administration of the compound or the combination comprising the compound and a second agent.

In some embodiments, the patient has not been treated with systemic chronic steroid therapy (>10 mg/day prednisone or equivalent) or any other immunosuppressive therapy within 7 days prior to the time of the first administration of the compound or the combination comprising the compound and a second agent.

In some embodiments, the patient has not been treated with radiotherapy within 2 weeks prior to the time of the first administration of the compound or the combination comprising the compound and a second agent.

In some embodiments, the patient has not been treated with any immunosuppressive medication that would interfere with the action of the compound or the combination comprising the compound and a second agent.

In some embodiments, the patient has not been using any live vaccines against infectious diseases within 4 weeks prior to the time of the first administration of the compound or the combination comprising the compound and a second agent.

In some embodiments, the patient has not been using hematopoietic colony-stimulating growth factors thrombopoietin mimetics or erythroid stimulating agents within <2 weeks prior to the time of the first administration of the compound or the combination comprising the compound and a second agent.

In some embodiments, the cancer being treated or prevented is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

In some embodiments, the present disclosure relates to pharmaceutical formulation comprising a compound selected from Compound I-156, Compound I-57, Compound I-87, Compound I-88, Compound I-265, and Compound I-112, and immunomodulator.

In another embodiment, the present disclosure relates to pharmaceutical formulation comprising a compound selected from Compound I-156, Compound I-57, Compound I-87, Compound I-88, Compound I-265, or a pharmaceutically acceptable salt, hydrate, solvate, prodrug, stereoisomer, or tautomer thereof, and Compound I-112, and an immune checkpoint inhibitor.

In another embodiment, the present disclosure relates to pharmaceutical formulation comprising (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and (b) a PD-1 inhibitor selected from PDR001, Nivolumab, Pembrolizumab, Pidilizumab, MEDI0680, REGN2810, TSR-042, PF-06801591, BGB-A317, BGB-108, INCSHR1210, and AMP-224.

In another embodiment, the present disclosure relates to pharmaceutical formulation comprising (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and (b) PDR001.

In some embodiments, the present disclosure relates to combination comprising a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and an immunomodulator.

In another embodiment, the present disclosure relates to combination comprising a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and an immune checkpoint inhibitor.

In another embodiment, the present disclosure relates to combination comprising (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and (b) a PD-1 inhibitor selected from PDR001, Nivolumab, Pembrolizumab, Pidilizumab, MEDI0680, REGN2810, TSR-042, PF-06801591, BGB-A317, BGB-108, INCSHR1210, and AMP-224.

In another embodiment, the present disclosure relates to combination comprising (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof, and (b) PDR001.

In another embodiment, the present disclosure relates to a method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) and an immunomodulator.

In another embodiment, the present disclosure relates to a method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) and an immune checkpoint inhibitor.

In another embodiment, the present disclosure relates to a method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a PD-1 inhibitor selected from PDR001, Nivolumab, Pembrolizumab, Pidilizumab, MEDI0680, REGN2810, TSR-042, PF-06801591, BGB-A317, BGB-108, INCSHR1210, and AMP-224.

In another embodiment, the present disclosure relates to a method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) PDR001.

In another embodiment, the present disclosure relates to a method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) and an immunomodulator, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

In another embodiment, the present disclosure relates to a method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) and an immune checkpoint inhibitor, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

In another embodiment, the present disclosure relates to a method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) a PD-1 inhibitor selected from PDR001, Nivolumab, Pembrolizumab, Pidilizumab, MEDI0680, REGN2810, TSR-042, PF-06801591, BGB-A317, BGB-108, INCSHR1210, and AMP-224, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

In another embodiment, the present disclosure relates to a method of treating or preventing cancer comprising administering to a patient in need thereof a combination comprising, (a) a compound selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof; and (b) PDR001, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

The Helios (IKZF2), Ikaros (IKZF1) and G1 to S phase transition 1 protein (GSPT1) degradation activity of the compounds of Formula (I′) and the synthesis of the compounds of Formula (I′) (e.g., general schemes, examples and procedures) were disclosed in WO2019/038717, which the entire content of which is incorporated herein by reference in its entirety. In some embodiments, the combination is administered simultaneously, separately, or over a period of time. In another embodiment, the combination is administered simultaneously or separately. In another embodiment, the combination is administered separately or over a period of time. In another embodiment, the combination is administered simultaneously.

In another embodiment, the combination is administered separately. In another embodiment, the combination is administered or over a period of time.

In another embodiment, the period of time will be at least for one week. In another embodiment, the period of time will be at least for one or more months.

In another embodiment of the disclosure, the compounds of the present disclosure are enantiomers. In some embodiments the compounds are the (S)-enantiomer. In other embodiments, the compounds are the (R)-enantiomer. In yet other embodiments, the compounds of the present disclosure may be (+) or (−) enantiomers.

It should be understood that all isomeric forms are included within the present disclosure, including mixtures thereof. If the compound contains a double bond, the substituent may be in the E or Z configuration. If the compound contains a disubstituted cycloalkyl, the cycloalkyl substituent may have a cis- or trans configuration. All tautomeric forms are also intended to be included.

Compounds of the disclosure, and pharmaceutically acceptable salts, hydrates, solvates, stereoisomers, and prodrugs thereof may exist in their tautomeric form (for example, as an amide or imino ether). All such tautomeric forms are contemplated herein as part of the present disclosure.

The compounds of the disclosure may contain asymmetric or chiral centers and, therefore, exist in different stereoisomeric forms. It is intended that all stereoisomeric forms of the compounds of the disclosure as well as mixtures thereof, including racemic mixtures, form part of the present disclosure. In addition, the present disclosure embraces all geometric and positional isomers. For example, if a compound of the disclosure incorporates a double bond or a fused ring, both the cis- and trans-forms, as well as mixtures, are embraced within the scope of the disclosure. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

The chiral centers of the compounds of the disclosure can have the S or R configuration as defined by the IUPAC 1974 Recommendations. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

The use of the terms “salt”, “solvate”, “ester,” “prodrug”, and the like, is intended to equally apply to the salt, solvate, ester, and prodrug of enantiomers, stereoisomers, rotamers, tautomers, positional isomers, racemates, or prodrugs of the inventive compounds.

The compounds of the disclosure may form salts, which are also within the scope of this disclosure. Reference to a compound of the Formula herein is generally understood to include reference to salts thereof, unless otherwise indicated.

Pharmaceutically acceptable solvates in accordance with the disclosure include those wherein the solvent of crystallization may be isotopically substituted, e.g., D₂O, d₆-acetone, d₆-DMSO.

The present disclosure relates to compounds, or combinations comprising same, which are modulators of IKZF2 protein levels. In one embodiment, the compounds of the present disclosure decrease IKZF2 protein levels. In yet one embodiment, the compounds of the present disclosure reduce IKZF2 protein levels. In another embodiment, the compounds of the present disclosure are degraders of IKZF2.

The present disclosure also relates to methods of using compounds, or combinations comprising compounds, which are modulators of IKZF2 protein levels. In one embodiment, the compounds of the present disclosure decrease IKZF2 protein levels. In yet one embodiment, the compounds of the present disclosure reduce IKZF2 protein levels. In another embodiment, the compounds of the present disclosure are degraders of IKZF2.

The present disclosure relates to compounds, or combinations comprising same, which are modulators of IKZF2 and IKZF4 protein levels. In one embodiment, the compounds of the present disclosure decrease IKZF2 and IKZF4 protein levels. In yet one embodiment, the compounds of the present disclosure reduce IKZF2 and IKZF4 protein levels. In another embodiment, the compounds of the present disclosure are degraders of IKZF2.

The present disclosure also relates to methods of using compounds, or combinations comprising compounds, which are modulators of IKZF2 and IKZF4 protein levels. In one embodiment, the compounds of the present disclosure decrease IKZF2 and IKZF4 protein levels. In yet one embodiment, the compounds of the present disclosure reduce IKZF2 and IKZF4 protein levels. In another embodiment, the compounds of the present disclosure are degraders of IKZF2.

In some embodiments, the compounds of the disclosure are selective over other proteins. As used herein “selective modulator”, “selective degrader”, or “selective compound” means, for example, a compound of the disclosure, that effectively modulates, decreases, or reduces the levels of a specific protein or degrades a specific protein to a greater extent than any other protein. A “selective modulator”, “selective degrader”, or “selective compound” can be identified, for example, by comparing the ability of a compound to modulate, decrease, or reduce the levels of or to degrade a specific protein to its ability to modulate, decrease, or reduce the levels of or to degrade other proteins. In some embodiments, the selectivity can be identified by measuring the EC₅₀ or IC₅₀ of the compounds. As used herein “modulator” or “degrader”, means, for example, a compound of the disclosure, which effectively modulates, decreases, or reduces the levels of a specific protein or degrades a specific protein.

In some embodiments, the compounds of the present application are selective IKZF2 modulators. As used herein “selective IKZF2 modulator”, “selective IKZF2 degrader”, or “selective IKZF2 compound” refers to a compound of the application, for example, that effectively modulates, decrease, or reduces the levels of IKZF2 protein or degrades IKZF2 protein to a greater extent than any other protein, particularly any protein (transcription factor) from the Ikaros protein family (e.g., IKZF1, IKZF3, IKZF4, and IKZF5).

A “selective IKZF2 modulator”, “selective IKZF2 degrader”, or “selective IKZF2 compound” can be identified, for example, by comparing the ability of a compound to modulate IKZF2 protein levels to its ability to modulate levels of other members of the Ikaros protein family or other proteins. For example, a substance may be assayed for its ability to modulate IKZF2 protein levels, as well as IKZF1, IKZF3, IKZF4, IKZF5, and other proteins. In some embodiments, the selectivity can be identified by measuring the EC₅₀ of the compounds. In some embodiments, a selective IKZF2 degrader is identified by comparing the ability of a compound to degrade IKZF2 to its ability to degrade other members of the Ikaros protein family or other proteins.

The compounds can be administered simultaneously (as a single preparation or separate preparation), sequentially, separately, or over a period of time to the other drug therapy or treatment modality. In general, a combination therapy envisions administration of two or more drugs during a single cycle or course of therapy.

### Second Therapeutic Agents Used in Combination Therapy

In one aspect, a 3-(1-oxoisoindolin-2-yl)piperidine-2,6-dione IKZF2 degrader compound or a pharmaceutically acceptable salt, hydrate, solvate, prodrug, stereoisomer, or tautomer thereof, of the present disclosure can be combined with other therapeutic agents (with one or more therapeutic agents (pharmaceutical combinations) or modalities), such as other anti-cancer agents, anti-allergic agents, anti-nausea agents (or anti-emetics), pain relievers, cytoprotective agents, and combinations thereof.

In some embodiments, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof of the present disclosure are administered in combination with one or more second agent(s) selected from a PD-1 inhibitor, a PD-L1 inhibitor, a LAG-3 inhibitor, a cytokine, an A2A antagonist, a GITR agonist, a TIM-3 inhibitor, a STING agonist, and a TLR7 agonist, to treat a disease, e.g., cancer.

In another embodiment, one or more chemotherapeutic agents are used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer, wherein said chemotherapeutic agents include, but are not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), epirubicin (Ellence®), oxaliplatin (Eloxatin®), exemestane (Aromasin®), letrozole (Femara®), and fulvestrant (Faslodex®).

In other embodiments, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more other anti-HER2 antibodies, e.g., trastuzumab, pertuzumab, margetuximab, or HT-19 described above, or with other anti-HER2 conjugates, e.g., ado-trastuzumab emtansine (also known as Kadcyla®, or T-DM1).

In other embodiments, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more tyrosine kinase inhibitors, including but not limited to, EGFR inhibitors, Her3 inhibitors, IGFR inhibitors, and Met inhibitors, for treating a disease, e.g., cancer.

For example, tyrosine kinase inhibitors include but are not limited to, Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (Gilotrif®); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS187724-61-4).

EGFR antibodies include but are not limited to, Cetuximab (Erbitux®); Panitumumab (Vectibix®); Matuzumab (EMD-72000); Nimotuzumab (hR3); Zalutumumab; TheraCIM h-R₃; MDX0447 (CAS 339151-96-1); and ch806 (mAb-806, CAS 946414-09-1).

Other HER2 inhibitors include but are not limited to, Neratinib (HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aa,5p,6aa)-octahydro-2-methylcyclopenta[c]pyrrol-5-yl]methoxy]-4-quinazolinamine (XL647, CAS 781613-23-8).

HER3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)—N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1Hpyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin 3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

IGFR inhibitors include but are not limited to, BMS-754807, XL-228, OSI-906, GSK0904529A, A-928605, AXL1717, KW-2450, MK0646, AMG479, IMCA12, MEDI-573, and BI836845. See e.g., Yee, JNCI, 104; 975 (2012) for review.

In another embodiment, the 3-(1-oxoisoindolin-2-yl)piperidine-2,6-dione IKZF2 degrader compounds of the present disclosure are used in combination with one or more proliferation signaling pathway inhibitors, including but not limited to, MEK inhibitors, BRAF inhibitors, PI3K/Akt inhibitors, SHP2 inhibitors, and also mTOR inhibitors, and CDK inhibitors, for treating a disease, e.g., cancer.

For example, mitogen-activated protein kinase (MEK) inhibitors include but are not limited to, XL-518 (also known as GDC-0973, CAS No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

BRAF inhibitors include, but are not limited to, Vemurafenib (or Zelboraf®), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®), or Ipilimumab (or MDX-010, MDX-101, or Yervoy).

Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC0941, RG7321, GNE0941, Pictrelisib, or Pictilisib; and described in PCT Publication Nos. WO 09/036082 and WO 09/055730); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethylcyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); (S)-N1-(4-methyl-5-(2-(1,1,1-trifluoro-2-methylpropan-2-yl)pyridin-4-yl)thiazol-2-yl)pyrrolidine-1,2-dicarboxamide (also known as BYL719 or Alpelisib); 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide (also known as GDC0032, RG7604, or Taselisib).

mTOR inhibitors include but are not limited to, Temsirolimus (Torisel®); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-a-aspartylL-serine-, inner salt (SF1126, CAS 936487-67-1).

CDK inhibitors include but are not limited to, Palbociclib (also known as PD-0332991, Ibrance®, 6-Acetyl-8-cyclopentyl-5-methyl-2-{[5-(1-piperazinyl)-2-pyridinyl]amino}pyrido[2,3-d]pyrimidin-7(8H)-one).

In yet another embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more pro-apoptotics, including but not limited to, IAP inhibitors, BCL2 inhibitors, MCL1 inhibitors, TRAIL agents, CHK inhibitors, for treating a disease, e.g., cancer.

For examples, IAP inhibitors include but are not limited to, LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, US2006/0014700, US2006/0025347, WO 06/069063, WO 06/010118, WO 06/017295, and WO08/134679, all of which are incorporated herein by reference.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab(CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinylpyrazolo[1,5-a]pyrimidin-7-amine (SCH900776, CAS 891494-63-6); 5-(3-Fluorophenyl)-3-ureidothiophene-2-carboxylic acid N-[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7,4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL (SEQ ID NO: 33)), and CBP501 ((d-Bpa)sws(d-Phe-F5)(d-Cha)rrrqrr).

In a further embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more immunomodulators (e.g., one or more of an activator of a costimulatory molecule or an inhibitor of an immune checkpoint molecule), for treating a disease, e.g., cancer.

In certain embodiments, the immunomodulator is an activator of a costimulatory molecule. In one embodiment, the agonist of the costimulatory molecule is selected from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

### GITR Agonists

In some embodiments, a GITR agonist is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the GITR agonist is GWN323 (Novartis), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

### Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of MAB7 disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-GITR antibody molecule comprises a heavy chain variable region (VH) comprising a VHCDR1 amino acid sequence of SEQ ID NO: 9, a VHCDR2 amino acid sequence of SEQ ID NO: 11, and a VHCDR3 amino acid sequence of SEQ ID NO: 13; and a light chain variable region (VL) comprising a VLCDR1 amino acid sequence of SEQ ID NO: 14, a VLCDR2 amino acid sequence of SEQ ID NO: 16, and a VLCDR3 amino acid sequence of SEQ ID NO: 18, each disclosed in Table 1.

In one embodiment, the anti-GITR antibody molecule comprises a VH comprising the amino acid sequence of SEQ ID NO: 1, or an amino acid sequence at least 85%, 90%, 95%, or 99% identical or higher to SEQ ID NO: 1. In one embodiment, the anti-GITR antibody molecule comprises a VL comprising the amino acid sequence of SEQ ID NO: 2, or an amino acid sequence at least 85%, 90%, 95%, or 99% identical or higher to SEQ ID NO: 2. In one embodiment, the anti-GITR antibody molecule comprises a VH comprising the amino acid sequence of SEQ ID NO: 1 and a VL comprising the amino acid sequence of SEQ ID NO: 2.

In one embodiment, the antibody molecule comprises a VH encoded by the nucleotide sequence of SEQ ID NO: 5, or a nucleotide sequence at least 85%, 90%, 95%, or 99% identical or higher to SEQ ID NO: 5. In one embodiment, the antibody molecule comprises a VL encoded by the nucleotide sequence of SEQ ID NO: 6, or a nucleotide sequence at least 85%, 90%, 95%, or 99% identical or higher to SEQ ID NO: 6. In one embodiment, the antibody molecule comprises a VH encoded by the nucleotide sequence of SEQ ID NO: 5 and a VL encoded by the nucleotide sequence of SEQ ID NO: 6.

In one embodiment, the anti-GITR antibody molecule comprises a heavy chain comprising the amino acid sequence of SEQ ID NO: 3, or an amino acid sequence at least 85%, 90%, 95%, or 99% identical or higher to SEQ ID NO: 3. In one embodiment, the anti-GITR antibody molecule comprises a light chain comprising the amino acid sequence of SEQ ID NO: 4, or an amino acid sequence at least 85%, 90%, 95%, or 99% identical or higher to SEQ ID NO: 4. In one embodiment, the anti-GITR antibody molecule comprises a heavy chain comprising the amino acid sequence of SEQ ID NO: 3 and a light chain comprising the amino acid sequence of SEQ ID NO: 4.

In one embodiment, the antibody molecule comprises a heavy chain encoded by the nucleotide sequence of SEQ ID NO: 7, or a nucleotide sequence at least 85%, 90%, 95%, or 99% identical or higher to SEQ ID NO: 7. In one embodiment, the antibody molecule comprises a light chain encoded by the nucleotide sequence of SEQ ID NO: 8, or a nucleotide sequence at least 85%, 90%, 95%, or 99% identical or higher to SEQ ID NO: 8. In one embodiment, the antibody molecule comprises a heavy chain encoded by the nucleotide sequence of SEQ ID NO: 7 and a light chain encoded by the nucleotide sequence of SEQ ID NO: 8.

The antibody molecules described herein can be made by vectors, host cells, and methods described in WO 2016/057846, incorporated by reference in its entirety.

TABLE 1
Amino acid and nucleotide sequences of exemplary anti-GITR antibody molecule

| | | |
|---|---|---|
| MAB7 | | |
| SEQ ID NO: 1 | VH | EVQLVESGGGLVQSGGSLRLSCAASGFSLSSYGVDWV |
| | | RQAPGKGLEWVGVIWGGGGTYYASSLMGRFTISRDNS |
| | | KNTLYLQMNSLRAEDTAVYYCARHAYGHDGGFAMD |
| | | YWGQGTLVTVSS |
| SEQ ID NO: 2 | VL | EIVMTQSPATLSVSPGERATLSCRASESVSSNVAWYQQ |
| | | RPGQAPRLLIYGASNRATGIPARFSGSGSGTDFTLTISRL |
| | | EPEDFAVYYCGQSYSYPFTFGQGTKLEIK |
| SEQ ID NO: 3 | Heavy | EVQLVESGGGLVQSGGSLRLSCAASGFSLSSYGVDWV |
| | Chain | RQAPGKGLEWVGVIWGGGGTYYASSLMGRFTISRDNS |
| | | KNTLYLQMNSLRAEDTAVYYCARHAYGHDGGFAMD |
| | | YWGQGTLVTVSSASTKGPSVFPLAPSSKSTSGGTAALG |
| | | CLVKDYFPEPVTVSWNSGALTSGVHTFPAVLQSSGLYS |
| | | LSSVVTVPSSSLGTQTYICNVNHKPSNTKVDKRVEPKS |
| | | CDKTHTCPPCPAPELLGGPSVFLFPPKPKDTLMISRTPE |
| | | VTCVVVDVSHEDPEVKFNWYVDGVEVHNAKTKPREE |
| | | QYNSTYRVVSVLTVLHQDWLNGKEYKCKVSNKALPA |
| | | PIEKTISKAKGQPREPQVYTLPPSREEMTKNQVSLTCLV |
| | | KGFYPSDIAVEWESNGQPENNYKTTPPVLDSDGSFFLY |
| | | SKLTVDKSRWQQGNVFSCSVMHEALHNHYTQKSLSLS |
| | | PGK |
| SEQ ID NO: 4 | Light | EIVMTQSPATLSVSPGERATLSCRASESVSSNVAWYQQ |
| | Chain | RPGQAPRLLIYGASNRATGIPARFSGSGSGTDFTLTISRL |
| | | EPEDFAVYYCGQSYSYPFTFGQGTKLEIKRTVAAPSVFI |
| | | FPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| SEQ ID NO: 5 | DNA | GAGGTGCAGCTGGTGGAATCTGGCGGCGGACTGGTG |
| | VH | CAGTCCGGCGGCTCTCTGAGACTGTCTTGCGCTGCCT |
| | | CCGGCTTCTCCCTGTCCTCTTACGGCGTGGACTGGGT |
| | | GCGACAGGCCCCTGGCAAGGGCCTGGAATGGGTGGG |
| | | AGTGATCTGGGGCGGAGGCGGCACCTACTACGCCTC |
| | | TTCCCTGATGGGCCGGTTCACCATCTCCCGGGACAAC |
| | | TCCAAGAACACCCTGTACCTGCAGATGAACTCCCTG |
| | | CGGGCCGAGGACACCGCCGTGTACTACTGCGCCAGA |
| | | CACGCCTACGGCCACGACGGCGGCTTCGCCATGGAT |
| | | TATTGGGGCCAGGGCACCCTGGTGACAGTGTCCTCC |
| SEQ ID NO: 6 | DNA | GAGATCGTGATGACCCAGTCCCCCGCCACCCTGTCT |
| | VL | GTGTCTCCCGGCGAGAGAGCCACCCTGAGCTGCAGA |
| | | GCCTCCGAGTCCGTGTCCTCCAACGTGGCCTGGTATC |
| | | AGCAGAGACCTGGTCAGGCCCCTCGGCTGCTGATCT |
| | | ACGGCGCCTCTAACCGGGCCACCGGCATCCCTGCCA |
| | | GATTCTCCGGCTCCGGCAGCGGCACCGACTTCACCCT |
| | | GACCATCTCCCGGCTGGAACCCGAGGACTTCGCCGT |
| | | GTACTACTGCGGCCAGTCCTACTCATACCCCTTCACC |
| | | TTCGGCCAGGGCACCAAGCTGGAAATCAAG |
| SEQ ID NO: 7 | DNA | GAGGTGCAGCTGGTGGAATCTGGCGGCGGACTGGTG |
| | Heavy | CAGTCCGGCGGCTCTCTGAGACTGTCTTGCGCTGCCT |
| | Chain | CCGGCTTCTCCCTGTCCTCTTACGGCGTGGACTGGGT |
| | | GCGACAGGCCCCTGGCAAGGGCCTGGAATGGGTGGG |
| | | AGTGATCTGGGGCGGAGGCGGCACCTACTACGCCTC |
| | | TTCCCTGATGGGCCGGTTCACCATCTCCCGGGACAAC |
| | | TCCAAGAACACCCTGTACCTGCAGATGAACTCCCTG |
| | | CGGGCCGAGGACACCGCCGTGTACTACTGCGCCAGA |
| | | CACGCCTACGGCCACGACGGCGGCTTCGCCATGGAT |
| | | TATTGGGGCCAGGGCACCCTGGTGACAGTGTCCTCC |
| | | GCTAGCACCAAGGGCCCAAGTGTGTTTCCCCTGGCC |
| | | CCCAGCAGCAAGTCTACTTCCGGCGGAACTGCTGCC |
| | | CTGGGTTGCCTGGTGAAGGACTACTTCCCCGAGCCC |
| | | GTGACAGTGTCCTGGAACTCTGGGGCTCTGACTTCCG |
| | | GCGTGCACACCTTCCCCGCCGTGCTGCAGAGCAGCG |
| | | GCCTGTACAGCCTGAGCAGCGTGGTGACAGTGCCCT |
| | | CCAGCTCTCTGGGAACCCAGACCTATATCTGCAACG |
| | | TGAACCACAAGCCCAGCAACACCAAGGTGGACAAG |
| | | AGAGTGGAGCCCAAGAGCTGCGACAAGACCCACAC |
| | | CTGCCCCCCCTGCCCAGCTCCAGAACTGCTGGGAGG |
| | | GCCTTCCGTGTTCCTGTTCCCCCCCAAGCCCAAGGAC |
| | | ACCCTGATGATCAGCAGGACCCCCGAGGTGACCTGC |
| | | GTGGTGGTGGACGTGTCCCACGAGGACCCAGAGGTG |
| | | AAGTTCAACTGGTACGTGGACGGCGTGGAGGTGCAC |
| | | AACGCCAAGACCAAGCCCAGAGAGGAGCAGTACAA |
| | | CAGCACCTACAGGGTGGTGTCCGTGCTGACCGTGCT |
| | | GCACCAGGACTGGCTGAACGGCAAAGAATACAAGT |
| | | GCAAAGTCTCCAACAAGGCCCTGCCAGCCCCAATCG |
| | | AAAAGACAATCAGCAAGGCCAAGGGCCAGCCACGG |
| | | GAGCCCCAGGTGTACACCCTGCCCCCCAGCCGGGAG |
| | | GAGATGACCAAGAACCAGGTGTCCCTGACCTGTCTG |
| | | GTGAAGGGCTTCTACCCCAGCGATATCGCCGTGGAG |
| | | TGGGAGAGCAACGGCCAGCCCGAGAACAACTACAA |
| | | GACCACCCCCCCAGTGCTGGACAGCGACGGCAGCTT |
| | | CTTCCTGTACAGCAAGCTGACCGTGGACAAGTCCAG |
| | | GTGGCAGCAGGGCAACGTGTTCAGCTGCAGCGTGAT |
| | | GCACGAGGCCCTGCACAACCACTACACCCAGAAGTC |
| | | CCTGAGCCTGAGCCCCGGCAAG |
| SEQ ID NO: 8 | DNA | GAGATCGTGATGACCCAGTCCCCCGCCACCCTGTCT |
| | Light | GTGTCTCCCGGCGAGAGAGCCACCCTGAGCTGCAGA |
| | Chain | GCCTCCGAGTCCGTGTCCTCCAACGTGGCCTGGTATC |
| | | AGCAGAGACCTGGTCAGGCCCCTCGGCTGCTGATCT |
| | | ACGGCGCCTCTAACCGGGCCACCGGCATCCCTGCCA |
| | | GATTCTCCGGCTCCGGCAGCGGCACCGACTTCACCCT |
| | | GACCATCTCCCGGCTGGAACCCGAGGACTTCGCCGT |
| | | GTACTACTGCGGCCAGTCCTACTCATACCCCTTCACC |
| | | TTCGGCCAGGGCACCAAGCTGGAAATCAAGCGTACG |
| | | GTGGCCGCTCCCAGCGTGTTCATCTTCCCCCCCAGCG |
| | | ACGAGCAGCTGAAGAGCGGCACCGCCAGCGTGGTGT |
| | | GCCTGCTGAACAACTTCTACCCCCGGGAGGCCAAGG |
| | | TGCAGTGGAAGGTGGACAACGCCCTGCAGAGCGGCA |
| | | ACAGCCAGGAGAGCGTCACCGAGCAGGACAGCAAG |
| | | GACTCCACCTACAGCCTGAGCAGCACCCTGACCCTG |
| | | AGCAAGGCCGACTACGAGAAGCATAAGGTGTACGCC |
| | | TGCGAGGTGACCCACCAGGGCCTGTCCAGCCCCGTG |
| | | ACCAAGAGCTTCAACAGGGGCGAGTGC |
| SEQ ID NO: 9 (KABAT) | HCDR1 | SYGVD |
| SEQ ID NO: 10 | HCDR1 | GFSLSSY |
| (CHOTHIA) | | |
| SEQ ID NO: 11 (KABAT) | HCDR2 | VIWGGGGTYYASSLMG |
| SEQ ID NO: 12 | HCDR2 | WGGGG |
| (CHOTHIA) | | |
| SEQ ID NO: 13 (KABAT) | HCDR3 | HAYGHDGGFAMDY |
| SEQ ID NO: 13 | HCDR3 | HAYGHDGGFAMDY |
| (CHOTHIA) | | |
| SEQ ID NO: 14 (KABAT) | LCDR1 | RASESVSSNVA |
| SEQ ID NO: 15 | LCDR1 | SESVSSN |
| (CHOTHIA) | | |
| SEQ ID NO: 16 (KABAT) | LCDR2 | GASNRAT |
| SEQ ID NO: 17 | LCDR2 | GAS |
| (CHOTHIA) | | |
| SEQ ID NO: 18 (KABAT) | LCDR3 | GQSYSYPFT |
| SEQ ID NO: 19 | LCDR3 | SYSYPF |
| (CHOTHIA) | | |

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 2.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 2
Amino acid sequence of other exemplary anti-GITR antibody molecules

| | | |
|---|---|---|
| BMS-986156 | | |
| SEQ ID NO: 20 | VH | QVQLVESGGGVVQPGRSLRLSCAASGFTFSSYGMHWVRQAP |
| | | GKGLEWVAVIWYEGSNKYYADSVKGRFTISRDNSKNTLYLQ |
| | | MNSLRAEDTAVYYCARGGSMVRGDYYYGMDVWGQGTTVT |
| | | VSS |
| SEQ ID NO: 21 | VL | AIQLTQSPSSLSASVGDRVTITCRASQGISSALAWYQQKPGKA |
| | | PKLLIYDASSLESGVPSRFSGSGSGTDFTLTISSLQPEDFATYY |
| | | CQQFNSYPYTFGQGTKLEIK |

In certain embodiments, the immunomodulator is an inhibitor of an immune checkpoint molecule. In one embodiment, the immunomodulator is an inhibitor of PD-1, PD-L1, PD-L2, CTLA4, TIM3, LAG3, VISTA, BTLA, TIGIT, LAIR1, CD160, 2B4 and/or TGFRbeta. In one embodiment, the inhibitor of an immune checkpoint molecule inhibits PD-1, PD-L1, LAG-3, TIM-3 or CTLA4, or any combination thereof. The term “inhibition” or “inhibitor” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40%, 50% or more is included by this term. Thus, inhibition need not be 100%.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)₂, Fv, or a single chain Fv fragment (scFv)). In yet other embodiments, the antibody molecule has a heavy chain constant region (Fc) selected from, e.g., the heavy chain constant regions of IgG1, IgG2, IgG3, IgG4, IgM, IgA1, IgA2, IgD, and IgE; particularly, selected from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of Fc receptor binding, antibody glycosylation, the number of cysteine residues, effector cell function, or complement function).

In certain embodiments, the antibody molecule is in the form of a bispecific or multispecific antibody molecule. In one embodiment, the bispecific antibody molecule has a first binding specificity to PD-1 or PD-L1 and a second binding specificity, e.g., a second binding specificity to TIM-3, LAG-3, or PD-L2. In one embodiment, the bispecific antibody molecule binds to PD-1 or PD-L1 and TIM-3. In another embodiment, the bispecific antibody molecule binds to PD-1 or PD-L1 and LAG-3. In another embodiment, the bispecific antibody molecule binds to PD-1 and PD-L1. In yet another embodiment, the bispecific antibody molecule binds to PD-1 and PD-L2. In another embodiment, the bispecific antibody molecule binds to TIM-3 and LAG-3. Any combination of the aforesaid molecules can be made in a multispecific antibody molecule, e.g., a trispecific antibody that includes a first binding specificity to PD-1 or PD-1, and a second and third binding specificities to two or more of TIM-3, LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of LAG-3, TIM-3 or CTLA4. In an exemplary embodiment, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule.

Other combinations of immunomodulators with a PD-1 inhibitor (e.g., one or more of PD-L2, CTLA4, TIM3, LAG3, VISTA, BTLA, TIGIT, LAIR1, CD160, 2B4 and/or TGFR) are also within the present disclosure. Any of the antibody molecules known in the art or disclosed herein can be used in the aforesaid combinations of inhibitors of checkpoint molecule.

### PD-1 Inhibitors

In some embodiments, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with a PD-1 inhibitor to treat a disease, e.g., cancer. In some embodiments, the PD-1 inhibitor is selected from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 3 (e.g., from the heavy and light chain variable region sequences of BAP049-Clone-E or BAP049-Clone-B disclosed in Table 3), or encoded by a nucleotide sequence shown in Table 3. In some embodiments, the CDRs are according to the Kabat definition (e.g., as set out in Table 3). In some embodiments, the CDRs are according to the Chothia definition (e.g., as set out in Table 3). In some embodiments, the CDRs are according to the combined CDR definitions of both Kabat and Chothia (e.g., as set out in Table 3). In one embodiment, the combination of Kabat and Chothia CDR of VH CDR1 comprises the amino acid sequence GYTFTTYWMH (SEQ ID NO: 213). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 3, or encoded by a nucleotide sequence shown in Table 3.

In one embodiment, the anti-PD-1 antibody molecule comprises a heavy chain variable region (VH) comprising a VHCDR1 amino acid sequence of SEQ ID NO: 22, a VHCDR2 amino acid sequence of SEQ ID NO: 23, and a VHCDR3 amino acid sequence of SEQ ID NO: 24; and a light chain variable region (VL) comprising a VLCDR1 amino acid sequence of SEQ ID NO: 31, a VLCDR2 amino acid sequence of SEQ ID NO: 32, and a VLCDR3 amino acid sequence of SEQ ID NO: 286, each disclosed in Table 3.

In one embodiment, the antibody molecule comprises a VH comprising a VHCDR1 encoded by the nucleotide sequence of SEQ ID NO: 45, a VHCDR2 encoded by the nucleotide sequence of SEQ ID NO: 46, and a VHCDR3 encoded by the nucleotide sequence of SEQ ID NO: 47; and a VL comprising a VLCDR1 encoded by the nucleotide sequence of SEQ ID NO: 50, a VLCDR2 encoded by the nucleotide sequence of SEQ ID NO: 51, and a VLCDR3 encoded by the nucleotide sequence of SEQ ID NO: 52, each disclosed in Table 3.

In one embodiment, the anti-PD-1 antibody molecule comprises a VH comprising the amino acid sequence of SEQ ID NO: 27, or an amino acid sequence at least 85%, 90%, 95%, or 99% identical or higher to SEQ ID NO: 27. In one embodiment, the anti-PD-1 antibody molecule comprises a VL comprising the amino acid sequence of SEQ ID NO: 41, or an amino acid sequence at least 85%, 90%, 95%, or 99% identical or higher to SEQ ID NO: 41. In one embodiment, the anti-PD-1 antibody molecule comprises a VL comprising the amino acid sequence of SEQ ID NO: 37, or an amino acid sequence at least 85%, 90%, 95%, or 99% identical or higher to SEQ ID NO: 37. In one embodiment, the anti-PD-1 antibody molecule comprises a VH comprising the amino acid sequence of SEQ ID NO: 27 and a VL comprising the amino acid sequence of SEQ ID NO: 41. In one embodiment, the anti-PD-1 antibody molecule comprises a VH comprising the amino acid sequence of SEQ ID NO: 27 and a VL comprising the amino acid sequence of SEQ ID NO: 37.

In one embodiment, the antibody molecule comprises a VH encoded by the nucleotide sequence of SEQ ID NO: 28, or a nucleotide sequence at least 85%, 90%, 95%, or 99% identical or higher to SEQ ID NO: 28. In one embodiment, the antibody molecule comprises a VL encoded by the nucleotide sequence of SEQ ID NO: 42 or 38, or a nucleotide sequence at least 85%, 90%, 95%, or 99% identical or higher to SEQ ID NO: 42 or 38. In one embodiment, the antibody molecule comprises a VH encoded by the nucleotide sequence of SEQ ID NO: 28 and a VL encoded by the nucleotide sequence of SEQ ID NO: 42 or 38.

In one embodiment, the anti-PD-1 antibody molecule comprises a heavy chain comprising the amino acid sequence of SEQ ID NO: 29, or an amino acid sequence at least 85%, 90%, 95%, or 99% identical or higher to SEQ ID NO: 29. In one embodiment, the anti-PD-1 antibody molecule comprises a light chain comprising the amino acid sequence of SEQ ID NO: 43, or an amino acid sequence at least 85%, 90%, 95%, or 99% identical or higher to SEQ ID NO: 43. In one embodiment, the anti-PD-1 antibody molecule comprises a light chain comprising the amino acid sequence of SEQ ID NO: 39, or an amino acid sequence at least 85%, 90%, 95%, or 99% identical or higher to SEQ ID NO: 39. In one embodiment, the anti-PD-1 antibody molecule comprises a heavy chain comprising the amino acid sequence of SEQ ID NO: 29 and a light chain comprising the amino acid sequence of SEQ ID NO: 43. In one embodiment, the anti-PD-1 antibody molecule comprises a heavy chain comprising the amino acid sequence of SEQ ID NO: 29 and a light chain comprising the amino acid sequence of SEQ ID NO: 39.

In one embodiment, the antibody molecule comprises a heavy chain encoded by the nucleotide sequence of SEQ ID NO: 30, or a nucleotide sequence at least 85%, 90%, 95%, or 99% identical or higher to SEQ ID NO: 30. In one embodiment, the antibody molecule comprises a light chain encoded by the nucleotide sequence of SEQ ID NO: 44 or 40, or a nucleotide sequence at least 85%, 90%, 95%, or 99% identical or higher to SEQ ID NO: 44 or 40. In one embodiment, the antibody molecule comprises a heavy chain encoded by the nucleotide sequence of SEQ ID NO: 30 and a light chain encoded by the nucleotide sequence of SEQ ID NO: 44 or 40.

The antibody molecules described herein can be made by vectors, host cells, and methods described in US 2015/0210769, incorporated by reference in its entirety.

TABLE 3
Amino acid and nucleotide sequences of exemplary anti-PD-1 antibody molecules

| | | |
|---|---|---|
| BAP049-Clone-B HC | | |
| SEQ ID NO: 22 (Kabat) | HCDR1 | TYWMH |
| SEQ ID NO: 23 (Kabat) | HCDR2 | NIYPGTGGSNFDEKFKN |
| SEQ ID NO: 24 (Kabat) | HCDR3 | WTTGTGAY |
| SEQ ID NO: 25 | HCDR1 | GYTFTTY |
| (Chothia) | | |
| SEQ ID NO: 26 | HCDR2 | YPGTGG |
| (Chothia) | | |
| SEQ ID NO: 24 | HCDR3 | WTTGTGAY |
| (Chothia) | | |
| SEQ ID NO: 27 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 28 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 29 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 30 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |
| BAP049-Clone-B LC | | |
| SEQ ID NO: 31 (Kabat) | LCDR1 | KSSQSLLDSGNQKNFLT |
| SEQ ID NO: 32 (Kabat) | LCDR2 | WASTRES |
| SEQ ID NO: 286 | LCDR3 | QNDYSYPYT |
| (Kabat) | | |
| SEQ ID NO: 34 | LCDR1 | SQSLLDSGNQKNF |
| (Chothia) | | |
| SEQ ID NO: 35 | LCDR2 | WAS |
| (Chothia) | | |
| SEQ ID NO: 36 | LCDR3 | DYSYPY |
| (Chothia) | | |
| SEQ ID NO: 37 | VL | EIVLTQSPATLSLSPGERATLSCKSSQSLLDSGNQKNFLTWY |
| | | QQKPGKAPKLLIYWASTRESGVPSRFSGSGSGTDFTFTISSLQ |
| | | PEDIATYYCQNDYSYPYTFGQGTKVEIK |
| SEQ ID NO: 38 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAG |
| SEQ ID NO: 39 | Light | EIVLTQSPATLSLSPGERATLSCKSSQSLLDSGNQKNFLTWY |
| | chain | QQKPGKAPKLLIYWASTRESGVPSRFSGSGSGTDFTFTISSLQ |
| | | PEDIATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 40 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |
| BAP049-Clone-E HC | | |
| SEQ ID NO: 22 (Kabat) | HCDR1 | TYWMH |
| SEQ ID NO: 23 (Kabat) | HCDR2 | NIYPGTGGSNFDEKFKN |
| SEQ ID NO: 24 (Kabat) | HCDR3 | WTTGTGAY |
| SEQ ID NO: 25 | HCDR1 | GYTFTTY |
| (Chothia) | | |
| SEQ ID NO: 26 | HCDR2 | YPGTGG |
| (Chothia) | | |
| SEQ ID NO: 24 | HCDR3 | WTTGTGAY |
| (Chothia) | | |
| SEQ ID NO: 27 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 28 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 29 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 30 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |
| BAP049-Clone-E LC | | |
| SEQ ID NO: 31 (Kabat) | LCDR1 | KSSQSLLDSGNQKNFLT |
| SEQ ID NO: 32 (Kabat) | LCDR2 | WASTRES |
| SEQ ID NO: 286 | LCDR3 | QNDYSYPYT |
| (Kabat) | | |
| SEQ ID NO: 34 | LCDR1 | SQSLLDSGNQKNF |
| (Chothia) | | |
| SEQ ID NO: 35 | LCDR2 | WAS |
| (Chothia) | | |
| SEQ ID NO: 36 | LCDR3 | DYSYPY |
| (Chothia) | | |
| SEQ ID NO: 41 | VL | EIVLTQSPATLSLSPGERATLSCKSSQSLLDSGNQKNFLTWY |
| | | QQKPGQAPRLLIYWASTRESGVPSRFSGSGSGTDFTFTISSLE |
| | | AEDAATYYCQNDYSYPYTFGQGTKVEIK |
| SEQ ID NO: 42 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAG |
| SEQ ID NO: 43 | Light | EIVLTQSPATLSLSPGERATLSCKSSQSLLDSGNQKNFLTWY |
| | chain | QQKPGQAPRLLIYWASTRESGVPSRFSGSGSGTDFTFTISSLE |
| | | AEDAATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 44 | DNA | GAGATCGTCCTGACTCAGTCACCCGCTACCCTGAGCCTGA |
| | | GCCCTGGCGAGCGGGCTACACTGAGCTGTAAATCTAGTC |
| | | AGTCACTGCTGGATAGCGGTAATCAGAAGAACTTCCTGA |
| | light | CCTGGTATCAGCAGAAGCCCGGTCAAGCCCCTAGACTGC |
| | chain | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |
| BAP049-Clone-B HC | | |
| SEQ ID NO: 45 (Kabat) | HCDR1 | ACCTACTGGATGCAC |
| SEQ ID NO: 46 (Kabat) | HCDR2 | AATATCTACCCCGGCACCGGCGGCTCTAACTTCGACGAG |
| | | AAGTTTAAGAAT |
| SEQ ID NO: 47 (Kabat) | HCDR3 | TGGACTACCGGCACAGGCGCCTAC |
| SEQ ID NO: 48 | HCDR1 | GGCTACACCTTCACTACCTAC |
| (Chothia) | | |
| SEQ ID NO: 49 | HCDR2 | TACCCCGGCACCGGCGGC |
| (Chothia) | | |
| SEQ ID NO: 47 | HCDR3 | TGGACTACCGGCACAGGCGCCTAC |
| (Chothia) | | |
| BAP049-Clone-B LC | | |
| SEQ ID NO: 50 (Kabat) | LCDR1 | AAATCTAGTCAGTCACTGCTGGATAGCGGTAATCAGAAG |
| | | AACTTCCTGACC |
| SEQ ID NO: 51 (Kabat) | LCDR2 | TGGGCCTCTACTAGAGAATCA |
| SEQ ID NO: 52 (Kabat) | LCDR3 | CAGAACGACTATAGCTACCCCTACACC |
| SEQ ID NO: 53 | LCDR1 | AGTCAGTCACTGCTGGATAGCGGTAATCAGAAGAACTTC |
| (Chothia) | | |
| SEQ ID NO: 54 | LCDR2 | TGGGCCTCT |
| (Chothia) | | |
| SEQ ID NO: 55 | LCDR3 | GACTATAGCTACCCCTAC |
| (Chothia) | | |
| BAP049-Clone-E HC | | |
| SEQ ID NO: 45 (Kabat) | HCDR1 | ACCTACTGGATGCAC |
| | | AATATCTACCCCGGCACCGGCGGCTCTAACTTCGACGAG |
| SEQ ID NO: 46 (Kabat) | HCDR2 | AAGTTTAAGAAT |
| SEQ ID NO: 47 (Kabat) | HCDR3 | TGGACTACCGGCACAGGCGCCTAC |
| SEQ ID NO: 48 | HCDR1 | GGCTACACCTTCACTACCTAC |
| (Chothia) | | |
| SEQ ID NO: 49 | HCDR2 | TACCCCGGCACCGGCGGC |
| (Chothia) | | |
| SEQ ID NO: 47 | HCDR3 | TGGACTACCGGCACAGGCGCCTAC |
| (Chothia) | | |
| BAP049-Clone-E LC | | |
| SEQ ID NO: 50 (Kabat) | LCDR1 | AAATCTAGTCAGTCACTGCTGGATAGCGGTAATCAGAAG |
| | | AACTTCCTGACC |
| SEQ ID NO: 51 (Kabat) | LCDR2 | TGGGCCTCTACTAGAGAATCA |
| SEQ ID NO: 52 (Kabat) | LCDR3 | CAGAACGACTATAGCTACCCCTACACC |
| SEQ ID NO: 53 | LCDR1 | AGTCAGTCACTGCTGGATAGCGGTAATCAGAAGAACTTC |
| (Chothia) | | |
| SEQ ID NO: 54 | LCDR2 | TGGGCCTCT |
| (Chothia) | | |
| SEQ ID NO: 55 | LCDR3 | GACTATAGCTACCCCTAC |
| (Chothia) | | |

### Other Exemplary PD-1 Inhibitors

In some embodiments, the anti-PD-1 antibody is Nivolumab (CAS Registry Number: 946414-94-4). Alternative names for Nivolumab include MDX-1106, MDX-1106-04, ONO—4538, BMS-936558 or OPDIVO®. Nivolumab is a fully human IgG4 monoclonal antibody, which specifically blocks PD1. Nivolumab (clone 5C4) and other human monoclonal antibodies that specifically bind to PD1 are disclosed in U.S. Pat. No. 8,008,449 and PCT Publication No. WO2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 4.

In other embodiments, the anti-PD-1 antibody is Pembrolizumab. Pembrolizumab (Trade name KEYTRUDA formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New EnglandJournal ofMedicine 369 (2): 134-44, PCT Publication No. WO2009/114335, and U.S. Pat. No. 8,354,509, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 4.

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in PCT Publication No. WO2009/101611, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 4.

Other anti-PD1 antibodies are disclosed in U.S. Pat. No. 8,609,089, US Publication No. 2010028330, and/or US Publication No. 20120114649, incorporated by reference in their entirety. Other anti-PD1 antibodies include AMP 514 (Amplimmune).

In one embodiment, the anti-PD-1 antibody molecule is MEDJ0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MED10680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANBO11. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence).

In some embodiments, the PD-1 inhibitor is AMP-224 (B7-DCIg (Amplimmune), e.g., disclosed in WO 2010/027827 and WO 2011/066342, incorporated by reference in their entirety).

TABLE 4
Amino acid sequences of other exemplary anti-PD-1 antibody molecules

| | | |
|---|---|---|
| Nivolumab | | |
| SEQ ID NO: 56 | Heavy | QVQLVESGGGVVQPGRSLRLDCKASGITFSNSGMHWVRQAPGKG |
| | chain | LEWVAVIWYDGSKRYYADSVKGRFTISRDNSKNTLFLQMNSLRA |
| | | EDTAVYYCATNDDYWGQGTLVTVSSASTKGPSVFPLAPCSRSTSE |
| | | STAALGCLVKDYFPEPVTVSWNSGALTSGVHTFPAVLQSSGLYSL |
| | | SSVVTVPSSSLGTKTYTCNVDHKPSNTKVDKRVESKYGPPCPPCP |
| | | APEFLGGPSVFLFPPKPKDTLMISRTPEVTCVVVDVSQEDPEVQFN |
| | | WYVDGVEVHNAKTKPREEQFNSTYRVVSVLTVLHQDWLNGKEY |
| | | KCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: 57 | Light | EIVLTQSPATLSLSPGERATLSCRASQSVSSYLAWYQQKPGQAPRL |
| | chain | LIYDASNRATGIPARFSGSGSGTDFTLTISSLEPEDFAVYYCQQSSN |
| | | WPRTFGQGTKVEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC |
| Pembrolizumab | | |
| SEQ ID NO: 58 | Heavy | QVQLVQSGVEVKKPGASVKVSCKASGYTFTNYYMYWVRQAPG |
| | chain | QGLEWMGGINPSNGGTNFNEKFKNRVTLTTDSSTTTAYMELKSL |
| | | QFDDTAVYYCARRDYRFDMGFDYWGQGTTVTVSSASTKGPSVF |
| | | PLAPCSRSTSESTAALGCLVKDYFPEPVTVSWNSGALTSGVHTFP |
| | | AVLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTVL |
| | | HQDWLNGKEYKCKVSNKGLPSSIEKTISKAKGQPREPQVYTLPPS |
| | | QEEMTKNQVSLTCLVKGFYPSDIAVEWESNGQPENNYKTTPPVL |
| | | DSDGSFFLYSRLTVDKSRWQEGNVFSCSVMHEALHNHYTQKSLS |
| | | LSLGK |
| SEQ ID NO: 59 | Light | EIVLTQSPATLSLSPGERATLSCRASKGVSTSGYSYLHWYQQKPG |
| | chain | QAPRLLIYLASYLESGVPARFSGSGSGTDFTLTISSLEPEDFAVYYC |
| | | QHSRDLPLTFGGGTKVEIKRTVAAPSVFIFPPSDEQLKSGTASVVC |
| | | LLNNFYPREAKVQWKVDNALQSGNSQESVTEQDSKDSTYSLSST |
| | | LTLSKADYEKHKVYACEVTHQGLSSPVTKSFNRGEC |
| Pidilizumab | | |
| SEQ ID NO: 60 | Heavy | QVQLVQSGSELKKPGASVKISCKASGYTFTNYGMNWVRQAPGQ |
| | chain | GLQWMGWINTDSGESTYAEEFKGRFVFSLDTSVNTAYLQITSLTA |
| | | EDTGMYFCVRVGYDALDYWGQGTLVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQ |
| | | DWLNGKEYKCKVSNKALPAPIEKTISKAKGQPREPQVYTLPPSRE |
| | | EMTKNQVSLTCLVKGFYPSDIAVEWESNGQPENNYKTTPPVLDS |
| | | DGSFFLYSKLTVDKSRWQQGNVFSCSVMHEALHNHYTQKSLSLS |
| | | PGK |
| SEQ ID NO: 61 | Light | EIVLTQSPSSLSASVGDRVTITCSARSSVSYMHWFQQKPGKAPKL |
| | chain | WIYRTSNLASGVPSRFSGSGSGTSYCLTINSLQPEDFATYYCQQRS |
| | | SFPLTFGGGTKLEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC |

### PD-L1 Inhibitors

In some embodiments, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with a PD-L1 inhibitor for treating a disease, e.g., cancer. In some embodiments, the PD-L1 inhibitor is selected from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

### Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of BAP058-Clone O or BAP058-Clone N disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In some embodiments, the CDRs are according to the combined CDR definitions of both Kabat and Chothia (e.g., as set out in Table 5). In one embodiment, the combination of Kabat and Chothia CDR of VH CDR1 comprises the amino acid sequence GYTFTSYWMY (SEQ ID NO: 214). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-PD-L1 antibody molecule comprises a heavy chain variable region (VH) comprising a VHCDR1 amino acid sequence of SEQ ID NO: 62, a VHCDR2 amino acid sequence of SEQ ID NO: 63, and a VHCDR3 amino acid sequence of SEQ ID NO: 64; and a light chain variable region (VL) comprising a VLCDR1 amino acid sequence of SEQ ID NO: 70, a VLCDR2 amino acid sequence of SEQ ID NO: 71, and a VLCDR3 amino acid sequence of SEQ ID NO: 72, each disclosed in Table 5.

In one embodiment, the anti-PD-L1 antibody molecule comprises a VH comprising a VHCDR1 encoded by the nucleotide sequence of SEQ ID NO: 89, a VHCDR2 encoded by the nucleotide sequence of SEQ ID NO: 90, and a VHCDR3 encoded by the nucleotide sequence of SEQ ID NO: 91; and a VL comprising a VLCDR1 encoded by the nucleotide sequence of SEQ ID NO: 94, a VLCDR2 encoded by the nucleotide sequence of SEQ ID NO: 95, and a VLCDR3 encoded by the nucleotide sequence of SEQ ID NO: 96, each disclosed in Table 5.

In one embodiment, the anti-PD-L1 antibody molecule comprises a VH comprising the amino acid sequence of SEQ ID NO: 67, or an amino acid sequence at least 85%, 90%, 95%, or 99% identical or higher to SEQ ID NO: 67. In one embodiment, the anti-PD-L1 antibody molecule comprises a VL comprising the amino acid sequence of SEQ ID NO: 77, or an amino acid sequence at least 85%, 90%, 95%, or 99% identical or higher to SEQ ID NO: 77. In one embodiment, the anti-PD-L1 antibody molecule comprises a VH comprising the amino acid sequence of SEQ ID NO: 81, or an amino acid sequence at least 85%, 90%, 95%, or 99% identical or higher to SEQ ID NO: 81. In one embodiment, the anti-PD-L 1 antibody molecule comprises a VL comprising the amino acid sequence of SEQ ID NO: 85, or an amino acid sequence at least 85%, 90%, 95%, or 99% identical or higher to SEQ ID NO: 85. In one embodiment, the anti-PD-L1 antibody molecule comprises a VH comprising the amino acid sequence of SEQ ID NO: 67 and a VL comprising the amino acid sequence of SEQ ID NO: 77. In one embodiment, the anti-PD-L1 antibody molecule comprises a VH comprising the amino acid sequence of SEQ ID NO: 81 and a VL comprising the amino acid sequence of SEQ ID NO: 85.

In one embodiment, the antibody molecule comprises a VH encoded by the nucleotide sequence of SEQ ID NO: 68, or a nucleotide sequence at least 85%, 90%, 95%, or 99% identical or higher to SEQ ID NO: 68. In one embodiment, the antibody molecule comprises a VL encoded by the nucleotide sequence of SEQ ID NO: 78, or a nucleotide sequence at least 85%, 90%, 95%, or 99% identical or higher to SEQ ID NO: 78. In one embodiment, the antibody molecule comprises a VH encoded by the nucleotide sequence of SEQ ID NO: 82, or a nucleotide sequence at least 85%, 90%, 95%, or 99% identical or higher to SEQ ID NO: 82. In one embodiment, the antibody molecule comprises a VL encoded by the nucleotide sequence of SEQ ID NO: 86, or a nucleotide sequence at least 85%, 90%, 95%, or 99% identical or higher to SEQ ID NO: 86. In one embodiment, the antibody molecule comprises a VH encoded by the nucleotide sequence of SEQ ID NO: 68 and a VL encoded by the nucleotide sequence of SEQ ID NO: 78. In one embodiment, the antibody molecule comprises a VH encoded by the nucleotide sequence of SEQ ID NO: 82 and a VL encoded by the nucleotide sequence of SEQ ID NO: 86.

In one embodiment, the anti-PD-L1 antibody molecule comprises a heavy chain comprising the amino acid sequence of SEQ ID NO: 69, or an amino acid sequence at least 85%, 90%, 95%, or 99% identical or higher to SEQ ID NO: 69. In one embodiment, the anti-PD-L1 antibody molecule comprises a light chain comprising the amino acid sequence of SEQ ID NO: 79, or an amino acid sequence at least 85%, 90%, 95%, or 99% identical or higher to SEQ ID NO: 79. In one embodiment, the anti-PD-L1 antibody molecule comprises a heavy chain comprising the amino acid sequence of SEQ ID NO: 83, or an amino acid sequence at least 85%, 90%, 95%, or 99% identical or higher to SEQ ID NO: 83. In one embodiment, the anti-PD-L1 antibody molecule comprises a light chain comprising the amino acid sequence of SEQ ID NO: 87, or an amino acid sequence at least 85%, 90%, 95%, or 99% identical or higher to SEQ ID NO: 87. In one embodiment, the anti-PD-L1 antibody molecule comprises a heavy chain comprising the amino acid sequence of SEQ ID NO: 69 and a light chain comprising the amino acid sequence of SEQ ID NO: 79. In one embodiment, the anti-PD-L1 antibody molecule comprises a heavy chain comprising the amino acid sequence of SEQ ID NO: 83 and a light chain comprising the amino acid sequence of SEQ ID NO: 87.

In one embodiment, the antibody molecule comprises a heavy chain encoded by the nucleotide sequence of SEQ ID NO: 76, or a nucleotide sequence at least 85%, 90%, 95%, or 99% identical or higher to SEQ ID NO: 76. In one embodiment, the antibody molecule comprises a light chain encoded by the nucleotide sequence of SEQ ID NO: 80, or a nucleotide sequence at least 85%, 90%, 95%, or 99% identical or higher to SEQ ID NO: 80. In one embodiment, the antibody molecule comprises a heavy chain encoded by the nucleotide sequence of SEQ ID NO: 84, or a nucleotide sequence at least 85%, 90%, 95%, or 99% identical or higher to SEQ ID NO: 84. In one embodiment, the antibody molecule comprises a light chain encoded by the nucleotide sequence of SEQ ID NO: 88, or a nucleotide sequence at least 85%, 90%, 95%, or 99% identical or higher to SEQ ID NO: 88. In one embodiment, the antibody molecule comprises a heavy chain encoded by the nucleotide sequence of SEQ ID NO: 76 and a light chain encoded by the nucleotide sequence of SEQ ID NO: 80. In one embodiment, the antibody molecule comprises a heavy chain encoded by the nucleotide sequence of SEQ ID NO: 84 and a light chain encoded by the nucleotide sequence of SEQ ID NO: 88.

The antibody molecules described herein can be made by vectors, host cells, and methods described in US 2016/0108123, incorporated by reference in its entirety.

TABLE 5
Amino acid and nucleotide sequences of exemplary anti-PD-L1 antibody molecules

| | | |
|---|---|---|
| BAP058-Clone O HC | | |
| SEQ ID NO: 62 (Kabat) | HCDR1 | SYWMY |
| SEQ ID NO: 63 (Kabat) | HCDR2 | RIDPNSGSTKYNEKFKN |
| SEQ ID NO: 64 (Kabat) | HCDR3 | DYRKGLYAMDY |
| SEQ ID NO: 65 | HCDR1 | GYTFTSY |
| (Chothia) | | |
| SEQ ID NO: 66 | HCDR2 | DPNSGS |
| (Chothia) | | |
| SEQ ID NO: 64 | HCDR3 | DYRKGLYAMDY |
| (Chothia) | | |
| SEQ ID NO: 67 | VH | EVQLVQSGAEVKKPGATVKISCKVSGYTFTSYWMYWV |
| | | RQARGQRLEWIGRIDPNSGSTKYNEKFKNRFTISRDNSK |
| | | NTLYLQMNSLRAEDTAVYYCARDYRKGLYAMDYWGQ |
| | | GTTVTVSS |
| SEQ ID NO: 68 | DNA VH | GAAGTGCAGCTGGTGCAGTCAGGCGCCGAAGTGAAG |
| | | AAACCCGGCGCTACCGTGAAGATTAGCTGTAAAGTCT |
| | | CAGGCTACACCTTCACTAGCTACTGGATGTACTGGGT |
| | | CCGACAGGCTAGAGGGCAAAGACTGGAGTGGATCGG |
| | | TAGAATCGACCCTAATAGCGGCTCTACTAAGTATAAC |
| | | GAGAAGTTTAAGAATAGGTTCACTATTAGTAGGGATA |
| | | ACTCTAAGAACACCCTGTACCTGCAGATGAATAGCCT |
| | | GAGAGCCGAGGACACCGCCGTCTACTACTGCGCTAGA |
| | | GACTATAGAAAGGGCCTGTACGCTATGGACTACTGGG |
| | | GTCAAGGCACTACCGTGACCGTGTCTTCA |
| SEQ ID NO: 69 | Heavy | EVQLVQSGAEVKKPGATVKISCKVSGYTFTSYWMYWV |
| | chain | RQARGQRLEWIGRIDPNSGSTKYNEKFKNRFTISRDNSK |
| | | NTLYLQMNSLRAEDTAVYYCARDYRKGLYAMDYWGQ |
| | | GTTVTVSSASTKGPSVFPLAPCSRSTSESTAALGCLVKD |
| | | YFPEPVTVSWNSGALTSGVHTFPAVLQSSGLYSLSSVVT |
| | | VPSSSLGTKTYTCNVDHKPSNTKVDKRVESKYGPPCPPC |
| | | PAPEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLTCLVKGFYPSDIAVEWES |
| | | NGQPENNYKTTPPVLDSDGSFFLYSRLTVDKSRWQEGN |
| | | VFSCSVMHEALHNHYTQKSLSLSLG |
| SEQ ID NO: 76 | DNA | GAAGTGCAGCTGGTGCAGTCAGGCGCCGAAGTGAAG |
| | heavy | AAACCCGGCGCTACCGTGAAGATTAGCTGTAAAGTCT |
| | chain | CAGGCTACACCTTCACTAGCTACTGGATGTACTGGGT |
| | | CCGACAGGCTAGAGGGCAAAGACTGGAGTGGATCGG |
| | | TAGAATCGACCCTAATAGCGGCTCTACTAAGTATAAC |
| | | GAGAAGTTTAAGAATAGGTTCACTATTAGTAGGGATA |
| | | ACTCTAAGAACACCCTGTACCTGCAGATGAATAGCCT |
| | | GAGAGCCGAGGACACCGCCGTCTACTACTGCGCTAGA |
| | | GACTATAGAAAGGGCCTGTACGCTATGGACTACTGGG |
| | | GTCAAGGCACTACCGTGACCGTGTCTTCAGCTAGCAC |
| | | TAAGGGCCCGTCCGTGTTCCCCCTGGCACCTTGTAGC |
| | | CGGAGCACTAGCGAATCCACCGCTGCCCTCGGCTGCC |
| | | TGGTCAAGGATTACTTCCCGGAGCCCGTGACCGTGTC |
| | | CTGGAACAGCGGAGCCCTGACCTCCGGAGTGCACACC |
| | | TTCCCCGCTGTGCTGCAGAGCTCCGGGCTGTACTCGCT |
| | | GTCGTCGGTGGTCACGGTGCCTTCATCTAGCCTGGGT |
| | | ACCAAGACCTACACTTGCAACGTGGACCACAAGCCTT |
| | | CCAACACTAAGGTGGACAAGCGCGTCGAATCGAAGT |
| | | ACGGCCCACCGTGCCCGCCTTGTCCCGCGCCGGAGTT |
| | | CCTCGGCGGTCCCTCGGTCTTTCTGTTCCCACCGAAGC |
| | | CCAAGGACACTTTGATGATTTCCCGCACCCCTGAAGT |
| | | GACATGCGTGGTCGTGGACGTGTCACAGGAAGATCCG |
| | | GAGGTGCAGTTCAATTGGTACGTGGATGGCGTCGAGG |
| | | TGCACAACGCCAAAACCAAGCCGAGGGAGGAGCAGT |
| | | TCAACTCCACTTACCGCGTCGTGTCCGTGCTGACGGT |
| | | GCTGCATCAGGACTGGCTGAACGGGAAGGAGTACAA |
| | | GTGCAAAGTGTCCAACAAGGGACTTCCTAGCTCAATC |
| | | GAAAAGACCATCTCGAAAGCCAAGGGACAGCCCCGG |
| | | GAACCCCAAGTGTATACCCTGCCACCGAGCCAGGAAG |
| | | AAATGACTAAGAACCAAGTCTCATTGACTTGCCTTGT |
| | | GAAGGGCTTCTACCCATCGGATATCGCCGTGGAATGG |
| | | GAGTCCAACGGCCAGCCGGAAAACAACTACAAGACC |
| | | ACCCCTCCGGTGCTGGACTCAGACGGATCCTTCTTCCT |
| | | CTACTCGCGGCTGACCGTGGATAAGAGCAGATGGCAG |
| | | GAGGGAAATGTGTTCAGCTGTTCTGTGATGCATGAAG |
| | | CCCTGCACAACCACTACACTCAGAAGTCCCTGTCCCT |
| | | CTCCCTGGGA |
| BAP058-Clone O LC | | |
| SEQ ID NO: 70 (Kabat) | LCDR1 | KASQDVGTAVA |
| SEQ ID NO: 71 (Kabat) | LCDR2 | WASTRHT |
| SEQ ID NO: 72 (Kabat) | LCDR3 | QQYNSYPLT |
| SEQ ID NO: 73 | LCDR1 | SQDVGTA |
| (Chothia) | | |
| SEQ ID NO: 74 | LCDR2 | WAS |
| (Chothia) | | |
| SEQ ID NO: 75 | LCDR3 | YNSYPL |
| (Chothia) | | |
| SEQ ID NO: 77 | VL | AIQLTQSPSSLSASVGDRVTITCKASQDVGTAVAWYLQK |
| | | PGQSPQLLIYWASTRHTGVPSRFSGSGSGTDFTFTISSLE |
| | | AEDAATYYCQQYNSYPLTFGQGTKVEIK |
| SEQ ID NO: 78 | DNA VL | GCTATTCAGCTGACTCAGTCACCTAGTAGCCTGAGCG |
| | | CTAGTGTGGGCGATAGAGTGACTATCACCTGTAAAGC |
| | | CTCTCAGGACGTGGGCACCGCCGTGGCCTGGTATCTG |
| | | CAGAAGCCTGGTCAATCACCTCAGCTGCTGATCTACT |
| | | GGGCCTCTACTAGACACACCGGCGTGCCCTCTAGGTT |
| | | TAGCGGTAGCGGTAGTGGCACCGACTTCACCTTCACT |
| | | ATCTCTTCACTGGAAGCCGAGGACGCCGCTACCTACT |
| | | ACTGTCAGCAGTATAATAGCTACCCCCTGACCTTCGG |
| | | TCAAGGCACTAAGGTCGAGATTAAG |
| SEQ ID NO: 79 | Light | AIQLTQSPSSLSASVGDRVTITCKASQDVGTAVAWYLQK |
| | chain | PGQSPQLLIYWASTRHTGVPSRFSGSGSGTDFTFTISSLE |
| | | AEDAATYYCQQYNSYPLTFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 80 | DNA light | GCTATTCAGCTGACTCAGTCACCTAGTAGCCTGAGCG |
| | chain | CTAGTGTGGGCGATAGAGTGACTATCACCTGTAAAGC |
| | | CTCTCAGGACGTGGGCACCGCCGTGGCCTGGTATCTG |
| | | CAGAAGCCTGGTCAATCACCTCAGCTGCTGATCTACT |
| | | GGGCCTCTACTAGACACACCGGCGTGCCCTCTAGGTT |
| | | TAGCGGTAGCGGTAGTGGCACCGACTTCACCTTCACT |
| | | ATCTCTTCACTGGAAGCCGAGGACGCCGCTACCTACT |
| | | ACTGTCAGCAGTATAATAGCTACCCCCTGACCTTCGG |
| | | TCAAGGCACTAAGGTCGAGATTAAGCGTACGGTGGCC |
| | | GCTCCCAGCGTGTTCATCTTCCCCCCCAGCGACGAGC |
| | | AGCTGAAGAGCGGCACCGCCAGCGTGGTGTGCCTGCT |
| | | GAACAACTTCTACCCCCGGGAGGCCAAGGTGCAGTGG |
| | | AAGGTGGACAACGCCCTGCAGAGCGGCAACAGCCAG |
| | | GAGAGCGTCACCGAGCAGGACAGCAAGGACTCCACC |
| | | TACAGCCTGAGCAGCACCCTGACCCTGAGCAAGGCCG |
| | | ACTACGAGAAGCATAAGGTGTACGCCTGCGAGGTGA |
| | | CCCACCAGGGCCTGTCCAGCCCCGTGACCAAGAGCTT |
| | | CAACAGGGGCGAGTGC |
| BAP058-Clone N HC | | |
| SEQ ID NO: 62 (Kabat) | HCDR1 | SYWMY |
| SEQ ID NO: 63 (Kabat) | HCDR2 | RIDPNSGSTKYNEKFKN |
| SEQ ID NO: 64 (Kabat) | HCDR3 | DYRKGLYAMDY |
| SEQ ID NO: 65 | HCDR1 | GYTFTSY |
| (Chothia) | | |
| SEQ ID NO: 66 | HCDR2 | DPNSGS |
| (Chothia) | | |
| SEQ ID NO: 64 | HCDR3 | DYRKGLYAMDY |
| (Chothia) | | |
| SEQ ID NO: 81 | VH | EVQLVQSGAEVKKPGATVKISCKVSGYTFTSYWMYWV |
| | | RQATGQGLEWMGRIDPNSGSTKYNEKFKNRVTITADKS |
| | | TSTAYMELSSLRSEDTAVYYCARDYRKGLYAMDYWGQ |
| | | GTTVTVSS |
| SEQ ID NO: 82 | DNA VH | GAAGTGCAGCTGGTGCAGTCAGGCGCCGAAGTGAAG |
| | | AAACCCGGCGCTACCGTGAAGATTAGCTGTAAAGTCT |
| | | CAGGCTACACCTTCACTAGCTACTGGATGTACTGGGT |
| | | CCGACAGGCTACCGGTCAAGGCCTGGAGTGGATGGGT |
| | | AGAATCGACCCTAATAGCGGCTCTACTAAGTATAACG |
| | | AGAAGTTTAAGAATAGAGTGACTATCACCGCCGATAA |
| | | GTCTACTAGCACCGCCTATATGGAACTGTCTAGCCTG |
| | | AGATCAGAGGACACCGCCGTCTACTACTGCGCTAGAG |
| | | ACTATAGAAAGGGCCTGTACGCTATGGACTACTGGGG |
| | | TCAAGGCACTACCGTGACCGTGTCTTCA |
| SEQ ID NO: 83 | Heavy | EVQLVQSGAEVKKPGATVKISCKVSGYTFTSYWMYWV |
| | chain | RQATGQGLEWMGRIDPNSGSTKYNEKFKNRVTITADKS |
| | | TSTAYMELSSLRSEDTAVYYCARDYRKGLYAMDYWGQ |
| | | GTTVTVSSASTKGPSVFPLAPCSRSTSESTAALGCLVKD |
| | | YFPEPVTVSWNSGALTSGVHTFPAVLQSSGLYSLSSVVT |
| | | VPSSSLGTKTYTCNVDHKPSNTKVDKRVESKYGPPCPPC |
| | | PAPEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLTCLVKGFYPSDIAVEWES |
| | | NGQPENNYKTTPPVLDSDGSFFLYSRLTVDKSRWQEGN |
| | | VFSCSVMHEALHNHYTQKSLSLSLG |
| SEQ ID NO: 84 | DNA | GAAGTGCAGCTGGTGCAGTCAGGCGCCGAAGTGAAG |
| | heavy | AAACCCGGCGCTACCGTGAAGATTAGCTGTAAAGTCT |
| | chain | CAGGCTACACCTTCACTAGCTACTGGATGTACTGGGT |
| | | CCGACAGGCTACCGGTCAAGGCCTGGAGTGGATGGGT |
| | | AGAATCGACCCTAATAGCGGCTCTACTAAGTATAACG |
| | | AGAAGTTTAAGAATAGAGTGACTATCACCGCCGATAA |
| | | GTCTACTAGCACCGCCTATATGGAACTGTCTAGCCTG |
| | | AGATCAGAGGACACCGCCGTCTACTACTGCGCTAGAG |
| | | ACTATAGAAAGGGCCTGTACGCTATGGACTACTGGGG |
| | | TCAAGGCACTACCGTGACCGTGTCTTCAGCTAGCACT |
| | | AAGGGCCCGTCCGTGTTCCCCCTGGCACCTTGTAGCC |
| | | GGAGCACTAGCGAATCCACCGCTGCCCTCGGCTGCCT |
| | | GGTCAAGGATTACTTCCCGGAGCCCGTGACCGTGTCC |
| | | TGGAACAGCGGAGCCCTGACCTCCGGAGTGCACACCT |
| | | TCCCCGCTGTGCTGCAGAGCTCCGGGCTGTACTCGCT |
| | | GTCGTCGGTGGTCACGGTGCCTTCATCTAGCCTGGGT |
| | | ACCAAGACCTACACTTGCAACGTGGACCACAAGCCTT |
| | | CCAACACTAAGGTGGACAAGCGCGTCGAATCGAAGT |
| | | ACGGCCCACCGTGCCCGCCTTGTCCCGCGCCGGAGTT |
| | | CCTCGGCGGTCCCTCGGTCTTTCTGTTCCCACCGAAGC |
| | | CCAAGGACACTTTGATGATTTCCCGCACCCCTGAAGT |
| | | GACATGCGTGGTCGTGGACGTGTCACAGGAAGATCCG |
| | | GAGGTGCAGTTCAATTGGTACGTGGATGGCGTCGAGG |
| | | TGCACAACGCCAAAACCAAGCCGAGGGAGGAGCAGT |
| | | TCAACTCCACTTACCGCGTCGTGTCCGTGCTGACGGT |
| | | GCTGCATCAGGACTGGCTGAACGGGAAGGAGTACAA |
| | | GTGCAAAGTGTCCAACAAGGGACTTCCTAGCTCAATC |
| | | GAAAAGACCATCTCGAAAGCCAAGGGACAGCCCCGG |
| | | GAACCCCAAGTGTATACCCTGCCACCGAGCCAGGAAG |
| | | AAATGACTAAGAACCAAGTCTCATTGACTTGCCTTGT |
| | | GAAGGGCTTCTACCCATCGGATATCGCCGTGGAATGG |
| | | GAGTCCAACGGCCAGCCGGAAAACAACTACAAGACC |
| | | ACCCCTCCGGTGCTGGACTCAGACGGATCCTTCTTCCT |
| | | CTACTCGCGGCTGACCGTGGATAAGAGCAGATGGCAG |
| | | GAGGGAAATGTGTTCAGCTGTTCTGTGATGCATGAAG |
| | | CCCTGCACAACCACTACACTCAGAAGTCCCTGTCCCT |
| | | CTCCCTGGGA |
| BAP058-Clone N LC | | |
| SEQ ID NO: 70 (Kabat) | LCDR1 | KASQDVGTAVA |
| SEQ ID NO: 71 (Kabat) | LCDR2 | WASTRHT |
| SEQ ID NO: 72 (Kabat) | LCDR3 | QQYNSYPLT |
| SEQ ID NO: 73 | LCDR1 | SQDVGTA |
| (Chothia) | | |
| SEQ ID NO: 74 | LCDR2 | WAS |
| (Chothia) | | |
| SEQ ID NO: 75 | LCDR3 | YNSYPL |
| (Chothia) | | |
| SEQ ID NO: 85 | VL | DVVMTQSPLSLPVTLGQPASISCKASQDVGTAVAWYQQ |
| | | KPGQAPRLLIYWASTRHTGVPSRFSGSGSGTEFTLTISSL |
| | | QPDDFATYYCQQYNSYPLTFGQGTKVEIK |
| SEQ ID NO: 86 | DNA VL | GACGTCGTGATGACTCAGTCACCCCTGAGCCTGCCCG |
| | | TGACCCTGGGGCAGCCCGCCTCTATTAGCTGTAAAGC |
| | | CTCTCAGGACGTGGGCACCGCCGTGGCCTGGTATCAG |
| | | CAGAAGCCAGGGCAAGCCCCTAGACTGCTGATCTACT |
| | | GGGCCTCTACTAGACACACCGGCGTGCCCTCTAGGTT |
| | | TAGCGGTAGCGGTAGTGGCACCGAGTTCACCCTGACT |
| | | ATCTCTTCACTGCAGCCCGACGACTTCGCTACCTACTA |
| | | CTGTCAGCAGTATAATAGCTACCCCCTGACCTTCGGT |
| | | CAAGGCACTAAGGTCGAGATTAAG |
| SEQ ID NO: 87 | Light | DVVMTQSPLSLPVTLGQPASISCKASQDVGTAVAWYQQ |
| | chain | KPGQAPRLLIYWASTRHTGVPSRFSGSGSGTEFTLTISSL |
| | | QPDDFATYYCQQYNSYPLTFGQGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| SEQ ID NO: 88 | DNA light | GACGTCGTGATGACTCAGTCACCCCTGAGCCTGCCCG |
| | chain | TGACCCTGGGGCAGCCCGCCTCTATTAGCTGTAAAGC |
| | | CTCTCAGGACGTGGGCACCGCCGTGGCCTGGTATCAG |
| | | CAGAAGCCAGGGCAAGCCCCTAGACTGCTGATCTACT |
| | | GGGCCTCTACTAGACACACCGGCGTGCCCTCTAGGTT |
| | | TAGCGGTAGCGGTAGTGGCACCGAGTTCACCCTGACT |
| | | ATCTCTTCACTGCAGCCCGACGACTTCGCTACCTACTA |
| | | CTGTCAGCAGTATAATAGCTACCCCCTGACCTTCGGT |
| | | CAAGGCACTAAGGTCGAGATTAAGCGTACGGTGGCC |
| | | GCTCCCAGCGTGTTCATCTTCCCCCCCAGCGACGAGC |
| | | AGCTGAAGAGCGGCACCGCCAGCGTGGTGTGCCTGCT |
| | | GAACAACTTCTACCCCCGGGAGGCCAAGGTGCAGTGG |
| | | AAGGTGGACAACGCCCTGCAGAGCGGCAACAGCCAG |
| | | GAGAGCGTCACCGAGCAGGACAGCAAGGACTCCACC |
| | | TACAGCCTGAGCAGCACCCTGACCCTGAGCAAGGCCG |
| | | ACTACGAGAAGCATAAGGTGTACGCCTGCGAGGTGA |
| | | CCCACCAGGGCCTGTCCAGCCCCGTGACCAAGAGCTT |
| | | CAACAGGGGCGAGTGC |
| BAP058-Clone O HC | | |
| SEQ ID NO: 89 (Kabat) | HCDR1 | agctactggatgtac |
| SEQ ID NO: 90 (Kabat) | HCDR2 | agaatcgaccctaatagcggctctactaagtataacgagaagtttaagaat |
| SEQ ID NO: 91 (Kabat) | HCDR3 | gactatagaaagggcctgtacgctatggactac |
| SEQ ID NO: 92 | HCDR1 | ggctacaccttcactagctac |
| (Chothia) | | |
| SEQ ID NO: 93 | HCDR2 | gaccctaatagcggctct |
| (Chothia) | | |
| SEQ ID NO: 91 | HCDR3 | gactatagaaagggcctgtacgctatggactac |
| (Chothia) | | |
| BAP058-Clone O LC | | |
| SEQ ID NO: 94 (Kabat) | LCDR1 | aaagcctctcaggacgtgggcaccgccgtggcc |
| SEQ ID NO: 95 (Kabat) | LCDR2 | tgggcctctactagacacacc |
| SEQ ID NO: 96 (Kabat) | LCDR3 | cagcagtataatagctaccccctgacc |
| SEQ ID NO: 97 | LCDR1 | tctcaggacgtgggcaccgcc |
| (Chothia) | | |
| SEQ ID NO: 98 | LCDR2 | tgggcctct |
| (Chothia) | | |
| SEQ ID NO: 99 | LCDR3 | tataatagctaccccctg |
| (Chothia) | | |
| BAP058-Clone N HC | | |
| SEQ ID NO: 89 (Kabat) | HCDR1 | agctactggatgtac |
| SEQ ID NO: 90 (Kabat) | HCDR2 | agaatcgaccctaatagcggctctactaagtataacgagaagtttaagaat |
| SEQ ID NO: 91 (Kabat) | HCDR3 | gactatagaaagggcctgtacgctatggactac |
| SEQ ID NO: 92 | HCDR1 | ggctacaccttcactagctac |
| (Chothia) | | |
| SEQ ID NO: 93 | HCDR2 | gaccctaatagcggctct |
| (Chothia) | | |
| SEQ ID NO: 91 | HCDR3 | gactatagaaagggcctgtacgctatggactac |
| (Chothia) | | |
| BAP058-Clone N LC | | |
| SEQ ID NO: 94 (Kabat) | LCDR1 | aaagcctctcaggacgtgggcaccgccgtggcc |
| SEQ ID NO: 95 (Kabat) | LCDR2 | tgggcctctactagacacacc |
| SEQ ID NO: 96 (Kabat) | LCDR3 | cagcagtataatagctaccccctgacc |
| SEQ ID NO: 97 | LCDR1 | tctcaggacgtgggcaccgcc |
| (Chothia) | | |
| SEQ ID NO: 98 | LCDR2 | tgggcctct |
| (Chothia) | | |
| SEQ ID NO: 99 | LCDR3 | tataatagctaccccctg |
| (Chothia) | | |

### Other Exemplary PD-L1 Inhibitors

In some embodiments, the PD-L1 inhibitor is anti-PD-L1 antibody. In some embodiments, the anti-PD-L1 inhibitor is selected from YW243.55.S70, MPDL3280A, MEDI-4736, or MDX-1105MSB-0010718C (also referred to as A09-246-2) disclosed in, e.g., WO 2013/0179174, and having a sequence disclosed herein (or a sequence substantially identical or similar thereto, e.g., a sequence at least 85%, 90%, 95% identical or higher to the sequence specified).

In one embodiment, the PD-L1 inhibitor is MDX-1105. MDX-1105, also known as BMS-936559, is an anti-PD-L1 antibody described in PCT Publication No. WO 2007/005874.

In one embodiment, the PD-L1 inhibitor is YW243.55.S70. The YW243.55.S70 antibody is an anti-PD-L1 described in PCT Publication No. WO 2010/077634.

In one embodiment, the PD-L1 inhibitor is MDPL3280A (Genentech/Roche) also known as Atezolizumabm, RG7446, RO5541267, YW243.55.S70, or TECENTRIQ™. MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S. Publication No.: 20120039906 incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizumab, e.g., as disclosed in Table 6.

In other embodiments, the PD-L2 inhibitor is AMP-224. AMP-224 is a PD-L2 Fc fusion soluble receptor that blocks the interaction between PD1 and B7-H1 (B7-DCJg; Amplimmune; e.g., disclosed in PCT Publication Nos. WO2010/027827 and WO2011/066342).

In one embodiment, the PD-L1 inhibitor is an anti-PD-L1 antibody molecule. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 6.

In one embodiment, the anti-PD-L 1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 6.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 6.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 6
Amino acid sequences of other exemplary anti-PD-L1 antibody molecules

| | | |
|---|---|---|
| Atezolizumab | | |
| SEQ ID NO: | Heavy | EVQLVESGGGLVQPGGSLRLSCAASGFTFSDSWIHWVRQAPGKGLE |
| 100 | chain | WVAWISPYGGSTYYADSVKGRFTISADTSKNTAYLQMNSLRAEDTA |
| | | VYYCARRHWPGGFDYWGQGTLVTVSSASTKGPSVFPLAPSSKSTSG |
| | | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYASTYRVVSVLTVLHQDWLNGKEYKCK |
| | | VSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| SEQ ID NO: | Light | DIQMTQSPSSLSASVGDRVTITCRASQDVSTAVAWYQQKPGKAPKL |
| 101 | chain | LIYSASFLYSGVPSRFSGSGSGTDFTLTISSLQPEDFATYYCQQYLYHP |
| | | ATFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKH |
| | | KVYACEVTHQGLSSPVTKSFNRGEC |
| Avelumab | | |
| SEQ ID NO: | Heavy | EVQLLESGGGLVQPGGSLRLSCAASGFTFSSYIMMWVRQAPGKGLE |
| 102 | chain | WVSSIYPSGGITFYADTVKGRFTISRDNSKNTLYLQMNSLRAEDTAV |
| | | YYCARIKLGTVTTVDYWGQGTLVTVSSASTKGPSVFPLAPSSKSTSG |
| | | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| SEQ ID NO: | Light | QSALTQPASVSGSPGQSITISCTGTSSDVGGYNYVSWYQQHPGKAPK |
| 103 | chain | LMIYDVSNRPSGVSNRFSGSKSGNTASLTISGLQAEDEADYYCSSYTS |
| | | SSTRVFGTGTKVTVLGQPKANPTVTLFPPSSEELQANKATLVCLISDF |
| | | YPGAVTVAWKADGSPVKAGVETTKPSKQSNNKYAASSYLSLTPEQ |
| | | WKSHRSYSCQVTHEGSTVEKTVAPTECS |
| Durvalumab | | |
| SEQ ID NO: | Heavy | EVQLVESGGGLVQPGGSLRLSCAASGFTFSRYWMSWVRQAPGKGL |
| 104 | chain | EWVANIKQDGSEKYYVDSVKGRFTISRDNAKNSLYLQMNSLRAEDT |
| | | AVYYCAREGGWFGELAFDYWGQGTLVTVSSASTKGPSVFPLAPSSK |
| | | STSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCPP |
| | | CPAPEFEGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPASIEKTISKAKGQPREPQVYTLPPSREEMTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| SEQ ID NO: | Light | EIVLTQSPGTLSLSPGERATLSCRASQRVSSSYLAWYQQKPGQAPRLL |
| 105 | chain | IYDASSRATGIPDRFSGSGSGTDFTLTISRLEPEDFAVYYCQQYGSLP |
| | | WTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKH |
| | | KVYACEVTHQGLSSPVTKSFNRGEC |
| BMS-936559 | | |
| SEQ ID NO: | VH | QVQLVQSGAEVKKPGSSVKVSCKTSGDTFSTYAISWVRQAPGQGLE |
| 106 | | WMGGIIPIFGKAHYAQKFQGRVTITADESTSTAYMELSSLRSEDTAV |
| | | YFCARKFHFVSGSPFGMDVWGQGTTVTVSS |
| SEQ ID NO: | VL | EIVLTQSPATLSLSPGERATLSCRASQSVSSYLAWYQQKPGQAPRLLI |
| 107 | | YDASNRATGIPARFSGSGSGTDFTLTISSLEPEDFAVYYCQQRSNWPT |
| | | FGQGTKVEIK |

### LAG-3 Inhibitors

In some embodiments, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with a LAG-3 inhibitor to treat a disease, e.g., cancer. In some embodiments, the LAG-3 inhibitor is selected from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of BAP050-Clone I or BAP050-Clone J disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In some embodiments, the CDRs are according to the combined CDR definitions of both Kabat and Chothia (e.g., as set out in Table 7). In one embodiment, the combination of Kabat and Chothia CDR of VH CDR1 comprises the amino acid sequence GFTLTNYGMN (SEQ ID NO: 173). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-LAG-3 antibody molecule comprises a heavy chain variable region (VH) comprising a VHCDR1 amino acid sequence of SEQ ID NO: 108, a VHCDR2 amino acid sequence of SEQ ID NO: 109, and a VHCDR3 amino acid sequence of SEQ ID NO: 110; and a light chain variable region (VL) comprising a VLCDR1 amino acid sequence of SEQ ID NO: 117, a VLCDR2 amino acid sequence of SEQ ID NO: 118, and a VLCDR3 amino acid sequence of SEQ ID NO: 119, each disclosed in Table 7.

In one embodiment, the anti-LAG-3 antibody molecule comprises a VH comprising a VHCDR1 encoded by the nucleotide sequence of SEQ ID NO: 143 or 144, a VHCDR2 encoded by the nucleotide sequence of SEQ ID NO: 145 or 146, and a VHCDR3 encoded by the nucleotide sequence of SEQ ID NO: 147 or 148; and a VL comprising a VLCDR1 encoded by the nucleotide sequence of SEQ ID NO: 153 or 154, a VLCDR2 encoded by the nucleotide sequence of SEQ ID NO: 155 or 156, and a VLCDR3 encoded by the nucleotide sequence of SEQ ID NO: 157 or 158, each disclosed in Table 7. In one embodiment, the anti-LAG-3 antibody molecule comprises a VH comprising a VHCDR1 encoded by the nucleotide sequence of SEQ ID NO: 165 or 144, a VHCDR2 encoded by the nucleotide sequence of SEQ ID NO: 166 or 146, and a VHCDR3 encoded by the nucleotide sequence of SEQ ID NO: 167 or 148; and a VL comprising a VLCDR1 encoded by the nucleotide sequence of SEQ ID NO: 153 or 154, a VLCDR2 encoded by the nucleotide sequence of SEQ ID NO: 155 or 156, and a VLCDR3 encoded by the nucleotide sequence of SEQ ID NO: 157 or 158, each disclosed in Table 7.

In one embodiment, the anti-LAG-3 antibody molecule comprises a VH comprising the amino acid sequence of SEQ ID NO: 113, or an amino acid sequence at least 85%, 90%, 95%, or 99% identical or higher to SEQ ID NO: 113. In one embodiment, the anti-LAG-3 antibody molecule comprises a VL comprising the amino acid sequence of SEQ ID NO: 125, or an amino acid sequence at least 85%, 90%, 95%, or 99% identical or higher to SEQ ID NO: 125. In one embodiment, the anti-LAG-3 antibody molecule comprises a VH comprising the amino acid sequence of SEQ ID NO: 131, or an amino acid sequence at least 85%, 90%, 95%, or 99% identical or higher to SEQ ID NO: 131. In one embodiment, the anti-LAG-3 antibody molecule comprises a VL comprising the amino acid sequence of SEQ ID NO: 137, or an amino acid sequence at least 85%, 90%, 95%, or 99% identical or higher to SEQ ID NO: 137. In one embodiment, the anti-LAG-3 antibody molecule comprises a VH comprising the amino acid sequence of SEQ ID NO: 113 and a VL comprising the amino acid sequence of SEQ ID NO: 125. In one embodiment, the anti-LAG-3 antibody molecule comprises a VH comprising the amino acid sequence of SEQ ID NO: 131 and a VL comprising the amino acid sequence of SEQ ID NO: 137.

In one embodiment, the antibody molecule comprises a VH encoded by the nucleotide sequence of SEQ ID NO: 114 or 115, or a nucleotide sequence at least 85%, 90%, 95%, or 99% identical or higher to SEQ ID NO: 114 or 115. In one embodiment, the antibody molecule comprises a VL encoded by the nucleotide sequence of SEQ ID NO: 126 or 127, or a nucleotide sequence at least 85%, 90%, 95%, or 99% identical or higher to SEQ ID NO: 126 or 127. In one embodiment, the antibody molecule comprises a VH encoded by the nucleotide sequence of SEQ ID NO: 132 or 133, or a nucleotide sequence at least 85%, 90%, 95%, or 99% identical or higher to SEQ ID NO: 132 or 133. In one embodiment, the antibody molecule comprises a VL encoded by the nucleotide sequence of SEQ ID NO: 138 or 139, or a nucleotide sequence at least 85%, 90%, 95%, or 99% identical or higher to SEQ ID NO: 138 or 139. In one embodiment, the antibody molecule comprises a VH encoded by the nucleotide sequence of SEQ ID NO: 114 or 115 and a VL encoded by the nucleotide sequence of SEQ ID NO: 126 or 127. In one embodiment, the antibody molecule comprises a VH encoded by the nucleotide sequence of SEQ ID NO: 132 or 133 and a VL encoded by the nucleotide sequence of SEQ ID NO: 138 or 139.

In one embodiment, the anti-LAG-3 antibody molecule comprises a heavy chain comprising the amino acid sequence of SEQ ID NO: 116, or an amino acid sequence at least 85%, 90%, 95%, or 99% identical or higher to SEQ ID NO: 116. In one embodiment, the anti-LAG-3 antibody molecule comprises a light chain comprising the amino acid sequence of SEQ ID NO: 128, or an amino acid sequence at least 85%, 90%, 95%, or 99% identical or higher to SEQ ID NO: 128. In one embodiment, the anti-LAG-3 antibody molecule comprises a heavy chain comprising the amino acid sequence of SEQ ID NO: 134, or an amino acid sequence at least 85%, 90%, 95%, or 99% identical or higher to SEQ ID NO: 134. In one embodiment, the anti-LAG-3 antibody molecule comprises a light chain comprising the amino acid sequence of SEQ ID NO: 140, or an amino acid sequence at least 85%, 90%, 95%, or 99% identical or higher to SEQ ID NO: 140. In one embodiment, the anti-LAG-3 antibody molecule comprises a heavy chain comprising the amino acid sequence of SEQ ID NO: 116 and a light chain comprising the amino acid sequence of SEQ ID NO: 128. In one embodiment, the anti-LAG-3 antibody molecule comprises a heavy chain comprising the amino acid sequence of SEQ ID NO: 134 and a light chain comprising the amino acid sequence of SEQ ID NO: 140.

In one embodiment, the antibody molecule comprises a heavy chain encoded by the nucleotide sequence of SEQ ID NO: 123 or 124, or a nucleotide sequence at least 85%, 90%, 95%, or 99% identical or higher to SEQ ID NO: 123 or 124. In one embodiment, the antibody molecule comprises a light chain encoded by the nucleotide sequence of SEQ ID NO: 129 or 130, or a nucleotide sequence at least 85%, 90%, 95%, or 99% identical or higher to SEQ ID NO: 129 or 130. In one embodiment, the antibody molecule comprises a heavy chain encoded by the nucleotide sequence of SEQ ID NO: 135 or 136, or a nucleotide sequence at least 85%, 90%, 95%, or 99% identical or higher to SEQ ID NO: 135 or 136. In one embodiment, the antibody molecule comprises a light chain encoded by the nucleotide sequence of SEQ ID NO: 141 or 142, or a nucleotide sequence at least 85%, 90%, 95%, or 99% identical or higher to SEQ ID NO: 141 or 142. In one embodiment, the antibody molecule comprises a heavy chain encoded by the nucleotide sequence of SEQ ID NO: 123 or 124 and a light chain encoded by the nucleotide sequence of SEQ ID NO: 129 or 130. In one embodiment, the antibody molecule comprises a heavy chain encoded by the nucleotide sequence of SEQ ID NO: 135 or 136 and a light chain encoded by the nucleotide sequence of SEQ ID NO: 141 or 142.

The antibody molecules described herein can be made by vectors, host cells, and methods described in US 2015/0259420, incorporated by reference in its entirety.

TABLE 7
Amino acid and nucleotide sequences of exemplary anti-LAG-3 antibody molecules

| | | |
|---|---|---|
| BAP050-Clone I HC | | |
| SEQ ID NO: 108 | HCDR1 | NYGMN |
| (Kabat) | | |
| SEQ ID NO: 109 | HCDR2 | WINTDTGEPTYADDFKG |
| (Kabat) | | |
| SEQ ID NO: 110 | HCDR3 | NPPYYYGTNNAEAMDY |
| (Kabat) | | |
| SEQ ID NO: 111 | HCDR1 | GFTLTNY |
| (Chothia) | | |
| SEQ ID NO: 112 | HCDR2 | NTDTGE |
| (Chothia) | | |
| SEQ ID NO: 110 | HCDR3 | NPPYYYGTNNAEAMDY |
| (Chothia) | | |
| SEQ ID NO: 113 | VH | QVQLVQSGAEVKKPGASVKVSCKASGFTLTNYGMNWVRQ |
| | | ARGQRLEWIGWINTDTGEPTYADDFKGRFVFSLDTSVSTAY |
| | | LQISSLKAEDTAVYYCARNPPYYYGTNNAEAMDYWGQGTT |
| | | VTVSS |
| SEQ ID NO: 114 | DNA VH | CAAGTGCAGCTGGTGCAGTCGGGAGCCGAAGTGAAGAAG |
| | | CCTGGAGCCTCGGTGAAGGTGTCGTGCAAGGCATCCGGA |
| | | TTCACCCTCACCAATTACGGGATGAACTGGGTCAGACAG |
| | | GCCCGGGGTCAACGGCTGGAGTGGATCGGATGGATTAAC |
| | | ACCGACACCGGGGAGCCTACCTACGCGGACGATTTCAAG |
| | | GGACGGTTCGTGTTCTCCCTCGACACCTCCGTGTCCACCG |
| | | CCTACCTCCAAATCTCCTCACTGAAAGCGGAGGACACCG |
| | | CCGTGTACTATTGCGCGAGGAACCCGCCCTACTACTACGG |
| | | AACCAACAACGCCGAAGCCATGGACTACTGGGGCCAGGG |
| | | CACCACTGTGACTGTGTCCAGC |
| SEQ ID NO: 115 | DNA VH | CAGGTGCAGCTGGTGCAGTCTGGCGCCGAAGTGAAGAAA |
| | | CCTGGCGCCTCCGTGAAGGTGTCCTGCAAGGCCTCTGGCT |
| | | TCACCCTGACCAACTACGGCATGAACTGGGTGCGACAGG |
| | | CCAGGGGCCAGCGGCTGGAATGGATCGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCT |
| SEQ ID NO: 116 | Heavy | QVQLVQSGAEVKKPGASVKVSCKASGFTLTNYGMNWVRQ |
| | chain | ARGQRLEWIGWINTDTGEPTYADDFKGRFVFSLDTSVSTAY |
| | | LQISSLKAEDTAVYYCARNPPYYYGTNNAEAMDYWGQGTT |
| | | VTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFLGGPSV |
| | | FLFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKNQV |
| | | SLTCLVKGFYPSDIAVEWESNGQPENNYKTTPPVLDSDGSFF |
| | | LYSRLTVDKSRWQEGNVFSCSVMHEALHNHYTQKSLSLSL |
| | | G |
| SEQ ID NO: 123 | DNA | CAAGTGCAGCTGGTGCAGTCGGGAGCCGAAGTGAAGAAG |
| | heavy | CCTGGAGCCTCGGTGAAGGTGTCGTGCAAGGCATCCGGA |
| | chain | TTCACCCTCACCAATTACGGGATGAACTGGGTCAGACAG |
| | | GCCCGGGGTCAACGGCTGGAGTGGATCGGATGGATTAAC |
| | | ACCGACACCGGGGAGCCTACCTACGCGGACGATTTCAAG |
| | | GGACGGTTCGTGTTCTCCCTCGACACCTCCGTGTCCACCG |
| | | CCTACCTCCAAATCTCCTCACTGAAAGCGGAGGACACCG |
| | | CCGTGTACTATTGCGCGAGGAACCCGCCCTACTACTACGG |
| | | AACCAACAACGCCGAAGCCATGGACTACTGGGGCCAGGG |
| | | CACCACTGTGACTGTGTCCAGCGCGTCCACTAAGGGCCC |
| | | GTCCGTGTTCCCCCTGGCACCTTGTAGCCGGAGCACTAGC |
| | | GAATCCACCGCTGCCCTCGGCTGCCTGGTCAAGGATTACT |
| | | TCCCGGAGCCCGTGACCGTGTCCTGGAACAGCGGAGCCC |
| | | TGACCTCCGGAGTGCACACCTTCCCCGCTGTGCTGCAGAG |
| | | CTCCGGGCTGTACTCGCTGTCGTCGGTGGTCACGGTGCCT |
| | | TCATCTAGCCTGGGTACCAAGACCTACACTTGCAACGTGG |
| | | ACCACAAGCCTTCCAACACTAAGGTGGACAAGCGCGTCG |
| | | AATCGAAGTACGGCCCACCGTGCCCGCCTTGTCCCGCGCC |
| | | GGAGTTCCTCGGCGGTCCCTCGGTCTTTCTGTTCCCACCG |
| | | AAGCCCAAGGACACTTTGATGATTTCCCGCACCCCTGAA |
| | | GTGACATGCGTGGTCGTGGACGTGTCACAGGAAGATCCG |
| | | GAGGTGCAGTTCAATTGGTACGTGGATGGCGTCGAGGTG |
| | | CACAACGCCAAAACCAAGCCGAGGGAGGAGCAGTTCAA |
| | | CTCCACTTACCGCGTCGTGTCCGTGCTGACGGTGCTGCAT |
| | | CAGGACTGGCTGAACGGGAAGGAGTACAAGTGCAAAGT |
| | | GTCCAACAAGGGACTTCCTAGCTCAATCGAAAAGACCAT |
| | | CTCGAAAGCCAAGGGACAGCCCCGGGAACCCCAAGTGTA |
| | | TACCCTGCCACCGAGCCAGGAAGAAATGACTAAGAACCA |
| | | AGTCTCATTGACTTGCCTTGTGAAGGGCTTCTACCCATCG |
| | | GATATCGCCGTGGAATGGGAGTCCAACGGCCAGCCGGAA |
| | | AACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGA |
| | | TGCATGAAGCCCTGCACAACCACTACACTCAGAAGTCCC |
| | | TGTCCCTCTCCCTGGGA |
| SEQ ID NO: 124 | DNA | CAGGTGCAGCTGGTGCAGTCTGGCGCCGAAGTGAAGAAA |
| | heavy | CCTGGCGCCTCCGTGAAGGTGTCCTGCAAGGCCTCTGGCT |
| | chain | TCACCCTGACCAACTACGGCATGAACTGGGTGCGACAGG |
| | | CCAGGGGCCAGCGGCTGGAATGGATCGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCTGCTTCTACCAAGGGGCCCA |
| | | GCGTGTTCCCCCTGGCCCCCTGCTCCAGAAGCACCAGCGA |
| | | GAGCACAGCCGCCCTGGGCTGCCTGGTGAAGGACTACTT |
| | | CCCCGAGCCCGTGACCGTGTCCTGGAACAGCGGAGCCCT |
| | | GACCAGCGGCGTGCACACCTTCCCCGCCGTGCTGCAGAG |
| | | CAGCGGCCTGTACAGCCTGAGCAGCGTGGTGACCGTGCC |
| | | CAGCAGCAGCCTGGGCACCAAGACCTACACCTGTAACGT |
| | | GGACCACAAGCCCAGCAACACCAAGGTGGACAAGAGGG |
| | | TGGAGAGCAAGTACGGCCCACCCTGCCCCCCCTGCCCAG |
| | | CCCCCGAGTTCCTGGGCGGACCCAGCGTGTTCCTGTTCCC |
| | | CCCCAAGCCCAAGGACACCCTGATGATCAGCAGAACCCC |
| | | CGAGGTGACCTGTGTGGTGGTGGACGTGTCCCAGGAGGA |
| | | CCCCGAGGTCCAGTTCAACTGGTACGTGGACGGCGTGGA |
| | | GGTGCACAACGCCAAGACCAAGCCCAGAGAGGAGCAGTT |
| | | TAACAGCACCTACCGGGTGGTGTCCGTGCTGACCGTGCTG |
| | | CACCAGGACTGGCTGAACGGCAAAGAGTACAAGTGTAAG |
| | | GTCTCCAACAAGGGCCTGCCAAGCAGCATCGAAAAGACC |
| | | ATCAGCAAGGCCAAGGGCCAGCCTAGAGAGCCCCAGGTC |
| | | TACACCCTGCCACCCAGCCAAGAGGAGATGACCAAGAAC |
| | | CAGGTGTCCCTGACCTGTCTGGTGAAGGGCTTCTACCCAA |
| | | GCGACATCGCCGTGGAGTGGGAGAGCAACGGCCAGCCCG |
| | | AGAACAACTACAAGACCACCCCCCCAGTGCTGGACAGCG |
| | | ACGGCAGCTTCTTCCTGTACAGCAGGCTGACCGTGGACA |
| | | AGTCCAGATGGCAGGAGGGCAACGTCTTTAGCTGCTCCG |
| | | TGATGCACGAGGCCCTGCACAACCACTACACCCAGAAGA |
| | | GCCTGAGCCTGTCCCTGGGC |
| BAP050-Clone I LC | | |
| SEQ ID NO: 117 | LCDR1 | SSSQDISNYLN |
| (Kabat) | | |
| SEQ ID NO: 118 | LCDR2 | YTSTLHL |
| (Kabat) | | |
| SEQ ID NO: 119 | LCDR3 | QQYYNLPWT |
| (Kabat) | | |
| SEQ ID NO: 120 | LCDR1 | SQDISNY |
| (Chothia) | | |
| SEQ ID NO: 121 | LCDR2 | YTS |
| (Chothia) | | |
| SEQ ID NO: 122 | LCDR3 | YYNLPW |
| (Chothia) | | |
| SEQ ID NO: 125 | VL | DIQMTQSPSSLSASVGDRVTITCSSSQDISNYLNWYLQKPGQ |
| | | SPQLLIYYTSTLHLGVPSRFSGSGSGTEFTLTISSLQPDDFATY |
| | | YCQQYYNLPWTFGQGTKVEIK |
| SEQ ID NO: 126 | DNA VL | GATATTCAGATGACTCAGTCACCTAGTAGCCTGAGCGCTA |
| | | GTGTGGGCGATAGAGTGACTATCACCTGTAGCTCTAGTCA |
| | | GGATATCTCTAACTACCTGAACTGGTATCTGCAGAAGCCC |
| | | GGTCAATCACCTCAGCTGCTGATCTACTACACTAGCACCC |
| | | TGCACCTGGGCGTGCCCTCTAGGTTTAGCGGTAGCGGTAG |
| | | TGGCACCGAGTTCACCCTGACTATCTCTAGCCTGCAGCCC |
| | | GACGACTTCGCTACCTACTACTGTCAGCAGTACTATAACC |
| | | TGCCCTGGACCTTCGGTCAAGGCACTAAGGTCGAGATTA |
| | | AG |
| SEQ ID NO: 127 | DNA VL | GACATCCAGATGACCCAGTCCCCCTCCAGCCTGTCTGCTT |
| | | CCGTGGGCGACAGAGTGACCATCACCTGTTCCTCCAGCC |
| | | AGGACATCTCCAACTACCTGAACTGGTATCTGCAGAAGC |
| | | CCGGCCAGTCCCCTCAGCTGCTGATCTACTACACCTCCAC |
| | | CCTGCACCTGGGCGTGCCCTCCAGATTTTCCGGCTCTGGC |
| | | TCTGGCACCGAGTTTACCCTGACCATCAGCTCCCTGCAGC |
| | | CCGACGACTTCGCCACCTACTACTGCCAGCAGTACTACAA |
| | | CCTGCCCTGGACCTTCGGCCAGGGCACCAAGGTGGAAAT |
| | | CAAG |
| SEQ ID NO: 128 | Light | DIQMTQSPSSLSASVGDRVTITCSSSQDISNYLNWYLQKPGQ |
| | chain | SPQLLIYYTSTLHLGVPSRFSGSGSGTEFTLTISSLQPDDFATY |
| | | YCQQYYNLPWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| SEQ ID NO: 129 | DNA | GATATTCAGATGACTCAGTCACCTAGTAGCCTGAGCGCTA |
| | light | GTGTGGGCGATAGAGTGACTATCACCTGTAGCTCTAGTCA |
| | chain | GGATATCTCTAACTACCTGAACTGGTATCTGCAGAAGCCC |
| | | GGTCAATCACCTCAGCTGCTGATCTACTACACTAGCACCC |
| | | TGCACCTGGGCGTGCCCTCTAGGTTTAGCGGTAGCGGTAG |
| | | TGGCACCGAGTTCACCCTGACTATCTCTAGCCTGCAGCCC |
| | | GACGACTTCGCTACCTACTACTGTCAGCAGTACTATAACC |
| | | TGCCCTGGACCTTCGGTCAAGGCACTAAGGTCGAGATTA |
| | | AGCGTACGGTGGCCGCTCCCAGCGTGTTCATCTTCCCCCC |
| | | CAGCGACGAGCAGCTGAAGAGCGGCACCGCCAGCGTGGT |
| | | GTGCCTGCTGAACAACTTCTACCCCCGGGAGGCCAAGGT |
| | | GCAGTGGAAGGTGGACAACGCCCTGCAGAGCGGCAACA |
| | | GCCAGGAGAGCGTCACCGAGCAGGACAGCAAGGACTCC |
| | | ACCTACAGCCTGAGCAGCACCCTGACCCTGAGCAAGGCC |
| | | GACTACGAGAAGCATAAGGTGTACGCCTGCGAGGTGACC |
| | | CACCAGGGCCTGTCCAGCCCCGTGACCAAGAGCTTCAAC |
| | | AGGGGCGAGTGC |
| SEQ ID NO: 130 | DNA | GACATCCAGATGACCCAGTCCCCCTCCAGCCTGTCTGCTT |
| | light | CCGTGGGCGACAGAGTGACCATCACCTGTTCCTCCAGCC |
| | chain | AGGACATCTCCAACTACCTGAACTGGTATCTGCAGAAGC |
| | | CCGGCCAGTCCCCTCAGCTGCTGATCTACTACACCTCCAC |
| | | CCTGCACCTGGGCGTGCCCTCCAGATTTTCCGGCTCTGGC |
| | | TCTGGCACCGAGTTTACCCTGACCATCAGCTCCCTGCAGC |
| | | CCGACGACTTCGCCACCTACTACTGCCAGCAGTACTACAA |
| | | CCTGCCCTGGACCTTCGGCCAGGGCACCAAGGTGGAAAT |
| | | CAAGCGTACGGTGGCCGCTCCCAGCGTGTTCATCTTCCCC |
| | | CCAAGCGACGAGCAGCTGAAGAGCGGCACCGCCAGCGTG |
| | | GTGTGTCTGCTGAACAACTTCTACCCCAGGGAGGCCAAG |
| | | GTGCAGTGGAAGGTGGACAACGCCCTGCAGAGCGGCAAC |
| | | AGCCAGGAGAGCGTCACCGAGCAGGACAGCAAGGACTC |
| | | CACCTACAGCCTGAGCAGCACCCTGACCCTGAGCAAGGC |
| | | CGACTACGAGAAGCACAAGGTGTACGCCTGTGAGGTGAC |
| | | CCACCAGGGCCTGTCCAGCCCCGTGACCAAGAGCTTCAA |
| | | CAGGGGCGAGTGC |
| BAP050-Clone J HC | | |
| SEQ ID NO: 108 | HCDR1 | NYGMN |
| (Kabat) | | |
| SEQ ID NO: 109 | HCDR2 | WINTDTGEPTYADDFKG |
| (Kabat) | | |
| SEQ ID NO: 110 | HCDR3 | NPPYYYGTNNAEAMDY |
| (Kabat) | | |
| SEQ ID NO: 111 | HCDR1 | GFTLTNY |
| (Chothia) | | |
| SEQ ID NO: 112 | HCDR2 | NTDTGE |
| (Chothia) | | |
| SEQ ID NO: 110 | HCDR3 | NPPYYYGTNNAEAMDY |
| (Chothia) | | |
| SEQ ID NO: 131 | VH | QVQLVQSGAEVKKPGASVKVSCKASGFTLTNYGMNWVRQ |
| | | APGQGLEWMGWINTDTGEPTYADDFKGRFVFSLDTSVSTA |
| | | YLQISSLKAEDTAVYYCARNPPYYYGTNNAEAMDYWGQG |
| | | TTVTVSS |
| SEQ ID NO: 132 | DNA VH | CAGGTGCAGCTGGTGCAGTCAGGCGCCGAAGTGAAGAAA |
| | | CCCGGCGCTAGTGTGAAAGTCAGCTGTAAAGCTAGTGGC |
| | | TTCACCCTGACTAACTACGGGATGAACTGGGTCCGCCAG |
| | | GCCCCAGGTCAAGGCCTCGAGTGGATGGGCTGGATTAAC |
| | | ACCGACACCGGCGAGCCTACCTACGCCGACGACTTTAAG |
| | | GGCAGATTCGTGTTTAGCCTGGACACTAGTGTGTCTACCG |
| | | CCTACCTGCAGATCTCTAGCCTGAAGGCCGAGGACACCG |
| | | CCGTCTACTACTGCGCTAGAAACCCCCCCTACTACTACGG |
| | | CACTAACAACGCCGAGGCTATGGACTACTGGGGTCAAGG |
| | | CACTACCGTGACCGTGTCTAGC |
| SEQ ID NO: 133 | DNA VH | CAGGTGCAGCTGGTGCAGTCTGGCGCCGAAGTGAAGAAA |
| | | CCTGGCGCCTCCGTGAAGGTGTCCTGCAAGGCCTCTGGCT |
| | | TCACCCTGACCAACTACGGCATGAACTGGGTGCGACAGG |
| | | CCCCTGGACAGGGCCTGGAATGGATGGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCT |
| SEQ ID NO: 134 | Heavy | QVQLVQSGAEVKKPGASVKVSCKASGFTLTNYGMNWVRQ |
| | chain | APGQGLEWMGWINTDTGEPTYADDFKGRFVFSLDTSVSTA |
| | | YLQISSLKAEDTAVYYCARNPPYYYGTNNAEAMDYWGQG |
| | | TTVTVSSASTKGPSVFPLAPCSRSTSESTAALGCLVKDYFPEP |
| | | VTVSWNSGALTSGVHTFPAVLQSSGLYSLSSVVTVPSSSLGT |
| | | KTYTCNVDHKPSNTKVDKRVESKYGPPCPPCPAPEFLGGPS |
| | | VFLFPPKPKDTLMISRTPEVTCVVVDVSQEDPEVQFNWYVD |
| | | GVEVHNAKTKPREEQFNSTYRVVSVLTVLHQDWLNGKEYK |
| | | CKVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVFSCSVMHEALHNHYTQKSLSLS |
| | | LG |
| SEQ ID NO: 135 | DNA | CAGGTGCAGCTGGTGCAGTCAGGCGCCGAAGTGAAGAAA |
| | heavy | CCCGGCGCTAGTGTGAAAGTCAGCTGTAAAGCTAGTGGC |
| | chain | TTCACCCTGACTAACTACGGGATGAACTGGGTCCGCCAG |
| | | GCCCCAGGTCAAGGCCTCGAGTGGATGGGCTGGATTAAC |
| | | ACCGACACCGGCGAGCCTACCTACGCCGACGACTTTAAG |
| | | GGCAGATTCGTGTTTAGCCTGGACACTAGTGTGTCTACCG |
| | | CCTACCTGCAGATCTCTAGCCTGAAGGCCGAGGACACCG |
| | | CCGTCTACTACTGCGCTAGAAACCCCCCCTACTACTACGG |
| | | CACTAACAACGCCGAGGCTATGGACTACTGGGGTCAAGG |
| | | CACTACCGTGACCGTGTCTAGCGCTAGCACTAAGGGCCC |
| | | GTCCGTGTTCCCCCTGGCACCTTGTAGCCGGAGCACTAGC |
| | | GAATCCACCGCTGCCCTCGGCTGCCTGGTCAAGGATTACT |
| | | TCCCGGAGCCCGTGACCGTGTCCTGGAACAGCGGAGCCC |
| | | TGACCTCCGGAGTGCACACCTTCCCCGCTGTGCTGCAGAG |
| | | CTCCGGGCTGTACTCGCTGTCGTCGGTGGTCACGGTGCCT |
| | | TCATCTAGCCTGGGTACCAAGACCTACACTTGCAACGTGG |
| | | ACCACAAGCCTTCCAACACTAAGGTGGACAAGCGCGTCG |
| | | AATCGAAGTACGGCCCACCGTGCCCGCCTTGTCCCGCGCC |
| | | GGAGTTCCTCGGCGGTCCCTCGGTCTTTCTGTTCCCACCG |
| | | AAGCCCAAGGACACTTTGATGATTTCCCGCACCCCTGAA |
| | | GTGACATGCGTGGTCGTGGACGTGTCACAGGAAGATCCG |
| | | GAGGTGCAGTTCAATTGGTACGTGGATGGCGTCGAGGTG |
| | | CACAACGCCAAAACCAAGCCGAGGGAGGAGCAGTTCAA |
| | | CTCCACTTACCGCGTCGTGTCCGTGCTGACGGTGCTGCAT |
| | | CAGGACTGGCTGAACGGGAAGGAGTACAAGTGCAAAGT |
| | | GTCCAACAAGGGACTTCCTAGCTCAATCGAAAAGACCAT |
| | | CTCGAAAGCCAAGGGACAGCCCCGGGAACCCCAAGTGTA |
| | | TACCCTGCCACCGAGCCAGGAAGAAATGACTAAGAACCA |
| | | AGTCTCATTGACTTGCCTTGTGAAGGGCTTCTACCCATCG |
| | | GATATCGCCGTGGAATGGGAGTCCAACGGCCAGCCGGAA |
| | | AACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGA |
| | | TGCATGAAGCCCTGCACAACCACTACACTCAGAAGTCCC |
| | | TGTCCCTCTCCCTGGGA |
| SEQ ID NO: 136 | DNA | CAGGTGCAGCTGGTGCAGTCTGGCGCCGAAGTGAAGAAA |
| | heavy | CCTGGCGCCTCCGTGAAGGTGTCCTGCAAGGCCTCTGGCT |
| | chain | TCACCCTGACCAACTACGGCATGAACTGGGTGCGACAGG |
| | | CCCCTGGACAGGGCCTGGAATGGATGGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCTGCTTCTACCAAGGGGCCCA |
| | | GCGTGTTCCCCCTGGCCCCCTGCTCCAGAAGCACCAGCGA |
| | | GAGCACAGCCGCCCTGGGCTGCCTGGTGAAGGACTACTT |
| | | CCCCGAGCCCGTGACCGTGTCCTGGAACAGCGGAGCCCT |
| | | GACCAGCGGCGTGCACACCTTCCCCGCCGTGCTGCAGAG |
| | | CAGCGGCCTGTACAGCCTGAGCAGCGTGGTGACCGTGCC |
| | | CAGCAGCAGCCTGGGCACCAAGACCTACACCTGTAACGT |
| | | GGACCACAAGCCCAGCAACACCAAGGTGGACAAGAGGG |
| | | TGGAGAGCAAGTACGGCCCACCCTGCCCCCCCTGCCCAG |
| | | CCCCCGAGTTCCTGGGCGGACCCAGCGTGTTCCTGTTCCC |
| | | CCCCAAGCCCAAGGACACCCTGATGATCAGCAGAACCCC |
| | | CGAGGTGACCTGTGTGGTGGTGGACGTGTCCCAGGAGGA |
| | | CCCCGAGGTCCAGTTCAACTGGTACGTGGACGGCGTGGA |
| | | GGTGCACAACGCCAAGACCAAGCCCAGAGAGGAGCAGTT |
| | | TAACAGCACCTACCGGGTGGTGTCCGTGCTGACCGTGCTG |
| | | CACCAGGACTGGCTGAACGGCAAAGAGTACAAGTGTAAG |
| | | GTCTCCAACAAGGGCCTGCCAAGCAGCATCGAAAAGACC |
| | | ATCAGCAAGGCCAAGGGCCAGCCTAGAGAGCCCCAGGTC |
| | | TACACCCTGCCACCCAGCCAAGAGGAGATGACCAAGAAC |
| | | CAGGTGTCCCTGACCTGTCTGGTGAAGGGCTTCTACCCAA |
| | | GCGACATCGCCGTGGAGTGGGAGAGCAACGGCCAGCCCG |
| | | AGAACAACTACAAGACCACCCCCCCAGTGCTGGACAGCG |
| | | ACGGCAGCTTCTTCCTGTACAGCAGGCTGACCGTGGACA |
| | | AGTCCAGATGGCAGGAGGGCAACGTCTTTAGCTGCTCCG |
| | | TGATGCACGAGGCCCTGCACAACCACTACACCCAGAAGA |
| | | GCCTGAGCCTGTCCCTGGGC |
| BAP050-Clone J LC | | |
| SEQ ID NO: 117 | LCDR1 | SSSQDISNYLN |
| (Kabat) | | |
| SEQ ID NO: 118 | LCDR2 | YTSTLHL |
| (Kabat) | | |
| SEQ ID NO: 119 | LCDR3 | QQYYNLPWT |
| (Kabat) | | |
| SEQ ID NO: 120 | LCDR1 | SQDISNY |
| (Chothia) | | |
| SEQ ID NO: 121 | LCDR2 | YTS |
| (Chothia) | | |
| SEQ ID NO: 122 | LCDR3 | YYNLPW |
| (Chothia) | | |
| SEQ ID NO: 137 | VL | DIQMTQSPSSLSASVGDRVTITCSSSQDISNYLNWYQQKPGK |
| | | APKLLIYYTSTLHLGIPPRFSGSGYGTDFTLTINNIESEDAAY |
| | | YFCQQYYNLPWTFGQGTKVEIK |
| SEQ ID NO: 138 | DNA VL | GATATTCAGATGACTCAGTCACCTAGTAGCCTGAGCGCTA |
| | | GTGTGGGCGATAGAGTGACTATCACCTGTAGCTCTAGTCA |
| | | GGATATCTCTAACTACCTGAACTGGTATCAGCAGAAGCC |
| | | CGGTAAAGCCCCTAAGCTGCTGATCTACTACACTAGCACC |
| | | CTGCACCTGGGAATCCCCCCTAGGTTTAGCGGTAGCGGCT |
| | | ACGGCACCGACTTCACCCTGACTATTAACAATATCGAGTC |
| | | AGAGGACGCCGCCTACTACTTCTGTCAGCAGTACTATAAC |
| | | CTGCCCTGGACCTTCGGTCAAGGCACTAAGGTCGAGATT |
| | | AAG |
| SEQ ID NO: 139 | DNA VL | GACATCCAGATGACCCAGTCCCCCTCCAGCCTGTCTGCTT |
| | | CCGTGGGCGACAGAGTGACCATCACCTGTTCCTCCAGCC |
| | | AGGACATCTCCAACTACCTGAACTGGTATCAGCAGAAGC |
| | | CCGGCAAGGCCCCCAAGCTGCTGATCTACTACACCTCCAC |
| | | CCTGCACCTGGGCATCCCCCCTAGATTCTCCGGCTCTGGC |
| | | TACGGCACCGACTTCACCCTGACCATCAACAACATCGAG |
| | | TCCGAGGACGCCGCCTACTACTTCTGCCAGCAGTACTACA |
| | | ACCTGCCCTGGACCTTCGGCCAGGGCACCAAGGTGGAAA |
| | | TCAAG |
| SEQ ID NO: 140 | Light | DIQMTQSPSSLSASVGDRVTITCSSSQDISNYLNWYQQKPGK |
| | chain | APKLLIYYTSTLHLGIPPRFSGSGYGTDFTLTINNIESEDAAY |
| | | YFCQQYYNLPWTFGQGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| SEQ ID NO: 141 | DNA | GATATTCAGATGACTCAGTCACCTAGTAGCCTGAGCGCTA |
| | light | GTGTGGGCGATAGAGTGACTATCACCTGTAGCTCTAGTCA |
| | chain | GGATATCTCTAACTACCTGAACTGGTATCAGCAGAAGCC |
| | | CGGTAAAGCCCCTAAGCTGCTGATCTACTACACTAGCACC |
| | | CTGCACCTGGGAATCCCCCCTAGGTTTAGCGGTAGCGGCT |
| | | ACGGCACCGACTTCACCCTGACTATTAACAATATCGAGTC |
| | | AGAGGACGCCGCCTACTACTTCTGTCAGCAGTACTATAAC |
| | | CTGCCCTGGACCTTCGGTCAAGGCACTAAGGTCGAGATT |
| | | AAGCGTACGGTGGCCGCTCCCAGCGTGTTCATCTTCCCCC |
| | | CCAGCGACGAGCAGCTGAAGAGCGGCACCGCCAGCGTGG |
| | | TGTGCCTGCTGAACAACTTCTACCCCCGGGAGGCCAAGG |
| | | TGCAGTGGAAGGTGGACAACGCCCTGCAGAGCGGCAACA |
| | | GCCAGGAGAGCGTCACCGAGCAGGACAGCAAGGACTCC |
| | | ACCTACAGCCTGAGCAGCACCCTGACCCTGAGCAAGGCC |
| | | GACTACGAGAAGCATAAGGTGTACGCCTGCGAGGTGACC |
| | | CACCAGGGCCTGTCCAGCCCCGTGACCAAGAGCTTCAAC |
| | | AGGGGCGAGTGC |
| SEQ ID NO: 142 | DNA | GACATCCAGATGACCCAGTCCCCCTCCAGCCTGTCTGCTT |
| | light | CCGTGGGCGACAGAGTGACCATCACCTGTTCCTCCAGCC |
| | chain | AGGACATCTCCAACTACCTGAACTGGTATCAGCAGAAGC |
| | | CCGGCAAGGCCCCCAAGCTGCTGATCTACTACACCTCCAC |
| | | CCTGCACCTGGGCATCCCCCCTAGATTCTCCGGCTCTGGC |
| | | TACGGCACCGACTTCACCCTGACCATCAACAACATCGAG |
| | | TCCGAGGACGCCGCCTACTACTTCTGCCAGCAGTACTACA |
| | | ACCTGCCCTGGACCTTCGGCCAGGGCACCAAGGTGGAAA |
| | | TCAAGCGTACGGTGGCCGCTCCCAGCGTGTTCATCTTCCC |
| | | CCCAAGCGACGAGCAGCTGAAGAGCGGCACCGCCAGCGT |
| | | GGTGTGTCTGCTGAACAACTTCTACCCCAGGGAGGCCAA |
| | | GGTGCAGTGGAAGGTGGACAACGCCCTGCAGAGCGGCAA |
| | | CAGCCAGGAGAGCGTCACCGAGCAGGACAGCAAGGACT |
| | | CCACCTACAGCCTGAGCAGCACCCTGACCCTGAGCAAGG |
| | | CCGACTACGAGAAGCACAAGGTGTACGCCTGTGAGGTGA |
| | | CCCACCAGGGCCTGTCCAGCCCCGTGACCAAGAGCTTCA |
| | | ACAGGGGCGAGTGC |
| BAP050-Clone I HC | | |
| SEQ ID NO: 143 | HCDR1 | AATTACGGGATGAAC |
| (Kabat) | | |
| SEQ ID NO: 144 | HCDR1 | AACTACGGCATGAAC |
| (Kabat) | | |
| SEQ ID NO: 145 | HCDR2 | TGGATTAACACCGACACCGGGGAGCCTACCTACGCGGAC |
| (Kabat) | | GATTTCAAGGGA |
| SEQ ID NO: 146 | HCDR2 | TGGATCAACACCGACACCGGCGAGCCTACCTACGCCGAC |
| (Kabat) | | GACTTCAAGGGC |
| SEQ ID NO: 147 | HCDR3 | AACCCGCCCTACTACTACGGAACCAACAACGCCGAAGCC |
| (Kabat) | | ATGGACTAC |
| SEQ ID NO: 148 | HCDR3 | AACCCCCCTTACTACTACGGCACCAACAACGCCGAGGCC |
| (Kabat) | | ATGGACTAT |
| SEQ ID NO: 149 | HCDR1 | GGATTCACCCTCACCAATTAC |
| (Chothia) | | |
| SEQ ID NO: 150 | HCDR1 | GGCTTCACCCTGACCAACTAC |
| (Chothia) | | |
| SEQ ID NO: 151 | HCDR2 | AACACCGACACCGGGGAG |
| (Chothia) | | |
| SEQ ID NO: 152 | HCDR2 | AACACCGACACCGGCGAG |
| (Chothia) | | |
| SEQ ID NO: 147 | HCDR3 | AACCCGCCCTACTACTACGGAACCAACAACGCCGAAGCC |
| (Chothia) | | ATGGACTAC |
| SEQ ID NO: 148 | HCDR3 | AACCCCCCTTACTACTACGGCACCAACAACGCCGAGGCC |
| (Chothia) | | ATGGACTAT |
| BAP050-Clone I LC | | |
| SEQ ID NO: 153 | LCDR1 | AGCTCTAGTCAGGATATCTCTAACTACCTGAAC |
| (Kabat) | | |
| SEQ ID NO: 154 | LCDR1 | TCCTCCAGCCAGGACATCTCCAACTACCTGAAC |
| (Kabat) | | |
| SEQ ID NO: 155 | LCDR2 | TACACTAGCACCCTGCACCTG |
| (Kabat) | | |
| SEQ ID NO: 156 | LCDR2 | TACACCTCCACCCTGCACCTG |
| (Kabat) | | |
| SEQ ID NO: 157 | LCDR3 | CAGCAGTACTATAACCTGCCCTGGACC |
| (Kabat) | | |
| SEQ ID NO: 158 | LCDR3 | CAGCAGTACTACAACCTGCCCTGGACC |
| (Kabat) | | |
| SEQ ID NO: 159 | LCDR1 | AGTCAGGATATCTCTAACTAC |
| (Chothia) | | |
| SEQ ID NO: 160 | LCDR1 | AGCCAGGACATCTCCAACTAC |
| (Chothia) | | |
| SEQ ID NO: 161 | LCDR2 | TACACTAGC |
| (Chothia) | | |
| SEQ ID NO: 162 | LCDR2 | TACACCTCC |
| (Chothia) | | |
| SEQ ID NO: 163 | LCDR3 | TACTATAACCTGCCCTGG |
| (Chothia) | | |
| SEQ ID NO: 164 | LCDR3 | TACTACAACCTGCCCTGG |
| (Chothia) | | |
| BAP050-Clone J HC | | |
| SEQ ID NO: 165 | HCDR1 | AACTACGGGATGAAC |
| (Kabat) | | |
| SEQ ID NO: 144 | HCDR1 | AACTACGGCATGAAC |
| (Kabat) | | |
| SEQ ID NO: 166 | HCDR2 | TGGATTAACACCGACACCGGCGAGCCTACCTACGCCGAC |
| (Kabat) | | GACTTTAAGGGC |
| SEQ ID NO: 146 | HCDR2 | TGGATCAACACCGACACCGGCGAGCCTACCTACGCCGAC |
| (Kabat) | | GACTTCAAGGGC |
| SEQ ID NO: 167 | HCDR3 | AACCCCCCCTACTACTACGGCACTAACAACGCCGAGGCT |
| (Kabat) | | ATGGACTAC |
| SEQ ID NO: 148 | HCDR3 | AACCCCCCTTACTACTACGGCACCAACAACGCCGAGGCC |
| (Kabat) | | ATGGACTAT |
| SEQ ID NO: 168 | HCDR1 | GGCTTCACCCTGACTAACTAC |
| (Chothia) | | |
| SEQ ID NO: 150 | HCDR1 | GGCTTCACCCTGACCAACTAC |
| (Chothia) | | |
| SEQ ID NO: 151 | HCDR2 | AACACCGACACCGGGGAG |
| (Chothia) | | |
| SEQ ID NO: 152 | HCDR2 | AACACCGACACCGGCGAG |
| (Chothia) | | |
| SEQ ID NO: 167 | HCDR3 | AACCCCCCCTACTACTACGGCACTAACAACGCCGAGGCT |
| (Chothia) | | ATGGACTAC |
| SEQ ID NO: 148 | HCDR3 | AACCCCCCTTACTACTACGGCACCAACAACGCCGAGGCC |
| (Chothia) | | ATGGACTAT |
| BAP050-Clone J LC | | |
| SEQ ID NO: 153 | LCDR1 | AGCTCTAGTCAGGATATCTCTAACTACCTGAAC |
| (Kabat) | | |
| SEQ ID NO: 154 | LCDR1 | TCCTCCAGCCAGGACATCTCCAACTACCTGAAC |
| (Kabat) | | |
| SEQ ID NO: 155 | LCDR2 | TACACTAGCACCCTGCACCTG |
| (Kabat) | | |
| SEQ ID NO: 156 | LCDR2 | TACACCTCCACCCTGCACCTG |
| (Kabat) | | |
| SEQ ID NO: 157 | LCDR3 | CAGCAGTACTATAACCTGCCCTGGACC |
| (Kabat) | | |
| SEQ ID NO: 158 | LCDR3 | CAGCAGTACTACAACCTGCCCTGGACC |
| (Kabat) | | |
| SEQ ID NO: 159 | LCDR1 | AGTCAGGATATCTCTAACTAC |
| (Chothia) | | |
| SEQ ID NO: 160 | LCDR1 | AGCCAGGACATCTCCAACTAC |
| (Chothia) | | |
| SEQ ID NO: 161 | LCDR2 | TACACTAGC |
| (Chothia) | | |
| SEQ ID NO: 162 | LCDR2 | TACACCTCC |
| (Chothia) | | |
| SEQ ID NO: 163 | LCDR3 | TACTATAACCTGCCCTGG |
| (Chothia) | | |
| SEQ ID NO: 164 | LCDR3 | TACTACAACCTGCCCTGG |
| (Chothia) | | |

### Other Exemplary LAG-3 Inhibitors

In one embodiment, the LAG-3 inhibitor is an anti-LAG-3 antibody molecule. In one embodiment, the LAG-3 inhibitor is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 8.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 8. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 8
Amino acid sequences of other exemplary anti-LAG-3 antibody molecules
BMS-986016

| | | |
|---|---|---|
| SEQ ID NO: | Heavy | QVQLQQWGAGLLKPSETLSLTCAVYGGSFSDYYWNWIRQPPG |
| 169 | chain | KGLEWIGEINHRGSTNSNPSLKSRVTLSLDTSKNQFSLKLRSVTA |
| | | ADTAVYYCAFGYSDYEYNWFDPWGQGTLVTVSSASTKGPSVF |
| | | PLAPCSRSTSESTAALGCLVKDYFPEPVTVSWNSGALTSGVHTF |
| | | PAVLQSSGLYSLSSVVTVPSSSLGTKTYTCNVDHKPSNTKVDKR |
| | | VESKYGPPCPPCPAPEFLGGPSVFLFPPKPKDTLMISRTPEVTCV |
| | | VVDVSQEDPEVQFNWYVDGVEVHNAKTKPREEQFNSTYRVVS |
| | | VLTVLHQDWLNGKEYKCKVSNKGLPSSIEKTISKAKGQPREPQ |
| | | VYTLPPSQEEMTKNQVSLTCLVKGFYPSDIAVEWESNGQPENN |
| | | YKTTPPVLDSDGSFFLYSRLTVDKSRWQEGNVFSCSVMHEALH |
| | | NHYTQKSLSLSLGK |
| SEQ ID NO: | Light chain | EIVLTQSPATLSLSPGERATLSCRASQSISSYLAWYQQKPGQAPR |
| 170 | | LLIYDASNRATGIPARFSGSGSGTDFTLTISSLEPEDFAVYYCQQ |
| | | RSNWPLTFGQGTNLEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |

IMP731

| | | |
|---|---|---|
| SEQ ID NO: | Heavy | QVQLKESGPGLVAPSQSLSITCTVSGFSLTAYGVNWVRQPPGKG |
| 171 | chain | LEWLGMIWDDGSTDYNSALKSRLSISKDNSKSQVFLKMNSLQT |
| | | DDTARYYCAREGDVAFDYWGQGTTLTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKKVEPK |
| | | SCDKTHTCPPCPAPELLGGPSVFLFPPKPKDTLMISRTPEVTCVV |
| | | VDVSHEDPEVKFNWYVDGVEVHNAKTKPREEQYNSTYRVVSV |
| | | LTVLHQDWLNGKEYKCKVSNKALPAPIEKTISKAKGQPREPQV |
| | | YTLPPSRDELTKNQVSLTCLVKGFYPSDIAVEWESNGQPENNYK |
| | | TTPPVLDSDGSFFLYSKLTVDKSRWQQGNVFSCSVMHEALHNH |
| | | YTQKSLSLSPGK |
| SEQ ID NO: | Light chain | DIVMTQSPSSLAVSVGQKVTMSCKSSQSLLNGSNQKNYLAWYQ |
| 172 | | QKPGQSPKLLVYFASTRDSGVPDRFIGSGSGTDFTLTISSVQAED |
| | | LADYFCLQHFGTPPTFGGGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRGE |
| | | C |

### TIM-3 Inhibitors

In certain embodiments, the inhibitor of an immune checkpoint molecule is an inhibitor of TIM-3. In some embodiments, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with a TIM-3 inhibitor to treat a disease, e.g., cancer. In some embodiments, the TIM-3 inhibitor is MGB453 (Novartis) or TSR-022 (Tesaro).

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of ABTIM3-hum11 or ABTIM3-hum03 disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 9). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-TIM-3 antibody molecule comprises a heavy chain variable region (VH) comprising a VHCDR1 amino acid sequence of SEQ ID NO: 174, a VHCDR2 amino acid sequence of SEQ ID NO: 175, and a VHCDR3 amino acid sequence of SEQ ID NO: 176; and a light chain variable region (VL) comprising a VLCDR1 amino acid sequence of SEQ ID NO: 183, a VLCDR2 amino acid sequence of SEQ ID NO: 184, and a VLCDR3 amino acid sequence of SEQ ID NO: 185, each disclosed in Table 9. In one embodiment, the anti-TIM-3 antibody molecule comprises a heavy chain variable region (VH) comprising a VHCDR1 amino acid sequence of SEQ ID NO: 174, a VHCDR2 amino acid sequence of SEQ ID NO: 193, and a VHCDR3 amino acid sequence of SEQ ID NO: 176; and a light chain variable region (VL) comprising a VLCDR1 amino acid sequence of SEQ ID NO: 183, a VLCDR2 amino acid sequence of SEQ ID NO: 184, and a VLCDR3 amino acid sequence of SEQ ID NO: 185, each disclosed in Table 9.

In one embodiment, the anti-TIM-3 antibody molecule comprises a VH comprising the amino acid sequence of SEQ ID NO: 179, or an amino acid sequence at least 85%, 90%, 95%, or 99% identical or higher to SEQ ID NO: 179. In one embodiment, the anti-TIM-3 antibody molecule comprises a VL comprising the amino acid sequence of SEQ ID NO: 189, or an amino acid sequence at least 85%, 90%, 95%, or 99% identical or higher to SEQ ID NO: 189. In one embodiment, the anti-TIM-3 antibody molecule comprises a VH comprising the amino acid sequence of SEQ ID NO: 195, or an amino acid sequence at least 85%, 90%, 95%, or 99% identical or higher to SEQ ID NO: 195. In one embodiment, the anti-TIM-3 antibody molecule comprises a VL comprising the amino acid sequence of SEQ ID NO: 199, or an amino acid sequence at least 85%, 90%, 95%, or 99% identical or higher to SEQ ID NO: 199. In one embodiment, the anti-TIM-3 antibody molecule comprises a VH comprising the amino acid sequence of SEQ ID NO: 179 and a VL comprising the amino acid sequence of SEQ ID NO: 189. In one embodiment, the anti-TIM-3 antibody molecule comprises a VH comprising the amino acid sequence of SEQ ID NO: 195 and a VL comprising the amino acid sequence of SEQ ID NO: 199.

In one embodiment, the antibody molecule comprises a VH encoded by the nucleotide sequence of SEQ ID NO: 180, or a nucleotide sequence at least 85%, 90%, 95%, or 99% identical or higher to SEQ ID NO: 180. In one embodiment, the antibody molecule comprises a VL encoded by the nucleotide sequence of SEQ ID NO: 190, or a nucleotide sequence at least 85%, 90%, 95%, or 99% identical or higher to SEQ ID NO: 190. In one embodiment, the antibody molecule comprises a VH encoded by the nucleotide sequence of SEQ ID NO: 196, or a nucleotide sequence at least 85%, 90%, 95%, or 99% identical or higher to SEQ ID NO: 196. In one embodiment, the antibody molecule comprises a VL encoded by the nucleotide sequence of SEQ ID NO: 200, or a nucleotide sequence at least 85%, 90%, 95%, or 99% identical or higher to SEQ ID NO: 200. In one embodiment, the antibody molecule comprises a VH encoded by the nucleotide sequence of SEQ ID NO: 180 and a VL encoded by the nucleotide sequence of SEQ ID NO: 190. In one embodiment, the antibody molecule comprises a VH encoded by the nucleotide sequence of SEQ ID NO: 196 and a VL encoded by the nucleotide sequence of SEQ ID NO: 200.

In one embodiment, the anti-TIM-3 antibody molecule comprises a heavy chain comprising the amino acid sequence of SEQ ID NO: 181, or an amino acid sequence at least 85%, 90%, 95%, or 99% identical or higher to SEQ ID NO: 181. In one embodiment, the anti-TIM-3 antibody molecule comprises a light chain comprising the amino acid sequence of SEQ ID NO: 191, or an amino acid sequence at least 85%, 90%, 95%, or 99% identical or higher to SEQ ID NO: 191. In one embodiment, the anti-TIM-3 antibody molecule comprises a heavy chain comprising the amino acid sequence of SEQ ID NO: 197, or an amino acid sequence at least 85%, 90%, 95%, or 99% identical or higher to SEQ ID NO: 197. In one embodiment, the anti-TIM-3 antibody molecule comprises a light chain comprising the amino acid sequence of SEQ ID NO: 201, or an amino acid sequence at least 85%, 90%, 95%, or 99% identical or higher to SEQ ID NO: 201. In one embodiment, the anti-TIM-3 antibody molecule comprises a heavy chain comprising the amino acid sequence of SEQ ID NO: 181 and a light chain comprising the amino acid sequence of SEQ ID NO: 191. In one embodiment, the anti-TIM-3 antibody molecule comprises a heavy chain comprising the amino acid sequence of SEQ ID NO: 197 and a light chain comprising the amino acid sequence of SEQ ID NO: 201.

In one embodiment, the antibody molecule comprises a heavy chain encoded by the nucleotide sequence of SEQ ID NO: 182, or a nucleotide sequence at least 85%, 90%, 95%, or 99% identical or higher to SEQ ID NO: 182. In one embodiment, the antibody molecule comprises a light chain encoded by the nucleotide sequence of SEQ ID NO: 192, or a nucleotide sequence at least 85%, 90%, 95%, or 99% identical or higher to SEQ ID NO: 192. In one embodiment, the antibody molecule comprises a heavy chain encoded by the nucleotide sequence of SEQ ID NO: 198, or a nucleotide sequence at least 85%, 90%, 95%, or 99% identical or higher to SEQ ID NO: 198. In one embodiment, the antibody molecule comprises a light chain encoded by the nucleotide sequence of SEQ ID NO: 202, or a nucleotide sequence at least 85%, 90%, 95%, or 99% identical or higher to SEQ ID NO: 202. In one embodiment, the antibody molecule comprises a heavy chain encoded by the nucleotide sequence of SEQ ID NO: 182 and a light chain encoded by the nucleotide sequence of SEQ ID NO: 192. In one embodiment, the antibody molecule comprises a heavy chain encoded by the nucleotide sequence of SEQ ID NO: 198 and a light chain encoded by the nucleotide sequence of SEQ ID NO: 202.

The antibody molecules described herein can be made by vectors, host cells, and methods described in US 2015/0218274, incorporated by reference in its entirety.

TABLE 9
Amino acid and nucleotide sequences of exemplary anti-TIM-3 antibody molecules
ABTIM3-hum11

| | | |
|---|---|---|
| SEQ ID NO: 174 | HCDR1 | SYNMH |
| (Kabat) | | |
| SEQ ID NO: 175 | HCDR2 | DIYPGNGDTSYNQKFKG |
| (Kabat) | | |
| SEQ ID NO: 176 | HCDR3 | VGGAFPMDY |
| (Kabat) | | |
| SEQ ID NO: 177 | HCDR1 | GYTFTSY |
| (Chothia) | | |
| SEQ ID NO: 178 | HCDR2 | YPGNGD |
| (Chothia) | | |
| SEQ ID NO: 176 | HCDR3 | VGGAFPMDY |
| (Chothia) | | |
| SEQ ID NO: 179 | VH | QVQLVQSGAEVKKPGSSVKVSCKASGYTFTSYNMHWVR |
| | | QAPGQGLEWMGDIYPGNGDTSYNQKFKGRVTITADKSTS |
| | | TVYMELSSLRSEDTAVYYCARVGGAFPMDYWGQGTTVT |
| | | VSS |
| SEQ ID NO: 180 | DNA VH | CAGGTGCAGCTGGTGCAGTCAGGCGCCGAAGTGAAGA |
| | | AACCCGGCTCTAGCGTGAAAGTTTCTTGTAAAGCTAGT |
| | | GGCTACACCTTCACTAGCTATAATATGCACTGGGTTCGC |
| | | CAGGCCCCAGGGCAAGGCCTCGAGTGGATGGGCGATAT |
| | | CTACCCCGGGAACGGCGACACTAGTTATAATCAGAAGT |
| | | TTAAGGGTAGAGTCACTATCACCGCCGATAAGTCTACT |
| | | AGCACCGTCTATATGGAACTGAGTTCCCTGAGGTCTGA |
| | | GGACACCGCCGTCTACTACTGCGCTAGAGTGGGCGGAG |
| | | CCTTCCCTATGGACTACTGGGGTCAAGGCACTACCGTG |
| | | ACCGTGTCTAGC |
| SEQ ID NO: 181 | Heavy | QVQLVQSGAEVKKPGSSVKVSCKASGYTFTSYNMHWVR |
| | chain | QAPGQGLEWMGDIYPGNGDTSYNQKFKGRVTITADKSTS |
| | | TVYMELSSLRSEDTAVYYCARVGGAFPMDYWGQGTTVT |
| | | VSSASTKGPSVFPLAPCSRSTSESTAALGCLVKDYFPEPVT |
| | | VSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHNH |
| | | YTQKSLSLSLG |
| SEQ ID NO: 182 | DNA | CAGGTGCAGCTGGTGCAGTCAGGCGCCGAAGTGAAGA |
| | heavy | AACCCGGCTCTAGCGTGAAAGTTTCTTGTAAAGCTAGT |
| | chain | GGCTACACCTTCACTAGCTATAATATGCACTGGGTTCGC |
| | | CAGGCCCCAGGGCAAGGCCTCGAGTGGATGGGCGATAT |
| | | CTACCCCGGGAACGGCGACACTAGTTATAATCAGAAGT |
| | | TTAAGGGTAGAGTCACTATCACCGCCGATAAGTCTACT |
| | | AGCACCGTCTATATGGAACTGAGTTCCCTGAGGTCTGA |
| | | GGACACCGCCGTCTACTACTGCGCTAGAGTGGGCGGAG |
| | | CCTTCCCTATGGACTACTGGGGTCAAGGCACTACCGTG |
| | | ACCGTGTCTAGCGCTAGCACTAAGGGCCCGTCCGTGTT |
| | | CCCCCTGGCACCTTGTAGCCGGAGCACTAGCGAATCCA |
| | | CCGCTGCCCTCGGCTGCCTGGTCAAGGATTACTTCCCGG |
| | | AGCCCGTGACCGTGTCCTGGAACAGCGGAGCCCTGACC |
| | | TCCGGAGTGCACACCTTCCCCGCTGTGCTGCAGAGCTCC |
| | | GGGCTGTACTCGCTGTCGTCGGTGGTCACGGTGCCTTCA |
| | | TCTAGCCTGGGTACCAAGACCTACACTTGCAACGTGGA |
| | | CCACAAGCCTTCCAACACTAAGGTGGACAAGCGCGTCG |
| | | AATCGAAGTACGGCCCACCGTGCCCGCCTTGTCCCGCG |
| | | CCGGAGTTCCTCGGCGGTCCCTCGGTCTTTCTGTTCCCA |
| | | CCGAAGCCCAAGGACACTTTGATGATTTCCCGCACCCC |
| | | TGAAGTGACATGCGTGGTCGTGGACGTGTCACAGGAAG |
| | | ATCCGGAGGTGCAGTTCAATTGGTACGTGGATGGCGTC |
| | | GAGGTGCACAACGCCAAAACCAAGCCGAGGGAGGAGC |
| | | AGTTCAACTCCACTTACCGCGTCGTGTCCGTGCTGACGG |
| | | TGCTGCATCAGGACTGGCTGAACGGGAAGGAGTACAAG |
| | | TGCAAAGTGTCCAACAAGGGACTTCCTAGCTCAATCGA |
| | | AAAGACCATCTCGAAAGCCAAGGGACAGCCCCGGGAA |
| | | CCCCAAGTGTATACCCTGCCACCGAGCCAGGAAGAAAT |
| | | GACTAAGAACCAAGTCTCATTGACTTGCCTTGTGAAGG |
| | | GCTTCTACCCATCGGATATCGCCGTGGAATGGGAGTCC |
| | | AACGGCCAGCCGGAAAACAACTACAAGACCACCCCTCC |
| | | GGTGCTGGACTCAGACGGATCCTTCTTCCTCTACTCGCG |
| | | GCTGACCGTGGATAAGAGCAGATGGCAGGAGGGAAAT |
| | | GTGTTCAGCTGTTCTGTGATGCATGAAGCCCTGCACAAC |
| | | CACTACACTCAGAAGTCCCTGTCCCTCTCCCTGGGA |
| SEQ ID NO: 183 | LCDR1 | RASESVEYYGTSLMQ |
| (Kabat) | | |
| SEQ ID NO: 184 | LCDR2 | AASNVES |
| (Kabat) | | |
| SEQ ID NO: 185 | LCDR3 | QQSRKDPST |
| (Kabat) | | |
| SEQ ID NO: 186 | LCDR1 | SESVEYYGTSL |
| (Chothia) | | |
| SEQ ID NO: 187 | LCDR2 | AAS |
| (Chothia) | | |
| SEQ ID NO: 188 | LCDR3 | SRKDPS |
| (Chothia) | | |
| SEQ ID NO: 189 | VL | AIQLTQSPSSLSASVGDRVTITCRASESVEYYGTSLMQWY |
| | | QQKPGKAPKLLIYAASNVESGVPSRFSGSGSGTDFTLTISS |
| | | LQPEDFATYFCQQSRKDPSTFGGGTKVEIK |
| SEQ ID NO: 190 | DNA VL | GCTATTCAGCTGACTCAGTCACCTAGTAGCCTGAGCGCT |
| | | AGTGTGGGCGATAGAGTGACTATCACCTGTAGAGCTAG |
| | | TGAATCAGTCGAGTACTACGGCACTAGCCTGATGCAGT |
| | | GGTATCAGCAGAAGCCCGGGAAAGCCCCTAAGCTGCTG |
| | | ATCTACGCCGCCTCTAACGTGGAATCAGGCGTGCCCTCT |
| | | AGGTTTAGCGGTAGCGGTAGTGGCACCGACTTCACCCT |
| | | GACTATCTCTAGCCTGCAGCCCGAGGACTTCGCTACCTA |
| | | CTTCTGTCAGCAGTCTAGGAAGGACCCTAGCACCTTCG |
| | | GCGGAGGCACTAAGGTCGAGATTAAG |
| SEQ ID NO: 191 | Light | AIQLTQSPSSLSASVGDRVTITCRASESVEYYGTSLMQWY |
| | chain | QQKPGKAPKLLIYAASNVESGVPSRFSGSGSGTDFTLTISS |
| | | LQPEDFATYFCQQSRKDPSTFGGGTKVEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| SEQ ID NO: 192 | DNA | GCTATTCAGCTGACTCAGTCACCTAGTAGCCTGAGCGCT |
| | light | AGTGTGGGCGATAGAGTGACTATCACCTGTAGAGCTAG |
| | chain | TGAATCAGTCGAGTACTACGGCACTAGCCTGATGCAGT |
| | | GGTATCAGCAGAAGCCCGGGAAAGCCCCTAAGCTGCTG |
| | | ATCTACGCCGCCTCTAACGTGGAATCAGGCGTGCCCTCT |
| | | AGGTTTAGCGGTAGCGGTAGTGGCACCGACTTCACCCT |
| | | GACTATCTCTAGCCTGCAGCCCGAGGACTTCGCTACCTA |
| | | CTTCTGTCAGCAGTCTAGGAAGGACCCTAGCACCTTCG |
| | | GCGGAGGCACTAAGGTCGAGATTAAGCGTACGGTGGCC |
| | | GCTCCCAGCGTGTTCATCTTCCCCCCCAGCGACGAGCA |
| | | GCTGAAGAGCGGCACCGCCAGCGTGGTGTGCCTGCTGA |
| | | ACAACTTCTACCCCCGGGAGGCCAAGGTGCAGTGGAAG |
| | | GTGGACAACGCCCTGCAGAGCGGCAACAGCCAGGAGA |
| | | GCGTCACCGAGCAGGACAGCAAGGACTCCACCTACAGC |
| | | CTGAGCAGCACCCTGACCCTGAGCAAGGCCGACTACGA |
| | | GAAGCATAAGGTGTACGCCTGCGAGGTGACCCACCAGG |
| | | GCCTGTCCAGCCCCGTGACCAAGAGCTTCAACAGGGGC |
| | | GAGTGC |

ABTIM3-hum03

| | | |
|---|---|---|
| SEQ ID NO: 174 | HCDR1 | SYNMH |
| (Kabat) | | |
| SEQ ID NO: 193 | HCDR2 | DIYPGQGDTSYNQKFKG |
| (Kabat) | | |
| SEQ ID NO: 176 | HCDR3 | VGGAFPMDY |
| (Kabat) | | |
| SEQ ID NO: 177 | HCDR1 | GYTFTSY |
| (Chothia) | | |
| SEQ ID NO: 194 | HCDR2 | YPGQGD |
| (Chothia) | | |
| SEQ ID NO: 176 | HCDR3 | VGGAFPMDY |
| (Chothia) | | |
| SEQ ID NO: 195 | VH | QVQLVQSGAEVKKPGASVKVSCKASGYTFTSYNMHWVR |
| | | QAPGQGLEWIGDIYPGQGDTSYNQKFKGRATMTADKSTS |
| | | TVYMELSSLRSEDTAVYYCARVGGAFPMDYWGQGTLVT |
| | | VSS |
| SEQ ID NO: 196 | DNA VH | CAGGTGCAGCTGGTGCAGTCAGGCGCCGAAGTGAAGA |
| | | AACCCGGCGCTAGTGTGAAAGTTAGCTGTAAAGCTAGT |
| | | GGCTATACTTTCACTTCTTATAATATGCACTGGGTCCGC |
| | | CAGGCCCCAGGTCAAGGCCTCGAGTGGATCGGCGATAT |
| | | CTACCCCGGTCAAGGCGACACTTCCTATAATCAGAAGT |
| | | TTAAGGGTAGAGCTACTATGACCGCCGATAAGTCTACT |
| | | TCTACCGTCTATATGGAACTGAGTTCCCTGAGGTCTGAG |
| | | GACACCGCCGTCTACTACTGCGCTAGAGTGGGCGGAGC |
| | | CTTCCCAATGGACTACTGGGGTCAAGGCACCCTGGTCA |
| | | CCGTGTCTAGC |
| SEQ ID NO: 197 | Heavy | QVQLVQSGAEVKKPGASVKVSCKASGYTFTSYNMHWVR |
| | chain | QAPGQGLEWIGDIYPGQGDTSYNQKFKGRATMTADKSTS |
| | | TVYMELSSLRSEDTAVYYCARVGGAFPMDYWGQGTLVT |
| | | VSSASTKGPSVFPLAPCSRSTSESTAALGCLVKDYFPEPVT |
| | | VSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHNH |
| | | YTQKSLSLSLG |
| SEQ ID NO: 198 | DNA | CAGGTGCAGCTGGTGCAGTCAGGCGCCGAAGTGAAGA |
| | heavy | AACCCGGCGCTAGTGTGAAAGTTAGCTGTAAAGCTAGT |
| | chain | GGCTATACTTTCACTTCTTATAATATGCACTGGGTCCGC |
| | | CAGGCCCCAGGTCAAGGCCTCGAGTGGATCGGCGATAT |
| | | CTACCCCGGTCAAGGCGACACTTCCTATAATCAGAAGT |
| | | TTAAGGGTAGAGCTACTATGACCGCCGATAAGTCTACT |
| | | TCTACCGTCTATATGGAACTGAGTTCCCTGAGGTCTGAG |
| | | GACACCGCCGTCTACTACTGCGCTAGAGTGGGCGGAGC |
| | | CTTCCCAATGGACTACTGGGGTCAAGGCACCCTGGTCA |
| | | CCGTGTCTAGCGCTAGCACTAAGGGCCCGTCCGTGTTCC |
| | | CCCTGGCACCTTGTAGCCGGAGCACTAGCGAATCCACC |
| | | GCTGCCCTCGGCTGCCTGGTCAAGGATTACTTCCCGGA |
| | | GCCCGTGACCGTGTCCTGGAACAGCGGAGCCCTGACCT |
| | | CCGGAGTGCACACCTTCCCCGCTGTGCTGCAGAGCTCC |
| | | GGGCTGTACTCGCTGTCGTCGGTGGTCACGGTGCCTTCA |
| | | TCTAGCCTGGGTACCAAGACCTACACTTGCAACGTGGA |
| | | CCACAAGCCTTCCAACACTAAGGTGGACAAGCGCGTCG |
| | | AATCGAAGTACGGCCCACCGTGCCCGCCTTGTCCCGCG |
| | | CCGGAGTTCCTCGGCGGTCCCTCGGTCTTTCTGTTCCCA |
| | | CCGAAGCCCAAGGACACTTTGATGATTTCCCGCACCCC |
| | | TGAAGTGACATGCGTGGTCGTGGACGTGTCACAGGAAG |
| | | ATCCGGAGGTGCAGTTCAATTGGTACGTGGATGGCGTC |
| | | GAGGTGCACAACGCCAAAACCAAGCCGAGGGAGGAGC |
| | | AGTTCAACTCCACTTACCGCGTCGTGTCCGTGCTGACGG |
| | | TGCTGCATCAGGACTGGCTGAACGGGAAGGAGTACAAG |
| | | TGCAAAGTGTCCAACAAGGGACTTCCTAGCTCAATCGA |
| | | AAAGACCATCTCGAAAGCCAAGGGACAGCCCCGGGAA |
| | | CCCCAAGTGTATACCCTGCCACCGAGCCAGGAAGAAAT |
| | | GACTAAGAACCAAGTCTCATTGACTTGCCTTGTGAAGG |
| | | GCTTCTACCCATCGGATATCGCCGTGGAATGGGAGTCC |
| | | AACGGCCAGCCGGAAAACAACTACAAGACCACCCCTCC |
| | | GGTGCTGGACTCAGACGGATCCTTCTTCCTCTACTCGCG |
| | | GCTGACCGTGGATAAGAGCAGATGGCAGGAGGGAAAT |
| | | GTGTTCAGCTGTTCTGTGATGCATGAAGCCCTGCACAAC |
| | | CACTACACTCAGAAGTCCCTGTCCCTCTCCCTGGGA |
| SEQ ID NO: 183 | LCDR1 | RASESVEYYGTSLMQ |
| (Kabat) | | |
| SEQ ID NO: 184 | LCDR2 | AASNVES |
| (Kabat) | | |
| SEQ ID NO: 185 | LCDR3 | QQSRKDPST |
| (Kabat) | | |
| SEQ ID NO: 186 | LCDR1 | SESVEYYGTSL |
| (Chothia) | | |
| SEQ ID NO: 187 | LCDR2 | AAS |
| (Chothia) | | |
| SEQ ID NO: 188 | LCDR3 | SRKDPS |
| (Chothia) | | |
| SEQ ID NO: 199 | VL | DIVLTQSPDSLAVSLGERATINCRASESVEYYGTSLMQWY |
| | | QQKPGQPPKLLIYAASNVESGVPDRFSGSGSGTDFTLTISS |
| | | LQAEDVAVYYCQQSRKDPSTFGGGTKVEIK |
| SEQ ID NO: 200 | DNA VL | GATATCGTCCTGACTCAGTCACCCGATAGCCTGGCCGTC |
| | | AGCCTGGGCGAGCGGGCTACTATTAACTGTAGAGCTAG |
| | | TGAATCAGTCGAGTACTACGGCACTAGCCTGATGCAGT |
| | | GGTATCAGCAGAAGCCCGGTCAACCCCCTAAGCTGCTG |
| | | ATCTACGCCGCCTCTAACGTGGAATCAGGCGTGCCCGA |
| | | TAGGTTTAGCGGTAGCGGTAGTGGCACCGACTTCACCC |
| | | TGACTATTAGTAGCCTGCAGGCCGAGGACGTGGCCGTC |
| | | TACTACTGTCAGCAGTCTAGGAAGGACCCTAGCACCTT |
| | | CGGCGGAGGCACTAAGGTCGAGATTAAG |
| SEQ ID NO: 201 | Light | DIVLTQSPDSLAVSLGERATINCRASESVEYYGTSLMQWY |
| | chain | QQKPGQPPKLLIYAASNVESGVPDRFSGSGSGTDFTLTISS |
| | | LQAEDVAVYYCQQSRKDPSTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| SEQ ID NO: 202 | DNA | GATATCGTCCTGACTCAGTCACCCGATAGCCTGGCCGTC |
| | light | AGCCTGGGCGAGCGGGCTACTATTAACTGTAGAGCTAG |
| | chain | TGAATCAGTCGAGTACTACGGCACTAGCCTGATGCAGT |
| | | GGTATCAGCAGAAGCCCGGTCAACCCCCTAAGCTGCTG |
| | | ATCTACGCCGCCTCTAACGTGGAATCAGGCGTGCCCGA |
| | | TAGGTTTAGCGGTAGCGGTAGTGGCACCGACTTCACCC |
| | | TGACTATTAGTAGCCTGCAGGCCGAGGACGTGGCCGTC |
| | | TACTACTGTCAGCAGTCTAGGAAGGACCCTAGCACCTT |
| | | CGGCGGAGGCACTAAGGTCGAGATTAAGCGTACGGTGG |
| | | CCGCTCCCAGCGTGTTCATCTTCCCCCCCAGCGACGAGC |
| | | AGCTGAAGAGCGGCACCGCCAGCGTGGTGTGCCTGCTG |
| | | AACAACTTCTACCCCCGGGAGGCCAAGGTGCAGTGGAA |
| | | GGTGGACAACGCCCTGCAGAGCGGCAACAGCCAGGAG |
| | | AGCGTCACCGAGCAGGACAGCAAGGACTCCACCTACAG |
| | | CCTGAGCAGCACCCTGACCCTGAGCAAGGCCGACTACG |
| | | AGAAGCATAAGGTGTACGCCTGCGAGGTGACCCACCAG |
| | | GGCCTGTCCAGCCCCGTGACCAAGAGCTTCAACAGGGG |
| | | CGAGTGC |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 10. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 10
Amino acid sequences of other exemplary anti-TIM-3 antibody molecules
APE5137

| | | |
|---|---|---|
| SEQ ID NO: | VH | EVQLLESGGGLVQPGGSLRLSCAAASGFTFSSYDMSWVRQAPGKGL |
| 203 | | DWVSTISGGGTYTYYQDSVKGRFTISRDNSKNTLYLQMNSLRAEDT |
| | | AVYYCASMDYWGQGTTVTVSSA |
| SEQ ID NO: | VL | DIQMTQSPSSLSASVGDRVTITCRASQSIRRYLNWYHQKPGKAPKLLI |
| 204 | | YGASTLQSGVPSRFSGSGSGTDFTLTISSLQPEDFAVYYCQQSHSAPL |
| | | TFGGGTKVEIKR |

APE5121

| | | |
|---|---|---|
| SEQ ID NO: | VH | EVQVLESGGGLVQPGGSLRLYCVASGFTFSGSYAMSWVRQAPGKGL |
| 205 | | EWVSAISGSGGSTYYADSVKGRFTISRDNSKNTLYLQMNSLRAEDTA |
| | | VYYCAKKYYVGPADYWGQGTLVTVSSG |
| SEQ ID NO: | VL | DIVMTQSPDSLAVSLGERATINCKSSQSVLYSSNNKNYLAWYQHKP |
| 206 | | GQPPKLLIYWASTRESGVPDRFSGSGSGTDFTLTISSLQAEDVAVYYC |
| | | QQYYSSPLTFGGGTKIEVK |

### Cytokines

In yet another embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more cytokines, including but not limited to, interferon, IL-2, IL-15, IL-7, or IL21. In certain embodiments, 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, are administered in combination with an IL-15/IL-15Rα complex. In some embodiments, the IL-15/IL-15Rα complex is selected from NIZ985 (Novartis), ATL-803 (Altor) or CYP0150 (Cytune).

Exemplary IL-15/IL-15Rα complexes

In one embodiment, the cytokine is IL-15 complexed with a soluble form of IL-15 receptor alpha (IL-15Ra). The IL-15/IL-15Rα complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the formulation comprises an amino acid sequence of SEQ ID NO: 207 in Table 11 or an amino acid sequence at least 85%, 90%, 95%, or 99% identical or higher to SEQ ID NO: 207, and the soluble form of human IL-15Rα comprises an amino acid sequence of SEQ ID NO: 208 in Table 11, or an amino acid sequence at least 85%, 90%, 95%, or 99% identical or higher to SEQ ID NO: 208, as described in WO 2014/066527, incorporated by reference in its entirety. The molecules described herein can be made by vectors, host cells, and methods described in WO 2007084342, incorporated by reference in its entirety.

TABLE 11
Amino acid and nucleotide sequences of exemplary IL-15/IL-15Ra complexes
NIZ985

| | | |
|---|---|---|
| SEQ ID NO: | Human IL-15 | NWVNVISDLKKIEDLIQSMHIDATLYTESDVHPSCKVTAMKCF |
| 207 | | LLELQVISLESGDASIHDTVENLIILANNSLSSNGNVTESGCKEC |
| | | EELEEKNIKEFLQSFVHIVQMFINTS |
| SEQ ID NO: | Human | ITCPPPMSVEHADIWVKSYSLYSRERYICNSGFKRKAGTSSLTE |
| 208 | Soluble IL- | CVLNKATNVAHWTTPSLKCIRDPALVHQRPAPPSTVTTAGVTP |
| | 15Ra | QPESLSPSGKEPAASSPSSNNTAATTAAIVPGSQLMPSKSPSTGT |
| | | TEISSHESSHGTPSQTTAKNWELTASASHQPPGVYPQG |

### Other Exemplary IL-15 IL-15Rα Complexes

In one embodiment, the IL-15/IL-15Rα complex is ALT-803, an IL-15/IL-15Rα Fc fusion protein (IL-15N72D:IL-15RaSu/Fc soluble complex). ALT-803 is described in WO 2008/143794, incorporated by reference in its entirety. In one embodiment, the IL-15/IL-15Rα Fc fusion protein comprises the sequences as disclosed in Table 12.

In one embodiment, the IL-15/IL-15Rα complex comprises IL-15 fused to the sushi domain of IL-15Rα (CYP0150, Cytune). The sushi domain of IL-15Rα refers to a domain beginning at the first cysteine residue after the signal peptide of IL-15Ra, and ending at the fourth cysteine residue after said signal peptide. The complex of IL-15 fused to the sushi domain of IL-15Rα is described in WO 2007/04606 and WO 2012/175222, incorporated by reference in their entirety. In one embodiment, the IL-15/IL-15Rα sushi domain fusion comprises the sequences as disclosed in Table 12.

TABLE 12
Amino acid sequences of other exemplary IL-15/IL-15Ra complexes
ALT-803

| | | |
|---|---|---|
| SEQ ID NO: | IL-15N72D | NWVNVISDLKKIEDLIQSMHIDATLYTESDVHPSCKVTAMKCF |
| 209 | | LLELQVISLESGDASIHDTVENLIILANDSLSSNGNVTESGCKEC |
| | | EELEEKNIKEFLQSFVHIVQMFINTS |
| SEQ ID NO: | IL-15RaSu/ Fc | ITCPPPMSVEHADIWVKSYSLYSRERYICNSGFKRKAGTSSLTE |
| 210 | | CVLNKATNVAHWTTPSLKCIREPKSCDKTHTCPPCPAPELLGG |
| | | PSVFLFPPKPKDTLMISRTPEVTCVVVDVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSRDELTKNQVSL |
| | | TCLVKGFYPSDIAVEWESNGQPENNYKTTPPVLDSDGSFFLYS |
| | | KLTVDKSRWQQGNVFSCSVMHEALHNHYTQKSLSLSPGK |

IL-15 / IL-15Ra sushi domain fusion (CYP0150)

| | | |
|---|---|---|
| SEQ ID NO: | Human IL-15 | NWVNVISDLKKIEDLIQSMHIDATLYTESDVHPSCKVTAMKCF |
| 211 | | LLELQVISLESGDASIHDTVENLIILANNSLSSNGNVTESGCKEC |
| | | EELEXKNIKEFLQSFVHIVQMFINTS |
| | | Where X is E or K |
| SEQ ID NO: | Human IL- | ITCPPPMSVEHADIWVKSYSLYSRERYICNSGFKRKAGTSSLTE |
| 212 | 15Ra sushi | CVLNKATNVAHWTTPSLKCIRDPALVHQRPAPP |
| | and hinge | |
| | domains | |

In yet another embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more agonists of toll like receptors (TLRs, e.g., TLR7, TLR8, TLR9) to treat a disease, e.g., cancer. In some embodiments, the 3-(1-oxoisoindolin-2-yl)piperidine-2,6-dione IKZF2 degrader compound of the present disclosure can be used in combination with a TLR7 agonist or a TLR7 agonist conjugate.

In some embodiments, the TLR7 agonist comprises a compound disclosed in International Application Publication No. WO2011/049677, which is hereby incorporated by reference in its entirety. In some embodiments, the TLR7 agonist comprises 3-(5-amino-2-(4-(2-(3,3-difluoro-3-phosphonopropoxy)ethoxy)-2-methylphenethyl)benzo[f][1,7]naphthyridin-8-yl)propanoic acid. In some embodiments, the TLR7 agonist comprises a compound of formula:

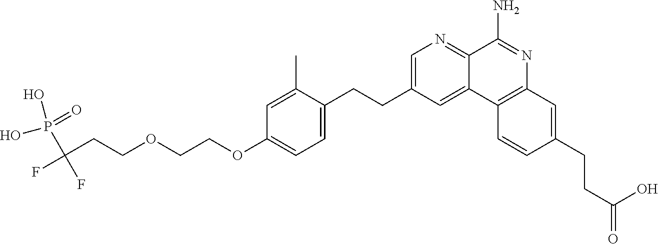 [SMILES: Cc1cc(OCCOCCC(F)(F)P(=O)(O)O)ccc1CCc1cnc2c(N)nc3cc(CCC(=O)O)ccc3c2c1]

In another embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more angiogenesis inhibitors to treat cancer, e.g., Bevacizumab (Avastin®), axitinib (Inlyta®); Brivanib alaninate (BMS-582664, (S)-((R)-1-(4-(4-Fluoro-2-methyl-1H-indol-5-yloxy)-5-methylpyrrolo[2,1-J][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); or Aflibercept (Eylea®).

In another embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more heat shock protein inhibitors to treat cancer, e.g., Tanespimycin (17-allylamino-17-demethoxygeldanamycin, also known as KOS-953 and 17-AAG, available from SIGMA, and described in U.S. Pat. No. 4,261,989); Retaspimycin (IPI504), Ganetespib (STA-9090); [6-Chloro-9-(4-methoxy-3,5-dimethylpyridin-2-ylmethyl)-9H-purin-2-yl]amine (BIIB021 or CNF2024, CAS 848695-25-0); trans-4-[[2-(Aminocarbonyl)-5-[4,5,6,7-tetrahydro-6,6-dimethyl-4-oxo-3-(trifluoromethyl)-1H-indazol-1-yl]phenyl]amino]cyclohexyl glycine ester (SNX5422 or PF04929113, CAS 908115-27-5); 5-[2,4-Dihydroxy-5-(1-methylethyl)phenyl]-N-ethyl-4-[4-(4-morpholinylmethyl)phenyl]-3-Isoxazolecarboxamide (AUY922, CAS 747412-49-3); or 17-Dimethylaminoethylamino-17-demethoxygeldanamycin (17-DMAG).

In yet another embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more HDAC inhibitors or other epigenetic modifiers. Exemplary HDAC inhibitors include, but not limited to, Voninostat (Zolinza®); Romidepsin (Istodax®); Treichostatin A (TSA); Oxamflatin; Vorinostat (Zolinza®, Suberoylanilide hydroxamic acid); Pyroxamide (syberoyl-3-aminopyridineamide hydroxamic acid); Trapoxin A (RF-1023A); Trapoxin B (RF-10238); Cyclo[(αS,2S)-α-amino-η-oxo-2-oxiraneoctanoyl-O-methyl-D-tyrosyl-L-isoleucyl-L-prolyl] (Cyl-1); Cyclo[(αS,2S)-α-amino-η-oxo-2-oxiraneoctanoyl-O-methyl-D-tyrosyl-L-isoleucyl-(2S)-2-piperidinecarbonyl] (Cyl-2); Cyclic[L-alanyl-D-alanyl-(2S)-η-oxo-L-α-aminooxiraneoctanoyl-D-prolyl] (HC-toxin); Cyclo[(aS,2S)-α-amino-η-oxo-2-oxiraneoctanoyl-D-phenylalanyl-L-leucyl-(2S)-2-piperidinecarbonyl] (WF-3161); Chlamydocin ((S)-Cyclic(2-methylalanyl-L-phenylalanyl-D-prolyl-η-oxo-L-α-aminooxiraneoctanoyl); Apicidin (Cyclo(8-oxo-L-2-aminodecanoyl-1-methoxy-L-tryptophyl-L-isoleucyl-D-2-piperidinecarbonyl); Romidepsin (Istodax®, FR-901228); 4-Phenylbutyrate; Spiruchostatin A; Mylproin (Valproic acid); Entinostat (MS-275, N-(2-Aminophenyl)-4-[N-(pyridine-3-yl-methoxycarbonyl)-amino-methyl]-benzamide); Depudecin (4,5:8,9-dianhydro-1,2,6,7,11-pentadeoxy-D-threo-D-ido-Undeca-1,6-dienitol); 4-(Acetylamino)-N-(2-aminophenyl)-benzamide (also known as CI-994); N1-(2-Aminophenyl)-N8-phenyl-octanediamide (also known as BML-210); 4-(Dimethylamino)-N-(7-(hydroxyamino)-7-oxoheptyl)benzamide (also known as M344); (E)-3-(4-(((2-(1H-indol-3-yl)ethyl)(2-hydroxyethyl)amino)-methyl)phenyl)-N-hydroxyacrylamide; Panobinostat(Farydak®); Mocetinostat, and Belinostat (also known as PXD101, Beleodaq®, or (2E)-N-Hydroxy-3-[3-(phenylsulfamoyl)phenyl]prop-2-enamide), or chidamide (also known as CS055 or HBI-8000, (E)-N-(2-amino-5-fluorophenyl)-4-((3-(pyridin-3-yl)acrylamido)methyl)benzamide). Other epigenetic modifiers include but not limited to inhibitors of EZH2 (enhancer of zeste homolog 2), EED (embryonic ectoderm development), or LSD1 (lysine-specific histone demethylase 1A or KDM1A).

In yet another embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more inhibitors of indoleamine-pyrrole 2,3-dioxygenase (IDO), for example, Indoximod (also known as NLG-8189), a-Cyclohexyl-5H-imidazo[5,1-a]isoindole-5-ethanol (also known as NLG919), or (4E)-4-[(3-Chloro-4-fluoroanilino)-nitrosomethylidene]-1,2,5-oxadiazol-3-amine (also known as INCB024360), to treat cancer.

### Chimeric Antigen Receptors

The present disclosure provides for the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof for use in combination with adoptive immunotherapy methods and reagents such as chimeric antigen receptor (CAR) immune effector cells, e.g., T cells, or chimeric TCR-transduced immune effector cells, e.g., T cells. This section describes CAR technology generally that is useful in combination with the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, and describes CAR reagents, e.g., cells and compositions, and methods.

In general, aspects of the present disclosure pertain to or include an isolated nucleic acid molecule encoding a chimeric antigen receptor (CAR), wherein the CAR comprises an antigen binding domain (e.g., antibody or antibody fragment, TCR or TCR fragment) that binds to a tumor antigen as described herein, a transmembrane domain (e.g., a transmembrane domain described herein), and an intracellular signaling domain (e.g., an intracellular signaling domain described herein) (e.g., an intracellular signaling domain comprising a costimulatory domain (e.g., a costimulatory domain described herein) and/or a primary signaling domain (e.g., a primary signaling domain described herein). In other aspects, the present disclosure includes: host cells containing the above nucleic acids and isolated proteins encoded by such nucleic acid molecules. CAR nucleic acid constructs, encoded proteins, containing vectors, host cells, pharmaceutical compositions, and methods of administration and treatment related to the present disclosure are disclosed in detail in International Patent Application Publication No. WO2015142675, which is incorporated by reference in its entirety.

In one aspect, the disclosure pertains to an isolated nucleic acid molecule encoding a chimeric antigen receptor (CAR), wherein the CAR comprises an antigen binding domain (e.g., antibody or antibody fragment, TCR or TCR fragment) that binds to a tumor-supporting antigen (e.g., a tumor-supporting antigen as described herein), a transmembrane domain (e.g., a transmembrane domain described herein), and an intracellular signaling domain (e.g., an intracellular signaling domain described herein) (e.g., an intracellular signaling domain comprising a costimulatory domain (e.g., a costimulatory domain described herein) and/or a primary signaling domain (e.g., a primary signaling domain described herein). In some embodiments, the tumor-supporting antigen is an antigen present on a stromal cell or a myeloid-derived suppressor cell (MDSC). In other aspects, the disclosure features polypeptides encoded by such nucleic acids and host cells containing such nucleic acids and/or polypeptides.

Alternatively, aspects of the disclosure pertain to isolated nucleic acid encoding a chimeric T cell receptor (TCR) comprising a TCR alpha and/or TCR beta variable domain with specificity for a cancer antigen described herein. See for example, Dembic et al., Nature, 320, 232-238 (1986), Schumacher, Nat. Rev. Immunol., 2, 512-519 (2002), Kershaw et al., Nat. Rev. Immunol., 5, 928-940 (2005), Xue et al., Clin. Exp. Immunol., 139, 167-172 (2005), Rossig et al., Mol. Ther., 10,5-18 (2004), and Murphy et al., Immunity, 22, 403-414 (2005); (Morgan et al. J. Immunol., 171, 3287-3295 (2003), Hughes et al., Hum. Gene Ther., 16,1-16 (2005), Zhao et al., J. Immunol., 174, 4415-4423 (2005), Roszkowski et al., Cancer Res., 65, 1570-1576 (2005), and Engels et al., Hum. Gene Ther., 16, 799-810 (2005); US2009/03046557, the contents of which are hereby incorporated by reference in their entirety. Such chimeric TCRs may recognize, for example, cancer antigens such as MART-1, gp-100, p53, and NY-ESO-1, MAGE A3/A6, MAGEA3, SSX2, HPV-16 E6 or HPV-16 E7. In other aspects, the disclosure features polypeptides encoded by such nucleic acids and host cells containing such nucleic acids and/or polypeptides.

Sequences of non-limiting examples of various components that can be part of a CAR are listed in Table 11a, where “aa” stands for amino acids, and “na” stands for nucleic acids that encode the corresponding peptide.

TABLE 11a
Sequences of various components of CAR (aa - amino acid sequence, na -
nucleic acid sequence).

| SEQ ID | | |
|---|---|---|
| NO: | description | Sequence |
| SEQ ID | EF-1 | CGTGAGGCTCCGGTGCCCGTCAGTGGGCAGAGCGCACATCGC |
| NO: 270 | promoter | CCACAGTCCCCGAGAAGTTGGGGGGAGGGGTCGGCAATTGAA |
| | (na) | CCGGTGCCTAGAGAAGGTGGCGCGGGGTAAACTGGGAAAGTG |
| | | ATGTCGTGTACTGGCTCCGCCTTTTTCCCGAGGGTGGGGGAGA |
| | | ACCGTATATAAGTGCAGTAGTCGCCGTGAACGTTCTTTTTCGC |
| | | AACGGGTTTGCCGCCAGAACACAGGTAAGTGCCGTGTGTGGT |
| | | TCCCGCGGGCCTGGCCTCTTTACGGGTTATGGCCCTTGCGTGC |
| | | CTTGAATTACTTCCACCTGGCTGCAGTACGTGATTCTTGATCCC |
| | | GAGCTTCGGGTTGGAAGTGGGTGGGAGAGTTCGAGGCCTTGC |
| | | GCTTAAGGAGCCCCTTCGCCTCGTGCTTGAGTTGAGGCCTGGC |
| | | CTGGGCGCTGGGGCCGCCGCGTGCGAATCTGGTGGCACCTTCG |
| | | CGCCTGTCTCGCTGCTTTCGATAAGTCTCTAGCCATTTAAAATT |
| | | TTTGATGACCTGCTGCGACGCTTTTTTTCTGGCAAGATAGTCTT |
| | | GTAAATGCGGGCCAAGATCTGCACACTGGTATTTCGGTTTTTG |
| | | GGGCCGCGGGCGGCGACGGGGCCCGTGCGTCCCAGCGCACAT |
| | | GTTCGGCGAGGCGGGGCCTGCGAGCGCGGCCACCGAGAATCG |
| | | GACGGGGGTAGTCTCAAGCTGGCCGGCCTGCTCTGGTGCCTGG |
| | | CCTCGCGCCGCCGTGTATCGCCCCGCCCTGGGCGGCAAGGCTG |
| | | GCCCGGTCGGCACCAGTTGCGTGAGCGGAAAGATGGCCGCTT |
| | | CCCGGCCCTGCTGCAGGGAGCTCAAAATGGAGGACGCGGCGC |
| | | TCGGGAGAGCGGGCGGGTGAGTCACCCACACAAAGGAAAAG |
| | | GGCCTTTCCGTCCTCAGCCGTCGCTTCATGTGACTCCACGGAG |
| | | TACCGGGCGCCGTCCAGGCACCTCGATTAGTTCTCGAGCTTTT |
| | | GGAGTACGTCGTCTTTAGGTTGGGGGGAGGGGTTTTATGCGAT |
| | | GGAGTTTCCCCACACTGAGTGGGTGGAGACTGAAGTTAGGCC |
| | | AGCTTGGCACTTGATGTAATTCTCCTTGGAATTTGCCCTTTTTG |
| | | AGTTTGGATCTTGGTTCATTCTCAAGCCTCAGACAGTGGTTCA |
| | | AAGTTTTTTTCTTCCATTTCAGGTGTCGTGA |
| SEQ ID | Leader (aa) | MALPVTALLLPLALLLHAARP |
| NO: 268 | | |
| SEQ ID | Leader (na) | ATGGCCCTGCCTGTGACAGCCCTGCTGCTGCCTCTGGCTCTGC |
| NO: | | TGCTGCATGCCGCTAGACCC |
| 287 | | |
| SEQ ID | Leader (na) | ATGGCCCTCCCTGTCACCGCCCTGCTGCTTCCGCTGGCTCTTCT |
| NO: | | GCTCCACGCCGCTCGGCCC |
| 288 | | |
| SEQ ID | CD 8 hinge | TTTPAPRPPTPAPTIASQPLSLRPEACRPAAGGAVHTRGLDFACD |
| NO: 250 | (aa) | |
| SEQ ID | CD8 hinge | ACCACGACGCCAGCGCCGCGACCACCAACACCGGCGCCCACC |
| NO: 254 | (na) | ATCGCGTCGCAGCCCCTGTCCCTGCGCCCAGAGGCGTGCCGGC |
| | | CAGCGGCGGGGGGCGCAGTGCACACGAGGGGGCTGGACTTCG |
| | | CCTGTGAT |
| SEQ ID | IgG4 hinge | ESKYGPPCPPCPAPEFLGGPSVFLFPPKPKDTLMISRTPEVTCVVV |
| NO: 253 | (aa) | DVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTPP |
| | | VLDSDGSFFLYSRLTVDKSRWQEGNVFSCSVMHEALHNHYTQK |
| | | SLSLSLGKM |
| SEQ ID | IgG4 hinge | GAGAGCAAGTACGGCCCTCCCTGCCCCCCTTGCCCTGCCCCCG |
| NO: 255 | (na) | AGTTCCTGGGCGGACCCAGCGTGTTCCTGTTCCCCCCCAAGCC |
| | | CAAGGACACCCTGATGATCAGCCGGACCCCCGAGGTGACCTG |
| | | TGTGGTGGTGGACGTGTCCCAGGAGGACCCCGAGGTCCAGTT |
| | | CAACTGGTACGTGGACGGCGTGGAGGTGCACAACGCCAAGAC |
| | | CAAGCCCCGGGAGGAGCAGTTCAATAGCACCTACCGGGTGGT |
| | | GTCCGTGCTGACCGTGCTGCACCAGGACTGGCTGAACGGCAA |
| | | GGAATACAAGTGTAAGGTGTCCAACAAGGGCCTGCCCAGCAG |
| | | CATCGAGAAAACCATCAGCAAGGCCAAGGGCCAGCCTCGGGA |
| | | GCCCCAGGTGTACACCCTGCCCCCTAGCCAAGAGGAGATGAC |
| | | CAAGAACCAGGTGTCCCTGACCTGCCTGGTGAAGGGCTTCTAC |
| | | CCCAGCGACATCGCCGTGGAGTGGGAGAGCAACGGCCAGCCC |
| | | GAGAACAACTACAAGACCACCCCCCCTGTGCTGGACAGCGAC |
| | | GGCAGCTTCTTCCTGTACAGCCGGCTGACCGTGGACAAGAGCC |
| | | GGTGGCAGGAGGGCAACGTCTTTAGCTGCTCCGTGATGCACG |
| | | AGGCCCTGCACAACCACTACACCCAGAAGAGCCTGAGCCTGT |
| | | CCCTGGGCAAGATG |
| SEQ ID | IgD hinge | RWPESPKAQASSVPTAQPQAEGSLAKATTAPATTRNTGRGGEEK |
| NO: 256 | (aa) | KKEKEKEEQEERETKTPECPSHTQPLGVYLLTPAVQDLWLRDKA |
| | | TFTCFVVGSDLKDAHLTWEVAGKVPTGGVEEGLLERHSNGSQSQ |
| | | HSRLTLPRSLWNAGTSVTCTLNHPSLPPQRLMALREPAAQAPVK |
| | | LSLNLLASSDPPEAASWLLCEVSGFSPPNILLMWLEDQREVNTSG |
| | | FAPARPPPQPGSTTFWAWSVLRVPAPPSPQPATYTCVVSHEDSRT |
| | | LLNASRSLEVSYVTDH |
| SEQ ID | IgD hinge | AGGTGGCCCGAAAGTCCCAAGGCCCAGGCATCTAGTGTTCCT |
| NO: 257 | (na) | ACTGCACAGCCCCAGGCAGAAGGCAGCCTAGCCAAAGCTACT |
| | | ACTGCACCTGCCACTACGCGCAATACTGGCCGTGGCGGGGAG |
| | | GAGAAGAAAAAGGAGAAAGAGAAAGAAGAACAGGAAGAGA |
| | | GGGAGACCAAGACCCCTGAATGTCCATCCCATACCCAGCCGC |
| | | TGGGCGTCTATCTCTTGACTCCCGCAGTACAGGACTTGTGGCT |
| | | TAGAGATAAGGCCACCTTTACATGTTTCGTCGTGGGCTCTGAC |
| | | CTGAAGGATGCCCATTTGACTTGGGAGGTTGCCGGAAAGGTA |
| | | CCCACAGGGGGGGTTGAGGAAGGGTTGCTGGAGCGCCATTCC |
| | | AATGGCTCTCAGAGCCAGCACTCAAGACTCACCCTTCCGAGAT |
| | | CCCTGTGGAACGCCGGGACCTCTGTCACATGTACTCTAAATCA |
| | | TCCTAGCCTGCCCCCACAGCGTCTGATGGCCCTTAGAGAGCCA |
| | | GCCGCCCAGGCACCAGTTAAGCTTAGCCTGAATCTGCTCGCCA |
| | | GTAGTGATCCCCCAGAGGCCGCCAGCTGGCTCTTATGCGAAGT |
| | | GTCCGGCTTTAGCCCGCCCAACATCTTGCTCATGTGGCTGGAG |
| | | GACCAGCGAGAAGTGAACACCAGCGGCTTCGCTCCAGCCCGG |
| | | CCCCCACCCCAGCCGGGTTCTACCACATTCTGGGCCTGGAGTG |
| | | TCTTAAGGGTCCCAGCACCACCTAGCCCCCAGCCAGCCACATA |
| | | CACCTGTGTTGTGTCCCATGAAGATAGCAGGACCCTGCTAAAT |
| | | GCTTCTAGGAGTCTGGAGGTTTCCTACGTGACTGACCATT |
| SEQ ID | GS | GGGGSGGGGS |
| NO: 258 | hinge/linker | |
| | (aa) | |
| SEQ ID | GS | GGTGGCGGAGGTTCTGGAGGTGGAGGTTCC |
| NO: 259 | hinge/linker | |
| | (na) | |
| SEQ ID | CD8 | IYIWAPLAGTCGVLLLSLVITLYC |
| NO: 251 | transmembrane | |
| | (aa) | |
| SEQ ID | CD8 | ATCTACATCTGGGCGCCCTTGGCCGGGACTTGTGGGGTCCTTC |
| NO: 252 | transmembrane | TCCTGTCACTGGTTATCACCCTTTACTGC |
| | (na) | |
| SEQ ID | CD8 | ATCTACATTTGGGCCCCTCTGGCTGGTACTTGCGGGGTCCTGC |
| NO: 289 | transmembrane | TGCTTTCACTCGTGATCACTCTTTACTGT |
| | (na) | |
| SEQ ID | 4-1BB | KRGRKKLLYIFKQPFMRPVQTTQEEDGCSCRFPEEEEGGCEL |
| NO: 264 | intracellular | |
| | domain (aa) | |
| SEQ ID | 4-1BB | AAACGGGGCAGAAAGAAACTCCTGTATATATTCAAACAACCA |
| NO: 266 | intracellular | TTTATGAGACCAGTACAAACTACTCAAGAGGAAGATGGCTGT |
| | domain (na) | AGCTGCCGATTTCCAGAAGAAGAAGAAGGAGGATGTGAACTG |
| SEQ ID | 4-1BB | AAGCGCGGTCGGAAGAAGCTGCTGTACATCTTTAAGCAACCC |
| NO: 290 | intracellular | TTCATGAGGCCTGTGCAGACTACTCAAGAGGAGGACGGCTGT |
| | domain (na) | TCATGCCGGTTCCCAGAGGAGGAGGAAGGCGGCTGCGAACTG |
| SEQ ID | CD27 (aa) | QRRKYRSNKGESPVEPAEPCRYSCPREEEGSTIPIQEDYRKPEPAC |
| NO: 265 | | SP |
| SEQ ID | CD27 (na) | Caacgaaggaaatatagatcaaacaaaggagaaagtcctgtggagcctgcagagccttgtcgttaca |
| NO: 267 | | gctgccccagggaggaggagggcagcaccatccccatccaggaggattaccgaaaaccggagcct |
| | | gcctgctccccc |
| SEQ ID | CD3-zeta | RVKFSRSADAPAYKQGQNQLYNELNLGRREEYDVLDKRRGRDP |
| NO: 260 | (aa) | EMGGKPRRKNPQEGLYNELQKDKMAEAYSEIGMKGERRRGKG |
| | | HDGLYQGLSTATKDTYDALHMQALPPR |
| SEQ ID | CD3-zeta | AGAGTGAAGTTCAGCAGGAGCGCAGACGCCCCCGCGTACAAG |
| NO: 262 | (na) | CAGGGCCAGAACCAGCTCTATAACGAGCTCAATCTAGGACGA |
| | | AGAGAGGAGTACGATGTTTTGGACAAGAGACGTGGCCGGGAC |
| | | CCTGAGATGGGGGGAAAGCCGAGAAGGAAGAACCCTCAGGA |
| | | AGGCCTGTACAATGAACTGCAGAAAGATAAGATGGCGGAGGC |
| | | CTACAGTGAGATTGGGATGAAAGGCGAGCGCCGGAGGGGCAA |
| | | GGGGCACGATGGCCTTTACCAGGGTCTCAGTACAGCCACCAA |
| | | GGACACCTACGACGCCCTTCACATGCAGGCCCTGCCCCCTCGC |
| SEQ ID | CD3-zeta | CGCGTGAAATTCAGCCGCAGCGCAGATGCTCCAGCCTACAAG |
| NO: 291 | (na) | CAGGGGCAGAACCAGCTCTACAACGAACTCAATCTTGGTCGG |
| | | AGAGAGGAGTACGACGTGCTGGACAAGCGGAGAGGACGGGA |
| | | CCCAGAAATGGGCGGGAAGCCGCGCAGAAAGAATCCCCAAG |
| | | AGGGCCTGTACAACGAGCTCCAAAAGGATAAGATGGCAGAAG |
| | | CCTATAGCGAGATTGGTATGAAAGGGGAACGCAGAAGAGGCA |
| | | AAGGCCACGACGGACTGTACCAGGGACTCAGCACCGCCACCA |
| | | AGGACACCTATGACGCTCTTCACATGCAGGCCCTGCCGCCTCG |
| | | G |
| SEQ ID | CD3-zeta | RVKFSRSADAPAYQQGQNQLYNELNLGRREEYDVLDKRRGRDP |
| NO: 261 | (aa) | EMGGKPRRKNPQEGLYNELQKDKMAEAYSEIGMKGERRRGKG |
| | | HDGLYQGLSTATKDTYDALHMQALPPR |
| SEQ ID | CD3-zeta | AGAGTGAAGTTCAGCAGGAGCGCAGACGCCCCCGCGTACCAG |
| NO: 263 | (na) | CAGGGCCAGAACCAGCTCTATAACGAGCTCAATCTAGGACGA |
| | | AGAGAGGAGTACGATGTTTTGGACAAGAGACGTGGCCGGGAC |
| | | CCTGAGATGGGGGGAAAGCCGAGAAGGAAGAACCCTCAGGA |
| | | AGGCCTGTACAATGAACTGCAGAAAGATAAGATGGCGGAGGC |
| | | CTACAGTGAGATTGGGATGAAAGGCGAGCGCCGGAGGGGCAA |
| | | GGGGCACGATGGCCTTTACCAGGGTCTCAGTACAGCCACCAA |
| | | GGACACCTACGACGCCCTTCACATGCAGGCCCTGCCCCCTCGC |
| SEQ ID | Linker (aa) | GGGGS |
| NO: 292 | | |
| SEQ ID | PD-1 | Pgwfldspdrpwnpptfspallvvtegdnatftcsfsntsesfvlnwyrmspsnqtdklaafpedrs |
| NO: 293 | extracellular | qpgqdcrfrvtqlpngrdfhmsvvrarrndsgtylcgaislapkaqikeslraelrvterraevptahp |
| | domain (aa) | spsprpagqfqtlv |
| SEQ ID | PD-1 | Cccggatggtttctggactctccggatcgcccgtggaatcccccaaccttctcaccggcactcttggttg |
| NO: 294 | extracellular | tgactgagggcgataatgcgaccttcacgtgctcgttctccaacacctccgaatcattcgtgctgaactg |
| | domain (na) | gtaccgcatgagcccgtcaaaccagaccgacaagctcgccgcgtttccggaagatcggtcgcaaccg |
| | | ggacaggattgtcggttccgcgtgactcaactgccgaatggcagagacttccacatgagcgtggtccg |
| | | cgctaggcgaaacgactccgggacctacctgtgcggagccatctcgctggcgcctaaggcccaaatc |
| | | aaagagagcttgagggccgaactgagagtgaccgagcgcagagctgaggtgccaactgcacatcca |
| | | tccccatcgcctcggcctgcggggcagtttcagaccctggtc |
| SEQ ID | PD-1 CAR | Malpvtalllplalllhaarppgwfldspdrpwnpptfspallvvtegdnatftcsfsntsesfvlnwy |
| NO: 295 | (aa) with | rmspsnqtdklaafpedrsqpgqdcrfrvtqlpngrdfhmsvvrarrndsgtylcgaislapkaqik |
| | signal | eslraelrvterraevptahpspsprpagqfqtlvtttpaprpptpaptiasqplslrpeacrpaaggavh |
| | | trgldfacdiyiwaplagtcgvlllslvitlyckrgrkkllyifkqpfmrpvqttqeedgcscrfpeeee |
| | | ggcelrvkfsrsadapaykqgqnqlynelnlgrreeydvldkrrgrdpemggkprrknpqeglyn |
| | | elqkdkmaeayseigmkgerrrgkghdglyqglstatkdtydalhmqalppr |
| SEQ ID | PD-1 CAR | Atggccctccctgtcactgccctgcttctccccctcgcactcctgctccacgccgctagaccacccgga |
| NO: 296 | (na) | tggtttctggactctccggatcgcccgtggaatcccccaaccttctcaccggcactcttggttgtgactga |
| | | gggcgataatgcgaccttcacgtgctcgttctccaacacctccgaatcattcgtgctgaactggtaccgc |
| | | atgagcccgtcaaaccagaccgacaagctcgccgcgtttccggaagatcggtcgcaaccgggacag |
| | | gattgtcggttccgcgtgactcaactgccgaatggcagagacttccacatgagcgtggtccgcgctagg |
| | | cgaaacgactccgggacctacctgtgcggagccatctcgctggcgcctaaggcccaaatcaaagaga |
| | | gcttgagggccgaactgagagtgaccgagcgcagagctgaggtgccaactgcacatccatccccatc |
| | | gcctcggcctgcggggcagtttcagaccctggtcacgaccactccggcgccgcgcccaccgactccg |
| | | gccccaactatcgcgagccagcccctgtcgctgaggccggaagcatgccgccctgccgccggaggt |
| | | gctgtgcatacccggggattggacttcgcatgcgacatctacatttgggctcctctcgccggaacttgtg |
| | | gcgtgctccttctgtccctggtcatcaccctgtactgcaagcggggtcggaaaaagcttctgtacattttc |
| | | aagcagcccttcatgaggcccgtgcaaaccacccaggaggaggacggttgctcctgccggttccccg |
| | | aagaggaagaaggaggttgcgagctgcgcgtgaagttctcccggagcgccgacgcccccgcctata |
| | | agcagggccagaaccagctgtacaacgaactgaacctgggacggcgggaagagtacgatgtgctgg |
| | | acaagcggcgcggccgggaccccgaaatggggggaagcctagaagaaagaaccctcaggaagg |
| | | cctgtataacgagctgcagaaggacaagatggccgaggcctactccgaaattgggatgaagggagag |
| | | cggcggaggggaaaggggcacgacggcctgtaccaaggactgtccaccgccaccaaggacacata |
| | | cgatgccctgcacatgcaggcccttccccctcgc |
| SEQ ID | Linker (aa) | (Gly-Gly-Gly-Ser)n, where n = 1-10 |
| NO: 297 | | |
| SEQ ID | Linker (aa) | (Gly4 Ser)4 |
| NO: 215 | | |
| SEQ ID | Linker (aa) | (Gly4 Ser)3 |
| NO: 216 | | |
| SEQ ID | Linker (aa) | (Gly3Ser) |
| NO: 297 | | |
| SEQ ID | poly A (na) | [a]₅₀₋₅₀₀₀ |
| NO: 298 | | |
| SEQ ID | PD1 CAR | Pgwfldspdrpwnpptfspallvvtegdnatftcsfsntsesfvlnwyrmspsnqtdklaafpedrs |
| NO: 299 | (aa) | gpgqdcrfrvtqlpngrdfhmsvvrarrndsgtylcgaislapkaqikeslraelrvterraevptahp |
| | | spsprpagqfqtlvtttpaprpptpaptiasqplslrpeacrpaaggavhtrgldfacdiyiwaplagtc |
| | | gvlllslvitlyckrgrkkllyifkqpfmrpvqttqeedgcscrfpeeeeggcelrvkfsrsadapayk |
| | | qgqnqlynelnlgrreeydvldkrrgrdpemggkprrknpqeglynelqkdkmaeayseigmk |
| | | gerrrgkghdglyqglstatkdtydalhmqalppr |
| SEQ ID | ICOS | TKKKYSSSVHDPNGEYMFMRAVNTAKKSRLTDVTL |
| NO: 300 | intracellular | |
| | domain (aa) | |
| SEQ ID | ICOS | ACAAAAAAGAAGTATTCATCCAGTGTGCACGACCCTAACGGT |
| NO: 301 | intracellular | GAATACATGTTCATGAGAGCAGTGAACACAGCCAAAAAATCC |
| | domain (na) | AGACTCACAGATGTGACCCTA |
| SEQ ID | ICOS TM | TTTPAPRPPTPAPTIASQPLSLRPEACRPAAGGAVHTRGLDFACDF |
| NO: 302 | domain (aa) | WLPIGCAAFVVVCILGCILICWL |
| SEQ ID | ICOS TM | ACCACGACGCCAGCGCCGCGACCACCAACACCGGCGCCCACC |
| NO: 303 | domain (na) | ATCGCGTCGCAGCCCCTGTCCCTGCGCCCAGAGGCGTGCCGGC |
| | | CAGCGGCGGGGGGCGCAGTGCACACGAGGGGGCTGGACTTCG |
| | | CCTGTGATTTCTGGTTACCCATAGGATGTGCAGCCTTTGTTGTA |
| | | GTCTGCATTTTGGGATGCATACTTATTTGTTGGCTT |
| SEQ ID | CD28 | RSKRSRLLHSDYMNMTPRRPGPTRKHYQPYAPPRDFAAYRS |
| NO: 304 | intracellular | |
| | domain (aa) | |
| SEQ ID | CD28 | AGGAGTAAGAGGAGCAGGCTCCTGCACAGTGACTACATGAAC |
| NO: 305 | intracellular | ATGACTCCCCGCCGCCCCGGGCCCACCCGCAAGCATTACCAGC |
| | domain (na) | CCTATGCCCCACCACGCGACTTCGCAGCCTATCGCTCC |

### Targets

The present disclosure provides cells, e.g., immune effector cells (e.g., T cells, NK cells), that comprise or at any time comprised a gRNA molecule or CRISPR system as described herein, that are further engineered to contain one or more CARs that direct the immune effector cells to undesired cells (e.g., cancer cells). This is achieved through an antigen binding domain on the CAR that is specific for a cancer associated antigen. There are two classes of cancer associated antigens (tumor antigens) that can be targeted by the CARs of the instant disclosure: (1) cancer associated antigens that are expressed on the surface of cancer cells; and (2) cancer associated antigens that itself is intracellular, however, a fragment of such antigen (peptide) is presented on the surface of the cancer cells by MHC (major histocompatibility complex).

In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu15Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO—1); Cancer/testis antigen 2 (LAGE-1a); Melanoma-associated antigen 1 (MAGE-Ai); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

A CAR described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. Without wishing to be bound by theory, in some embodiments, the CAR-expressing cells destroy the tumor-supporting cells, thereby indirectly inhibiting tumor growth or survival.

In embodiments, the stromal cell antigen is chosen from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is chosen from one or more of: CD33, CD11b, C14, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C14, CD15, and CD66b.

### Antigen Binding Domain Structures

In some embodiments, the antigen binding domain of the encoded CAR molecule comprises an antibody, an antibody fragment, an scFv, a Fv, a Fab, a (Fab′)2, a single domain antibody (SDAB), a VH or VL domain, a camelid VHH domain or a bi-functional (e.g. bi-specific) hybrid antibody (e.g., Lanzavecchia et al., Eur. J. Immunol. 17, 105 (1987)).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact.

In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO: 217). In one embodiment, the linker can be (Gly₄Ser)₄ (SEQ ID NO: 215) or (Gly₄Ser)₃(SEQ ID NO: 216). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Va and VP genes from a T cell clone linked by a linker (e.g., a flexible peptide). This approach is very useful to cancer associated target that itself is intracellular, however, a fragment of such antigen (peptide) is presented on the surface of the cancer cells by MHC.

In certain embodiments, the encoded antigen binding domain has a binding affinity KD of 10⁻⁴ M to 10⁻⁸ M.

In one embodiment, the encoded CAR molecule comprises an antigen binding domain that has a binding affinity KD of 10⁻⁴ M to 10⁻⁸ M, e.g., 10⁻⁵ M to 10⁻⁷ M, e.g., 10⁻⁶ M or 10⁻⁷ M, for the target antigen. In one embodiment, the antigen binding domain has a binding affinity that is at least five-fold, 10-fold, 20-fold, 30-fold, 50-fold, 100-fold or 1,000-fold less than a reference antibody, e.g., an antibody described herein. In one embodiment, the encoded antigen binding domain has a binding affinity at least 5-fold less than a reference antibody (e.g., an antibody from which the antigen binding domain is derived). In one aspect such antibody fragments are functional in that they provide a biological response that can include, but is not limited to, activation of an immune response, inhibition of signal-transduction origination from its target antigen, inhibition of kinase activity, and the like, as will be understood by a skilled artisan. In one aspect, the antigen binding domain of the CAR is a scFv antibody fragment that is humanized compared to the murine sequence of the scFv from which it is derived.

In one aspect, the antigen binding domain of a CAR of the disclosure (e.g., a scFv) is encoded by a nucleic acid molecule whose sequence has been codon optimized for expression in a mammalian cell. In one aspect, entire CAR construct of the disclosure is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

### Antigen Binding Domains (and the Targeted Antigens)

In one embodiment, an antigen binding domain against CD19 is an antigen binding portion, e.g., CDRs, of a CAR, antibody or antigen-binding fragment thereof described in, e.g., PCT publication WO2012/079000; PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190.

In one embodiment, an antigen binding domain against mesothelin is an antigen binding portion, e.g., CDRs, of an antibody, antigen-binding fragment or CAR described in, e.g., PCT publication WO2015/090230. In one embodiment, an antigen binding domain against mesothelin is an antigen binding portion, e.g., CDRs, of an antibody, antigen-binding fragment, or CAR described in, e.g., PCT publication WO1997/025068, WO1999/028471, WO2005/014652, WO2006/099141, WO2009/045957, WO2009/068204, WO2013/142034, WO2013/040557, or WO2013/063419. In one embodiment, an antigen binding domain against mesothelin is an antigen binding portion, e.g., CDRs, of an antibody, antigen-binding fragment, or CAR described in WO/2015/090230.

In one embodiment, an antigen binding domain against CD123 is an antigen binding portion, e.g., CDRs, of an antibody, antigen-binding fragment or CAR described in, e.g., PCT publication WO2014/130635. In one embodiment, an antigen binding domain against CD123 is an antigen binding portion, e.g., CDRs, of an antibody, antigen-binding fragment, or CAR described in, e.g., PCT publication WO2014/138805, WO2014/138819, WO2013/173820, WO2014/144622, WO2001/66139, WO2010/126066, WO2014/144622, or US2009/0252742. In one embodiment, an antigen binding domain against CD123 is an antigen binding portion, e.g., CDRs, of an antibody, antigen-binding fragment, or CAR described in WO/2016/028896.

In one embodiment, an antigen binding domain against EGFRvIII is an antigen binding portion, e.g., CDRs, of an antibody, antigen-binding fragment or CAR described in, e.g., WO/2014/130657.

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD). In one embodiment, an antigen binding domain against CLL-1 is an antigen binding portion, e.g., CDRs, of an antibody, antigen-binding fragment, or CAR described in WO/2016/014535.

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6),Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014). In one embodiment, an antigen binding domain against CD33 is an antigen binding portion, e.g., CDRs, of an antibody, antigen-binding fragment, or CAR described in WO/2016/014576.

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18,14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody described in, e.g., WO2012163805, WO200112812, and WO2003062401. In one embodiment, an antigen binding domain against BCMA is an antigen binding portion, e.g., CDRs, of an antibody, antigen-binding fragment, or CAR described in WO/2016/014565.

In one embodiment, an antigen binding domain against Tn antigen is an antigen binding portion, e.g., CDRs, of an antibody described in, e.g., U.S. Pat. No. 8,440,798, Brooks et al., PNAS 107(22):10056-10061 (2010), and Stone et al., Oncolmmunology 1(6):863-873(2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastoenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-IIRa is an antigen binding portion, e.g., CDRs, of an antibody available from Abcam (cat #ab55262) or Novus Biologicals (cat #EPR5446). In another embodiment, an antigen binding domain again IL-IIRa is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013(2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab.

In one embodiment, an antigen binding domain against NCAM is an antigen binding portion, e.g., CDRs, of the antibody clone 2-2B: MAB5324 (EMD Millipore).

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007 In one embodiment, an antigen binding domain against tyrosinase is an antigen binding portion, e.g., CDRs, of an antibody described in, e.g., U.S. Pat. No. 5,843,674; or U.S. Ser. No. 19/950,504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., US20100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J.15(3):243-9 (1998), Lou et al., Proc Natl Acad Sci USA 111(7):2482-2487 (2014); MBrl: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences) In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signaling Technology; or antibody HPA017748—Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, CA Dec. 6-9 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1xCD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 Nov; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32. In one embodiment, an antigen binding domain against FCRL5 is an antigen binding portion, e.g., CDRs, of the anti-FcRL5 antibody described in, for example, WO2001/038490, WO/2005/117986, WO2006/039238, WO2006/076691, WO2010/114940, WO2010/120561, or WO2014/210064.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

In an embodiment, the antigen-binding domain of a CAR, e.g., a CAR expressed by a cell of the disclosure, binds to CD19. CD19 is found on B cells throughout differentiation of the lineage from the pro/pre-B cell stage through the terminally differentiated plasma cell stage. In an embodiment, the antigen binding domain is a murine scFv domain that binds to human CD19, e.g., the antigen binding domain of CTL019 (e.g., SEQ ID NO: 218). In an embodiment, the antigen binding domain is a humanized antibody or antibody fragment, e.g., scFv domain, derived from the murine CTL019 scFv. In an embodiment, the antigen binding domain is a human antibody or antibody fragment that binds to human CD19. Exemplary scFv domains (and their sequences, e.g., CDRs, VL and VH sequences) that bind to CD19 are provided in Table 12a. The scFv domain sequences provided in Table 12a include a light chain variable region (VL) and a heavy chain variable region (VH). The VL and VH are attached by a linker comprising the sequence GGGGSGGGGSGGGGS (SEQ ID NO: 216), e.g., in the following orientation: VL-linker-VH.

TABLE 12a
Antigen Binding domains that bind CD19

| | | | SEQ |
|---|---|---|---|
| | | | ID |
| Antigen | Name | Amino Acid Sequence | NO: |
| CD19 | muCTL019 | DIQMTQTTSSLSASLGDRVTISCRASQDISKYLNWYQQKPDGTV | 218 |
| | | KLLIYHTSRLHSGVPSRFSGSGSGTDYSLTISNLEQEDIATYFCQQ | |
| | | GNTLPYTFGGGTKLEITGGGGSGGGGSGGGGSEVKLQESGPGL | |
| | | VAPSQSLSVTCTVSGVSLPDYGVSWIRQPPRKGLEWLGVIWGSE | |
| | | TTYYNSALKSRLTIIKDNSKSQVFLKMNSLQTDDTAIYYCAKHY | |
| | | YYGGSYAMDYWGQGTSVTVSS | |
| CD19 | huscFv1 | EIVMTQSPATLSLSPGERATLSCRASQDISKYLNWYQQKPGQAP | 219 |
| | | RLLIYHTSRLHSGIPARFSGSGSGTDYTLTISSLQPEDFAVYFCQQ | |
| | | GNTLPYTFGQGTKLEIKGGGGSGGGGSGGGGSQVQLQESGPGL | |
| | | VKPSETLSLTCTVSGVSLPDYGVSWIRQPPGKGLEWIGVIWGSE | |
| | | TTYYSSSLKSRVTISKDNSKNQVSLKLSSVTAADTAVYYCAKH | |
| | | YYYGGSYAMDYWGQGTLVTVSS | |
| CD19 | huscFv2 | EIVMTQSPATLSLSPGERATLSCRASQDISKYLNWYQQKPGQAP | 220 |
| | | RLLIYHTSRLHSGIPARFSGSGSGTDYTLTISSLQPEDFAVYFCQQ | |
| | | GNTLPYTFGQGTKLEIKGGGGSGGGGSGGGGSQVQLQESGPGL | |
| | | VKPSETLSLTCTVSGVSLPDYGVSWIRQPPGKGLEWIGVIWGSE | |
| | | TTYYQSSLKSRVTISKDNSKNQVSLKLSSVTAADTAVYYCAKH | |
| | | YYYGGSYAMDYWGQGTLVTVSS | |
| CD19 | huscFv3 | QVQLQESGPGLVKPSETLSLTCTVSGVSLPDYGVSWIRQPPGKG | 221 |
| | | LEWIGVIWGSETTYYSSSLKSRVTISKDNSKNQVSLKLSSVTAA | |
| | | DTAVYYCAKHYYYGGSYAMDYWGQGTLVTVSSGGGGSGGGG | |
| | | SGGGGSEIVMTQSPATLSLSPGERATLSCRASQDISKYLNWYQQ | |
| | | KPGQAPRLLIYHTSRLHSGIPARFSGSGSGTDYTLTISSLQPEDFA | |
| | | VYFCQQGNTLPYTFGQGTKLEIK | |
| CD19 | huscFv4 | QVQLQESGPGLVKPSETLSLTCTVSGVSLPDYGVSWIRQPPGKG | 222 |
| | | LEWIGVIWGSETTYYQSSLKSRVTISKDNSKNQVSLKLSSVTAA | |
| | | DTAVYYCAKHYYYGGSYAMDYWGQGTLVTVSSGGGGSGGGG | |
| | | SGGGGSEIVMTQSPATLSLSPGERATLSCRASQDISKYLNWYQQ | |
| | | KPGQAPRLLIYHTSRLHSGIPARFSGSGSGTDYTLTISSLQPEDFA | |
| | | VYFCQQGNTLPYTFGQGTKLEIK | |
| CD19 | huscFv5 | EIVMTQSPATLSLSPGERATLSCRASQDISKYLNWYQQKPGQAP | 223 |
| | | RLLIYHTSRLHSGIPARFSGSGSGTDYTLTISSLQPEDFAVYFCQQ | |
| | | GNTLPYTFGQGTKLEIKGGGGSGGGGSGGGGSGGGGSQVQLQE | |
| | | SGPGLVKPSETLSLTCTVSGVSLPDYGVSWIRQPPGKGLEWIGVI | |
| | | WGSETTYYSSSLKSRVTISKDNSKNQVSLKLSSVTAADTAVYYC | |
| | | AKHYYYGGSYAMDYWGQGTLVTVSS | |
| CD19 | huscFv6 | EIVMTQSPATLSLSPGERATLSCRASQDISKYLNWYQQKPGQAP | 224 |
| | | RLLIYHTSRLHSGIPARFSGSGSGTDYTLTISSLQPEDFAVYFCQQ | |
| | | GNTLPYTFGQGTKLEIKGGGGSGGGGSGGGGSGGGGSQVQLQE | |
| | | SGPGLVKPSETLSLTCTVSGVSLPDYGVSWIRQPPGKGLEWIGVI | |
| | | WGSETTYYQSSLKSRVTISKDNSKNQVSLKLSSVTAADTAVYY | |
| | | CAKHYYYGGSYAMDYWGQGTLVTVSS | |
| CD19 | huscFv7 | QVQLQESGPGLVKPSETLSLTCTVSGVSLPDYGVSWIRQPPGKG | 225 |
| | | LEWIGVIWGSETTYYSSSLKSRVTISKDNSKNQVSLKLSSVTAA | |
| | | DTAVYYCAKHYYYGGSYAMDYWGQGTLVTVSSGGGGSGGGG | |
| | | SGGGGSGGGGSEIVMTQSPATLSLSPGERATLSCRASQDISKYL | |
| | | NWYQQKPGQAPRLLIYHTSRLHSGIPARFSGSGSGTDYTLTISSL | |
| | | QPEDFAVYFCQQGNTLPYTFGQGTKLEIK | |
| CD19 | huscFv8 | QVQLQESGPGLVKPSETLSLTCTVSGVSLPDYGVSWIRQPPGKG | 226 |
| | | LEWIGVIWGSETTYYQSSLKSRVTISKDNSKNQVSLKLSSVTAA | |
| | | DTAVYYCAKHYYYGGSYAMDYWGQGTLVTVSSGGGGSGGGG | |
| | | SGGGGSGGGGSEIVMTQSPATLSLSPGERATLSCRASQDISKYL | |
| | | NWYQQKPGQAPRLLIYHTSRLHSGIPARFSGSGSGTDYTLTISSL | |
| | | QPEDFAVYFCQQGNTLPYTFGQGTKLEIK | |
| CD19 | huscFv9 | EIVMTQSPATLSLSPGERATLSCRASQDISKYLNWYQQKPGQAP | 227 |
| | | RLLIYHTSRLHSGIPARFSGSGSGTDYTLTISSLQPEDFAVYFCQQ | |
| | | GNTLPYTFGQGTKLEIKGGGGSGGGGSGGGGSGGGGSQVQLQE | |
| | | SGPGLVKPSETLSLTCTVSGVSLPDYGVSWIRQPPGKGLEWIGVI | |
| | | WGSETTYYNSSLKSRVTISKDNSKNQVSLKLSSVTAADTAVYY | |
| | | CAKHYYYGGSYAMDYWGQGTLVTVSS | |
| CD19 | HuscFv10 | QVQLQESGPGLVKPSETLSLTCTVSGVSLPDYGVSWIRQPPGKG | 228 |
| | | LEWIGVIWGSETTYYNSSLKSRVTISKDNSKNQVSLKLSSVTAA | |
| | | DTAVYYCAKHYYYGGSYAMDYWGQGTLVTVSSGGGGSGGGG | |
| | | SGGGGSGGGGSEIVMTQSPATLSLSPGERATLSCRASQDISKYL | |
| | | NWYQQKPGQAPRLLIYHTSRLHSGIPARFSGSGSGTDYTLTISSL | |
| | | QPEDFAVYFCQQGNTLPYTFGQGTKLEIK | |
| CD19 | HuscFv11 | EIVMTQSPATLSLSPGERATLSCRASQDISKYLNWYQQKPGQAP | 229 |
| | | RLLIYHTSRLHSGIPARFSGSGSGTDYTLTISSLQPEDFAVYFCQQ | |
| | | GNTLPYTFGQGTKLEIKGGGGSGGGGSGGGGSQVQLQESGPGL | |
| | | VKPSETLSLTCTVSGVSLPDYGVSWIRQPPGKGLEWIGVIWGSE | |
| | | TTYYNSSLKSRVTISKDNSKNQVSLKLSSVTAADTAVYYCAKH | |
| | | YYYGGSYAMDYWGQGTLVTVSS | |
| CD19 | HuscFv12 | QVQLQESGPGLVKPSETLSLTCTVSGVSLPDYGVSWIRQPPGKG | 230 |
| | | LEWIGVIWGSETTYYNSSLKSRVTISKDNSKNQVSLKLSSVTAA | |
| | | DTAVYYCAKHYYYGGSYAMDYWGQGTLVTVSSGGGGSGGGG | |
| | | SGGGGSEIVMTQSPATLSLSPGERATLSCRASQDISKYLNWYQQ | |
| | | KPGQAPRLLIYHTSRLHSGIPARFSGSGSGTDYTLTISSLQPEDFA | |
| | | VYFCQQGNTLPYTFGQGTKLEIK | |

The sequences of the CDR sequences of the scFv domains of the CD19 antigen binding domains provided in Table 12a are shown in Table 12b for the heavy chain variable domains and in Table 12c for the light chain variable domains. “ID” stands for the respective SEQ ID NO for each CDR.

TABLE 12b
Heavy Chain Variable Domain CDRs

| Description | FW | HCDR1 | ID | HCDR2 | ID | HCDR3 | ID |
|---|---|---|---|---|---|---|---|
| murine_CART19 | | GVSLPDYGVS | 306 | VIWGSETTYYNSALKS | 307 | HYYYGGSYAMDY | 231 |
| humanized_CART19 a | VH4 | GVSLPDYGVS | 306 | VIWGSETTYYSSSLKS | 308 | HYYYGGSYAMDY | 231 |
| humanized_CART19 b | VH4 | GVSLPDYGVS | 306 | VIWGSETTYYQSSLKS | 309 | HYYYGGSYAMDY | 231 |
| humanized_CART19 c | VH4 | GVSLPDYGVS | 306 | VIWGSETTYYNSSLKS | 310 | HYYYGGSYAMDY | 231 |

TABLE 12c
Light Chain Variable Domain CDRs

| Description | FW | LCDR1 | ID | LCDR2 | ID | LCDR3 | ID |
|---|---|---|---|---|---|---|---|
| murine_CART19 | | RASQDISKYLN | 311 | HTSRLHS | 312 | QQGNTLPYT | 232 |
| humanized_CART19 a | VK3 | RASQDISKYLN | 311 | HTSRLHS | 312 | QQGNTLPYT | 232 |
| humanized_CART19 b | VK3 | RASQDISKYLN | 311 | HTSRLHS | 312 | QQGNTLPYT | 232 |
| humanized_CART19 c | VK3 | RASQDISKYLN | 311 | HTSRLHS | 312 | QQGNTLPYT | 232 |

In an embodiment, the antigen binding domain comprises an anti-CD19 antibody, or fragment thereof, e.g., a scFv. For example, the antigen binding domain comprises a variable heavy chain and a variable light chain listed in Table 12d. The linker sequence joining the variable heavy and variable light chains can be any of the linker sequences described herein, or alternatively, can be GSTSGSGKPGSGEGSTKG (SEQ ID NO: 233). The light chain variable region and heavy chain variable region of a scFv can be, e.g., in any of the following orientations: light chain variable region-linker-heavy chain variable region or heavy chain variable region-linker-light chain variable region.

TABLE 12d
Additional Anti-CD19 antibody binding domains

| Ab | | |
|---|---|---|
| Name | VH Sequence | VL Sequence |
| SJ25-C1 | QVQLLESGAELVRPGSSVKISCKAS | ELVLTQSPKFMSTSVGDRVSVTCKAS |
| | GYAFSSYWMNWVKQRPGQGLEWI | QNVGTNVAWYQQKPGQSPKPLIYSA |
| | GQIYPGDGDTNYNGKFKGQATLTA | TYRNSGVPDRFTGSGSGTDFTLTITNV |
| | DKSSSTAYMQLSGLTSEDSAVYSC | QSKDLADYFYFCQYNRYPYTSGGGT |
| | ARKTISSVVDFYFDYWGQGTTVT | KLEIKRRS (SEQ ID NO: 235) |
| | (SEQ ID NO: 234) | |

| | ScFv Sequence |
|---|---|
| SJ25-C1 | QVQLLESGAELVRPGSSVKISCKASGYAFSSYWMNWVKQRPGQGLEWIGQI |
| scFv | YPGDGDTNYNGKFKGQATLTADKSSSTAYMQLSGLTSEDSAVYSCARKTISS |
| | VVDFYFDYWGQGTTVTGSTSGSGKPGSGEGSTKGELVLTQSPKFMSTSVGDR |
| | VSVTCKASQNVGTNVAWYQQKPGQSPKPLIYSATYRNSGVPDRFTGSGSGTD |
| | FTLTITNVQSKDLADYFYFCQYNRYPYTSGGGTKLEIKRRS (SEQ ID NO: 236) |

In one embodiment, the CD19 binding domain comprises one or more (e.g., all three) light chain complementary determining region 1 (LC CDR1), light chain complementary determining region 2 (LC CDR2), and light chain complementary determining region 3 (LC CDR3) of a CD19 binding domain described herein, e.g., provided in Table 12a or 15, and/or one or more (e.g., all three) heavy chain complementary determining region 1 (HC CDR1), heavy chain complementary determining region 2 (HC CDR2), and heavy chain complementary determining region 3 (HC CDR3) of a CD19 binding domain described herein, e.g., provided in Table 12a or 16. In one embodiment, the CD19 binding domain comprises one, two, or all of LC CDR1, LC CDR2, and LC CDR3 of any amino acid sequences as provided in Table 12c, incorporated herein by reference; and one, two or all of HC CDR1, HC CDR2, and HC CDR3 of any amino acid sequences as provided in Table 12b.

Any known CD19 CAR, e.g., the CD19 antigen binding domain of any known CD19 CAR, in the art can be used in accordance with the instant disclosure to construct a CAR. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10. In one embodiment, an antigen binding domain against CD19 is an antigen binding portion, e.g., CDRs, of a CAR, antibody or antigen-binding fragment thereof described in, e.g., PCT publication WO2012/079000; PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190.

In an embodiment, the antigen-binding domain of CAR, e.g., a CAR expressed by a cell of the disclosure, binds to BCMA. BCMA is found preferentially expressed in mature B lymphocytes. In an embodiment, the antigen binding domain is a murine scFv domain that binds to human BCMA. In an embodiment, the antigen binding domain is a humanized antibody or antibody fragment, e.g., scFv domain that binds human BCMA. In an embodiment, the antigen binding domain is a human antibody or antibody fragment that binds to human BCMA. In embodiments, exemplary BCMA CAR constructs are generated using the VH and VL sequences from PCT Publication WO2012/0163805 (the contents of which are hereby incorporated by reference in its entirety). In embodiments, additional exemplary BCMA CAR constructs are generated using the VH and VL sequences from PCT Publication WO2016/014565 (the contents of which are hereby incorporated by reference in its entirety). In embodiments, additional exemplary BCMA CAR constructs are generated using the VH and VL sequences from PCT Publication WO2014/122144 (the contents of which are hereby incorporated by reference in its entirety). In embodiments, additional exemplary BCMA CAR constructs are generated using the CAR molecules, and/or the VH and VL sequences from PCT Publication WO2016/014789 (the contents of which are hereby incorporated by reference in its entirety). In embodiments, additional exemplary BCMA CAR constructs are generated using the CAR molecules, and/or the VH and VL sequences from PCT Publication WO2014/089335 (the contents of which are hereby incorporated by reference in its entirety). In embodiments, additional exemplary BCMA CAR constructs are generated using the CAR molecules, and/or the VH and VL sequences from PCT Publication WO2014/140248 (the contents of which are hereby incorporated by reference in its entirety).

Any known BCMA CAR, e.g., the BMCA antigen binding domain of any known BCMA CAR, in the art can be used in accordance with the instant disclosure. For example, those described herein.

### Exemplary CAR Molecules

In one aspect, a CAR, e.g., a CAR expressed by the cell of the disclosure, comprises a CAR molecule comprising an antigen binding domain that binds to a B cell antigen, e.g., as described herein, such as CD19 or BCMA.

In one embodiment, the CAR comprises a CAR molecule comprising a CD19 antigen binding domain (e.g., a murine, human or humanized antibody or antibody fragment that specifically binds to CD 19), a transmembrane domain, and an intracellular signaling domain (e.g., an intracellular signaling domain comprising a costimulatory domain and/or a primary signaling domain).

Exemplary CAR molecules described herein are provided in Table 12e. The CAR molecules in Table 12e comprise a CD19 antigen binding domain, e.g., an amino acid sequence of any CD19 antigen binding domain provided in Table 12a.

TABLE 12e
Exemplary CD19 CAR molecules

| | | | SEQ |
|---|---|---|---|
| Antigen | Name | Amino Acid Sequence | ID NO: |
| CD19 | CTL019 | MALPVTALLLPLALLLHAARPDIQMTQTTSSLSASLGDRVTISCR | 237 |
| | | ASQDISKYLNWYQQKPDGTVKLLIYHTSRLHSGVPSRFSGSGSGT | |
| | | DYSLTISNLEQEDIATYFCQQGNTLPYTFGGGTKLEITGGGGSGG | |
| | | GGSGGGGSEVKLQESGPGLVAPSQSLSVTCTVSGVSLPDYGVSW | |
| | | IRQPPRKGLEWLGVIWGSETTYYNSALKSRLTIIKDNSKSQVFLK | |
| | | MNSLQTDDTAIYYCAKHYYYGGSYAMDYWGQGTSVTVSSTTTP | |
| | | APRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |
| CD19 | CAR | MALPVTALLLPLALLLHAARPEIVMTQSPATLSLSPGERATLSCR | 238 |
| | 1 | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSQVQLQESGPGLVKPSETLSLTCTVSGVSLPDYGVSW | |
| | | IRQPPGKGLEWIGVIWGSETTYYSSSLKSRVTISKDNSKNQVSLK | |
| | | LSSVTAADTAVYYCAKHYYYGGSYAMDYWGQGTLVTVSSTTT | |
| | | PAPRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |
| CD19 | CAR | MALPVTALLLPLALLLHAARPEIVMTQSPATLSLSPGERATLSCR | 239 |
| | 2 | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSQVQLQESGPGLVKPSETLSLTCTVSGVSLPDYGVSW | |
| | | IRQPPGKGLEWIGVIWGSETTYYQSSLKSRVTISKDNSKNQVSLK | |
| | | LSSVTAADTAVYYCAKHYYYGGSYAMDYWGQGTLVTVSSTTT | |
| | | PAPRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |
| CD19 | CAR | MALPVTALLLPLALLLHAARPQVQLQESGPGLVKPSETLSLTCTV | 240 |
| | 3 | SGVSLPDYGVSWIRQPPGKGLEWIGVIWGSETTYYSSSLKSRVTI | |
| | | SKDNSKNQVSLKLSSVTAADTAVYYCAKHYYYGGSYAMDYWG | |
| | | QGTLVTVSSGGGGSGGGGSGGGGSEIVMTQSPATLSLSPGERAT | |
| | | LSCRASQDISKYLNWYQQKPGQAPRLLIYHTSRLHSGIPARFSGS | |
| | | GSGTDYTLTISSLQPEDFAVYFCQQGNTLPYTFGQGTKLEIKTTTP | |
| | | APRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |
| CD19 | CAR | MALPVTALLLPLALLLHAARPQVQLQESGPGLVKPSETLSLTCTV | 241 |
| | 4 | SGVSLPDYGVSWIRQPPGKGLEWIGVIWGSETTYYQSSLKSRVTI | |
| | | SKDNSKNQVSLKLSSVTAADTAVYYCAKHYYYGGSYAMDYWG | |
| | | QGTLVTVSSGGGGSGGGGSGGGGSEIVMTQSPATLSLSPGERAT | |
| | | LSCRASQDISKYLNWYQQKPGQAPRLLIYHTSRLHSGIPARFSGS | |
| | | GSGTDYTLTISSLQPEDFAVYFCQQGNTLPYTFGQGTKLEIKTTTP | |
| | | APRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |
| CD19 | CAR | MALPVTALLLPLALLLHAARPEIVMTQSPATLSLSPGERATLSCR | 242 |
| | 5 | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSGGGGSQVQLQESGPGLVKPSETLSLTCTVSGVSLPD | |
| | | YGVSWIRQPPGKGLEWIGVIWGSETTYYSSSLKSRVTISKDNSKN | |
| | | QVSLKLSSVTAADTAVYYCAKHYYYGGSYAMDYWGQGTLVTV | |
| | | SSTTTPAPRPPTPAPTIASQPLSLRPEACRPAAGGAVHTRGLDFAC | |
| | | DIYIWAPLAGTCGVLLLSLVITLYCKRGRKKLLYIFKQPFMRPVQ | |
| | | TTQEEDGCSCRFPEEEEGGCELRVKFSRSADAPAYKQGQNQLYN | |
| | | ELNLGRREEYDVLDKRRGRDPEMGGKPRRKNPQEGLYNELQKD | |
| | | KMAEAYSEIGMKGERRRGKGHDGLYQGLSTATKDTYDALHMQ | |
| | | ALPPR | |
| CD19 | CAR | MALPVTALLLPLALLLHAARPEIVMTQSPATLSLSPGERATLSCR | 243 |
| | 6 | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSGGGGSQVQLQESGPGLVKPSETLSLTCTVSGVSLPD | |
| | | YGVSWIRQPPGKGLEWIGVIWGSETTYYQSSLKSRVTISKDNSKN | |
| | | QVSLKLSSVTAADTAVYYCAKHYYYGGSYAMDYWGQGTLVTV | |
| | | SSTTTPAPRPPTPAPTIASQPLSLRPEACRPAAGGAVHTRGLDFAC | |
| | | DIYIWAPLAGTCGVLLLSLVITLYCKRGRKKLLYIFKQPFMRPVQ | |
| | | TTQEEDGCSCRFPEEEEGGCELRVKFSRSADAPAYKQGQNQLYN | |
| | | ELNLGRREEYDVLDKRRGRDPEMGGKPRRKNPQEGLYNELQKD | |
| | | KMAEAYSEIGMKGERRRGKGHDGLYQGLSTATKDTYDALHMQ | |
| | | ALPPR | |
| CD19 | CAR | MALPVTALLLPLALLLHAARPQVQLQESGPGLVKPSETLSLTCTV | 244 |
| | 7 | SGVSLPDYGVSWIRQPPGKGLEWIGVIWGSETTYYSSSLKSRVTI | |
| | | SKDNSKNQVSLKLSSVTAADTAVYYCAKHYYYGGSYAMDYWG | |
| | | QGTLVTVSSGGGGSGGGGSGGGGSGGGGSEIVMTQSPATLSLSP | |
| | | GERATLSCRASQDISKYLNWYQQKPGQAPRLLIYHTSRLHSGIPA | |
| | | RFSGSGSGTDYTLTISSLQPEDFAVYFCQQGNTLPYTFGQGTKLEI | |
| | | KTTTPAPRPPTPAPTIASQPLSLRPEACRPAAGGAVHTRGLDFAC | |
| | | DIYIWAPLAGTCGVLLLSLVITLYCKRGRKKLLYIFKQPFMRPVQ | |
| | | TTQEEDGCSCRFPEEEEGGCELRVKFSRSADAPAYKQGQNQLYN | |
| | | ELNLGRREEYDVLDKRRGRDPEMGGKPRRKNPQEGLYNELQKD | |
| | | KMAEAYSEIGMKGERRRGKGHDGLYQGLSTATKDTYDALHMQ | |
| | | ALPPR | |
| CD19 | CAR | MALPVTALLLPLALLLHAARPQVQLQESGPGLVKPSETLSLTCTV | 245 |
| | 8 | SGVSLPDYGVSWIRQPPGKGLEWIGVIWGSETTYYQSSLKSRVTI | |
| | | SKDNSKNQVSLKLSSVTAADTAVYYCAKHYYYGGSYAMDYWG | |
| | | QGTLVTVSSGGGGSGGGGSGGGGSGGGGSEIVMTQSPATLSLSP | |
| | | GERATLSCRASQDISKYLNWYQQKPGQAPRLLIYHTSRLHSGIPA | |
| | | RFSGSGSGTDYTLTISSLQPEDFAVYFCQQGNTLPYTFGQGTKLEI | |
| | | KTTTPAPRPPTPAPTIASQPLSLRPEACRPAAGGAVHTRGLDFAC | |
| | | DIYIWAPLAGTCGVLLLSLVITLYCKRGRKKLLYIFKQPFMRPVQ | |
| | | TTQEEDGCSCRFPEEEEGGCELRVKFSRSADAPAYKQGQNQLYN | |
| | | ELNLGRREEYDVLDKRRGRDPEMGGKPRRKNPQEGLYNELQKD | |
| | | KMAEAYSEIGMKGERRRGKGHDGLYQGLSTATKDTYDALHMQ | |
| | | ALPPR | |
| CD19 | CAR | MALPVTALLLPLALLLHAARPEIVMTQSPATLSLSPGERATLSCR | 246 |
| | 9 | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSGGGGSQVQLQESGPGLVKPSETLSLTCTVSGVSLPD | |
| | | YGVSWIRQPPGKGLEWIGVIWGSETTYYNSSLKSRVTISKDNSKN | |
| | | QVSLKLSSVTAADTAVYYCAKHYYYGGSYAMDYWGQGTLVTV | |
| | | SSTTTPAPRPPTPAPTIASQPLSLRPEACRPAAGGAVHTRGLDFAC | |
| | | DIYIWAPLAGTCGVLLLSLVITLYCKRGRKKLLYIFKQPFMRPVQ | |
| | | TTQEEDGCSCRFPEEEEGGCELRVKFSRSADAPAYKQGQNQLYN | |
| | | ELNLGRREEYDVLDKRRGRDPEMGGKPRRKNPQEGLYNELQKD | |
| | | KMAEAYSEIGMKGERRRGKGHDGLYQGLSTATKDTYDALHMQ | |
| | | ALPPR | |
| CD19 | CAR | MALPVTALLLPLALLLHAARPEIVMTQSPATLSLSPGERATLSCR | 247 |
| | 10 | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSGGGGSQVQLQESGPGLVKPSETLSLTCTVSGVSLPD | |
| | | YGVSWIRQPPGKGLEWIGVIWGSETTYYNSSLKSRVTISKDNSKN | |
| | | QVSLKLSSVTAADTAVYYCAKHYYYGGSYAMDYWGQGTLVTV | |
| | | SSTTTPAPRPPTPAPTIASQPLSLRPEACRPAAGGAVHTRGLDFAC | |
| | | DIYIWAPLAGTCGVLLLSLVITLYCKRGRKKLLYIFKQPFMRPVQ | |
| | | TTQEEDGCSCRFPEEEEGGCELRVKFSRSADAPAYKQGQNQLYN | |
| | | ELNLGRREEYDVLDKRRGRDPEMGGKPRRKNPQEGLYNELQKD | |
| | | KMAEAYSEIGMKGERRRGKGHDGLYQGLSTATKDTYDALHMQ | |
| | | ALPPR | |
| CD19 | CAR | MALPVTALLLPLALLLHAARPQVQLQESGPGLVKPSETLSLTCTV | 248 |
| | 11 | SGVSLPDYGVSWIRQPPGKGLEWIGVIWGSETTYYNSSLKSRVTI | |
| | | SKDNSKNQVSLKLSSVTAADTAVYYCAKHYYYGGSYAMDYWG | |
| | | QGTLVTVSSGGGGSGGGGSGGGGSGGGGSEIVMTQSPATLSLSP | |
| | | GERATLSCRASQDISKYLNWYQQKPGQAPRLLIYHTSRLHSGIPA | |
| | | RFSGSGSGTDYTLTISSLQPEDFAVYFCQQGNTLPYTFGQGTKLEI | |
| | | KTTTPAPRPPTPAPTIASQPLSLRPEACRPAAGGAVHTRGLDFAC | |
| | | DIYIWAPLAGTCGVLLLSLVITLYCKRGRKKLLYIFKQPFMRPVQ | |
| | | TTQEEDGCSCRFPEEEEGGCELRVKFSRSADAPAYKQGQNQLYN | |
| | | ELNLGRREEYDVLDKRRGRDPEMGGKPRRKNPQEGLYNELQKD | |
| | | KMAEAYSEIGMKGERRRGKGHDGLYQGLSTATKDTYDALHMQ | |
| | | ALPPR | |
| CD19 | CAR | MALPVTALLLPLALLLHAARPEIVMTQSPATLSLSPGERATLSCR | 249 |
| | 12 | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSQVQLQESGPGLVKPSETLSLTCTVSGVSLPDYGVSW | |
| | | IRQPPGKGLEWIGVIWGSETTYYNSSLKSRVTISKDNSKNQVSLK | |
| | | LSSVTAADTAVYYCAKHYYYGGSYAMDYWGQGTLVTVSSTTT | |
| | | PAPRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |

In one aspect, a CAR, e.g., a CAR expressed by the cell of the disclosure, comprises a CAR molecule comprising an antigen binding domain that binds to BCMA, e.g., comprises a BCMA antigen binding domain (e.g., a murine, human or humanized antibody or antibody fragment that specifically binds to BCMA, e.g., human BCMA), a transmembrane domain, and an intracellular signaling domain (e.g., an intracellular signaling domain comprising a costimulatory domain and/or a primary signaling domain).

Exemplary CAR molecules of a CAR described herein are provided in Table 1 of WO2016/014565, which is incorporated by reference herein.

### Transmembrane Domains

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3,4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR e.g., in one embodiment, the transmembrane domain may be from the same protein that the signalling domain, costimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one aspect, the transmembrane domain is capable of signalling to the intracellular domain(s) whenever the CAR has bound to a target. A transmembrane domain of particular use in this disclosure may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO—3), BLAME (SLAMF8), SELPLG (CD162), LTBR, PAG/Cbp, NKG2D, NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge. In one embodiment, the hinge or spacer comprises (e.g., consists of) the amino acid sequence of SEQ ID NO: 250. In one aspect, the transmembrane domain comprises (e.g., consists of) a transmembrane domain of SEQ ID NO: 251.

In certain embodiments, the encoded transmembrane domain comprises an amino acid sequence of a CD8 transmembrane domain having at least one, two or three modifications but not more than 20, 10 or 5 modifications of the amino acid sequence of SEQ ID NO: 251, or a sequence with at least 95% identity to the amino acid sequence of SEQ ID NO: 251. In one embodiment, the encoded transmembrane domain comprises the sequence of SEQ ID NO: 251.

In other embodiments, the nucleic acid molecule encoding the CAR comprises a nucleotide sequence of a CD8 transmembrane domain, e.g., comprising the sequence of SEQ ID NO: 252 or SEQ ID NO: 289, or a sequence with at least 95% identity thereof.

In certain embodiments, the encoded antigen binding domain is connected to the transmembrane domain by a hinge region. In one embodiment, the encoded hinge region comprises the amino acid sequence of a CD8 hinge, e.g., SEQ ID NO: 250; or the amino acid sequence of an IgG4 hinge, e.g., SEQ ID NO: 253 or a sequence with at least 95% identity to SEQ ID NO: 250 or SEQ ID NO: 253. In other embodiments, the nucleic acid sequence encoding the hinge region comprises the sequence of SEQ ID NO: 254 or SEQ ID NO: 255, corresponding to a CD8 hinge or an IgG4 hinge, respectively, or a sequence with at least 95% identity to SEQ ID NO: 254 or 255.

In one aspect, the hinge or spacer comprises an IgG4 hinge. For example, in one embodiment, the hinge or spacer comprises a hinge of the amino acid sequence ESKYGPPCPPCPAPEFLGGPSVFLFPPKPKDTLMISRTPEVTCVVVDVSQEDPEVQFNWYVDGVE VHNAKTKPREEQFNSTYRVVSVLTVLHQDWLNGKEYKCKVSNKGLPSSIEKTISKAKGQPREPQ VYTLPPSQEEMTKNQVSLTCLVKGFYPSDIAVEWESNGQPENNYKTTPPVLDSDGSFFLYSRLTV DKSRWQEGNVFSCSVMHEALHNHYTQKSLSLSLGKM (SEQ ID NO: 253). In some embodiments, the hinge or spacer comprises a hinge encoded by the nucleotide sequence of

(SEQ ID NO: 255)
GAGAGCAAGTACGGCCCTCCCTGCCCCCCTTGCCCTGCCCCCGAGTTCCT
GGGCGGACCCAGCGTGTTCCTGTTCCCCCCCAAGCCCAAGGACACCCTGA
TGATCAGCCGGACCCCCGAGGTGACCTGTGTGGTGGTGGACGTGTCCCAG
GAGGACCCCGAGGTCCAGTTCAACTGGTACGTGGACGGCGTGGAGGTGCA
CAACGCCAAGACCAAGCCCCGGGAGGAGCAGTTCAATAGCACCTACCGGG
TGGTGTCCGTGCTGACCGTGCTGCACCAGGACTGGCTGAACGGCAAGGAA
TACAAGTGTAAGGTGTCCAACAAGGGCCTGCCCAGCAGCATCGAGAAAAC
CATCAGCAAGGCCAAGGGCCAGCCTCGGGAGCCCCAGGTGTACACCCTGC
CCCCTAGCCAAGAGGAGATGACCAAGAACCAGGTGTCCCTGACCTGCCTG
GTGAAGGGCTTCTACCCCAGCGACATCGCCGTGGAGTGGGAGAGCAACGG
CCAGCCCGAGAACAACTACAAGACCACCCCCCCTGTGCTGGACAGCGACG
GCAGCTTCTTCCTGTACAGCCGGCTGACCGTGGACAAGAGCCGGTGGCAG
GAGGGCAACGTCTTTAGCTGCTCCGTGATGCACGAGGCCCTGCACAACCA
CTACACCCAGAAGAGCCTGAGCCTGTCCCTGGGCAAGATG.

In one aspect, the hinge or spacer comprises an IgD hinge. For example, in one embodiment, the hinge or spacer comprises a hinge of the amino acid sequence of RWPESPKAQASSVPTAQPQAEGSLAKATTAPATTRNTGRGGEEKKKEKEKEEQEERETKTPECP SHTQPLGVYLLTPAVQDLWLRDKATFTCFVVGSDLKDAHLTWEVAGKVPTGGVEEGLLERHSN GSQSQHSRLTLPRSLWNAGTSVTCTLNHPSLPPQRLMALREPAAQAPVKLSLNLLASSDPPEAAS WLLCEVSGFSPPNILLMWLEDQREVNTSGFAPARPPPQPGSTTFWAWSVLRVPAPPSPQPATYTC VVSHEDSRTLLNASRSLEVSYVTDH (SEQ ID NO: 256). In some embodiments, the hinge or spacer comprises a hinge encoded by the nucleotide sequence of

(SEQ ID NO: 257)
AGGTGGCCCGAAAGTCCCAAGGCCCAGGCATCTAGTGTTCCTACTGCACA
GCCCCAGGCAGAAGGCAGCCTAGCCAAAGCTACTACTGCACCTGCCACTA
CGCGCAATACTGGCCGTGGCGGGGAGGAGAAGAAAAAGGAGAAAGAGAAA
GAAGAACAGGAAGAGAGGGAGACCAAGACCCCTGAATGTCCATCCCATAC
CCAGCCGCTGGGCGTCTATCTCTTGACTCCCGCAGTACAGGACTTGTGGC
TTAGAGATAAGGCCACCTTTACATGTTTCGTCGTGGGCTCTGACCTGAAG
GATGCCCATTTGACTTGGGAGGTTGCCGGAAAGGTACCCACAGGGGGGGT
TGAGGAAGGGTTGCTGGAGCGCCATTCCAATGGCTCTCAGAGCCAGCACT
CAAGACTCACCCTTCCGAGATCCCTGTGGAACGCCGGGACCTCTGTCACA
TGTACTCTAAATCATCCTAGCCTGCCCCCACAGCGTCTGATGGCCCTTAG
AGAGCCAGCCGCCCAGGCACCAGTTAAGCTTAGCCTGAATCTGCTCGCCA
GTAGTGATCCCCCAGAGGCCGCCAGCTGGCTCTTATGCGAAGTGTCCGGC
TTTAGCCCGCCCAACATCTTGCTCATGTGGCTGGAGGACCAGCGAGAAGT
GAACACCAGCGGCTTCGCTCCAGCCCGGCCCCCACCCCAGCCGGGTTCTA
CCACATTCTGGGCCTGGAGTGTCTTAAGGGTCCCAGCACCACCTAGCCCC
CAGCCAGCCACATACACCTGTGTTGTGTCCCATGAAGATAGCAGGACCCT
GCTAAATGCTTCTAGGAGTCTGGAGGTTTCCTACGTGACTGACCATT.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO: 258). In some embodiments, the linker is encoded by the nucleotide sequence of GGTGGCGGAGGTTCTGGAGGTGGAGGTTCC (SEQ ID NO: 259).

In one aspect, the hinge or spacer comprises a KIR2DS2 hinge.

### Signaling Domains

In embodiments of the disclosure having an intracellular signaling domain, such a domain can contain, e.g., one or more of a primary signaling domain and/or a costimulatory signaling domain. In some embodiments, the intracellular signaling domain comprises a sequence encoding a primary signaling domain. In some embodiments, the intracellular signaling domain comprises a costimulatory signaling domain. In some embodiments, the intracellular signaling domain comprises a primary signaling domain and a costimulatory signaling domain.

The intracellular signaling sequences within the cytoplasmic portion of the CAR of the disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

### Primary Signaling Domains

A primary signaling domain regulates primary activation of the TCR complex either in a stimulatory way, or in an inhibitory way. Primary intracellular signaling domains that act in a stimulatory manner may contain signaling motifs, which are known as immunoreceptor tyrosine-based activation motifs or ITAMs.

Examples of ITAM containing primary intracellular signaling domains that are of particular use in the disclosure include those of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In one embodiment, a CAR of the disclosure comprises an intracellular signaling domain, e.g., a primary signaling domain of CD3-zeta.

In one embodiment, the encoded primary signaling domain comprises a functional signaling domain of CD3 zeta. The encoded CD3 zeta primary signaling domain can comprise an amino acid sequence having at least one, two or three modifications but not more than 20, 10 or 5 modifications of the amino acid sequence of SEQ ID NO: 260 or SEQ ID NO: 261, or a sequence with at least 95% identity to the amino acid sequence of SEQ ID NO: 260 or SEQ ID NO: 261. In some embodiments, the encoded primary signaling domain comprises the sequence of SEQ ID NO: 260 or SEQ ID NO: 261. In other embodiments, the nucleic acid sequence encoding the primary signaling domain comprises the sequence of SEQ ID NO: 262, SEQ ID NO: 291, or SEQ ID NO: 263, or a sequence with at least 95% identity thereof.

### Costimulatory Signaling Domains

In some embodiments, the encoded intracellular signaling domain comprises a costimulatory signaling domain. For example, the intracellular signaling domain can comprise a primary signaling domain and a costimulatory signaling domain. In some embodiments, the encoded costimulatory signaling domain comprises a functional signaling domain of a protein chosen from one or more of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

In certain embodiments, the encoded costimulatory signaling domain comprises an amino acid sequence having at least one, two or three modifications but not more than 20, 10 or 5 modifications of the amino acid sequence of SEQ ID NO: 264 or SEQ ID NO: 265, or a sequence with at least 95% identity to the amino acid sequence of SEQ ID NO: 264 or SEQ ID NO: 265. In one embodiment, the encoded costimulatory signaling domain comprises the sequence of SEQ ID NO: 264 or SEQ ID NO: 265. In other embodiments, the nucleic acid sequence encoding the costimulatory signaling domain comprises the sequence of SEQ ID NO: 266, SEQ ID NO: 290, or SEQ ID NO: 267, or a sequence with at least 95% identity thereof.

In other embodiments, the encoded intracellular domain comprises the sequence of SEQ ID NO: 264 or SEQ ID NO: 265 and the sequence of SEQ ID NO: 260 or SEQ ID NO: 261, wherein the sequences comprising the intracellular signaling domain are expressed in the same frame and as a single polypeptide chain.

In certain embodiments, the nucleic acid sequence encoding the intracellular signaling domain comprises the sequence of SEQ ID NO: 266, SEQ ID NO: 290, or SEQ ID NO: 267, or a sequence with at least 95% identity thereof, and the sequence of SEQ ID NO: 262, SEQ ID NO: 291, or SEQ ID NO: 263, or a sequence with at least 95% identity thereof.

In some embodiments, the nucleic acid molecule further encodes a leader sequence. In one embodiment, the leader sequence comprises the sequence of SEQ ID NO: 268.

In one aspect, the intracellular signalling domain is designed to comprise the signalling domain of CD3-zeta and the signalling domain of CD28. In one aspect, the intracellular signalling domain is designed to comprise the signaling domain of CD3-zeta and the signaling domain of 4-1BB. In one aspect, the signaling domain of 4-1BB is a signaling domain of SEQ ID NO: 264. In one aspect, the signaling domain of CD3-zeta is a signaling domain of SEQ ID NO: 260.

In one aspect, the intracellular signaling domain is designed to comprise the signaling domain of CD3-zeta and the signaling domain of CD27. In one aspect, the signaling domain of CD27 comprises the amino acid sequence of QRRKYRSNKGESPVEPAEPCRYSCPREEEGSTIPIQEDYRKPEPACSP (SEQ ID NO: 265). In one aspect, the signaling domain of CD27 is encoded by the nucleic acid sequence of

(SEQ ID NO: 267)
Caacgaaggaaatatagatcaaacaaaggagaaagtcctgtggagcctgc
agagccttgtcgttacagctgccccagggaggaggagggcagcaccatcc
ccatccaggaggattaccgaaaaccggagcctgcctgctccccc.

### Vectors

In another aspect, the disclosure pertains to a vector comprising a nucleic acid sequence encoding a CAR described herein. In one embodiment, the vector is chosen from a DNA vector, an RNA vector, a plasmid, a lentivirus vector, adenoviral vector, or a retrovirus vector. In one embodiment, the vector is a lentivirus vector. These vectors or portions thereof may, among other things, be used to create template nucleic acids, as described herein for use with the CRISPR systems as described herein. Alternatively, the vectors may be used to deliver nucleic acid directly to the cell, e.g., the immune effector cell, e.g., the T cell, e.g., the allogeneic T cell, independent of the CRISPR system.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (W), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the disclosure is an adenoviral vector (A5/35). In another embodiment, the expression of nucleic acids encoding CARs can be accomplished using of transposons such as sleeping beauty, crisper, CAS9, and zinc finger nucleases. See below June et al. 2009Nature Reviews Immunology 9.10: 704-716, is incorporated herein by reference.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Disclosed herein are methods for producing an in vitro transcribed RNA CAR. The present disclosure also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO: 269). RNA so produced can efficiently transfect different kinds of cells. In one aspect, the template includes sequences for the CAR.

### Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, cells of the disclosure, e.g., T or NK cells, e.g., allogeneic T cells, e.g., described herein, (e.g., that express a CAR described herein) are generated by contacting the cells with (a) a composition comprising one or more gRNA molecules, e.g., as described herein, and one or more Cas molecules, e.g., a Cas9 molecule, e.g., as described herein, and (b) nucleic acid comprising sequence encoding a CAR, e.g., described herein (such as a template nucleic acid molecule as described herein). Without being bound by theory, said composition of (a), above, will induce a break at or near the genomic DNA targeted by the targeting domain of the gRNA molecule(s), and the nucleic acid of (b) will incorporate, e.g., partially or wholly, into the genome at or near said break, such that upon integration, the encoded CAR molecule is expressed. In embodiments, expression of the CAR will be controlled by promoters or other regulatory elements endogenous to the genome (e.g., the promoter controlling expression from the gene in which the nucleic acid of (b) was inserted). In other embodiments, the nucleic acid of (b) further comprises a promoter and/or other regulatory elements, e.g., as described herein, e.g., an EF1-alpha promoter, operably linked to the sequence encoding the CAR, such that upon integration, expression of the CAR is controlled by that promoter and/or other regulatory elements. Additional features of the disclosure relating to use of CRISPR/Cas9 systems, e.g., as described herein, to direct incorporation of nucleic acid sequence encoding a CAR, e.g., as described herein, are described elsewhere in this application, e.g., in the section relating to gene insertion and homologous recombination. In embodiments, the composition of a) above is a composition comprising RNPs comprising the one or more gRNA molecules. In embodiments, RNPs comprising gRNAs targeting unique target sequences are introduced into the cell simultaneously, e.g., as a mixture of RNPs comprising the one or more gRNAs. In embodiments, RNPs comprising gRNAs targeting unique target sequences are introduced into the cell sequentially.

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Promoters

In one embodiment, the vector further comprises a promoter. In some embodiments, the promoter is chosen from an EF-1 promoter, a CMV IE gene promoter, an EF-1α promoter, an ubiquitin C promoter, or a phosphoglycerate kinase (PGK) promoter. In one embodiment, the promoter is an EF-1 promoter. In one embodiment, the EF-1 promoter comprises the sequence of SEQ ID NO: 270.

### Host Cells for CAR Expression

As noted above, in some aspects the disclosure pertains to a cell, e.g., an immune effector cell, (e.g., a population of cells, e.g., a population of immune effector cells) comprising a nucleic acid molecule, a CAR polypeptide molecule, or a vector as described herein.

In certain aspects of the present disclosure, immune effector cells, e.g., T cells, can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, CD25+depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11 b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-γ, TNFα, IL-17A, IL-2, IL-3, IL-4, GM-CSF, IL-10, IL-13, granzyme B, and perform, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion/ml, 8 billion/ml, 7 billion/ml, 6 billion/ml, or 5 billion/ml is used. In one aspect, a concentration of 1 billion cells/ml is used. In yet one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect, a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

In some aspects, the cells of the disclosure (e.g., the immune effector cells of the disclosure, e.g., the CAR-expressing cells of the disclosure) are induced pluripotent stem cells (“iPSCs”) or embryonic stem cells (ESCs), or are T cells generated from (e.g., differentiated from) said iPSC and/or ESC. iPSCs can be generated, for example, by methods known in the art, from peripheral blood T lymphocytes, e.g., peripheral blood T lymphocytes isolated from a healthy volunteer. As well, such cells may be differentiated into T cells by methods known in the art. See e.g., Themeli M. et al., Nat. Biotechnol., 31, pp. 928-933 (2013); doi: 10. 1038/nbt.2678; WO2014/165707, the contents of each of which are incorporated herein by reference in their entirety.

In another embodiment, the 3-(1-oxoisoindolin-2-yl)piperidine-2,6-dione IKZF2 degrader compounds, or a pharmaceutically acceptable salt, hydrate, solvate, prodrug, stereoisomer, or tautomer thereof, of the present disclosure are used in combination with one or more of the therapeutic agents listed in Table 13 or listed in the patent and patent applications cited in Table 13, to treat cancer. Each publication listed in Table 13 is herein incorporated by reference in its entirety, including all structural formulae therein.

TABLE 13

| Second | | | |
|---|---|---|---|
| agent | Generic Name | | Patents/Patent Application |
| No. | Tradename | Compound Structure | Publications |
| A1 | Sotrastaurin | 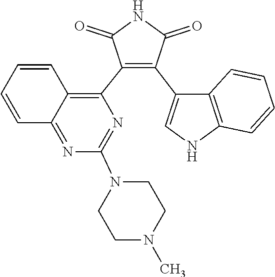 | EP 1682103 US 2007/142401 WO 2005/039549 |
| A2 | Nilotinib HCl monohydrate TASIGNA ® | 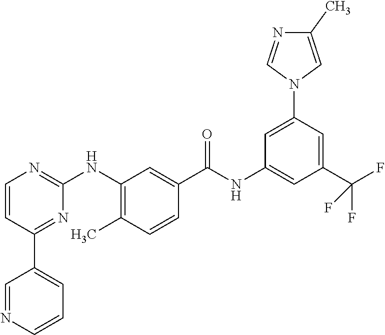 [SMILES: Cc1cn(-c2cc(NC(=O)c3ccc(C)c(Nc4nccc(-c5cccnc5)n4)c3)cc(C(F)(F)F)c2)cn1] | WO 2004/005281 US 7,169,791 |
| A3 | | 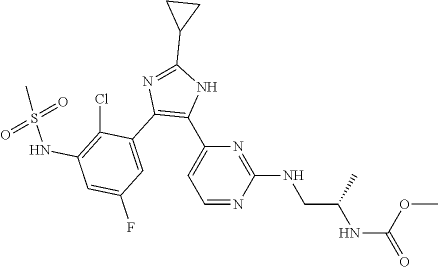 [SMILES: COC(=O)N[C@@H](C)CNc1nccc(-c2[nH]c(C3CC3)nc2-c2cc(F)cc(NS(C)(=O)=O)c2Cl)n1] | WO 2011/023773 |
| A4 | | 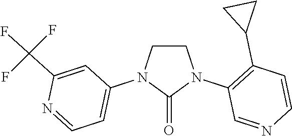 [SMILES: O=C1N(c2ccnc(C(F)(F)F)c2)CCN1c1cnccc1C1CC1] | WO 2012/149413 |
| A6 | | 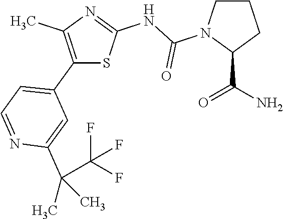 [SMILES: Cc1nc(NC(=O)N2CCC[C@H]2C(N)=O)sc1-c1ccnc(C(C)(C)C(F)(F)F)c1] | WO 2010/029082 |
| A7 | | 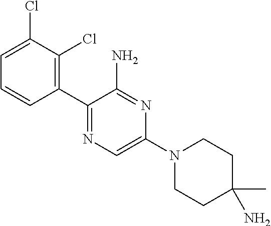 [SMILES: CC1(N)CCN(c2cnc(-c3cccc(Cl)c3Cl)c(N)n2)CC1] | WO 2015/107493 |
| A8 | | | WO 2015/107495 |
| A9 | | 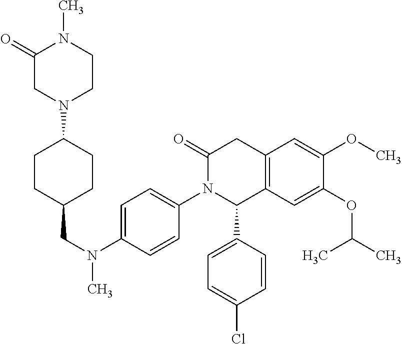 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2] | WO 2011/076786 |
| A10 | Deferasirox EXJADE ® | 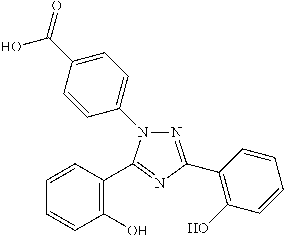 [SMILES: O=C(O)c1ccc(-n2nc(-c3ccccc3O)nc2-c2ccccc2O)cc1] | WO 1997/049395 |
| A11 | Letrozole FEMARA ® | 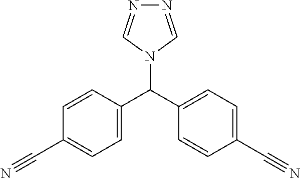 [SMILES: N#Cc1ccc(C(c2ccc(C#N)cc2)n2cnnc2)cc1] | US 4,978,672 |
| A12 | | 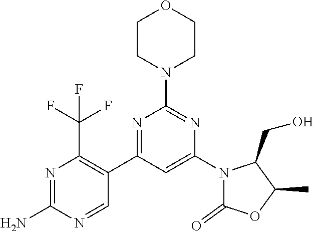 [SMILES: C[C@H]1OC(=O)N(c2cc(-c3cnc(N)nc3C(F)(F)F)nc(N3CCOCC3)n2)[C@H]1CO] | WO 2013/124826 US 2013/0225574 |
| A13 | | 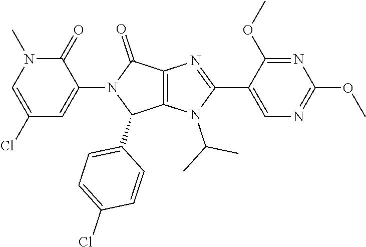 [SMILES: COc1ncc(-c2nc3c(n2C(C)C)[C@H](c2ccc(Cl)cc2)N(c2cc(Cl)cn(C)c2=O)C3=O)c(OC)n1] | WO 2013/111105 |
| A14 | | 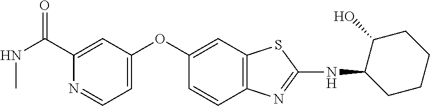 [SMILES: CNC(=O)c1cc(Oc2ccc3nc(N[C@@H]4CCCC[C@H]4O)sc3c2)ccn1] | WO 2007/121484 |
| A15 | Imatinib mesylate GLEEVEC ® | 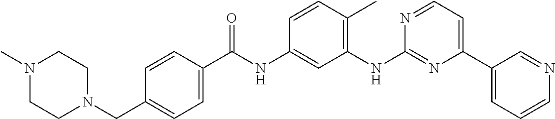 [SMILES: Cc1ccc(NC(=O)c2ccc(CN3CCN(C)CC3)cc2)cc1Nc1nccc(-c2cccnc2)n1] | WO 1999/003854 |
| A16 | Capmatinib | 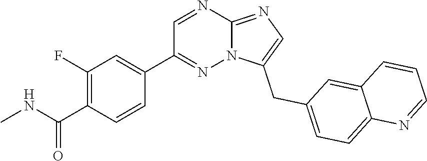 [SMILES: CNC(=O)c1ccc(-c2cnc3ncc(Cc4ccc5ncccc5c4)n3n2)cc1F] | EP 2099447 US 7,767,675 US 8,420,645 |
| A17 | Ruxolitinib Phosphate JAKAFI ® | 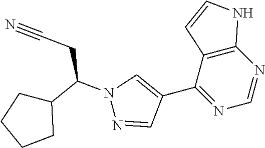 [SMILES: N#CC[C@H](C1CCCC1)n1cc(-c2ncnc3[nH]ccc23)cn1] | WO 2007/070514; EP 2474545 US 7,598,257; WO 2014/018632 |
| A18 | Panobinostat | 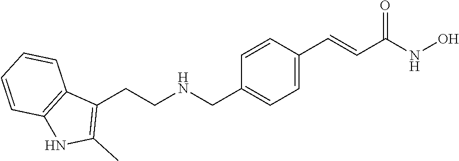 [SMILES: Cc1[nH]c2ccccc2c1CCNCc1ccc(/C=C/C(=O)NO)cc1] | WO 2014/072493 WO 2002/022577 EP 1870399 |
| A20 | | 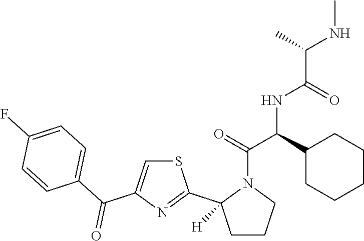 [SMILES: CN[C@@H](C)C(=O)N[C@H](C(=O)N1CCC[C@H]1c1nc(C(=O)c2ccc(F)cc2)cs1)C1CCCCC1] | WO 2008/016893 EP 2051990 US 8,552,003 |
| A21 | | 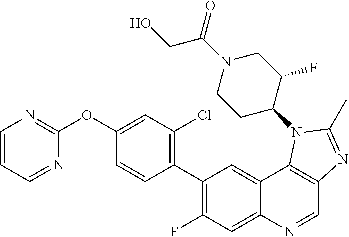 [SMILES: Cc1nc2cnc3cc(F)c(-c4ccc(Oc5ncccn5)cc4Cl)cc3c2n1[C@H]1CCN(C(=O)CO)C[C@@H]1F] | WO2015/022662 |
| A22 | ceritinib ZYKADIA ™ | 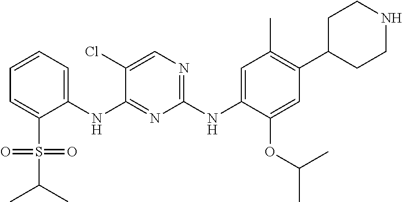 [SMILES: Cc1cc(Nc2ncc(Cl)c(Nc3ccccc3S(=O)(=O)C(C)C)n2)c(OC(C)C)cc1C1CCNCC1] | WO 2008/073687 US 8,039,479 |
| A23 | Ribociclib KISQALI ® | 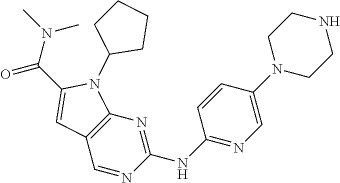 [SMILES: CN(C)C(=O)c1cc2cnc(Nc3ccc(N4CCNCC4)cn3)nc2n1C1CCCC1] | US 8,415,355 US 8,685,980 |
| A24 | | 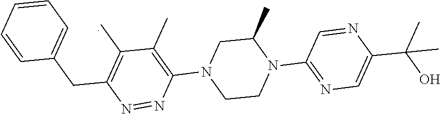 | WO 2010/007120 |
| A26 | | 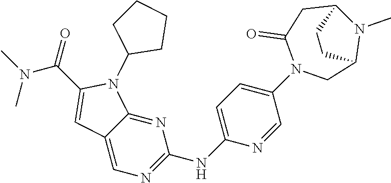 | WO 2011/101409 |
| A27 | | Human monoclonal antibody to HER3 | WO 2012/022814; EP 2606070 US 8,735,551 |
| A28 | | Antibody Drug Conjugate (ADC) | WO 2014/160160 |
| A29 | | Monoclonal antibody or Fab to M-CSF | WO 2004/045532 |
| A30 | Midostaurin | 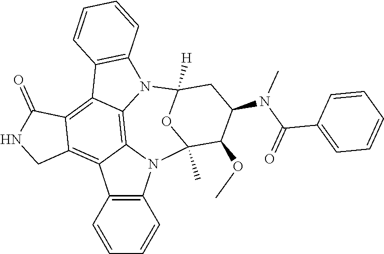 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4] | WO 2003/037347; EP 1441737 US 2012/252785 |
| A31 | Everolimus AFINITOR ® | 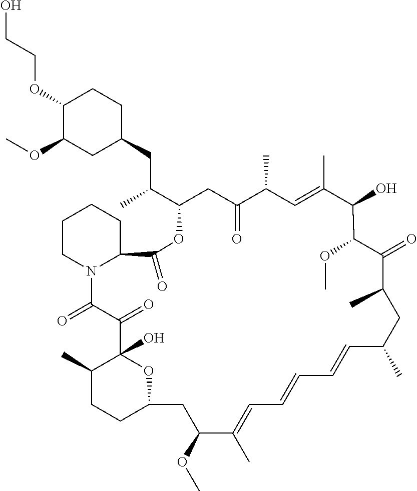 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] | WO 1994/009010 WO 2014/085318 |
| A32 | | 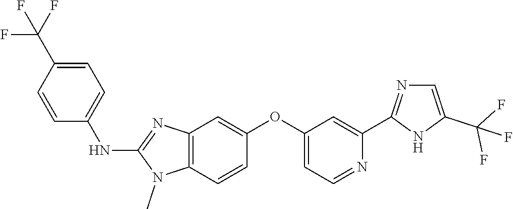 [SMILES: Cn1c(Nc2ccc(C(F)(F)F)cc2)nc2cc(Oc3ccnc(-c4ncc(C(F)(F)F)[nH]4)c3)ccc21] | WO 2007/030377; US 7,482,367 |
| A34 | | 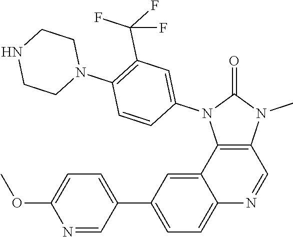 | WO 2006/122806 |
| A35 | | 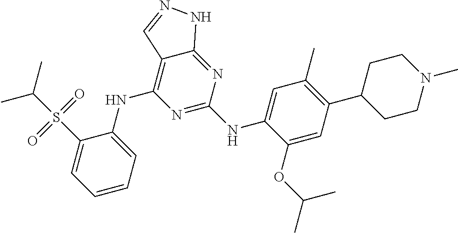 [SMILES: Cc1cc(Nc2nc(Nc3ccccc3S(=O)(=O)C(C)C)c3cn[nH]c3n2)c(OC(C)C)cc1C1CCN(C)CC1] | WO 2008/073687; US 8,372,858 |
| A36 | Valspodar AMDRAY ™ | 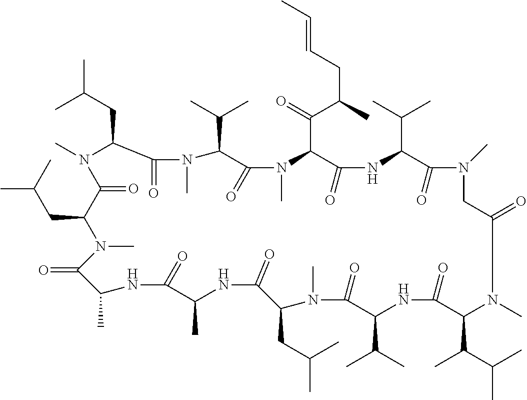 [SMILES: C/C=C/C[C@@H](C)C(=O)[C@H]1C(=O)N[C@@H](C(C)C)C(=O)N(C)CC(=O)N(C)[C@@H](C(C)C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] | EP 296122 |
| A37 | Vatalanib succinate | 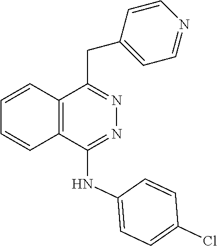 [SMILES: Clc1ccc(Nc2nnc(Cc3ccncc3)c3ccccc23)cc1] | WO 98/35958 |
| A38 | | 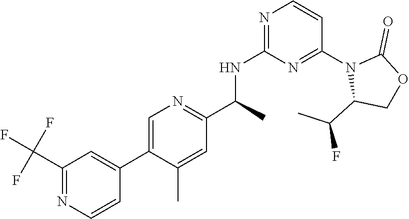 [SMILES: Cc1cc([C@H](C)Nc2nccc(N3C(=O)OC[C@@H]3[C@H](C)F)n2)ncc1-c1ccnc(C(F)(F)F)c1] | WO 2014/141104 |
| A39 | Asciminib | 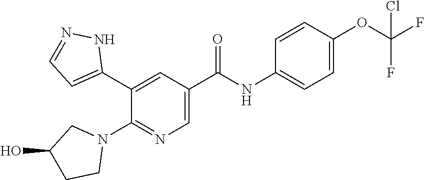 [SMILES: O=C(Nc1ccc(OC(F)(F)Cl)cc1)c1cnc(N2CC[C@@H](O)C2)c(-c2ccn[nH]2)c1] | WO 2013/171639 WO 2013/171640 WO 2013/171641 WO 2013/171642 |
| A42 | | 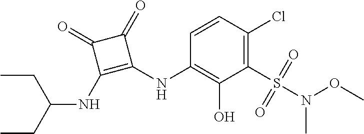 [SMILES: CCC(CC)Nc1c(Nc2ccc(Cl)c(S(=O)(=O)N(C)OC)c2O)c(=O)c1=O] | WO 2010/015613 WO 2013030803 US 7,989,497 |
| A43 | | | WO 2017/025918 WO 2011/121418 US 8,796,284 |
| A44 | | 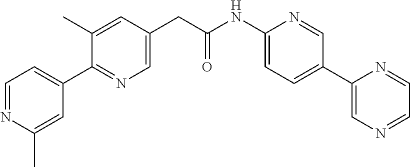 [SMILES: Cc1cc(-c2ncc(CC(=O)Nc3ccc(-c4cnccn4)cn3)cc2C)ccn1] | WO 2010/101849 |
| A45 | | 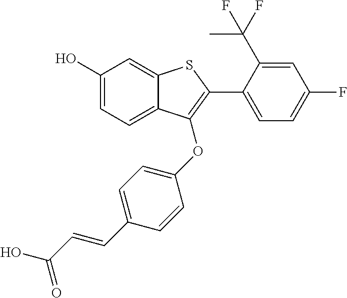 [SMILES: CC(F)(F)c1cc(F)ccc1-c1sc2cc(O)ccc2c1Oc1ccc(/C=C/C(=O)O)cc1] | WO 2014/130310 |
| A46 | trametinib | 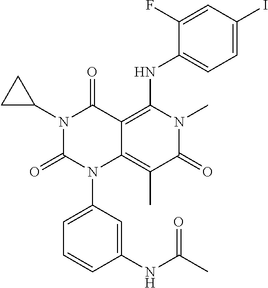 [SMILES: CC(=O)Nc1cccc(-n2c(=O)n(C3CC3)c(=O)c3c(Nc4ccc(I)cc4F)n(C)c(=O)c(C)c32)c1] | WO 2005/121142 US 7,378,423 |
| A47 | dabrafenib | 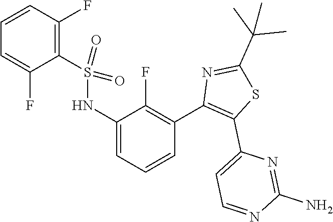 [SMILES: CC(C)(C)c1nc(-c2cccc(NS(=O)(=O)c3c(F)cccc3F)c2F)c(-c2ccnc(N)n2)s1] | WO 2009/137391 US 7,994,185 |
| A49 | octreotide | 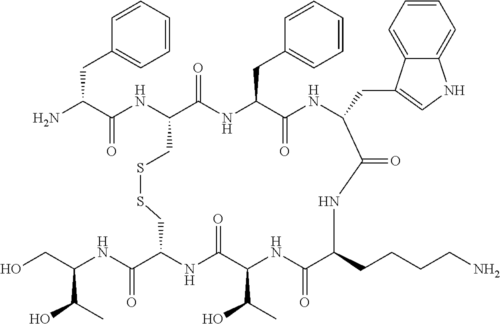 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O] | US 4,395,403 EP 0 029 579 |
| A50 | | 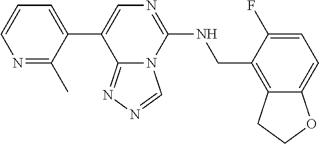 [SMILES: Cc1ncccc1-c1cnc(NCc2c(F)ccc3c2CCO3)n2cnnc12] | WO 2016/103155 US 9580437 EP 3237418 |
| A51 | | 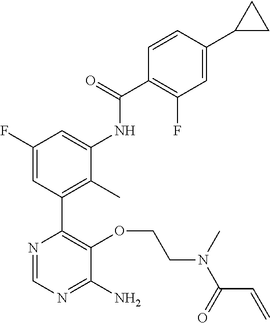 [SMILES: C=CC(=O)N(C)CCOc1c(N)ncnc1-c1cc(F)cc(NC(=O)c2ccc(C3CC3)cc2F)c1C] | US 9,512,084 WO/2015/079417 |
| A52 | | 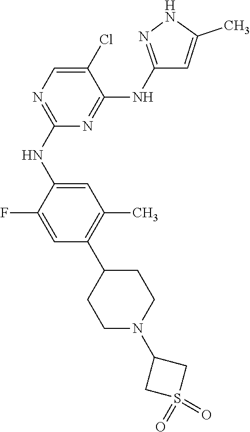 [SMILES: Cc1cc(Nc2nc(Nc3cc(C)c(C4CCN(C5CS(=O)(=O)C5)CC4)cc3F)ncc2Cl)n[nH]1] | WO 2010/002655 US 8,519,129 |
| A53 | | 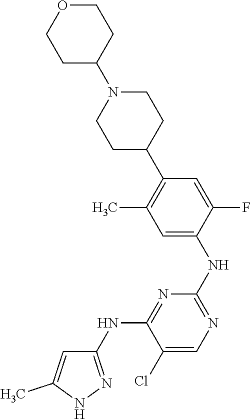 [SMILES: Cc1cc(Nc2nc(Nc3cc(C)c(C4CCN(C5CCOCC5)CC4)cc3F)ncc2Cl)n[nH]1] | WO 2010/002655; US 8,519,129 |
| A54 | | 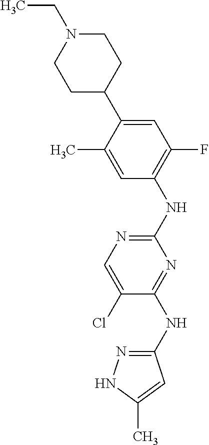 [SMILES: CCN1CCC(c2cc(F)c(Nc3ncc(Cl)c(Nc4cc(C)[nH]n4)n3)cc2C)CC1] | WO 2010/002655 |

### Estrogen Receptor Antagonists

In some embodiments, an estrogen receptor (ER) antagonist is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9(7): 465-479). ER is a hormone-activated transcription factor important for e.g., the growth, development and physiology of the human reproductive system. ER is activated by, e.g., the hormone estrogen (17beta estradiol). ER expression and signaling is implicated in cancers (e.g., breast cancer), e.g., ER positive (ER+) breast cancer. In some embodiments, the SERD is chosen from LSZ102, fulvestrant, brilanestrant, or elacestrant.

### Exemplary Estrogen Receptor Antagonists

In some embodiments, the SERD comprises a compound disclosed in International Application Publication No. WO 2014/130310, which is hereby incorporated by reference in its entirety. In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid.

### Other Exemplar Estrogen Receptor Antagonists

In some embodiments, the SERD comprises fulvestrant (CAS Registry Number: 129453-61-8), or a compound disclosed in International Application Publication No. WO 2001/051056, which is hereby incorporated by reference in its entirety. Fulvestrant is also known as ICI 182780, ZM 182780, FASLODEX®, or (7α,17β)-7-{9-[(4,4,5,5,5-pentafluoropentyl)sulfinyl]nonyl}estra-1,3,5(10)-triene-3,17-diol. Fulvestrant is a high affinity estrogen receptor antagonist with an IC50 of 0.29 nM.

In some embodiments, the SERD comprises elacestrant (CAS Registry Number: 722533-56-4), or a compound disclosed in U.S. Pat. No. 7,612,114, which is incorporated by reference in its entirety. Elacestrant is also known as RAD1901, ER-306323 or (6R)-6-{2-[Ethyl({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26(9):948-56.

In some embodiments, the SERD is brilanestrant (CAS Registry Number: 1365888-06-7), or a compound disclosed in International Application Publication No. WO 2015/136017, which is incorporated by reference in its entirety. Brilanestrant is also known as GDC-0810, ARN810, RG-6046, RO-7056118 or (2E)-3-{4-[(1E)-2-(2-chloro-4-fluorophenyl)-1-(1H-indazol-5-yl)but-1-en-1-yl]phenyl}prop-2-enoic acid. Brilanestrant is a next-generation, orally bioavailable selective SERD with an IC50 of 0.7 nM. Brilanestrant is also disclosed, e.g., in Lai A. et al. (2015) Journal of Medicinal Chemistry 58 (12): 4888-4904.

In some embodiments, the SERD is chosen from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58(12) 4883-4887. Other exemplary estrogen receptor antagonists are disclosed, e.g., in WO 2011/156518, WO 2011/159769, WO 2012/037410, WO 2012/037411, and US 2012/0071535, all of which are hereby incorporated by reference in their entirety.

### CDK4/6 Inhibitors

In some embodiments, an inhibitor of Cyclin-Dependent Kinases 4 or 6 (CDK4/6) is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the CDK4/6 inhibitor is chosen from ribociclib, abemaciclib (Eli Lilly), or palbociclib.

### Exemplary CDK4/6 Inhibitors

In some embodiments, the CDK4/6 inhibitor comprises ribociclib (CAS Registry Number: 1211441-98-3), or a compound disclosed in U.S. Pat. Nos. 8,415,355 and 8,685,980, which are incorporated by reference in their entirety.

In some embodiments, the CDK4/6 inhibitor comprises a compound disclosed in International Application Publication No. WO 2010/020675 and U.S. Pat. Nos. 8,415,355 and 8,685,980, which are incorporated by reference in their entirety.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

### Other Exemplary CDK4 6 Inhibitors

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4-[4-fluoro-2-methyl-1-(1-methylethyl)-1H-benzimidazol-6-yl]-2-pyrimidinamine. Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl-5-methyl-2-{[5-(1-piperazinyl)-2-pyridinyl]amino}pyrido[2,3-d]pyrimidin-7(8H)-one. Palbociclib inhibits CDK4 with an IC50 of 11 nM, and inhibits CDK6 with an IC50 of 16 nM, and is disclosed, e.g., in Finn et al. (2009) Breast Cancer Research 11(5):R77.

### CXCR2 Inhibitors

In some embodiments, an inhibitor of chemokine (C—X—C motif) receptor 2 (CXCR2) is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide, danirixin, reparixin, or navarixin.

### Exemplary CXCR2 Inhibitors

In some embodiments, the CXCR2 inhibitor comprises a compound disclosed in U.S. Pat. Nos. 7,989,497, 8,288,588, 8,329,754, 8,722,925, 9,115,087, U.S. Application Publication Nos. US 2010/0152205, US 2011/0251205 and US 2011/0251206, and International Application Publication Nos. WO 2008/061740, WO 2008/061741, WO 2008/062026, WO 2009/106539, WO2010/063802, WO 2012/062713, WO 2013/168108, WO 2010/015613 and WO 2013/030803. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide choline salt. In some embodiments, the CXCR2 inhibitor is 2-Hydroxy-N,N,N-trimethylethan-1-aminium 3-chloro-6-({3,4-dioxo-2-[(pentan-3-yl)amino]cyclobut-1-en-1-yl}amino)-2-(N-methoxy-N-methylsulfamoyl)phenolate (i.e., 6-chloro-3-((3,4-dioxo-2-(pentan-3-ylamino)cyclobut-1-en-1-yl)amino)-2-hydroxy-N-methoxy-N-methylbenzenesulfonamide choline salt) and has the following chemical structure:

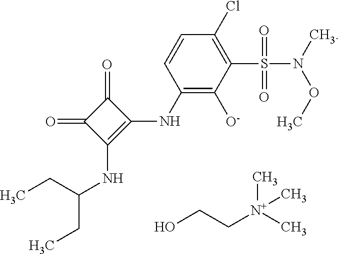

### Other Exemplary CXCR2 Inhibitors

In some embodiments, the CXCR2 inhibitor comprises danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea. Danirixin is disclosed, e.g., in Miller et al. Eur J Drug Metab Pharmacokinet (2014) 39:173-181; and Miller et al. BMC Pharmacology and Toxicology (2015), 16:18.

In some embodiments, the CXCR2 inhibitor comprises reparixin (CAS Registry Number: 266359-83-5). Reparixin is also known as repertaxin or (2R)-2-[4-(2-methylpropyl)phenyl]-N-methylsulfonylpropanamide. Reparixin is a non-competitive allosteric inhibitor of CXCR1/2. Reparixin is disclosed, e.g., in Zarbock et al. Br J Pharmacol. 2008; 155(3):357-64.

In some embodiments, the CXCR2 inhibitor comprises navarixin. Navarixin is also known as MK-7123, SCH 527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide. Navarixin is disclosed, e.g., in Ning et al.

### Mol Cancer Ther. 2012; 11(6):1353-64.

### CSF-1/1R Binding Agents

In some embodiments, a CSF-1/1R binding agent is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

### Exemplary CSF-1 Binding Agents

In some embodiments, the CSF-1/1R binding agent comprises an inhibitor of macrophage colony-stimulating factor (M-CSF). M-CSF is also sometimes known as CSF-1. In certain embodiments, the CSF-1/1R binding agent is an antibody to CSF-1 (e.g., MCS110). In other embodiments, the CSF-1/1R binding agent is an inhibitor of CSF-1R (e.g., BLZ945).

In some embodiments, the CSF-1/1R binding agent comprises a monoclonal antibody or Fab to M-CSF (e.g., MCS110/H-RX1), or a binding agent to CSF-1 disclosed in International Application Publication Nos. WO 2004/045532 and WO 2005/068503, including H-RX1 or 5H4 (e.g., an antibody molecule or Fab fragment against M-CSF) and U.S. Pat. No. 9,079,956, which applications and patent are incorporated by reference in their entirety.

TABLE 13a
Amino acid and nucleotide sequences of an exemplary anti-M-CSF antibody molecule
(MCS110)

| | |
|---|---|
| (H-RX1) HC | QVQLQESGPGLVKPSQTLSLTCTVSDYSITSDYAWNWIRQFPGKGLEWMGYI |
| | SYSGSTSYNPSLKSRITISRDTSKNQFSLQLNSVTAADTAVYYCASFDYAHAM |
| | DYWGQGTTVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNT |
| | KVDKRVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLTVDKSR |
| | WQQGNVFSCSVMHEALHNHYTQKSLSLSPGK (SEQ ID NO: 271) |
| (H-RX1) LC | DIVLTQSPAFLSVTPGEKVTFTCQASQSIGTSIHWYQQKTDQAPKLLIKYASES |
| | ISGIPSRFSGSGSGTDFTLTISSVEAEDAADYYCQQINSWPTTFGGGTKLEIKRT |
| | VAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGE |
| | C (SEQ ID NO: 272) |
| Heavy Chain | SDYAWN (SEQ ID NO: 273) |
| CDR1 (Kabat) | |
| Heavy Chain | YISYSGSTSYNPSLKS (SEQ ID NO: 274) |
| CDR2 (Kabat) | |
| Heavy Chain | FDYAHAMDY (SEQ ID NO: 275) |
| CDR3 (Kabat) | |
| Light Chain | QASQSIGTSIH (SEQ ID NO: 276) |
| CDR1 (Kabat) | |
| Light Chain | YASESIS (SEQ ID NO: 277) |
| CDR2 (Kabat) | |
| Light Chain | QQINSWPTT (SEQ ID NO: 278) |
| CDR3 (Kabat) | |

In another embodiment, the CSF-1/1R binding agent comprises a CSF-1R tyrosine kinase inhibitor, 4-((2-(((1R,2R)-2-hydroxycyclohexyl)amino)benzo[d]thiazol-6-yl)oxy)-N-methylpicolinamide (BLZ945), or a compound disclosed in International Application Publication No. WO 2007/121484, and U.S. Pat. Nos. 7,553,854, 8,173,689, and 8,710,048, which are incorporated by reference in their entirety.

### Other Exemplary CSF-1/1R Binding Agents

In some embodiments, the CSF-1/1R binding agent comprises pexidartinib (CAS Registry Number 1029044-16-3). Pexidrtinib is also known as PLX3397 or 5-((5-chloro-1H-pyrrolo[2,3-b]pyridin-3-yl)methyl)-N-((6-(trifluoromethyl)pyridin-3-yl)methyl)pyridin-2-amine. Pexidartinib is a small-molecule receptor tyrosine kinase (RTK) inhibitor of KIT, CSF1R and FLT3. FLT3, CSF1R and FLT3 are overexpressed or mutated in many cancer cell types and play major roles in tumor cell proliferation and metastasis. PLX3397 can bind to and inhibit phosphorylation of stem cell factor receptor (KIT), colony-stimulating factor-1 receptor (CSF1R) and FMS-like tyrosine kinase 3 (FLT3), which may result in the inhibition of tumor cell proliferation and down-modulation of macrophages, osteoclasts and mast cells involved in the osteolytic metastatic disease.

In some embodiments, the CSF-1/1R binding agent is emactuzumab. Emactuzumab is also known as RG7155 or RO5509554. Emactuzumab is a humanized IgG1 mAb targeting CSF1R. In some embodiments, the CSF-1/1R binding agent is FPA008. FPA008 is a humanized mAb that inhibits CSF1R.

### A2aR Antagonists

In some embodiments, an adenosine A2a receptor (A2aR) antagonist (e.g., an inhibitor of A2aR pathway, e.g., an adenosine inhibitor, e.g., an inhibitor of A2aR or CD-73) is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the A2aR antagonist is selected from PBF509 (NIR178) (Palobiofarma/Novartis), CPI444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), and Preladenant/SCH 420814 (Merck/Schering).

### Exemplary A2aR Antagonists

In some embodiments, the A2aR antagonist comprises PBF509 (NIR178) or a compound disclosed in U.S. Pat. No. 8,796,284 or in International Application Publication No. WO 2017/025918, herein incorporated by reference in their entirety. PBF509 (NIR178) is also known as NIR178.

### Other Exemplary A2aR Antagonists

In certain embodiments, the A2aR antagonist comprises CP1444/V81444. CPI-444 and other A2aR antagonists are disclosed in International Application Publication No. WO 2009/156737, herein incorporated by reference in its entirety. In certain embodiments, the A2aR antagonist is (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine. In certain embodiments, the A2aR antagonist is (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof. In certain embodiments, the A2aR antagonist is 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine.

In certain embodiments, the A2aR antagonist is AZD4635/HTL-1071. A2aR antagonists are disclosed in International Application Publication No. WO 2011/095625, herein incorporated by reference in its entirety. In certain embodiments, the A2aR antagonist is 6-(2-chloro-6-methylpyridin-4-yl)-5-(4-fluorophenyl)-1,2,4-triazin-3-amine.

In certain embodiments, the A2aR antagonist is ST-4206 (Leadiant Biosciences). In certain embodiments, the A2aR antagonist is an A2aR antagonist described in U.S. Pat. No. 9,133,197, herein incorporated by reference in its entirety.

In certain embodiments, the A2aR antagonist is an A2aR antagonist described in U.S. Pat. Nos. 8,114,845 and 9,029,393, U.S. Application Publication Nos. 2017/0015758 and 2016/0129108, herein incorporated by reference in their entirety.

In some embodiments, the A2aR antagonist is istradefylline (CAS Registry Number: 155270-99-8). Istradefylline is also known as KW-6002 or 8-[(E)-2-(3,4-dimethoxyphenyl)vinyl]-1,3-diethyl-7-methyl-3,7-dihydro-1H-purine-2,6-dione. Istradefylline is disclosed, e.g., in LeWitt et al. (2008) Annals of Neurology 63 (3): 295-302).

In some embodiments, the A2aR antagonist is tozadenant (Biotie). Tozadenant is also known as SYN115 or 4-hydroxy-N-(4-methoxy-7-morpholin-4-yl-1,3-benzothiazol-2-yl)-4-methylpiperidine-1-carboxamide. Tozadenant blocks the effect of endogenous adenosine at the A2a receptors, resulting in the potentiation of the effect of dopamine at the D2 receptor and inhibition of the effect of glutamate at the mGluR5 receptor. In some embodiments, the A2aR antagonist is preladenant (CAS Registry Number: 377727-87-2). Preladenant is also known as SCH 420814 or 2-(2-Furanyl)-7-[2-[4-[4-(2-methoxyethoxy)phenyl]-1-piperazinyl]ethyl]7H-pyrazolo[4,3-e][1,2,4]triazolo[1,5-c]pyrimidine-5-amine. Preladenant was developed as a drug that acted as a potent and selective antagonist at the adenosine A2A receptor.

In some embodiments, the A2aR antagonist is vipadenan. Vipadenan is also known as BIIB014, V2006, or 3-[(4-amino-3-methylphenyl)methyl]-7-(furan-2-yl)triazolo[4,5-d]pyrimidin-5-amine. Other exemplary A2aR antagonists include, e.g., ATL-444, MSX-3, SCH-58261, SCH-412,348, SCH-442,416, VER-6623, VER-6947, VER-7835, CGS-15943, and ZM-241,385.

In some embodiments, the A2aR antagonist is an A2aR pathway antagonist (e.g., a CD-73 inhibitor, e.g., an anti-CD73 antibody) is MEDI9447. MEDI9447 is a monoclonal antibody specific for CD73.

Targeting the extracellular production of adenosine by CD73 may reduce the immunosuppressive effects of adenosine. MEDI9447 was reported to have a range of activities, e.g., inhibition of CD73 ectonucleotidase activity, relief from AMP-mediated lymphocyte suppression, and inhibition of syngeneic tumor growth. MEDI9447 can drive changes in both myeloid and lymphoid infiltrating leukocyte populations within the tumor microenvironment. These changes include, e.g., increases in CD8 effector cells and activated macrophages, as well as a reduction in the proportions of myeloid-derived suppressor cells (MDSC) and regulatory T lymphocytes.

### IDO Inhibitors

In some embodiments, an inhibitor of indoleamine 2,3-dioxygenase (IDO) and/or tryptophan 2,3-dioxygenase (TDO) is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod O, (1-methyl-D-tryptophan), a-cyclohexyl-5H-Imidazo[5,1-a]isoindole-5-ethanol (also known as NLG919), indoximod, and BMS-986205 (formerly F001287).

### Exemplary IDO Inhibitors

In some embodiments, the IDO/TDO inhibitor is indoximod (New Link Genetics). Indoximod, the D isomer of 1-methyl-tryptophan, is an orally administered small-molecule indoleamine 2,3-dioxygenase (IDO) pathway inhibitor that disrupts the mechanisms by which tumors evade immune-mediated destruction.

In some embodiments, the IDO/TDO inhibitor is NLG919 (New Link Genetics). NLG919 is a potent IDO (indoleamine-(2,3)-dioxygenase) pathway inhibitor with Ki/EC50 of 7 nM/75 nM in cell-free assays.

In some embodiments, the IDO/TDO inhibitor is epacadostat (CAS Registry Number: 1204669-58-8). Epacadostat is also known as INCB24360 or INCB024360 (Incyte). Epacadostat is a potent and selective indoleamine 2,3-dioxygenase (IDO1) inhibitor with IC50 of 10 nM, highly selective over other related enzymes such as ID02 or tryptophan 2,3-dioxygenase (TDO).

In some embodiments, the IDO/TDO inhibitor is F001287 (Flexus/BMS). F001287 is a small molecule inhibitor of indoleamine 2,3-dioxygenase 1 (IDO1).

### STING Agonists

In some embodiments, a STING agonist is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage. In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

In some embodiments, the STING agonist is MK-1454 (Merck). MK-1454 is a cyclic dinucleotide Stimulator of Interferon Genes (STING) agonist that activates the STING pathway. Exemplary STING agonist are disclosed, e.g., in PCT Publication No. WO 2017/027645.

### Galectin Inhibitors

In some embodiments, a Galectin, e.g., Galectin-1 or Galectin-3, inhibitor is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is chosen from an anti-Galectin antibody molecule, GR-MD-02 (Galectin Therapeutics), Galectin-3C (Mandal Med), Anginex, or OTX-008 (OncoEthix, Merck). Galectins are a family of proteins that bind to beta galactosidase sugars.

The Galectin family of proteins comprises at least of Galectin-1, Galectin-2, Galectin-3, Galectin-4, Galectin-7, and Galectin-8. Galectins are also referred to as S-type lectins, and are soluble proteins with, e.g., intracellular and extracellular functions. Galectin-1 and Galectin-3 are highly expressed in various tumor types. Galectin-1 and Galectin-3 can promote angiogenesis and/or reprogram myeloid cells toward a pro-tumor phenotype, e.g., enhance immunosuppression from myeloid cells. Soluble Galectin-3 can also bind to and/or inactivate infiltrating T cells.

### Exemplary Galectin Inhibitors

In some embodiments, a Galectin inhibitor is an antibody molecule. In an embodiment, an antibody molecule is a monospecific antibody molecule and binds a single epitope. E.g., a monospecific antibody molecule having a plurality of immunoglobulin variable domain sequences, each of which binds the same epitope. In an embodiment, the Galectin inhibitor is an anti-Galectin, e.g., anti-Galectin-1 or anti-Galectin-3, antibody molecule. In some embodiments, the Galectin inhibitor is an anti-Galectin-1 antibody molecule. In some embodiments, the Galectin inhibitor is an anti-Galectin-3 antibody molecule.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, the Galectin inhibitor is a multispecific antibody molecule. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope. In an embodiment, the Galectin inhibitor is a bispecific antibody molecule. In an embodiment, the first epitope is located on Galectin-1, and the second epitope is located on Galectin-3.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH—CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In other embodiments, the anti-Galectin, e.g., anti-Galectin-1 or anti-Galectin-3, antibody molecule (e.g., a monospecific, bispecific, or multispecific antibody molecule) is covalently linked, e.g., fused, to another partner e.g., a protein, e.g., as a fusion molecule for example a fusion protein. In one embodiment, a bispecific antibody molecule has a first binding specificity to a first target (e.g., to Galectin-1), a second binding specificity to a second target (e.g., Galectin-3).

This invention provides an isolated nucleic acid molecule encoding the above antibody molecule, vectors and host cells thereof. The nucleic acid molecule includes but is not limited to RNA, genomic DNA and cDNA.

In some embodiments, a Galectin inhibitor is a peptide, e.g., protein, which can bind to, and inhibit Galectin, e.g., Galectin-1 or Galectin-3, function. In some embodiments, the Galectin inhibitor is a peptide which can bind to, and inhibit Galectin-3 function. In some embodiments, the Galectin inhibitor is the peptide Galectin-3C. In some embodiments, the Galectin inhibitor is a Galectin-3 inhibitor disclosed in U.S. Pat. No. 6,770,622, which is hereby incorporated by reference in its entirety.

Galectin-3C is an N-terminal truncated protein of Galectin-3, and functions, e.g., as a competitive inhibitor of Galectin-3. Galectin-3C prevents binding of endogenous Galectin-3 to e.g., laminin on the surface of, e.g., cancer cells, and other beta-galactosidase glycoconjugates in the extracellular matrix (ECM). Galectin-3C and other exemplary Galectin inhibiting peptides are disclosed in U.S. Pat. No. 6,770,622.

In some embodiments, Galectin-3C comprises the amino acid sequence of SEQ ID NO: 279, or arpGp-107,DNA amino acid substantially identical (e.g., 90, 95 or 99%) identical thereto.

(SEQ ID NO: 279)
GAPAGPLIVPYNLPLPGGVVPRMLITILGTVKPNANRIALDFQRGNDVAF
HFNPRFNENNRRVIVCNTKLDNNWGREERQSVFPFESGKPFKIQVLVEPD
HFKVAVNDAHLLQYNHRVKKLNEISKLGISGDIDITSASYTMI.

In some embodiments, the Galectin inhibitor is a peptide, which can bind to, and inhibit Galectin-1 function. In some embodiments, the Galectin inhibitor is the peptide Anginex: Anginex is an anti-angiongenic peptide that binds Galectin-1 (Salomonsson E, et al., (2011) Journal of Biological Chemistry, 286(16):13801-13804). Binding of Anginex to Galectin-1 can interfere with, e.g., the pro-angiongenic effects of Galectin-1.

In some embodiments, the Galectin, e.g., Galectin-1 or Galectin-3, inhibitor is a non-peptidic topomimetic molecule. In some embodiments, the non-peptidic topomimetic Galectin inhibitor is OTX-008 (OncoEthix). In some embodiments, the non-peptidic topomimetic is a non-peptidic topomimetic disclosed in U.S. Pat. No. 8,207,228, which is herein incorporated by reference in its entirety. OTX-008, also known as PTX-008 or Calixarene 0118, is a selective allosteric inhibitor of Galectin-1. OTX-008 has the chemical name: N-[2-(dimethylamino)ethyl]-2-{[26,27,28-tris({[2-(dimethylamino)ethyl]carbamoyl}methoxy)pentacyclo[19.3.1.1,7.1,15,]octacosa-1(25),3(28),4,6,9(27),1012,15,17,19(26),21,23-dodecaen-25-yl]oxy}acetamide.

In some embodiments, the Galectin, e.g., Galectin-1 or Galectin-3, inhibitor is a carbohydrate based compound. In some embodiments, the Galectin inhibitor is GR-MD-02 (Galectin Therapeutics). In some embodiments, GR-MD-02 is a Galectin-3 inhibitor. GR-MD-02 is a galactose-pronged polysaccharide also referred to as, e.g., a galactoarabino-rhamnogalaturonate. GR-MD-02 and other galactose-pronged polymers, e.g., galactoarabino-rhamnogalaturonates, are disclosed in U.S. Pat. No. 8,236,780 and U.S. Publication 2014/0086932, the entire contents of which are herein incorporated by reference in their entirety.

### MEK Inhibitors

In some embodiments, a MEK inhibitor is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the MEK inhibitor is chosen from Trametinib, selumetinib, AS703026, BIX 02189, BIX 02188, CI-1040, PD0325901, PD98059, U0126, XL-518, G-38963, or G02443714. In some embodiments, the MEK inhibitor is Trametinib.

### Exemplar MEK Inhibitors

In some embodiments, the MEK inhibitor is trametinib. Trametinib is also known as JTP-74057, TMT212, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, or Mekinist (CAS Number 871700-17-3).

### Other Exemplary MEK Inhibitors

In some embodiments the MEK inhibitor comprises selumetinib which has the chemical name: (5-[(4-bromo-2-chlorophenyl)amino]-4-fluoro-N-(2-hydroxyethoxy)-1-methyl-1H-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436).

In some embodiments, the MEK inhibitor comprises N-[(2R)-2,3-Dihydroxypropoxy]-3,4-difluoro-2-[(2-fluoro-4-iodophenyl)amino]-benzamide (also known as PD0325901), e.g., as described in PCT Publication No. WO2002006213).

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis[amino[(2-aminophenyl)thio]methylene]-butanedinitrile (also known as U0126), e.g., as described in U.S. Pat. No. 2,779,780).

In some embodiments, the MEK inhibitor comprises XL-518 (also known as GDC-0973) which has a CAS No. 1029872-29-4 and is available from ACC Corp.

In some embodiments, the MEK inhibitor comprises G-38963.

In some embodiments, the MEK inhibitor comprises G02443714 (also known as AS703206)

Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

c-MET Inhibitors

In some embodiments, a c-MET inhibitor is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein.

In some embodiments, the c-MET inhibitor is chosen from capmatinib (INC280), JNJ-3887605, AMG 337, LY2801653, MSC2156119J, crizotinib, tivantinib, or golvatinib.

Exemplary c-MET Inhibitors

In some embodiments, the c-MET inhibitor comprises capmatinib (INC280), or a compound described in U.S. Pat. Nos. 7,767,675, and 8,461,330, which are incorporated by reference in their entirety.

Other Exemplary c-MET-Inhibitors

In some embodiments, the c-MET inhibitor comprises JNJ-38877605. JNJ-38877605 is an orally available, small molecule inhibitor of c-Met. JNJ-38877605 selectively binds to c-MET, thereby inhibiting c-MET phosphorylation and disrupting c-Met signal transduction pathways. In some embodiments, the c-Met inhibitor is AMG 208. AMG 208 is a selective small-molecule inhibitor of c-MET. AMG 208 inhibits the ligand-dependent and ligand-independent activation of c-MET, inhibiting its tyrosine kinase activity, which may result in cell growth inhibition in tumors that overexpress c-Met.

In some embodiments, the c-Met inhibitor comprises AMG 337. AMG 337 is an orally bioavailable inhibitor of c-Met. AMG 337 selectively binds to c-MET, thereby disrupting c-MET signal transduction pathways.

In some embodiments, the c-Met inhibitor comprises LY2801653. LY2801653 is an orally available, small molecule inhibitor of c-Met. LY2801653 selectively binds to c-MET, thereby inhibiting c-MET phosphorylation and disrupting c-Met signal transduction pathways.

In some embodiments, c-Met inhibitor comprises MSC2156119J. MSC2156119J is an orally bioavailable inhibitor of c-Met. MSC2156119J selectively binds to c-MET, which inhibits c-MET phosphorylation and disrupts c-Met-mediated signal transduction pathways.

In some embodiments, the c-MET inhibitor is capmatinib. Capmatinib is also known as INCB028060. Capmatinib is an orally bioavailable inhibitor of c-MET. Capmatinib selectively binds to c-Met, thereby inhibiting c-Met phosphorylation and disrupting c-Met signal transduction pathways.

In some embodiments, the c-MET inhibitor comprises crizotinib. Crizotinib is also known as PF-02341066. Crizotinib is an orally available aminopyridine-based inhibitor of the receptor tyrosine kinase anaplastic lymphoma kinase (ALK) and the c-Met/hepatocyte growth factor receptor (HGFR). Crizotinib, in an ATP-competitive manner, binds to and inhibits ALK kinase and ALK fusion proteins. In addition, crizotinib inhibits c-Met kinase, and disrupts the c-Met signaling pathway. Altogether, this agent inhibits tumor cell growth.

In some embodiments, the c-MET inhibitor comprises golvatinib. Golvatinib is an orally bioavailable dual kinase inhibitor of c-MET and VEGFR-2 with potential antineoplastic activity. Golvatinib binds to and inhibits the activities of both c-MET and VEGFR-2, which may inhibit tumor cell growth and survival of tumor cells that overexpress these receptor tyrosine kinases.

In some embodiments, the c-MET inhibitor is tivantinib. Tivantinib is also known as ARQ 197. Tivantinib is an orally bioavailable small molecule inhibitor of c-MET. Tivantinib binds to the c-MET protein and disrupts c-Met signal transduction pathways, which may induce cell death in tumor cells overexpressing c-MET protein or expressing constitutively activated c-Met protein.

### TGF-β Inhibitors

In some embodiments, a transforming growth factor beta (also known as TGF-β TGFβ, TGFb, or TGF-beta, used interchangeably herein) inhibitor is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In certain embodiments, a combination described herein comprises a transforming growth factor beta (also known as TGF-β TGFβ, TGFb, or TGF-beta, used interchangeably herein) inhibitor.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

Under normal conditions, TGF-β maintains homeostasis and limits the growth of epithelial, endothelial, neuronal and hematopoietic cell lineages, e.g., through the induction of anti-proliferative and apoptotic responses. Canonical and non-canonical signaling pathways are involved in cellular responses to TGF-β. Activation of the TGF-β/Smad canonical pathway can mediate the anti-proliferative effects of TGF-β. The non-canonical TGF-β pathway can activate additional intra-cellular pathways, e.g., mitogen-activated protein kinases (MAPK), phosphatidylinositol 3 kinase/Protein Kinase B, Rho-like GTPases (Tian et al. Cell Signal. 2011; 23(6):951-62; Blobe et al. N Engl J Med. 2000; 342(18):1350-8), thus modulating epithelial to mesenchymal transition (EMT) and/or cell motility.

Alterations of TGF-β signaling pathway are associated with human diseases, e.g., cancers, cardio-vascular diseases, fibrosis, reproductive disorders, and wound healing. Without wishing to be bound by theory, it is believed that in some embodiments, the role of TGF-β in cancer is dependent on the disease setting (e.g., tumor stage and genetic alteration) and/or cellular context. For example, in late stages of cancer, TGF-β can modulate a cancer-related process, e.g., by promoting tumor growth (e.g., inducing EMT), blocking anti-tumor immune responses, increasing tumor-associated fibrosis, or enhancing angiogenesis (Wakefield and Hill Nat Rev Cancer. 2013; 13(5):328-41). In certain embodiments, a combination comprising a TGF-β inhibitor described herein is used to treat a cancer in a late stage, a metastatic cancer, or an advanced cancer.

Preclinical evidence indicates that TGF-β plays an important role in immune regulation (Wojtowicz-Praga Invest New Drugs. 2003; 21(1):21-32; Yang et al. Trends Immunol. 2010; 31(6):220-7). TGF-β can down-regulate the host-immune response via several mechanisms, e.g., shift of the T-helper balance toward Th2 immune phenotype; inhibition of anti-tumoral Th1 type response and M1-type macrophages; suppression of cytotoxic CD8+ T lymphocytes (CTL), NK lymphocytes and dendritic cell functions, generation of CD4+CD25+ T-regulatory cells; or promotion of M2-type macrophages with pro-tumoral activity mediated by secretion of immunosuppressive cytokines (e.g., IL10 or VEGF), pro-inflammatory cytokines (e.g., IL6, TNFα, or IL1) and generation of reactive oxygen species (ROS) with genotoxic activity (Yang et al. Trends Immunol. 2010; 31(6):220-7; Truty and Urrutia Pancreatology. 2007; 7(5-6):423-35; Achyut et al Gastroenterology. 2011; 141(4):1167-78).

### Exemplary TGF-β Inhibitors

In some embodiments, the TGF-β inhibitor comprises XOMA 089, or a compound disclosed in International Application Publication No. WO 2012/167143, which is incorporated by reference in its entirety.

XOMA 089 is also known as XPA.42.089. XOMA 089 is a fully human monoclonal antibody that specifically binds and neutralizes TGF-beta 1 and 2 ligands.

The heavy chain variable region of XOMA 089 has the amino acid sequence of: QVQLVQSGAEVKKPGSSVKVSCKASGGTFSSYAISWVRQAPGQGLEWMGGIIPIFGTANYAQKF QGRVTITADESTSTAYMELSSLRSEDTAVYYCARGLWEVRALPSVYWGQGTLVTVSS (SEQ ID NO: 284) (disclosed as SEQ ID NO: 6 in WO 2012/167143). The light chain variable region of XOMA 089 has the amino acid sequence of:

(SEQ ID NO: 285)
SYELTQPPSVSVAPGQTARITCGANDIGSKSVHWYQQKAGQAPVLVVSED
IIRPSGIPERISGSNSGNTATLTISRVEAGDEADYYCQVWDRDSDQYVFG
TGTKVTVLG (disclosed as SEQ ID NO: 8 in WO 2012/
167143).

XOMA 089 binds with high affinity to the human TGF-β isoforms. Generally, XOMA 089 binds with high affinity to TGF-β1 and TGF-β2, and to a lesser extent to TGF-β3. In Biacore assays, the K_{D} of XOMA 089 on human TGF-β is 14.6 pM for TGF-β1, 67.3 pM for TGF-β2, and 948 pM for TGF-β3. Given the high affinity binding to all three TGF-β isoforms, in certain embodiments, XOMA 089 is expected to bind to TGF-β1, 2 and 3 at a dose of XOMA 089 as described herein. XOMA 089 cross-reacts with rodent and cynomolgus monkey TGF-β and shows functional activity in vitro and in vivo, making rodent and cynomolgus monkey relevant species for toxicology studies.

### Other Exemplary TGF-Q Inhibitors

In some embodiments, the TGF-β inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of:

(SEQ ID NO: 280)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK.

The light chain of fresolimumab has the amino acid sequence of:

(SEQ ID NO: 281)
ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

Fresolimumab is disclosed, e.g., in International Application Publication No. WO 2006/086469, and U.S. Pat. Nos. 8,383,780 and 8,591,901, which are incorporated by reference in their entirety.

### IL-1β Inhibitors

The Interleukin-1 (IL-1) family of cytokines is a group of secreted pleotropic cytokines with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). The IL-1 family comprises, inter alia, IL-1 beta (IL-1b), and IL-1alpha (IL-1a). IL-1b is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1b, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1b results in a decrease in tumor burden and metastasis (Voronov et al. (2003) Proc.

Natl. Acad. Sci. U.S.A. p. 2645-50).

In some embodiments, an interleukin-1 beta (IL-1β) inhibitor is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. In some embodiments, the IL-1β inhibitor is chosen from canakinumab, gevokizumab, Anakinra, or Rilonacept. In some embodiments, the IL-1β inhibitor is canakinumab.

Exemplary IL-1β inhibitors

In some embodiments, the IL-IP inhibitor is canakinumab. Canakinumab is also known as ACZ885 or ILARIS®. Canakinumab is a human monoclonal IgG1/K antibody that neutralizes the bioactivity of human IL-1β.

Canakinumab is disclosed, e.g., in WO 2002/16436, U.S. Pat. No. 7,446,175, and EP 1313769. The heavy chain variable region of canakinumab has the amino acid sequence of: MEFGLSWVFLVALLRGVQCQVQLVESGGGVVQPGRSLRLSCAASGFTFSVYGMNWVRQAPGK GLEWVAIIWYDGDNQYYADSVKGRFTISRDNSKNTLYLQMNGLRAEDTAVYYCARDLRTGPFD YWGQGTLVTVSS (SEQ ID NO: 282) (disclosed as SEQ ID NO: 1 in U.S. Pat. No. 7,446,175). The light chain variable region of canakinumab has the amino acid sequence of:

(SEQ ID NO: 283)
MLPSQLIGFLLLWVPASRGEIVLTQSPDFQSVTPKEKVTITCRASQSIGS
SLHWYQQKPDQSPKLLIKYASQSFSGVPSRFSGSGSGTDFTLTINSLEAE
DAAAYYCHQSSSLPFTFGPGTKVDIK (disclosed as SEQ ID
NO: 2 in U.S. Pat. No. 7,446,175).

Canakinumab has been used, e.g., for the treatment of Cryopyrin Associated Periodic Syndromes (CAPS), in adults and children, for the treatment of systemic juvenile idiopathic arthritis (SJIA), for the symptomatic treatment of acute gouty arthritis attacks in adults, and for other IL-1β driven inflammatory diseases. Without wishing to be bound by theory, it is believed that in some embodiments, IL-1β inhibitors, e.g., canakinumab, can increase anti-tumor immune response, e.g., by blocking one or more functions of IL-1b including, e.g., recruitment of immunosuppressive neutrophils to the tumor microenvironment, stimulation of tumor angiogenesis, and/or promotion of metastasis (Dinarello (2010) Eur. J. Immunol. p. 599-606).

In some embodiments, the combination described herein includes an IL-1β inhibitor, canakinumab, or a compound disclosed in WO 2002/16436, and an inhibitor of an immune checkpoint molecule, e.g., an inhibitor of PD-1 (e.g., an anti-PD-1 antibody molecule). IL-1 is a secreted pleotropic cytokine with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). IL-1b is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1b, derived from the tumor microenvironment and by malignant cells, promotes tumor cell proliferation, increases invasiveness and dampens anti-tumor immune response, in part by recruiting inhibitory neutrophils (Apte et al. (2006) CancerMetastasis Rev. p. 387-408; Miller et al. (2007) J. Immunol. p. 6933-42). Experimental data indicate that inhibition of IL-1b results in a decrease in tumor burden and metastasis (Voronov et al. (2003) Proc. Natl. Acad. Sci. U.S.A. p. 2645-50). Canakinumab can bind IL-1b and inhibit IL-1-mediated signaling. Accordingly, in certain embodiments, an IL-1β inhibitor, e.g., canakinumab, enhances, or is used to enhance, an immune-mediated anti-tumor effect of an inhibitor of PD-1 (e.g., an anti-PD-1 antibody molecule).

In some embodiments, the IL-1β inhibitor, canakinumab, or a compound disclosed in WO 2002/16436, and the inhibitor of an immune checkpoint molecule, e.g., an inhibitor of PD-1 (e.g., an anti-PD-1 antibody molecule), each is administered at a dose and/or on a time schedule, that in combination, achieves a desired anti-tumor activity.

### MDM2 Inhibitors

In some embodiments, a mouse double minute 2 homolog (MDM2) inhibitor is used in combination with 3-(1-oxoisoindolin-2-yl)piperidine-2,6-dione IKZF2 degrader compounds or a pharmaceutically acceptable salt, hydrate, solvate, prodrug, stereoisomer, or tautomer thereof, for treating a disease, e.g., cancer. The human homolog of MDM2 is also known as HDM2. In some embodiments, an MDM2 inhibitor described herein is also known as a HDM2 inhibitor. In some embodiments, the MDM2 inhibitor is chosen from HDM201 or CGM097.

In an embodiment the MDM2 inhibitor comprises (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (also known as CGM097) or a compound disclosed in PCT Publication No. WO 2011/076786 to treat a disorder, e.g., a disorder described herein). In one embodiment, a therapeutic agent disclosed herein is used in combination with CGM097.

In an embodiment, an MDM2 inhibitor comprises an inhibitor of p53 and/or a p53/Mdm2 interaction. In an embodiment, the MDM2 inhibitor comprises (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (also known as HDM201), or a compound disclosed in PCT Publication No. WO2013/111105 to treat a disorder, e.g., a disorder described herein. In one embodiment, a therapeutic agent disclosed herein is used in combination with HDM201. In some embodiments, HDM201 is administered orally.

In one embodiment, the combination disclosed herein is suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest.

### Administration, Pharmaceutical Compositions, and Dosing

In another aspect, the present invention provides pharmaceutically acceptable compositions or formulations which comprise a therapeutically-effective amount a 3-(1-oxoisoindolin-2-yl)piperidine-2,6-dione IKZF2 degrader compound, or a pharmaceutically acceptable salt, hydrate, solvate, prodrug, stereoisomer, or tautomer thereof, of the present disclosure and second agent, formulated together with one or more pharmaceutically acceptable carriers (additives) and/or diluents.

Administration of the disclosed compounds, formulations, and combinations can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a compound, a formulation, or combination of the disclosure and a pharmaceutically acceptable carrier, such as a) a diluent, e.g., purified water, triglyceride oils, such as hydrogenated or partially hydrogenated vegetable oil, or mixtures thereof, com oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride, and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes, and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, and/or PEG200.

Liquid, particularly injectable, compositions can, for example, be prepared by dissolution, dispersion, etc. For example, the disclosed compound, formulation, or combination is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds, formulations, and combinations.

The disclosed compounds, formulations, and combinations can be also formulated as a suppository that can be prepared from fatty emulsions or suspensions; using polyalkylene glycols such as propylene glycol, as the carrier.

The disclosed compounds, formulations, and combinations can also be administered in the form of liposome delivery systems, such as small unilamellar vesicles, large unilamellar vesicles, and multilamellar vesicles. Liposomes can be formed from a variety of phospholipids, containing cholesterol, stearylamine or phosphatidylcholines.

In some embodiments, a film of lipid components is hydrated with an aqueous solution of drug to a form lipid layer encapsulating the drug, as described in U.S. Pat. No. 5,262,564 which is hereby incorporated by reference in its entirety.

Disclosed compounds, formulations, and combinations can also be delivered by the use of monoclonal antibodies as individual carriers to which the disclosed compounds are coupled. The disclosed compounds, formulations, and combinations can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds, formulations, and combinations can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

In one embodiment, the disclosure provides a kit comprising two or more separate pharmaceutical compositions, at least one of which contains a compound, formulation or combination of the present disclosure. In one embodiment, the kit comprises means for separately retaining said compositions, such as a container, divided bottle, or divided foil packet. An example of such a kit is a blister pack, as typically used for the packaging of tablets, capsules and the like.

The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

The dosage regimen utilizing the disclosed compound, formulation, or combination is selected in accordance with a variety of factors including type, species, age, weight, sex, and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Non-Clinical Pharmacology

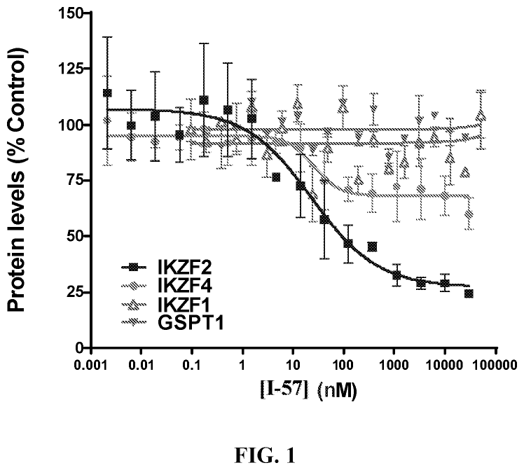

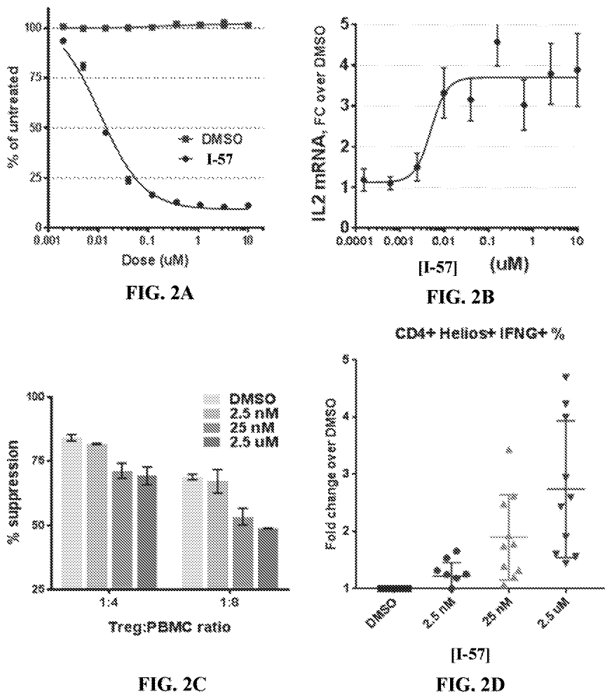

I-57 induces potent and specific degradation of IKZF2, but not of the related Ikaros family member IKZF1 (Ikaros) (FIG. 1). I-57 induces potent degradation of IKZF2 in primary human and monkey peripheral blood mononuclear cells (PBMCs) in vitro (FIG. 2A). Although the specific transcriptional targets of IKZF2 remain to be defined, IKZF2 binds to and represses transcription from the IL-2 promoter region (Baine 2013). Indeed, upon TCR stimulation, Jurkat cells expressed more IL-2 mRNA and soluble protein in a I-57 dose-dependent manner. In this experiment, the AC50 was ˜4 nM (˜1.7 ng/mL), consistent with the AC50 of degradation in these cells (FIG. 2B), suggesting that I-57-mediated IKZF2 degradation is sufficient to regulate the expression of IKZF2 transcriptional targets ([I-57 Investigator's Brochure]). Downstream biologic consequences of IKZF2 degradation have also been shown in primary human Tregs expanded in vitro which showed reduced capacity to suppress Teff proliferation in the presence of I-57 (FIG. 2C). Concurrently, the IKZF2 protein level in these Treg cells was reduced proportionally to the I-57 dose, although the proportion of IKZF2-expressing cells remained unchanged. This is consistent with the AC50 ˜4 nM (˜1.7 ng/mL) observed for IKZF2 degradation in human PBMC and suggests a strong correlation between degradation and biological effects. Finally, an in vitro assay recapitulating hallmarks of T cell dysfunction/exhaustion, by sequential TCR stimulation of isolated primary human T cells, was used to test the hypothesis that I-57 degradation would rescue the exhausted phenotype and enhance Teff cell activity. In this assay, I-57 degraded IKZF2 in a dose-dependent manner, and a concomitant increase in interferon gamma (IFN□) production by IKZF2 positive cells was detected, supporting the hypothesis that I-57 could promote Teff function (FIG. 2D). I-57 demonstrates specificity for IKZF2 degradation. In addition to IKZF1, other known cereblon substrates including translation termination factor G1 to S phase transition 1 (GSPT1) are not degraded by I-57 (FIG. 1). I-57 does degrade related Ikaros family member IKZF4 though with less potency than IKZF2 (FIG. 1). IKZF4 has been shown to have a similar role to IKZF2 in Treg cells, including Foxp3-dependent gene silencing (Pan et al. 2009). IKZF4 knockdown in Treg cells abrogated their suppressive function and, in an in vivo model of colitis, also demonstrated partial conversion to effector function. Taken together, these data suggest that partial degradation of IKZF4 may support the mechanism of action of IKZF2 degradation by I-57 ([I-57 Investigator's Brochure]).

Other potential degradation targets of I-57 were evaluated in 293T cells using whole cell proteomic analysis. The analysis suggested that out of the nearly 8000 proteins identified, the proteins TMEM97 and FIZ1 were degraded in these cells. There is no description in the literature of an adverse phenotype associated with genetic deficiency of TMEM97, in mice or in humans, suggesting the toxicology risk associated with systemic pharmacological degradation of this protein may be low, in accordance with our pre-clinical toxicology findings to date. Subsequent analyses have confirmed that FIZ1 is degraded by about 50% at a 10 μM concentration of I-57 ([I-57 Investigator's Brochure]). FIZ1, a zinc finger protein with 11 C2H2-type zinc fingers, interacts with receptor tyrosine kinase Flt3 (Wolf et al. 1999), which has been shown to play a role in proliferation and survival of hematopoietic progenitor cells as well as differentiation of early B-lymphoid progenitors, dendritic cells, and natural killer cells (McKenna et al. 2000). FIZ1 is expressed in the neural retina where it transcriptionally represses the differentiation of photoreceptors. FIZ1 is also expressed in non-ocular tissues with unknown function. The in vivo consequence of a modest decrease in FIZ1 is not known, however, there are no findings in toxicology studies suggestive of modulation of this protein.

Collectively, these data demonstrate that I-57 is a potent and selective degrader of IKZF2 in vitro, and can impact Treg and Teff cell biology at doses in the nM range.

### Nonclinical Pharmacology (In Vivo)

Of the models used to characterize the pharmacology of I-57 in vivo, two are described below: 1) Healthy donor human PBMCs (hPBMC) adoptively transferred (AdT) to immune compromised mice harboring MDA-MB231 xenografts (AdT model); and 2) the cynomolgus monkey. Single and multiple dose PK/PD studies were conducted to inform the relationship between plasma I-57 concentrations and IKZF2 degradation in cell populations including CD4+FOXP3+Treg cells. I-57 is not active in mice due to a one-amino acid difference between rodent and human CRBN protein (Kronke et al, 2015). For this reason, studies directly assessing anti-tumor efficacy of I-57 in mice were not conducted.

Degradation of IKZF2 by I-57 is species-specific

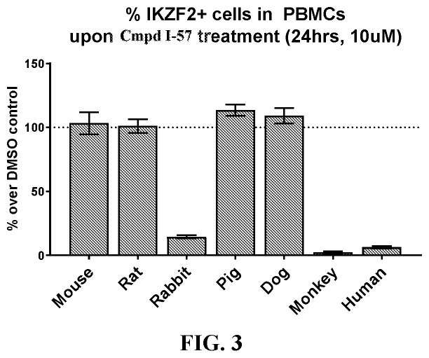

I-57 mediated degradation of IKZF2 was assessed in primary PBMCs obtained from rabbit, dog, pig, cynomolgus monkey, and human, and in primary splenocytes of mouse and rat. Degradation was observed in human, monkey and rabbit PBMCs, but not in PBMCs or splenocytes from either mouse, rat, dog or pig (FIG. 3). Species-specificity due to species specific alteration CRBN primary structure have previously been described with other compounds known to interact with the CRBN ubiquitin ligase complex to elicit protein specific degradation (Krönke et al, 2015).

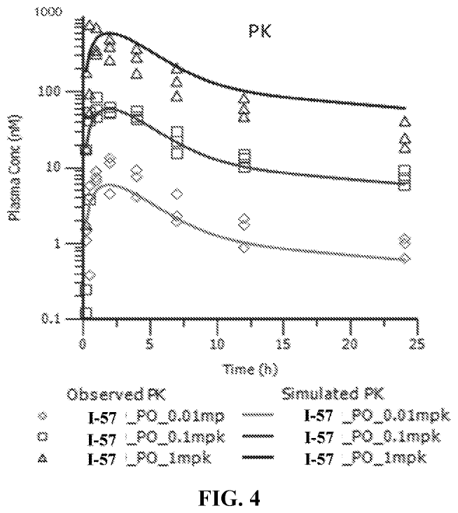

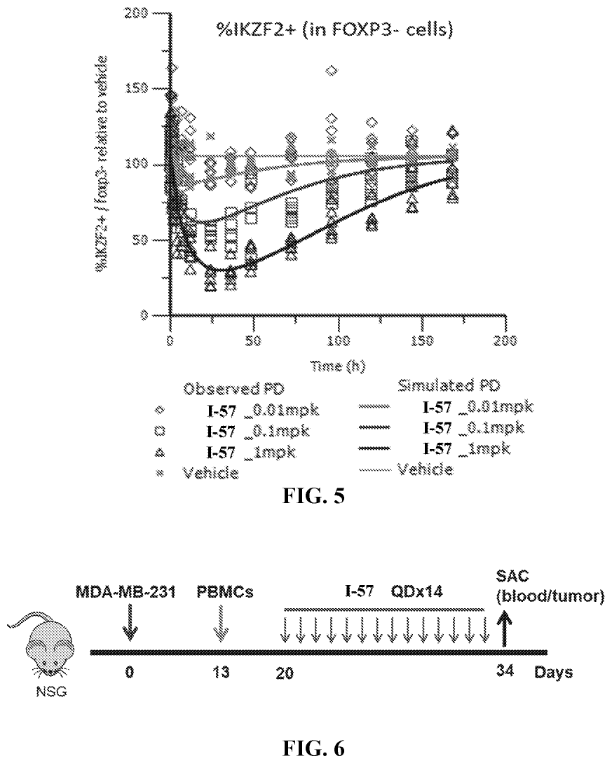

The PK/PD relationship of I-57 was examined in cynomolgus monkeys after a single oral dose of 0.01, 0.1 or 1 mg/kg. I-57 plasma concentration and IKZF2 expression (as determined by flow cytometry) in FOXP3+ T cells from PBMCs were determined (FIG. 4 and FIG. 5). Observed exposures to I-57 were slightly less than dose proportional, see Table 13.

TABLE 13
Unbound exposure of I-57 (n = 3, mean ± SD)
in cynomolgus monkey after single oral dose

| | Cmax | Cmax/Dose | AUC24 h | AUC24 h/Dose |
|---|---|---|---|---|
| Dose (mg/kg) | (ng/mL) | (ng/mL)/(mg/kg) | (ng · h/mL) | ((ng · h/mL)/(mg/kg)) |
| 0.01 | 2.11 ± 0.69 | 211 ± 69.1 | 14.1 ± 4.72 | 1410 ± 472 |
| 0.1 | 13.4 ± 2.77 | 134 ± 27.7 | 102 ± 13.2 | 1020 ± 136 |
| 1 | 102 ± 45.3 | 102 ± 45.3 | 610 ± 236 | 610 ± 236 |

Decreased (percentage change) IKZF2 positive FOXP3-cells, as determined by flow cytometry, were detectable at 4h post dose in the 1 mg/kg and 0.1 mg/kg groups, reaching maximum at 12-24 hrs post-dose and maintaining this maximum for 12-24 hrs. This effect was dose dependent. Progressive recovery was observed over the following days; total recovery was not achieved by day 7 post-dose at the highest dose (1 mg/kg), suggesting long-lasting impact of I-57 on peripheral T cells.

### I-57 PK/PD in a Repeated Oral Dose Study in the MDA-MB231 Xenograft Human PBMC Adoptive Transfer Model

The PK/PD relationship of I-57 was examined in the hPBMC AdT model system. Human PBMCs were adoptively transferred into female NSG mice harboring established MDA-MB231 xenografts (FIG. 6). This model was selected based on the observation that IKZF2 positive Tregs (CD4+FOXP3+) were readily identifiable in peripheral blood and infiltrated in the tumor xenografts.

Mice were administered I-57 daily for 14 consecutive days at the doses shown. Longitudinal I-57 plasma concentrations were assessed (Table 14). Briefly, I-57 demonstrated dose proportional increase in overall AUC and Cmax from 0.3 to 3 mg/kg while over-dose proportional increase in AUC and Cmax was seen between 3 and 30 mg/kg. No significant increase in exposure by repeated dosing was observed.

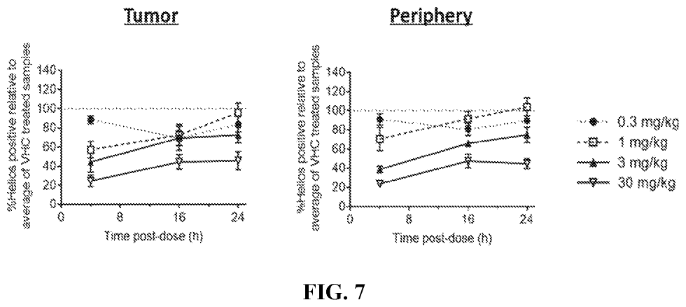

The impact of I-57 on IKZF2 protein expression in human Tregs in the periphery and infiltrated in MDA-MB231 xenografts was assessed via flow cytometry at multiple time points after the last I-57 dose. I-57 treatment resulted in robust dose and exposure-dependent IKZF2 degradation, i.e. reduction of the percentage of IKZF2 positive Tregs, in tumor and peripheral blood (FIG. 7). Maximum effects were generally observed 4-16 h post dose. At a given dose level, the magnitude and duration of IKZF2 reduction in Tregs was generally consistent across tissues (tumor, spleen and blood). This observation suggests that I-57 mediated IKZF2 degradation in peripheral Tregs can be used as a surrogate biomarker for degradation in tumor infiltrating Tregs.

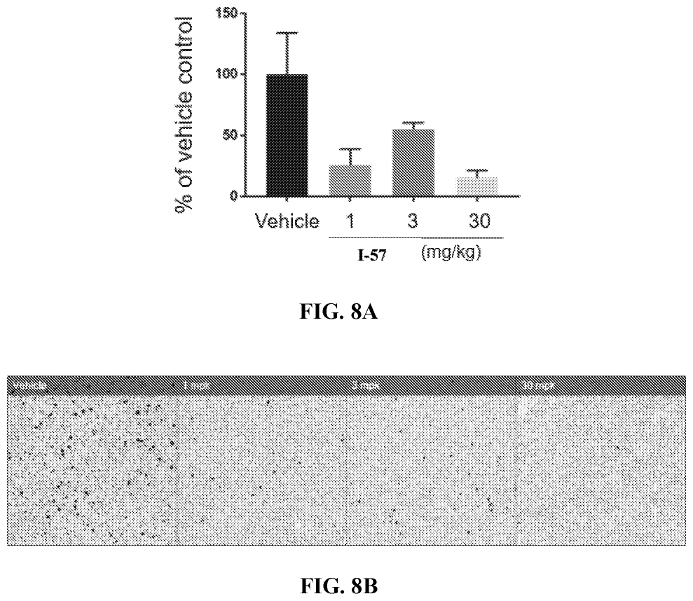

IKZF2 protein levels in total tumor-infiltrating lymphocytes were also assessed by immunohistochemistry (IHC) at 24 h post the 14th dose of 1, 3 or 30 mg/kg I-57. Robust reduction in IKZF2 levels was detected at all dose levels with approximately 85% degradation observed at 30 mg/kg. Overall, IKZF2 degradation assessed by IHC was generally consistent with our findings in Tregs by flow cytometry, though dose dependency was less apparent. Together, the data support the finding of profound I-57-mediated IKZF2 degradation in tumor-infiltrating lymphocytes (FIG. 8A and FIG. 8B).

Based on the data from this hPBMC AdT mouse model, unbound I-57 (73% percent plasma binding in the mouse) exposures (AUCinf) of 7, 30, 157 and 3474 ng*h/mL are required to achieve the reduction of IKZF2 positivity (via flow cytometry) in tumor Tregs by 31, 43, 56, and 75%, respectively. The relationship between the magnitude and duration of I-57-mediated IKZF2 degradation and subsequent modulation of therapeutically relevant downstream biology is not yet well understood.

TABLE 14
Measured unbound I-57 PK parameters post 1 or 13 daily
oral doses of 0.3, 1, 3 or 30 mg/kg (73% PPB in mouse)

| | Post 1^{st} dose | | Post 13^{th} dose | |
|---|---|---|---|---|
| p.o. Dose (mg/kg) | Cₘₐₓ (ng/mL) | AUC_{inf} (ng*h/mL) | Cₘₐₓ (ng/mL) | AUC_{inf} (ng*h/mL) |
| 0.3 | 2.9 | 7.1 | 1.1 | 7.5 |
| 1 | 8.8 | 30 | 4.6 | 20 |
| 3 | 53 | 157 | 27 | 92 |
| 30 | 1250 | 3470 | 1300 | 4270 |

### I-57 PK/PD and Impact on an Immunization Response Upon Repeated Daily Dosing in Cynomolgus Monkeys

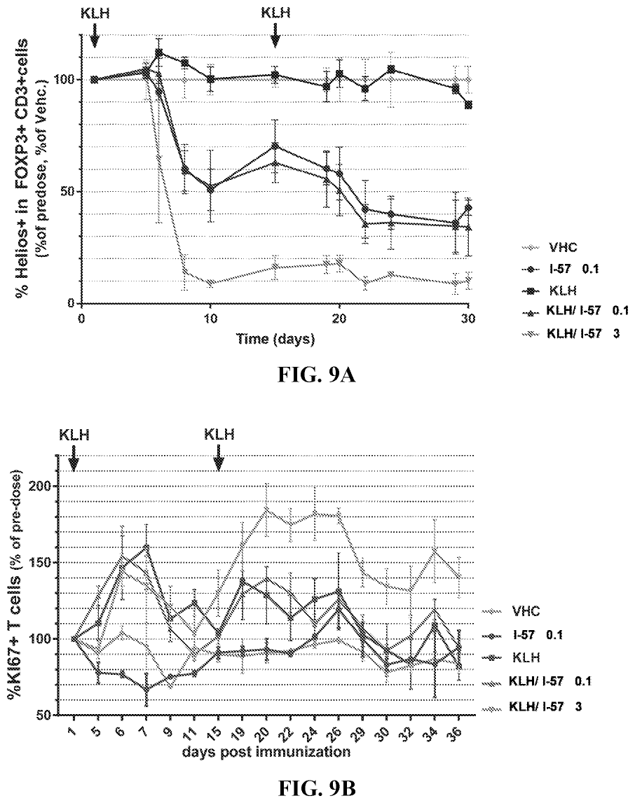

The PK/PD relationship of I-57 was further examined in cynomolgus monkeys after repeated daily oral doses of 0.1 and 3 mg/kg. In this study, a cohort of animals were initially immunized with an antigen-adjuvant mixture (KLH/Squalene). The experiment was designed to assess the effect of I-57 exposure, and the resulting IKZF2 degradation, on immune response during immunization to test the hypothesis that IKZF2 degradation would result in increased proliferation of stimulated T-cells. In the experiment, daily I-57 oral treatment was initiated 5 days after immunization. An immunization booster dose was administered on day 15. I-57 plasma concentration and IKZF2 expression (as determined by flow cytometry) in FOXP3+ T cells from PBMCs were determined (FIG. 9A and FIG. 9B). The exposure to I-57 in this experiment is summarized in Table 15

TABLE 15
Unbound exposure of I-57 in cynomolgus
monkey after multiple daily oral dose

| | | Dose | | | | AUC0-24/ |
|---|---|---|---|---|---|---|
| | | Level | | Cmax/D | AUC0-24 | D [(ng · |
| Interval | Dose | (mg/kg/ | Cmax | [(ng/mL)/ | (ng · | h/mL)/ |
| (Day) | Group | day) | (ng/mL) | (mg/kg)] | h/mL) | (mg/kg)] |
| 5 | 2^{a} | 0.1 | 7.10 | 71.0 | 56.8 | 567 |
| | 4^{b} | 0.1 | 11.0 | 110 | 63.5 | 634 |
| | 5^{b} | 3 | 523 | 175 | 2840 | 946 |
| 29 | 2^{a} | 0.1 | 13.1 | 131 | 78.1 | 782 |
| | 4^{b} | 0.1 | 18.0 | 180 | 96.4 | 965 |
| | 5^{b} | 3 | 504 | 169 | 3460 | 1150 |

^{a}Group 2 was administered only I-57 on Study Days 5-29
^{b}Groups 4 and 5 were administered I-57 on Study Days 5-29, and 0.5 mL KLH + 0.5 mL adjuvant Study Days 1 and 15

Consistent with previous findings, IKZF2 degradation was detected 24 h after the first dose (at day 6) (FIG. 9A and FIG. 9B). The levels of IKZF2 decreased further upon repeat dosing and reached a steady-state level approximately 72 h after initiation of treatment. This level of degradation was maintained throughout the remainder of the treatment (24 days).

In order to measure the impact of I-57 treatment on the immune response following immunization, serum anti-KLH IG levels were measured longitudinally, and levels of T cell activation were measured in PBMCs by flow cytometry. Anti-IgG titers elicited by the immunization were high at baseline due to the potent immunization method, and were not further enhanced by I-57 treatment ([I-57 Investigator Brochure]). In contrast, the proportion of proliferative peripheral T cells (denoted by Ki67 staining) was increased in the highest dose group (3 mg/kg) in the recall response phase, compared to immunization alone. Levels of Ki67 remained elevated in this group until the end of the study, suggesting I-57 treatment led to a sustained increase in immune responsiveness in these animals.

IKZF2 degradation was profound at the 0.1 mg/kg dose level, but the maximal effect of I-57 on T cell responsiveness was observed at the 3 mg/kg dose. Exposure levels for 3 mg/kg in cynomolgus monkeys are anticipated to correspond to ˜100 mg QD in humans.

### Nonclinical Pharmacokinetics and Metabolism

The in vivo non-clinical PK profile of I-57 was investigated in mouse, rat, dog and cynomolgus monkey using I-57 free base. Following i.v. administration, I-57 exhibited low to moderate blood clearance (CL), moderate to high volume of distribution (Vss), and a moderate terminal half-life (T1/2) in all species tested. After p.o. administration, the absorption of I-57 was rapid with peak blood concentration (Tmax) occurring between 1 and 4 hours. Good bioavailability was obtained in the mouse (53%), rat (90%), dog (91%) and monkey (89%). In high dose PK and toxicology studies with I-57 the exposures in mouse, rat and monkey increased with dose. In mouse and rat, this increase in AUC was roughly dose proportional between 3 mg/kg and 100 mg/kg (mouse) or 300 mg/kg (rat), whereas in monkey it was slightly over-proportional between 10 mg/kg and 100 mg/kg and less than dose proportional above 100 mg/kg. No significant increase of exposure was observed in rat or monkey following multiple dosing (0.6- to 1.1-fold and 1.2- to 1.6-fold in rat and monkey, respectively).

The binding to plasma protein of I-57 in vitro was moderate in all species, with an observed difference between rodents (73% in mouse, 76% in rat) and non-rodents (52% in dog and monkey, 54% in human). Blood-to-plasma ratios were in the range of 0.7-1.8 (human 1.1-1.3). A very limited distribution to the brain was observed in the mouse, with a brain/plasma ratio of 0.07. Based on results with non-radiolabeled I-57 in the dog, a significant amount of unchanged I-57 was renally eliminated (˜20% of dose).

Based on in vitro metabolism studies in liver microsomes and hepatocytes across species, I-57 was very stable and no appreciable metabolic turnover was observed. In vivo in the dog, very minor amounts of the N-dealkylation product and products derived from hydrolysis of the glutarimide moiety were detected in plasma. While the contribution of CYP-mediated oxidative metabolism to systemic clearance is unknown, a preliminary assessment of the enzymes involved was conducted using human recombinant CYPs in vitro. CYP3A4 was found to primarily mediate these reactions. I-57 was found to be a P-gp substrate.

I-57 showed weak inhibition of CYP2D6 with IC50 of ˜65 μM, and very little and no inhibition of CYP1A2, CYP2A6, CYP2B6, CYP2C₈, CYP2C₉, CYP2C₁₉, CYP2E1, and CYP3A at I-57 concentrations of up to 100 μM. I-57 also showed no apparent time-dependent inhibition of CYP1A2, CYP2B6, CYP2C₈, CYP2C₉, CYP2C₁₉, CYP2D6 or CYP3A at I-57 concentrations of up to 100 μM. Based on a PXR reporter gene assay, I-57's induction potential for CYP3A appears to be low.

### Example 2: Nonclinical Toxicology

The non-clinical safety profile of I-57 was established in vitro, and in vivo in rats and cynomolgus monkeys in studies up to 4-weeks duration. Based on the outcome of these studies, QTc interval prolongation was identified as the principal safety signal for I-57. There were other toxicities such as diarrhea and vomiting in monkeys, and mortality, clinical signs and target organ toxicities in rats; however, these occurred at exposures far above those expected to result in clinical activity.

The IC50 for in vitro hERG inhibition by I-57 was 7.1 μM. In monkeys, ECG data from a single dose, and a 4-week GLP repeat dose study showed a dose dependent increase in QTc interval, of at least 30 mSec at doses >10 mg/kg. The QTc effect was of highest magnitude between 0.5-5.5 hours coinciding with Tmax, and resolved as the I-57 plasma levels declined. No cardiac electrical instability or wave abnormality was observed. Furthermore, no QT effects were noted at 3 mg/kg. The free Cmax in monkeys at 3 mg/kg is 40-times higher than the free Cmax predicted at the clinical starting dose of 20 mg. The risk of possible QTc interval prolongation at the starting dose is therefore considered low. QT prolongation may become clinically significant during escalation but are not anticipated to be dose limiting until doses at or above 640 mg. Monitoring and other measures to mitigate potential risk of arrhythmia is addressed in Example 3 below.

In monkeys, transient vomiting and diarrhea were seen after single doses of 100 and 300 mg/kg, and in rats, mortality occurred in females at I-57 doses >300 mg/kg/day and in males at 1000 mg/kg. Clinical signs, decreases in body weight and food consumption, and clinical pathology changes reflecting inflammation, renal and liver injury, and stress were generally evident in the pre-terminal animals. Kidney toxicity was evident in rats at doses >100 mg/kg/day and was characterized by increased weight, hematuria, proteinuria, with or without increased blood urea and creatinine, degeneration, regeneration and inflammation at 300 mg/kg/day. Degenerative changes were present in the urinary bladder only at 300 mg/kg/day in a 5-day rat study. At 300 mg/kg/day, there was mucosal hyperplasia, inflammation and degeneration in the stomach attributed to possible irritation, and necrosis in colon in a few individual rats only in the 4-week rat study. Findings observed in the 4-week rat GLP study were fully reversed or showed ongoing recovery following a 1-month recovery period.

It is important to note that the exposure (AUC) associated with diarrhea and vomiting in monkeys at 100 mg/kg, and mortality and toxicity described in rats at 300 mg/kg, is high and unlikely to be achieved clinically. Similar exposure in the clinic would require doses in human of approximately 2,000 and 5,000 mg QD based on the human PK model. Activity is anticipated well below this dose level.

Other notable safety risks of I-57 include potential risks of teratogenicity, based on the structural similarity with thalidomide and autoimmunity with long-term exposure based on data from IKZF2 knockout mice. Additionally, a recent publication reported that genetic dysregulation of IKZF2 in developing mice resulted in hearing loss and damage to the cochlea outer hair cells (Chessum et al, 2018). Therefore, there is a theoretical safety risk of ototoxicity with administration of I-57.

I-57 showed no genotoxic or phototoxic potential and there were no CNS or respiratory signs seen in toxicology studies.

Based on the results of the available toxicology data, I-57 appears to have an acceptable safety profile with toxicities that are monitorable and considered reversible.

### Example 3: A Phase I/Ib, Open-Label, Multi-Center, Study of I-57 as a Single Agent and in Combination with PDR001 in Patients with Advanced Solid Tumors

A study will be run to characterize the safety and tolerability of I-57 and I-57 in combination with PDR001 in subjects with NSCLC or melanoma who have received prior anti-PD-1/PD-L1 therapy, or subjects with NPC. After the determination of the MTD/RD for a particular treatment arm, dose expansion will further assess safety, tolerability, PK/PD, and anti-tumor activity of each regimen at the MTD/RD. The study will characterize the safety and tolerability of I-57 as well as I-57+ PDR001 and identify recommended doses and regimens for future studies, by assessing the incidence and severity of AEs, SAEs, dose adjustments as well as laboratory values, vital signs and ECGs.

Preliminary anti-tumor activity of I-57 as well as I-57+PDR001, will also be evaluated by assessing the BOR and PFS. The pharmacokinetic profile of each investigational drug will be characterized, the immunogenicity of PDR001 will be accessed, and the pharmacodynamics (PD) of each investigational drug in each treatment regimen will be assessed, by assessing drug concentration within serum/plasma, anti-drug antibodies and the changes from baseline of PD markers in PBMCs and tumor tissue, respectively.

In addition, changes from baseline of PD markers in blood and tumor tissue (IKZF2, TILs, CD8, PD-L1, and FOXP3), changes in expression of immune cell markers in tumor biopsy material, changes from baseline in PD markers such as cytokines and activated immune cells in peripheral blood, and tumor mutational burden in cell-free DN will also be evaluated.

### 3.1 Study Design

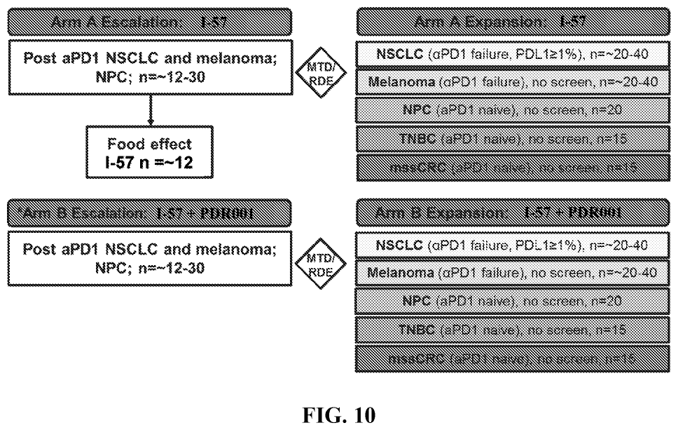

This is a FIH, open-label, phase I/Ib, multi-center study which consists of 2 dose escalation parts (Arms A and B), each followed by an expansion part. The first escalation part (Arm A) will be conducted with single agent I-57 in subjects diagnosed with NSCLC or melanoma who have received prior anti-PD-1/PD-L1 therapy, or subjects with NPC. The dose escalation part of I-57 single agent may also include preliminary analysis of food effect on I-57 exposure. Once the MTD/RD of single agent I-57 is determined, the study will continue with an expansion part with single agent I-57 in defined subject populations. The second dose escalation part (Arm B) will be performed with the combination of I-57 and PDR001 in the same indications, which were included in the single agent escalation, followed by an expansion part with the combination in the same patient populations as the single agent expansion part (FIG. 10). The dose escalation of the combination of I-57 and PDR001 may begin once a safe dose of I-57 has been identified, and will begin with a I-57 dose at least one dose level lower than that safe dose. This dose must satisfy the EWOC criteria.

For the first three subjects in each dose escalation part (arm A and arm B), a staggered approach will be utilized for enrollment and will occur as follows:
1st subject dosed, wait at least 48 hours
2nd subject dosed, wait at least 48 hours
3rd subject dosed

Following completion of this staggered dosing of the first three subjects, subsequent subjects will be treated without staggering. I-57 will be initially administered orally once per day. PDR001 will be administered i.v. once every month. Study drug will continue until a subject experiences unacceptable toxicity, progressive disease as per iRECIST/RECIST v1.1 or treatment is discontinued at the discretion of the investigator or the subject. The study design is summarized in FIG. 10. Alternative dosing schedules (e.g. less frequent dosing) may be implemented during the study if supported by available non-clinical and clinical data including preliminary PK, PD and efficacy findings, including data from other PDR001 studies.

### 3.2 Dose Escalation

Single agent I-57 (Arm A)—During the single agent dose escalation, subjects with NSCLC, melanoma or NPC will be treated with single agent I-57 until the MTD/RD is reached. At least 20 subjects are required during dose escalation to define the MTD/RD.

The dose escalation part of I-57 single agent also includes exploratory food effect cohorts to compare the effect of food on PK profiles of I-57 under fasted and fed conditions.

Combination with I-57 and PDR001 (Arm B)—During the combination dose escalation, subjects with NSCLC, melanoma or NPC will be treated with I-57 in combination with PDR001 until an RD is determined. At least 12 subjects are required during dose escalation to define an MTD/RD.

For all dose escalation arms, to assure that the RD does not exceed the MTD, the dose escalation will be guided by an adaptive Bayesian Hierarchical logistic regression model (BHLRM) following the EWOC principle.

### 3.3 Dose Expansion

Once the RD(s) have been determined in the escalation parts, additional subjects will be enrolled in the respective expansion parts in order to further characterize the PK, PD safety profile of study drug and to assess the preliminary anti-tumor activity of single agent I-57 and I-57 in combination with PDR001.

Dose expansion arms may begin only after consideration by the Investigators and Novartis of all available toxicity information (including adverse events and laboratory abnormalities that are not DLTs), the assessment of risk to future subjects from the BHLRM, and the available PK, preliminary efficacy, and PD information.

In the expansion part, subjects will be assigned to different groups depending on the tumor type as shown in FIG. 10. Each NSCLC, melanoma and NPC group will enroll approximately 20 subjects, unless enrolling 20 subjects to any of these groups is not logistically feasible, in which case enrollment may be stopped before 20 subjects are treated in that group. The initial cohorts of NSCLC will include only subjects with PD-L1≥1% in order to enrich for patients with infiltrated tumors. This selection criteria will increase the number of patient tumors with CD8≥2% to 35% compared to 25% in all comers. The arms enrolling mssCRC and TNBC subjects will enroll approximately 15 subjects, unless enrolling 15 subjects is not logistically feasible, in which case enrollment may be stopped before 15 subjects are treated in that group.

Additional subjects (up to 40) may be enrolled into the NSCLC and melanoma expansion arms to capture an adequate number of subjects with tumor infiltration (defined as subjects with tumoral CD8≥2%). This flexibility will ensure that the hypothesis that I-57 is active in patients with infiltrated tumors will be tested. Enrollment in either of these arms may be halted before completion of enrollment based on review of data from the initial safety cohort or the ongoing review of data from the expansion cohorts.

### 3.4 Study Periods

Screening period—Subjects will be evaluated against study inclusion and exclusion criteria discussed herein below in Sections 3.14 and 3.15. For NSCLC subjects participating in study parts requiring PD-L1≥1%, status of PD-L1 will be determined by the local institution. An archival or newly obtained tumor biopsy sample will be required to be submitted to a Novartis designated central laboratory or biomarker assessment at screening/baseline for all subjects enrolled in dose escalation and dose expansion.

### 3.5 Treatment Period

The treatment period will begin on Cycle 1 Day 1. For the purpose of scheduling and evaluations, a treatment cycle will consist of 28 days.

Follow-up (FU) period—For subjects in Arm A: Subjects will be followed for safety evaluations 30 days after the last dose of study drug. For subjects in Arm B: Subjects will be followed for safety evaluations 150 days after the last administration of PDR001 or 30 days after the last administration of I-57, whichever occurs later.

Disease progression FU— Subjects who discontinue the study for any reasons other than disease progression as per RECIST v1.1 or iRECIST will be followed for progression of disease or until the initiation of new anti-cancer therapy.

### 3.6 Definition of End of Study

The end of study will be when 80% of planned subjects in the expansion part have completed the follow-up for disease progression or discontinued the study for any reason, or if the study is terminated early. In addition, in Arm A, all subjects will have completed treatment after the 30 day safety follow-up. In Arm B, all subjects will have completed treatment after the 30 day safety follow-up for I-57 or after the 150 day safety follow-up after the last administration of PDR001, whichever occurs on a later day.

### 3.7 Early Study Termination

The study can be terminated at any time for any reason by Novartis. Should this be necessary, the subject should be seen as soon as possible and the same assessments should be performed for a discontinued or withdrawn subject. The investigator may be informed of additional procedures to be followed in order to ensure that adequate consideration is given to the protection of the subject's interests. The investigator will be responsible for informing IRB/IEC/REB of the early termination of the study.

### 3.8 Rational for Study Design

The design of this phase I/Ib, open label study was chosen to characterize the safety and tolerability of I-57 and I-57 in combination with PDR001 in subjects with NSCLC or melanoma who have received prior anti-PD-1/PD-L1 therapy, or subjects with NPC, and determine a recommended dose and regimen for future studies. The dose escalation allows the MTD/RD of I-57 and I-57 in combination with PDR001 to be established and will be guided by a Bayesian Hierarchical Logistic Regression Model (BHLRM).

The BHLRM is a well-established method to estimate the MTD/RD in cancer subjects. The adaptive BHLRM will be guided by the escalation with overdose control (EWOC) principle to control the risk of DLT in future subjects on study. The use of Bayesian response adaptive models for small datasets has been accepted by EMEA (“Guideline on clinical trials in small populations”, Feb. 1, 2007) and endorsed by numerous publications (Babb et al., “Cancer phase I clinical trials: efficient dose escalation with overdose control,” Stat Med. 17(10):1103-20, 1998); (Neuenschwander et al. 2008); (Neuenschwander et al. 2010); (Neuenschwander et al. 2014), and its development and appropriate use is one aspect of the FDA's Critical Path Initiative. The decisions on new dose levels are made by the Investigators and Novartis study personnel in a dose escalation meeting based upon the review of subject tolerability and safety information (including the BHLRM/summaries of DLT risk) along with PK, PD and preliminary activity information available at the time of the decision (Section 3.27).

### 3.9 Rationale for Dose/Regimen and Duration of Treatment

This is the first trial that will evaluate I-57 in humans. The starting dose for I-57, for subjects enrolled in this trial, is 20 mg p.o. administered once daily. The selection of the starting dose follows the ICH S9 guidelines. Preclinical pharmacology and PK/PD data also informed the selection of the starting dose (shown in Table 18 and Table 19).

Preclinical safety data in 4-week GLP toxicology studies in rats (30, 100 and 300 mg/kg/day) and cynomolgus monkeys (3, 10 and 30 mg/kg/day) provide maximum recommended starting dose (MRSD) of 100 mg in humans. Based on non-clinical animal models including AdT studies, cynomolgus vaccination studies and cynomolgus PK/PD studies (See Example 1) I-57 at 20 mg is expected to result in submaximal IKZF2 degradation in PBMCs and tumor. This is considered safe and will permit PK/PD characterization and determination of the lowest dose of I-57 at which maximal detectable IKZF2 degradation is achieved. Furthermore, non-clinical data demonstrate that downstream biologic consequences (Ki67 changes in CD3+ T-cells in vaccination study) of IKZF2 degradation are likely to be seen only with near maximal degradation at drug exposures above those anticipated to be achieved at the starting dose of 20 mg, but which are predicted to be achieved within 2-3 dose escalation steps.

In summary, the starting dose of 20 mg p.o. daily:
a) Is below 100 mg p.o. daily, the highest recommended safe starting dose determined by GLP studies in cynomolgus monkeys
b) Is expected to result in submaximal degradation of IKZF2 in PBMCs and tumor
c) Will not likely result in significant biologic activity downstream of IKZF2 degradation.

Intrapatient dose escalation may be allowed (see Section 3.29).

### 3.10 Rationale for Choice of Combination Drugs

The efficacy of PD-1 targeted therapy is limited by both FoxP3+ Treg activity and Teff dysfunction (Sharma et al. 2017). Because I-57 may inhibit Treg activity and/or increase the activity of dysfunctional Teff cells, combining I-57 with PD-1 targeted therapy may result in increased efficacy, including in subjects primarily resistant to single agent anti-PD-1/PD-L1 therapy. Clinical data suggest that PDR001 activity and safety are similar to approved agents ([PDR001 investigator brochure]). No PK interaction is anticipated. PDR001 will be used at the previously determined RD of 400 mg iv every four weeks.

### 3.11 Purpose and Timing of Design Adaptations

The dose escalation design foresees that decisions on future dose levels based on available data are taken at the end of each cohort. These are described in Section 3.27.

The MTD/RD for each combination treatment arm will be selected based on review by Novartis study personnel and Investigators of available safety and tolerability information (including the DLT risk assessment from the BHLRM using EWOC) along with PK, PD and efficacy data. The expansion part may then begin as specified in Section 3.3.

### 3.12 Risks and Benefits

Subjects enrolled in this study must have already received and failed standard of care therapy for their indication. These subjects therefore have limited treatment options. The therapy tested in this study may result in anti-tumor immune response. While there may be no clinical benefit to participating subjects, the trial offers the opportunity for subjects to receive potentially beneficial novel investigative therapy for incurable disease.

Appropriate eligibility criteria and specific dose-limiting toxicity definitions, as well as specific dose modification and stopping rules, are included in this protocol. Recommended guidelines for prophylactic or supportive management of study-drug induced adverse events are provided in Section 3.18a, Section 3.19, Section 3.20, and Section 3.21. The risk to subjects in this trial may be mitigated by compliance with the eligibility criteria and study procedures, as well as close clinical monitoring. As in any clinical study, there may be unforeseen risks with any of the combinations studied which could be serious.

Women of child bearing potential and sexually active males must be informed that taking the study treatment may involve unknown risks to the fetus if pregnancy were to occur during the study and must agree that in order to participate in the study they must adhere to the strict contraception requirements outlined in the exclusion criteria. If there is any question that the subject will not reliably comply, they should not be entered or continue in the study.

### 3.13 Population

This study will be conducted in adult patients with advanced metastatic cancer. The investigator or designee must ensure that only patients who meet all the following inclusion and none of the exclusion criteria are offered treatment in the study.

### 3.14-Inclusion Criteria

Subjects eligible for inclusion in this study must meet all of the following criteria:
1. Signed informed consent must be obtained prior to participation in the study.
2. Patients must be ≥18 years of age at the time of informed consent form (ICF) signature. For Japan only: written consent is necessary both from the patient and his/her legal representative if he/she is under the age of 20 years.
3. Patients with advanced/metastatic cancer who have progressed despite having received standard therapy in the metastatic setting or are intolerant to standard therapy, and for whom no effective standard therapy is available
4. In expansion: patient with measurable disease as determined by RECIST version 1.1,
5. Dose escalation, patients must fit into one of the following groups:
   a) NSCLC, previously treated with an anti-PD-1/PD-L1 therapy;
   b) Melanoma, previously treated with an anti-PD-1/PD-L1 therapy;
   c) NPC.
Dose expansion part, patients must fit into one of the following groups:
   a) NSCLC, primarily refractory to anti-PD-1/PD-L1 therapy with documented PD-L1≥1%;
   b) Melanoma, primarily refractory to anti-PD-1/PD-L1 therapy;
   c) NPC, naive to anti-PD-1/PD-L1 therapy;
   d) mssCRC, naive to anti-PD-1/PD-L1 therapy;
   e) TNBC, naive to anti-PD-1/PD-L1 therapy.

Primarily refractory is defined as duration of therapy with a regimen which includes an anti-PD-1/PD-L1 agent ≤6 months prior to disease progression and no objective evidence of significant radiologic response during treatment.
6. ECOG Performance Status <2
7. Patients must have a site of disease amenable to core needle biopsy, and be a candidate for tumor biopsy according to the treating institution's guidelines. Patients must be willing to undergo a new tumor biopsy at baseline, and during therapy on the study. Exceptions may be considered after documented discussion with Novartis. Patients with available archival tumor tissue obtained ≤6 months prior to study treatment initiation do not need to undergo a new tumor biopsy at screening, if the patient has not received any anti-cancer therapy since the biopsy was taken, and if adequate tissue is available.

### 3.15 Exclusion Criteria

Subjects meeting any of the following criteria are not eligible for inclusion in this study.
1. Prior treatment with an IKZF2 targeted agent
2. Presence of symptomatic central nervous system (CNS) metastases, or CNS metastases that require local CNS-directed therapy (such as radiotherapy or surgery), or increasing doses of corticosteroids within 2 weeks prior to study entry. Patients with treated brain metastases should be neurologically stable for at least 4 weeks prior to study entry and off steroids for at least 2 weeks before administration of any study treatment.
3. History of severe hypersensitivity reactions to any ingredient of study drug(s) or other mAbs and/or their excipients.
4. Patient with out of range laboratory values defined as:
   a) Creatinine clearance (calculated using Cockcroft-Gault formula, or measured)<40 mL/min;
   b) Total bilirubin >1.5×ULN, except for patients with Gilbert's syndrome who are excluded if total bilirubin >3.0×ULN or direct bilirubin >1.5×ULN;
   c) Alanine aminotransferase (ALT) >3×ULN, except for patients that have tumor involvement of the liver, who are excluded if ALT >5×ULN;
   d) Aspartate aminotransferase (AST) >3×ULN, except for patients that have tumor involvement of the liver, who are excluded if AST >5×ULN;
   e) Absolute neutrophil count (ANC)<1.0×109/L;
   f) Platelet count <75×109/L (growth factor or transfusion support may not be used to meet entry criterion);
   g) Hemoglobin (Hgb)<8 g/dL (growth factor or transfusion support may not be used to meet entry criterion);
   h) Potassium, magnesium, calcium or phosphate abnormality CTCAE >grade 1.
5. Clinically significant cardiac disease or impaired cardiac function, including any of the following:
   a) Clinically significant and/or uncontrolled heart disease such as congestive heart failure requiring treatment (NYHA grade >2), uncontrolled hypertension or clinically significant arrhythmia;
   b) On screening: QTcF >450 msec (male), or >460 msec (female);
   c) QTc not assessable;
   d) Congenital long QT syndrome;
   e) History of familial long QT syndrome or known family history of as Torsades de Pointes;
   f) Acute myocardial infarction or unstable angina pectoris <3 months prior to study entry.
6. Active, known or suspected autoimmune disease. Patients with vitiligo, type I diabetes mellitus, residual hypothyroidism due to an autoimmune condition only requiring hormone replacement, psoriasis not requiring systemic treatment, or other conditions not expected to recur, are permitted to enroll. Patients previously exposed to anti-PD-1/PD-L1 treatment who are adequately treated for skin rash or replacement therapy for endocrinopathies should not be excluded.
7. History of or current interstitial lung disease or pneumonitis grade >2.
8. Active infection requiring systemic antibiotic therapy. Patients requiring systemic antibiotics for infection must have completed therapy before screening is initiated.
9. HIV infection.
10. Hepatitis B virus (HBV) or hepatitis C virus (HCV) infection.
11. Malignant disease, other than that being treated in this study. Exceptions to this exclusion include the following: malignancies that were treated curatively at least two years before starting study treatment which have not recurred; basal cell carcinoma of the skin or squamous cell carcinoma of the skin that has undergone potentially curative therapy or in situ cervical cancer or other tumors that will not affect life expectancy.
12. Any medical condition that would, in the investigator's judgment, prevent the patient's participation in the clinical study due to safety concerns, compliance with clinical study procedures or interpretation of study results.
13. Treatment with cytotoxic or targeted antineoplastics within 3 weeks of initiation of study treatment. For cytotoxic agents that have major delayed toxicities a washout period of one cycle is indicated (examples are nitrosoureas and mitomycin C which typically require a 6 week washout). Prior antibodies or immunotherapies require a 4 week washout.
14. Systemic chronic steroid therapy (>10 mg/day prednisone or equivalent) or any other immunosuppressive therapy within 7 days of the first dose of study treatment. Topical, inhaled, nasal and ophthalmic steroids are allowed.
15. Patients receiving systemic treatment with any immunosuppressive medication that would interfere with the action of the study drugs, other than replacement-dose corticosteroids in the setting of adrenal insufficiency.
16. Use of any live vaccines against infectious diseases within 4 weeks of initiation of study treatment.
17. Major surgery within 2 weeks of the first dose of study treatment (mediastinoscopy, insertion of a central venous access device, and insertion of a feeding tube are not considered major surgery).
18. Radiotherapy within 2 weeks of the first dose of study drug, except for palliative radiotherapy to a limited field, such as for the treatment of bone pain or a focally painful tumor mass. During the expansion part, to allow evaluation of response to treatment, patients must have remaining measurable disease that has not been irradiated.
19. Participation in an interventional, investigational study within 2 weeks prior to the first dose of study treatment.
20. Presence of CTCAE ≥grade 2 toxicity (except alopecia, peripheral neuropathy and ototoxicity, which are excluded if ≥CTCAE grade 3) due to prior cancer therapy.
21. Use of hematopoietic colony-stimulating growth factors (e.g. G-CSF, GM-CSF, M-CSF), thrombopoietin mimetics or erythroid stimulating agents <2 weeks prior to start of study treatment. If thrombopoietin mimetics or erythroid stimulating agents were initiated more than 2 weeks prior to the first dose of study treatment and the patient is on a stable dose, they can be maintained.
22. Pregnant or lactating women, where pregnancy is defined as the state of a female after conception and until the termination of gestation, confirmed by a positive hCG laboratory test.
23. Sexually active males unless they use a condom during intercourse while taking I-57 and for 30 days after last dose of I-57 and should not father a child in this period. A condom is required to be used also by vasectomized men in order to prevent delivery of the drug via seminal fluid. In addition, male participants must not donate sperm for the time period specified above.
24. Women of child-bearing potential, defined as all women physiologically capable of becoming pregnant, unless they are using two highly effective methods of contraception while taking study treatment and for:
   a) 30 days after the last dose of I-57; or
   b) 150 days after the last dose of PDR001, whichever is longer.
Highly effective contraception methods include:
   c) Total abstinence (when this is in line with the preferred and usual lifestyle of the subject. Periodic abstinence (e.g., calendar, ovulation, symptothermal, post-ovulation methods) and withdrawal are not acceptable methods of contraception
   d) Female sterilization (have had surgical bilateral oophorectomy with or without hysterectomy), total hysterectomy, or tubal ligation at least six weeks before taking study treatment. In case of oophorectomy alone, only when the reproductive status of the woman has been confirmed by follow up hormone level assessment
   e) Male sterilization (at least 6 months prior to screening). For female subjects on the study, the vasectomized male partner should be the sole partner for that subject;
   f) Use of oral, (estrogen and progesterone), injected or implanted hormonal methods of contraception or placement of an intrauterine device (IUD) or intrauterine system (IUS), or other forms of hormonal contraception that have comparable efficacy (failure rate <1%), for example hormone vaginal ring or transdermal hormone contraception.
   g) Barrier method of contraception: condom or occlusive cap (diaphragm or cervical/vault caps) with spermicidal foam/gel/film/cream/vaginal suppository

In case of use of oral contraception women should have been stable on the same pill for a minimum of 3 months before taking study treatment.

Women are considered post-menopausal and not of child bearing potential if they have had over 12 months of natural (spontaneous) amenorrhea with an appropriate clinical profile (e.g. age appropriate [generally age from 40 to 59 years], history of vasomotor symptoms [e.g. hot flush]) in the absence of other medical justification or have had surgical bilateral oophorectomy (with or without hysterectomy), total hysterectomy or tubal ligation at least six weeks ago. In the case of oophorectomy alone, only when the reproductive status of the woman has been confirmed by follow up hormone level assessment is she considered not of child bearing potential. If local regulations deviate from the contraception methods listed above to prevent pregnancy, local regulations apply and will be described in the ICF.

In addition, patients eligible for the I-57 combined with PDR001 treatment arm must not meet any of the following criteria:

25. Patients who required discontinuation of treatment due to treatment-related toxicities with anti-PD-1 prior therapy.

### 3.16 Treatment—Investigational and Control Drugs

For this study, the term “investigational drug” and “study treatment” refers to I-57 and/or PDR001. Both study drugs will be provided by Novartis. PDR001 will be supplied in liquid in vial formulation. I-57 will be supplied as a capsule. All dosages prescribed and dispensed to subjects and all dose changes during the study must be recorded on the Dosage Administration Record eCRF.

TABLE 16
Dose and treatment schedule

| Investigational/ | | | | | |
|---|---|---|---|---|---|
| Control Drug | | | | Frequency | |
| (Name and | Pharmaceutical | Route of | | and/or | |
| Strength) | Dosage Form | Administration | Dose | Regimen | Supply Type |
| Spartalizumab | Concentrate for | Intravenous use | 400 mg | Every 4 | Open label |
| (PDR001) | Solution for | | (fixed | weeks | subject specific |
| 100 mg/4.0 ml LIVI | infusion | | dose) | (Q4W) | supply; vials |
| (Liquid in vial) | | | | | |
| I-57 | Capsule | Oral use | 20 mg | Daily (QD) * | Open label |
| 2 mg, 10 mg, 40 mg | | | (starting | | patient specific |
| | | | dose) | | supply; bottles |

* Alternate dosing regimens may be implemented

Exploration of alternative doses and/or dosing regimens of I-57 may be examined in escalation, even after initiation of the expansion part at the RD. If enrolling simultaneously, subjects would be assigned in an alternating fashion to cohorts of the same disease group across all the sites in this global study.

### 3.17 Treatment Duration

A subject may continue treatment with I-57 or I-57+PDR001 until the subject experiences unacceptable toxicity, confirmed disease progression per iRECIST and/or treatment is discontinued at the discretion of the investigator or the subject. If more than 28 consecutive doses of I-57 or 2 doses of PDR001 have to be skipped due to drug-related toxicities, then the drugs should be permanently discontinued. If a subject who misses more than 28 consecutive doses of I-57 or 2 consecutive doses of PDR001 due to a drug-related toxicity is experiencing clinical benefit, and in the opinion of the investigator it is in the subject's best interest to remain on study, then the subject may continue the treatment(s) after documented discussion with Novartis.

Following a toxicity-related dose interruption and any subsequent protocol-specified adjustment in dose or schedule during treatment, subjects who still do not tolerate combination study treatment may be considered for single-agent treatment. Single-agent treatment may be initiated with either drug, after resolution of the adverse event to <Grade 1, if in the opinion of the investigator, it is in the subject's best interest to remain on study based on clinical benefit/risk assessment, and with documented approval from Novartis.

### 3.18 Treatment Beyond Disease Progression

Subjects treated with I-57 alone or I-57 in combination with PDR001 will be permitted to continue study treatment beyond initial disease progression as per RECIST v1.1 or iRECIST criteria provided they meet each the following criteria: (a) Benefit assessed by the investigator; (b) No rapid disease progression; and (c) Tolerance of study treatment. Subjects enrolled to Arm A may also crossover to Arm B following progression.

In addition, treatment beyond disease progression should not jeopardize critical interventions to treat/prevent severe complications, or prevent subjects from receiving adequate care. Subjects who meet the above criteria and continue treatment beyond initial disease progression will continue all study procedures as outlined in the visit scheduled assessments. In case of clinical deterioration or suspicion of disease progression, a follow-up imaging assessment should be performed promptly rather than waiting for the next scheduled assessment. Subjects with evidence of further disease progression on an imaging assessment or who are no longer deriving clinical benefit will be discontinued.

### 3.18a Concomitant Therapy

In general, concomitant medications and therapies deemed necessary for the supportive care (e.g. such as anti-emetics, anti-diarrhea) and safety of the subject are allowed. Subjects must be told to notify the investigational site about any new medications, herbal remedies and dietary supplements he/she takes after the start of the study treatment. All medications (other than study treatment) and significant non-drug therapies (including physical therapy, herbal/natural medications and blood transfusions) administered during the study must be listed on the Prior and Concomitant Medications or the Surgical and Medical Procedures CRF. Prior antineoplastic therapies including medications, radiotherapy, and surgery are to be recorded on the separate Prior Antineoplastic Therapy eCRF during screening.

Permitted concomitant medications—Treatment with hematopoietic colony-stimulating growth factors (e.g. G-CSF, GM-CSF, M-CSF) may not be initiated during the DLT observation window (see Section 3.30) in the dose escalation part of the study, unless the subject has already experienced a DLT. Treatment with erythroid stimulating agents (ESAs) may not be initiated during the DLT observation window in the dose escalation part of the study, unless the subject has already experienced a DLT. If a subject is using ESA prior to enrollment (at least 2 weeks before start of study treatment), they may continue at the same dose.

Anticoagulation therapy is permitted if the subjects are already at stable doses for >2 weeks at time of first dose and adequate laboratory tests are performed as clinically indicated per investigator's discretion and according to local practices. Subjects who develop a new requirement for anticoagulant therapy during the conduct of the study may remain on study after documented discussion with the Novartis medical monitor. However, ongoing anticoagulant therapy should be temporarily discontinued to allow tumor sample according to the institutional guidelines. If anticoagulants cannot safely be temporarily discontinued then the subject will be exempt from the on treatment tumor biopsy, after documented discussion with the Novartis medical monitor.

Anti-hypertensives are allowed as concomitant medications; however, because transient hypotension has occurred during infusions of monoclonal antibodies, consideration should be given to withholding anti-hypertensive medications for 12 hours prior to treatment with the study drug PDR001. Permitted medications to be used with caution and/or action during I-57 treatment are listed in Table 17. These medications should be excluded from patient use if possible. If they must be given based on the investigator's judgment, then use with caution and consider a treatment interruption if the concomitant medication is only needed for a short time.

TABLE 17
List of medications to be used with caution during study drug treatment with I-57

| Mechanism of Interaction | Drug Name |
|---|---|
| Strong inhibitors of CYP3A | boceprevir, clarithromycin, conivaptan, grapefruit juice, indinavir, |
| | itraconazole, ketoconazole, lopinavir/ritonavir, mibefradil, |
| | nefazodone, nelfinavir, posaconazole, ritonavir, saquinavir, |
| | sequinavir/ritonavir, telaprevir, telithromycin, voriconazole, |
| | indinavir/ritonavir, tipranoavir/ritonavir, cobicistat, troleandomycin, |
| | danoprevir/ritonavir, eltegravir/ritonavir, |
| Strong Inducers of CYP3A | Avasimibe, carbamazepine, mitotane, phenobarbital, phenytoin, |
| | rifabutin, rifampin (rifampicin), St. John's wort (hypericum |
| | perforatum) rifabutin, enzalutamide |

The overall contribution of CYP-mediated oxidative metabolism to the clearance of I-57 is unclear, however CYP3A4 was found to be primarily responsible. Therefore, drugs that strongly inhibit CYP3A4 should be used with caution.

### 3.19-Infusion Reactions

Subjects should not receive any pre-medications before the first infusion of the investigational drug PDR001. If a subject experienced an infusion reaction, he/she may receive pre-medications on subsequent dosing days after consultation with the Novartis medical monitor. Pre-medications should be chosen per institutional practice at the discretion of the treating physician. If ≥2 subjects experience Grade 2 infusion reactions in a combination treatment dose-escalation cohort on C1D1 or if >25% of subjects experience mild infusion reactions during dose escalation, then mandatory primary prophylaxis regimens (i.e., before dosing on C1D1) will be instituted after discussion and agreement among principal investigators and Novartis. Prophylaxis regimens will include both paracetamol/acetaminophen and an antihistamine.

Acute allergic reactions should be treated as needed per institutional practice or the dose modification guideline (Sections 3.35-3.38). In the event of anaphylactic/anaphylactoid reactions, this includes any therapy necessary to restore normal cardiopulmonary status. If a subject experiences a Grade ≥3 anaphylactic/anaphylactoid reaction, the subject will discontinue study treatment. Such acute allergic reactions will be reported to the Sponsor in an expedited manner. These should be designated as reportable SAEs regardless of hospitalization, as medically important events. Subjects should be treated in a facility equipped for cardiopulmonary resuscitation. Appropriate resuscitation equipment should be available at the bedside and a physician readily available. The CTCAE category of “Infusion-related reaction” should be used to describe PDR001 infusion reactions, unless the investigator considers another category such as “Allergic reaction,” or “Anaphylaxis,” more appropriate in a specific situation.

### 3.20 Use of Bisphosphonates

Bisphosphonates are generally allowed for the management of bone metastasis and osteoporosis. However, chronic concomitant bisphosphonate therapy for the prevention of bone metastasis is not permitted. If bisphosphonate therapy is to be started after the first dose of study drug, prior consultation and approval by Novartis is required and the reason for its use must be clearly documented.

Bisphosphonates and denosumab are generally allowed with the following comments: The use of bisphosphonates is allowed regardless of indication provided subjects have been on stable doses optimally for at least 4 weeks prior to the start of treatment. Subjects requiring initiation of bisphosphonate treatment during the course of the study should be assessed by appropriate image modalities to exclude disease progression; if disease progression is documented, the subject should discontinue study treatment.

Chronic concomitant bisphosphonate/denosumab therapy for the prevention of bone metastasis is not permitted. Bisphosphonate/denosumab therapy for the treatment of osteoporosis is permitted. Bisphosphonate/denosumab therapy for the prevention of skeletal related events for subjects with bone metastases is permitted. If a subject needs to start receiving bisphosphonate/denosumab therapy after the first dose of study drug, disease progression should be formally ruled out by appropriate imaging prior to the initiation of bisphosphonates

### 3.21 Prohibited Medication

During the course of the study, subjects may not receive other additional investigational drugs, agents, devices, chemotherapy, or any other therapies that may be active against cancer. However, limited-field palliative radiotherapy to non-target lesion(s) may be allowed as concomitant therapy after documented discussion with Novartis. Such local therapies administered during the study treatment must be listed on the corresponding CRF. Study treatment must be interrupted during radiotherapy. Medications with a known risk for QT prolongation are prohibited (reference QTdrugs.org). Hematopoietic colony-stimulating growth factors (e.g. G-CSF, GM-CSF, M-CSF) are prohibited during the DLT period and may be used during the study only following documented discussion with the Novartis medical monitor.

The use of systemic steroid therapy (at doses greater than 10 mg/day prednisone or equivalent) and other immunosuppressive drugs is not allowed, with the exception of: (a) Prophylactic use for subjects with imaging contrast dye allergy; (b) Replacement-dose steroids (defined as 10 mg/day (or lower dose) of prednisone or equivalent dose of corticosteroids) in the setting of adrenal insufficiency; (c) Transient exacerbations of chronic inflammatory conditions such as COPD. Steroids must be reduced to 10 mg/day (or lower dose) of prednisone or equivalent dose of corticosteroids prior to the next administration of study treatment; and (d) Upon treatment of study treatment-related infusion reactions or study treatment-related irAEs, steroids must be reduced to 10 mg/day (or lower dose) of prednisone or equivalent dose of corticosteroids prior to the next study treatment administration.

Topical, inhaled, nasal and ophthalmic steroids are allowed. The use of live vaccines is not allowed through the whole duration of the study. Inactivated vaccines are allowed.

### 3.22 Subject Numbering

Each subject is identified in the study by a Subject Number (Subject No.), that is assigned when the subject is first enrolled for screening and is retained as the primary identifier for the subject throughout his/her entire participation in the trial. The Subject No. consists of the Center Number (Center No.) (as assigned by Novartis to the investigative site) with a sequential subject number suffixed to it, so that each subject is numbered uniquely across the entire database. Upon signing the informed consent form, the subject is assigned to the next sequential Subject No. available.

### 3.23 Treatment Assignment, Randomization, Blinding

No randomization will be performed in this study. The assignment of a subject to a particular cohort will be coordinated by the sponsor. Treatment will be open to subjects, investigator staff, persons performing the assessments, and the clinical trial team.

### Dose Escalation Guidelines

### 3.24 Starting Dose of I-57 as a Single Agent (Arm A)

The starting dose of I-57 selected for this study is supported by 4-week GLP toxicology studies performed in cynomolgus monkeys. In contrast to the rat, this is a relevant model to test the on-target toxicity of I-57 because I-57 can degrade monkey, but not rat, IKZF2. GLP toxicity studies identified 30 mg/kg as the HNSTD in monkey and supported a starting dose up to 100 mg QD in human (BSA scaled HED and using a safety factor of 6). The selected dose of 20 mg is anticipated to result in a significant but sub-maximal IKZF2 degradation in human PBMCs and TILs (50-90%), based on PK/PD modeling of I-57 exposure and IKZF2 degradation in cynomolgus PBMCs. More IKZF2 degradation may be required to change downstream biology. Therefore, 20 mg is considered safe. Escalation from this starting dose will facilitate the determination of the minimal required dose which provides optimal IKZF2 degradation to impact downstream biological activity.

### 3.25 Starting Dose of I-57 and PDR001 Combination (Arm B)

In the combination arm (Arm B), the starting dose of I-57 will be determined based on data from the single agent arm of the study (Arm A) and will be at least one dose level below the highest dose of single agent I-57 determined to be safe and tolerable. The starting dose of PDR001, when administered with I-57, will be the RD for PDR001 as determined in PDR001X2101, 400 mg i.v. administered every 4 weeks (Q4W). This dose has been determined to be active and safe and will not be escalated.

### 3.26 Provisional Dose Levels

Four dose escalations are planned in this study; Table 18 and Table 19 describe the starting doses and the provisional dose levels of the single-agent I-57 and the combinations of I-57 and PDR001 respectively, which may be evaluated during this trial. With the exception of Arm A starting dose level 1, actual dose levels will be determined based on available toxicity, pharmacokinetic and pharmacodynamic data, guided by the BHLRM (Sections 3.57-3.61). Dose escalation will continue until one or more MTDs or RDs are determined. Table 18 and Table 19 describes the starting dose and the dose levels that may be evaluated during this trial.

TABLE 18
Provisional dose levels (Arm A: single agent I-57)

| Dose level | Proposed daily dose* | Increment from previous dose |
|---|---|---|
| −2** | 4 mg | −60% |
| −1** | 10 mg | −50% |
| 1 | 20 mg | (starting dose) |
| 2 | 40 mg | 100% |
| 3 | 80 mg | 100% |
| 4 | 160 mg | 100% |
| 5 | 320 mg | 100% |

*It is possible for additional and/or intermediate dose levels to be added during the course of the study Cohorts may be added at any dose level below the MTD in order to better understand safety, PK or PD.
**Dose level −1 and −2 represent treatment doses for subjects requiring a dose reduction from the starting dose level.

TABLE 20
Provisional dose levels (Arm B:
combinations of I-57 and PDR001)

| Dose level | Proposed daily dose I-57* | Proposed dose PDR001 |
|---|---|---|
| −2** | 4 mg | 400 mg Q4W |
| −1** | 10 mg | 400 mg Q4W |
| 1 | 20 mg | 400 mg Q4W |
| 2 | 40 mg | 400 mg Q4W |
| 3 | 80 mg | 400 mg Q4W |
| 4 | 160 mg | 400 mg Q4W |
| 5 | 320 mg | 400 mg Q4W |

*It is possible for additional and/or intermediate dose levels to be added during the course of the study Cohorts may be added at any dose level below the MTD in order to better understand safety, PK or PD. Multiple dose levels below the MTD may be evaluated simultaneously in order to obtain PK and PD data across a range of doses.
**Dose level −1 and −2 represent treatment doses for subjects requiring a dose reduction from starting dose level.

### 3.27 Guidelines for Dose Escalation and Dose Selection for the Expansion Part

The dose escalation is conducted in order to establish the dose(s) of I-57 single agent or the combination of I-57+PDR001 to be used in the expansion part. Specifically, it is the one or more doses that in the view of Investigators and Novartis study personnel have the most appropriate benefit-risk as assessed by the review of safety, tolerability, PK, any available efficacy, and PD, taking into consideration the maximum tolerated dose (MTD). The MTD is the highest dose estimated to have less than 25% risk of causing a dose-limiting toxicity (DLT) during the DLT evaluation period in more than 33% of treated subjects. The dose(s) selected for the expansion part can be any dose equal to or less than the MTD, and may be declared without identifying the MTD.

Each dose escalation cohort will start with 3 to 6 newly treated subjects. They must have adequate exposure and follow-up to be considered evaluable for dose escalation decisions (The Dose-Determining Set (DDS) includes all subjects from the FAS (escalation part) who met the minimum exposure criterion and had sufficient safety evaluations, or experienced a dose limiting toxicity (DLT) during cycle 1). Initiation of dosing between the first subjects (up to the first 3) in a cohort at a dose level or combination of doses higher than any dose previously tested and shown to be safe will be staggered by at least 48 hours. Dose escalation decisions will be made when all subjects in a cohort have completed the DLT evaluation period or discontinued. Decisions will be based on a synthesis of all relevant data available from all dose levels evaluated in the ongoing study, including safety information, PK, available PD and preliminary efficacy.

Any dose escalation decisions made by investigators and Novartis personnel will not exceed the dose level satisfying the EWOC principle by the Bayesian hierarchical logistic regression model (BHLRM). In all cases, the dose for the next escalation cohort will not exceed a 100% increase from the previously tested safe dose. Smaller increases in dose may be recommended by the Investigators and Sponsor upon consideration of all of the available clinical data.

To better understand the safety, tolerability, PK, PD or anti-cancer activity of I-57 single agent and the combination of I-57+PDR001 before or while proceeding with further escalation, enrichment cohorts of 1 to 6 subjects may be enrolled at any dose level at or below the highest dose previously tested and shown to be safe. To reduce the risk of exposing subjects to an overly toxic dose, if 2 subjects experience a DLT in a new cohort, the BHLRM will be updated with the most up-to-date new information from all cohorts, without waiting for all subjects from the current cohort to complete the evaluation period. If the 2 DLTs occur in an escalation cohort, enrollment to that cohort will stop, and the next cohort will be opened at a lower dose level that satisfies the EWOC criteria. If the 2 DLTs occur in an enrichment cohort, then upon re-evaluation of all relevant data, additional subjects may be enrolled into the open cohorts only if the dose still meets the EWOC criteria. Alternatively, if recruitment to the same dose cannot continue, a new cohort of subjects may be recruited to a lower dose that satisfies the EWOC criteria.

Even in the event that a dose is not deemed acceptable for newly enrolled subject, ongoing subjects may continue treatment at that dose level at the discretion of the investigator and Novartis if it is in the best interests of the subject. Besides the scenario of 2 DLTs, the current dose being tested may be de-escalated based on new safety findings, including but not limited to observing a DLT, before a cohort is completed. Subsequent to a decision to de-escalate, re-escalation may occur if data in subsequent cohorts supports this (EWOC criteria are satisfied). All dose decisions must be agreed by Investigators and Novartis study personnel.

### 3.28 Implementation of Dose Escalation Decisions

To implement dose escalation decisions, the available toxicity information (including adverse events and laboratory abnormalities that are not DLTs), the assessment of risk to future patients from the BHLRM along with the EWOC principle, and the available PK and PD information will all be evaluated. Drug administration at the next higher dose level may not proceed until results of the previous dose level are evaluated and show that it is permissible to proceed to a higher dose level.

### 3.29 Intra-Subject Dose Escalation

Intra-subject dose escalation is not permitted at any time within the first 4 cycles of treatment or in expansion. After the 4th cycle is completed, individual subjects may be considered for treatment at a dose of I-57 higher than the dose to which they were initially assigned. The same guidelines apply to subjects receiving single agent I-57 or I-57 in combination with PDR001. In order for a subject to be treated at a higher dose of I-57, he or she must have tolerated the lower dose for at least 4 cycles of therapy (i.e. he or she must not have experienced any study drug related toxicity CTCAE grade ≥2 at the originally assigned dose). Moreover, the new, higher dose with which the subject is to be treated must be a dose that has completed evaluation and has not exceeded the maximum tolerated dose (MTD). There is no limit to the number of times a subject may have his or her dose of I-57 increased. For any further increase after the initial intra-subject dose escalation, the following rules apply: the subject must have experienced no CTCAE grade ≥2, I-57-related toxicity over at least two cycles of therapy at the lower dose, and the higher dose being considered must have been evaluated and shown not to exceed the MTD. Consultation and agreement with Novartis must occur prior to any intra-subject dose escalation occurring. Data from the first cycle of treatment at the new dose level will not be formally included into the statistical model describing the relationship between dose and occurrence of DLT.

### 3.30 Definitions of Dose Limiting Toxicities (DLTs)

A dose-limiting toxicity (DLT) is defined as an adverse event or abnormal laboratory value where the relationship to I-57 cannot be ruled out, and is not primarily related to disease, disease progression, intercurrent illness, or concomitant medications that occurs within the DLT evaluation period with single agent I-57 or I-57 combined with PDR001 and meets any of the criteria included in Table 20. The Investigator must notify Novartis immediately ofany unexpected CTCAE Grade 3 AEs or laboratory abnormalities. Prior to enrolling subjects into a higher dose level, CTCAE Grade >2 AEs will be reviewed for all subjects at the current dose level The investigator must notify the sponsor immediately of any unexpected CTCAE grade G 3 adverse events or laboratory abnormalities.

TABLE 20
Criteria for defining dose-limiting toxicities
During dose escalation, any Grade 3 or Grade 4 AEs v5.0 related
to study treatment occurring during Cycle 1 are DLTs, EXCEPT:

| | |
|---|---|
| Fatigue | Grade 3 fatigue that resolves to ≤ Grade 1 within 7 days. |
| Fever, Infection | Grade 3 infection or fever, in the absence of neutropenia that resolves |
| | within 7 days. |
| Hypertension | Grade 3 hypertension that resolves within 7 days, with or without the |
| | addition of anti-hypertensive therapy. |
| Gastrointestinal | Grade 3 nausea and/or vomiting that resolves to ≤ Grade 1 within 48 |
| | hours of starting optimal anti-emetic therapy or without intervention. |
| | Grade 3 diarrhea that resolves within 7 days after starting optimal anti- |
| | diarrhea treatment, or without intervention, where colitis is not |
| | suspected. |
| Hepatic | Grade 3 ALT or AST in the absence of significantly increased |
| | bilirubin that resolves to ≤ Grade 1 within 7 days |
| Amylase and lipase | Grade ≥3 amylase or lipase. |
| Dermatologic | Grade 3 non bullous rash without epidermal detachment that resolves |
| | to ≤ Grade 1 within 7 days of starting treatment. |
| Hematology | Grade 3 neutropenia without fever or other clinical symptoms that |
| | resolves to ≤ Grade 1 within 7 days. |
| | Grade 3 thrombocytopenia without clinically significant bleeding. |
| | Grade 3 anemia that resolves within 7 days in the absence of |
| | transfusion. |
| | Lymphopenia of any grade is not a DLT. |
| Electrolytes | Grade 3 electrolyte abnormalities that resolve to ≤ Grade 1 within 7 |
| | days after starting supplementation. |
| Musculoskeletal | Grade 3 asymptomatic increase in creatine kinase that resolves within |
| | 14 days in the absence of evidence of cardiac involvement. |
| Immune-related toxicities* | In general, a Grade 3 immune-related AE that resolves to ≤ Grade 1 |
| | within 7 days of starting appropriate treatment is not a DLT. |

The following Grade 2 AEs related to study treatment are Considered DLTs:

| | |
|---|---|
| Ocular disorders | Grade 2 eye pain or reduction of visual acuity is a DLT if it does not |
| | respond to topical therapy and does not improve to Grade 1 severity |
| | within 2 weeks of the initiation of topical therapy OR requires |
| | systemic treatment. |
| Pneumonitis | Grade 2 pneumonitis is a DLT if it does not resolve to ≤ Grade 1 |
| | within 7 days of starting corticosteroids. |
| Myocarditis | Grade 2 myocarditis is a DLT. |
| Colitis | Grade 2 colitis is a DLT if it persists >7 days despite treatment with |
| | corticosteroids. |
| Hepatic | Grade 2 ALT or AST accompanied by bilirubin >1.5 × ULN is a DLT. |
| Dermatologic | Grade 2 bullous disease that does not resolve to ≤ Grade 1 within 7 |
| | days of starting corticosteroids is a DLT. |
| Other adverse events | Other clinically significant toxicities, including a single event or |
| | multiple occurrences of the same event that lead to a dosing delay |
| | of >7 days in cycle 1, may be considered to be DLTs by the Investigators |
| | and Novartis, even if not CTCAE Grade 3 or higher. |

*Depending on the nature of the AE, there may be cases where immune-related Grade 2-3 AEs of any duration warrant declaration of a DLT and permanent study discontinuation. DLT determination not already outlined in this table will be made on a case-by case basis after Investigator discussion with the Novartis Medical Monitor.

### 3.31 Dose Modifications

For subjects who do not tolerate the protocol-specified dosing schedule, 1-57 dose or schedule adjustments are permitted in order to allow the subject to continue the study treatment. The following guidelines need to be applied:
If a subject experiences an AE meeting the criteria for DLT as outlined in Section 3.30 (including events occurring after C1), treatment must be withheld. A decision to resume treatment following the occurrence such an event may be taken after documented discussion with the Novartis medical monitor, weighing the relative risk/benefit on a case-by-case basis. If the AE recurs at the same or higher grade, permanent discontinuation of study treatment is required.
For clinical management of suspected immune-related events, reference to consensus management guidelines is recommended such as those provided in the National Comprehensive Cancer Network (NCCN) Guidelines for the Management of Immunotherapy-Related Toxicities (available at: https://www.nccn.org/professionals/physician_gls/default.aspx#immunotherapy), the American Society for Clinical Oncology clinical practice guideline for Management of Immune-Related Adverse Events in Subjects Treated With Immune Checkpoint Inhibitor Therapy (National Comprehensive Cancer Network (NCCN) Guidelines for the Management of Immunotherapy-Related Toxicities, Brahmer, J 2018) or the European Society for Medical Oncology (ESMO) Clinical Practice Guidelines for Management of Toxicities from Immunotherapy (European Society for Medical Oncology (ESMO) Clinical Practice Guidelines for Management of Toxicities from Immunotherapy, Haanen, et al. 2017). Note that in general, study treatment should be interrupted for grade 3 and 4 toxicities and for a subset of lower grade toxicities.
Consider early referral to specialists with expertise in the diagnosis and management of immune-related AEs to thoroughly investigate events of uncertain etiology.
Events not included in the study protocol or the reference guidance documents should be managed per institutional preference.

Dose reductions are not permitted for PDR001. Table 21 for dose modification guidelines. Study treatment may be delayed due to toxicities. The study treatment may resume once the adverse event has resolved, as described in Table 21, and the start of the cycle will be shifted accordingly.

### 3.32 Dose Modification During Dose Escalation

If a subject experiences a DLT (Cycle 1), then treatment with all study drugs for this subject must be interrupted. If the toxicity resolves to grade 1 or baseline within 1 week of onset, treatment may be resumed at the same or a lower dose level at the Investigator's discretion and following discussion with Novartis. If a subject experiences an intolerable grade 2 or ≥grade 3 AE related to study treatment during food effect run-in period then treatment must be interrupted. If the toxicity resolves to grade 1 or baseline within 1 week of onset, then the Investigator may give the next run-in I-57 dose, or start the treatment phase of the study, at the Investigator's discretion and following discussion with Novartis.

3.33 Dose Modification after Cycle 1 of Dose Escalation or During Dose Expansion

If a subject experiences a study related grade 3 or 4 AE after cycle 1 in dose escalation or at any point during dose expansion, then treatment with all investigational drugs for this subject should be interrupted, with exceptions (as noted in Table 21). For all toxicity grades, if the toxicity resolves to the extent required in the Table 21, treatment may be resumed at the same or a lower dose level, unless otherwise specified, at the Investigator's discretion and following discussion with the Sponsor.

For toxicities related to study medications that result in treatment delays of more than 7 but not more than 28 days, treatment for the study drug(s) may be resumed at a lower dose level. Up to three dose reductions are permitted per patient for I-57. If a subject requires more than three dose reductions, then this study drug must be discontinued. If a patient requires a dose interruption of >28 days from the intended day of the next scheduled dose, then the patient must be discontinued from the study, unless the patient is receiving clinical benefit (such patients must be discussed with Novartis before continuation beyond the 28 day window) (refer to Section 3.18). In this event, more frequent follow-up as outlined in Cycle 1 to monitor this toxicity may be appropriate. For each subject, once a dose level reduction has occurred, the dose level may not be re-escalated during subsequent treatment cycles. All dose reductions must be discussed with and approved by the sponsor.

Outside of the DLT period, for adverse events of potential immune-related etiology (irAE) that do not recover to the extent required in the Table 21 at a dose of immunosuppression of ≤10 mg/day prednisone or equivalent and/or requiring continuation of other immunosuppressive drugs within 12 weeks after initiation of immunosuppressive therapy, PDR001 must be permanently discontinued. For patients who permanently discontinue one of the study drugs of a treatment combination, the other study drug may be continued to be administered. The other drug may be continued to be dosed at the same dose.

TABLE 21
Criteria for dose reduction/interruption and re-initiation of single
agent I-57 or I-57 plus PDR001 treatment for adverse drug reactions

| | |
|---|---|
| Worst toxicity CTCAE^{a} grade | Recommended Dose Modification |

Cytokine Release Syndrome (CRS)

| | |
|---|---|
| Grade 2 | See instructions for Grade 2 Infusion Reaction below. |
| Grade 3 or Grade 4 | Discontinue study treatment. |
| | Follow-up CRS as per institutional guidelines. |
| | Take blood for cytokine measurements as specified in Section |
| | 3.40 - Laboratory evaluations. |

Ocular (uveitis, eye pain, blurred vision)

| | |
|---|---|
| Grade 1 | Continue study treatment without dose modification. |
| | Ophthalmology consultation. |
| Grade 2 | Hold study treatment. |
| | Urgent ophthalmology consultation. |
| | Upon resolution to ≤ Grade 1 may consider resuming study |
| | treatment without dose reduction after discussion with the |
| | Novartis Medical Monitor and in consultation with |
| | ophthalmology. |
| Grade 3 or Grade 4 | Discontinue study treatment. |
| | Urgent ophthalmology consultation. |

Pulmonary (pneumonitis)

| | |
|---|---|
| Grade 1 | Consider study treatment hold. |
| | Manage per institutional practice. |
| | Consider resuming study treatment upon radiographic evidence |
| | of improvement. |
| Grade 2 | Hold study treatment. |
| | Pulmonary and infection workup. |
| | Upon resolution to ≤ Grade 1, may resume study treatment |
| | without dose modification. |
| Grade 3 or Grade 4 | Discontinue study treatment. |

Cardiovascular
ECG QTc-Interval prolonged

| | |
|---|---|
| Grade 2 | Check electrolytes, exclude other causes for QT change, |
| | increase monitoring. |
| Grade 3 | Hold I-57. |
| | Upon resolution to Grade ≤1 or baseline within ≤7 days, may |
| | resume I-57 without dose modification after discussion with the |
| | Novartis Medical Monitor. Baseline ECG refers to the ECG(s) |
| | collected at C1D1. |
| | Dose reduction is required after second occurrence. |
| Grade 4 | Discontinue study treatment. |

Hypertension

| | |
|---|---|
| Grade 3 | Hold I-57. |
| | Upon resolution to Grade ≤1 or baseline within ≤7 days, may |
| | resume I-57 without dose modification after discussion with the |
| | Novartis Medical Monitor. |
| | Dose reduction is required after second occurrence. |
| Grade 4 | Discontinue study treatment. |

Other cardiovascular disorders

| | |
|---|---|
| Grade 2 (except myocarditis) | Hold study treatment. |
| | Upon resolution to Grade ≤1 or baseline, may resume study |
| | treatment without dose modification after discussion with the |
| | Novartis Medical Monitor. |
| Grade 2 myocarditis, or | Discontinue study treatment. |
| Grade ≥3 other cardiac disorders | |
| related to study treatment | |

Ear and labyrinth disorders
Hearing impaired

| | |
|---|---|
| Grade 2 | Hold I-57. |
| | Upon resolution to Grade ≤1 or baseline, may resume I-57 |
| | with dose modification after discussion with the Novartis |
| | Medical Monitor. |
| Grade 3 or Grade 4 | Discontinue study treatment. |

Gastrointestinal
Diarrhea/Colitis*

| | |
|---|---|
| Grade 1 | May continue study treatment without dose modification. |
| | Manage per institutional standard guidelines which should |
| | include anti-diarrheal treatment, consideration of corticosteroid |
| | therapy, and hydration. |
| Grade 2 | Hold study treatment. |
| | GI consultation. |
| | Upon resolution to ≤ Grade 1 and tapering of steroid |
| | requirement to ≤10 mg prednisone per day, resume study |
| | treatment without dose modification after discussion with the |
| | Novartis Medical Monitor. |
| Grade 3 | Hold study treatment. |
| | GI consultation. |
| | Upon resolution to ≤ Grade 1 and tapering of steroid |
| | requirement to ≤10 mg prednisone per day, consider resuming |
| | study treatment after discussion with the Novartis Medical |
| | Monitor. |
| Grade 4 | Discontinue study treatment. |

AST and/or ALT elevation

| | |
|---|---|
| Grade 2 AST and/or ALT | Hold study treatment. |
| | Manage per institutional practice. |
| | Upon resolution to ≤ Grade 1 or baseline, consider resuming |
| | study treatment without dose modification. |
| Grade 2 transaminitis with | Discontinue study treatment. |
| bilirubin >1.5 × ULN (unless | |
| Gilbert's syndrome) | |
| Grade 3 AST and/or ALT | Hold study treatment. |
| | Manage per institutional practices. |
| | Upon resolution to ≤ Grade 1 or baseline within 7 days, |
| | consider resuming study treatment without dose modification |
| | after discussion with the Novartis Medical Monitor. |
| | Otherwise, discontinue study treatment. |
| Grade 4 AST and/or ALT | Discontinue study treatment. |

Isolated total bilirubin elevation**

| | |
|---|---|
| Grade 2 | Hold study treatment. |
| | Upon resolution to ≤ Grade 1 or baseline, may continue study |
| | treatment without dose modification. |
| Grade 3 | Hold study treatment. |
| | Upon resolution to ≤ Grade 1 or baseline, may consider |
| | resuming study treatment after discussion with the Novartis |
| | Medical Monitor. |
| Grade 4 | See footnote**. Otherwise, discontinue study treatment. |

Asymptomatic amylase and/or lipase elevation***

| | |
|---|---|
| Grade 3 or Grade 4 | Continue study treatment. |
| | If levels do not resolve to ≤ Grade 2 within ≤14 days after the |
| | initial report, hold study treatment. |
| | Upon resolution to ≤ Grade 2, may resume study treatment |
| | without dose modification, after discussion with the Novartis |
| | Medical Monitor. |

Pancreatitis

| | |
|---|---|
| Grade 2/radiologic evidence | Hold study treatment. |
| | Manage per institutional practice. |
| | Upon resolution to ≤ Grade 1, may resume study treatment |
| | without dose modification, if no clinical evidence of |
| | pancreatitis and after discussion with the Novartis Medical |
| | Monitor. |
| Grade 3 or Grade 4 | Discontinue study treatment. |

Renal
Serum creatinine

| | |
|---|---|
| Grade 2 | Hold study treatment. |
| | Manage per institutional practice. |
| | Upon resolution to ≤ Grade 1, may resume study treatment |
| | without dose modification after discussion with the Novartis |
| | Medical Monitor. |
| Grade 3 or 4 | Discontinue study treatment. |

Musculoskeletal

| | |
|---|---|
| Grade 2 or Grade 3 | Hold study treatment. |
| | Consider resuming study treatment without dose modification |
| | upon resolution to ≤ Grade 1 with appropriate management. |
| Grade 4 | Discontinue study treatment. |
| | In some cases, resuming study treatment may be considered |
| | after discussion with the Novartis Medical Monitor and |
| | consultation with a rheumatologist. |

Endocrine
Hypothyroidism or hyperthyroidism

| | |
|---|---|
| Grade 2 | May continue study treatment without dose modification. |
| | Management according to institutional practice. |
| Grade 3 | Hold study treatment. |
| | Upon resolution to Grade ≤1 with appropriate management, |
| | may resume study treatment without dose modification. |
| Grade 4 | May resume therapy following resolution or control with |
| | physiologic hormone replacement. |

Other endocrine disorders

| | |
|---|---|
| Grade 2 and Grade 3 | Hold study treatment. |
| | Upon resolution to Grade ≤1 with appropriate management, |
| | may resume study treatment without dose modification. |
| Grade 4 | Hold study treatment. |
| | Grade 4 treatment-related endocrinopathies, such as |
| | adrenal insufficiency, adrenocorticotropic hormone |
| | (ACTH) deficiency, hyper- or hypothyroidism, or glucose |
| | intolerance, which resolve or are adequately controlled with |
| | physiologic hormone replacement (corticosteroids, thyroid |
| | hormones) or glucose-controlling agents, respectively, may not |
| | require discontinuation after discussion with and approval from |
| | the Novartis Medical Monitor. |

Neurology

| | |
|---|---|
| Grade 1 | Consider study treatment hold, particularly for clinical |
| | suspicion of Guillain-Barre syndrome, encephalitis, aseptic |
| | meningitis, transverse myelitis, or peripheral neuropathy. |
| Grade 2 | Hold study treatment. |
| | In some cases, resuming study treatment may be |
| | considered after discussion with the Novartis Medical |
| | Monitor. |
| Grade 3 or Grade 4 | Discontinue study treatment. |

Dermatology (rash)

| | |
|---|---|
| Grade 1 | Continue study treatment without dose modification. Topical |
| | steroids, antihistamines, topical emollients |
| Grade 2 | Consider holding study treatment. |
| | Topical or oral steroids, antihistamines. |
| | If study treatment is held and resolution to ≤ Grade 1, resume |
| | study treatment without dose modification. |
| Grade 3 or Grade 4 | Hold study treatment. |
| | Manage per institutional practice. |
| | After resolution to ≤ Grade 1, consider resuming study |
| | treatment after discussion with the Novartis Medical Monitor. |
| Bullous dermatitis | Hold study treatment. |
| | Grade 1-2 bullous dermatitis: discussion with the Novartis |
| | Medical Monitor is required before considering resuming study |
| | treatment. |
| | Grade 3 bullous dermatitis: consider resuming therapy after |
| | expert consultation and documented discussion with the |
| | Novartis medical monitor. |
| | Grade 4 bullous dermatitis: discontinue study treatment |
| Stevens-Johnson syndrome (SJS), | Permanently discontinue study treatment |
| or Lyell syndrome/toxic epidermal | |
| necrolysis (TEN) | |

Hematology
Neutropenia (ANC)

| | |
|---|---|
| Grade 3 or Grade 4 | Hold study treatment. |
| | Upon resolution to ≤ Grade 2 or baseline within ≤7 days, |
| | resume study treatment without dose modification, after |
| | discussion with the Novartis Medical Monitor. |

Febrile neutropenia

| | |
|---|---|
| Grade 3 or Grade 4 | Hold study treatment. |
| | Upon resolution of fever and improvement of neutropenia to ≤ |
| | Grade 2 or baseline, resume study treatment without dose |
| | modification, after discussion with the Novartis Medical |
| | Monitor. |

Thrombocytopenia

| | |
|---|---|
| Grade 3 | Hold study treatment. |
| | Upon resolution to ≤ Grade 2 or baseline, resume study |
| | treatment without dose modification. |
| | For Grade 3 associated with major bleeding, discontinue study |
| | treatment. |
| Grade 4 | Discontinue study treatment. |

Anemia

| | |
|---|---|
| Grade 3 or Grade 4 | Hold study treatment. |
| | Upon resolution to ≤ Grade 2 or baseline within ≤7 days, |
| | resume study treatment without dose modification. |

Lymphopenia

| | |
|---|---|
| Any grade | Treatment-related lymphopenia does not require study |
| | treatment hold or discontinuation. |

Infusion reaction or Hypersensitivity reaction (Applies to PDR001 only)

| | |
|---|---|
| Grade 1 | Decrease the infusion rate until recovery of symptoms. |
| Grade 2 | Stop infusion immediately, and keep line open. Provide |
| | supportive care and medications for symptomatic relief, as per |
| | institutional practice. Corticosteroids may be administered, as |
| | needed. |
| | Resume the infusion once infusion reaction resolves (within 8 |
| | hours of initial start of infusion), administering prophylactic |
| | pre-medication as per institutional practice. Ensure that there is |
| | a minimum observation period of 1 hour prior to restarting the |
| | infusion. |
| | May restart the infusion at the same dose level, but at 50% of |
| | previous rate under continuous observation. |
| | If the AE recurs at the reinitiated slow rate of infusion, and |
| | despite pre-medication, then discontinue subject from study. |
| Grade 3 and Grade 4 | Discontinue infusion immediately, and discontinue study |
| | treatment. |
| | Provide supportive care and manage as per institutional |
| | guidelines. |

Other laboratory adverse events, not specified elsewhere in table and not included in the
Consensus guidelines

| | |
|---|---|
| Grade 3 | Hold study treatment. |
| | Upon resolution to ≤ Grade 1, resume study treatment without |
| | dose modification. |
| Grade 4 | Isolated Grade 4 electrolyte abnormalities not associated with |
| | clinical sequelae and corrected after appropriate management |
| | within 72 hours of their onset do not require discontinuation. |
| | In the case of Grade 4 electrolyte imbalances associated with |
| | clinical sequelae, or not resolved to ≤ Grade 1 within 72 hours |
| | despite appropriate management, discontinue study treatment. |

Other non-laboratory adverse events, not specified elsewhere in table and not included in the
Consensus guidelines

| | |
|---|---|
| Grade 2 | Consider study treatment hold, at Investigator discretion. |
| | Upon resolution to ≤ Grade 1, resume study treatment without |
| | dose modification. |
| Grade 3 | Hold study treatment. |
| | Upon resolution to ≤ Grade 1, resuming study treatment must |
| | be discussed with the Novartis Medical Monitor. |
| Grade 4 | Discontinue study treatment. |

All dose modifications should be based on the worst preceding toxicity.
^{a}Common Toxicity Criteria for Adverse Events (CTCAE)
*Note:
anti-diarrheal medication is recommended at the first sign of abdominal cramping, loose stools or overt diarrhea.
**Note:
If Grade 3 or 4 hyperbilirubinemia is due to the indirect (non-conjugated) component only, and hemolysis as the etiology has been ruled out as per institutional guidelines (e.g., review of peripheral blood smear and haptoglobin determination), then delay study treatment until resolved ≤ Grade 1, and resume study treatment at the discretion of the investigator.
***Note:
A CT scan or other imaging study to assess the pancreas, liver, and gallbladder must be performed within one week of the first occurrence of any ≥ Grade 3 of amylase and/or lipase.

### 3.34 Follow-Up for Toxicities

The emergence of Immune-Related AE (irAE) may be anticipated based on the mechanism of action of immunomodulatory therapies. An irAE is any clinically significant adverse event affecting any organ that is associated with study drug exposure, is consistent with an immune-mediated mechanism, and where alternative explanations have been investigated and ruled out or are considered to be unlikely. Serologic, histologic (tumor sample) and immunological assessments should be performed as deemed appropriate by the Investigator or specialist consultant to verify the immune-related nature of the AE. An empiric trial of corticosteroids may also contribute to understanding the etiology of a potential irAE.

Subjects whose treatment is interrupted or permanently discontinued due to an irAE, AE or clinically significant laboratory value, must be followed-up at least once a week (or more frequently if required by institutional practices, or if clinically indicated) for 4 weeks, and subsequently at approximately 4-week intervals, until resolution or stabilization of the event, whichever comes first. All subjects must be followed up for irAEs, AEs and SAEs for 30 days following the last dose of I-57 and 150 days following the last dose of PDR001.

### 3.35 Treatment Compliance

When I-57 is taken orally at home, the investigator must promote compliance by instructing the subject to take the study treatment exactly as prescribed and by stating that compliance is necessary for the subject's safety and the validity of the study. The subject must also be instructed to contact the investigator if he/she is unable for any reason to take the study treatment as prescribed. Compliance will be assessed by the investigator and/or study personnel at each visit using pill counts (if applicable) and information provided by the subject. This information should be captured in the source document at each visit. All study treatment infused, injected dispensed and returned must be recorded in the Drug Accountability Log. Pharmacokinetic parameters (measures of treatment exposure) will be determined in all subjects treated with I-57 single agent and the combination of I-57+PDR001 as detailed in the pharmacokinetics section.

### 3.36 Preparation and Dispensation

Each study site will be supplied with study drug in packaging as described under investigational drugs section (Section 3.16). A unique medication number is printed on the study medication label. As per the treatment assigned to the subject, investigator staff will select the study treatment to dispense, and/or infuse to the subject. The study medication to be dispensed to the patient has a 2-part label (base plus tear-off label), immediately before dispensing the package to the subject, site personnel will detach the outer part of the label from the package and affix it to the subject's source document. PDR001 (100 mg concentrate for solution for infusion) will be administered intravenously as a 30 minute infusion (up to 2 hours, if clinically indicated). Further instructions for the preparation and dispensation of PDR001 are described in the Pharmacy Manual.

### 3.37 Handling of Study Treatment

Study treatment must be received by a designated person at the study site, handled and stored safely and properly and kept in a secured location to which only the investigator and designated site personnel have access. Upon receipt, all study treatment must be stored according to the instructions specified on the labels and in the Investigator's Brochure. Clinical supplies are to be dispensed only in accordance with the protocol. Technical complaints are to be reported to the respective Novartis CO Quality Assurance. Medication labels will be in the local language and comply with the legal requirements of each country. They will include storage conditions for the study treatment but no information about the subject except for the medication number.

The investigator must maintain an accurate record of the shipment and dispensing of study treatment in a drug accountability log. Monitoring of drug accountability will be performed by monitors during site visits or remotely and at the completion of the trial. Subjects will be asked to return all unused study treatment and packaging at the end of the study or at the time of discontinuation of study treatment. At the conclusion of the study, and as appropriate during the course of the study, the investigator will return all unused study treatment, packaging, drug labels, and a copy of the completed drug accountability log to the Novartis monitor or to the Novartis address provided in the investigator folder at each site.

### 3.38 Instruction for Prescribing and Taking Study Treatment

Dosing considerations for I-57-I-57will be dispensed by sites to the patients at study visits scheduled on Day 1 of each 28-day cycle. Patients should take I-57 as instructed (i.e.: once daily (QD)) at approximately the same time each day. On days that PK samples are obtained, the patient should take I-57 during the clinic visit after the pre-dose PK samples and prior to post-dose PK samples, when instructed by the study staff

Dosing regimens are provided for each study drug in Table 16. Should new evidence from this study or other studies indicate that alternate dosing regimens may be preferred then those regimens may be explored. Patients should be instructed to swallow whole capsules and not to chew or open them. If vomiting occurs following the dosing of study treatment, re-dosing is not permitted and dosing should resume at the next scheduled dose. If the vomiting occurs on full PK sampling days within the first 6 h post-dosing, this event should be recorded on the dose administration PK electronic Case Report Form (eCRF) page, as well as on the AE page, as appropriate. Patients should be instructed not to make up missed doses. A missed dose is defined as a case when the full dose is not taken within 4 hours after the approximate time of the usual daily dosing. That day's dose should be omitted and the patient should continue treatment with the next scheduled dose.

If vomiting occurs during the course of treatment, no re-dosing of the patient is allowed before the next scheduled dose. The occurrence and frequency of any vomiting and/or diarrhea (or increase in stool frequency) during a treatment cycle must be noted in the adverse events section of the eCRF. Any doses that are missed (not taken within 6 hours of the intended time for QD regimen) should be skipped and should not be replaced or made up on a subsequent day. After cycle 2 study drug(s) may be gradually moved to evening dosing should the investigator determine this to be preferable. Patients must avoid any herbal medication preparations/medications, dietary supplements starting from 7 days prior to first dose. Vitamin supplements are permitted.

I-57 single agent under fasted conditions—Patients should be instructed to take the I-57 capsules with a large glass of water (˜250 mL or 8 oz) at approximately the same time in the morning, except on days when blood collection is scheduled at the clinic, at which time the patients should take their doses in the clinic. I-57 should be taken on an empty stomach, at least 1 hour before or 2 hours after a meal.

I-57+PDR001 dosing—For the combination arm of the study, PDR001 will be administered via i.v. infusion over 30 minutes once every 4 weeks. PDR001 infusion should be administered 30 minutes prior to I-57 administration. PDR001 infusions can be extended to up to 2 hours if clinically indicated and the break between I-57 administration and PDR001 infusion can be up to 8 hours if clinically indicated. Subjects should be closely observed and vital signs should be monitored more frequently if clinically indicated, during and for at least 2 hours after the first two PDR001 infusions. The same may be applied for the subsequent PDR001 infusions if medically indicated.

A scheduled dose (of either single agent I-57 or the combination of I-57 and PDR001) may be delayed to recover from an unresolved AE. If a subject requires a dose interruption of >28 days from the intended day of the scheduled dose due to an unresolved AE related to study drug(s), then the subject must discontinue study treatment, unless the subject is receiving clinical benefit and in the opinion of the investigator it is in the subject's best interest to remain on study treatment. The subject may restart treatment after discussion with Novartis. Dose modifications should be followed as described in Section 3.30-Section 3.33. The safety assessments should be performed as outlined in Sections 3.40-3.48 and Tables 24-30 according to the actual day of infusion.

### 3.39 Efficacy Assessment

Tumor response will be determined locally according to two sets of criteria: 1) RECIST v1.1; and 2) iRECIST The local investigator's assessment will be used for the analysis of response according to both RECIST v1.1 and iRECIST, and for treatment decision making.

At screening, all subjects will undergo CT with i.v. contrast of the chest, abdomen and pelvis. If there is clinical evidence of disease in the neck, a CT with i.v. contrast of the neck may also be performed. Brain, bone or neck imaging should be completed if relevant metastatic disease is suspected. MRI should only be used to evaluate sites of disease that are not adequately imaged by CT. If a subject is intolerant of iodine-based contrast agents, CTs may be performed without contrast. MRI may be used to evaluate sites of disease where a CT without i.v. contrast is not adequate. Visible skin lesions and easily palpable subcutaneous tumors may be measured by physical examination using a ruler or calipers. Ultrasound should not be used to measure sites of disease. Any potentially measurable lesion that has been previously treated with radiotherapy should be considered as a non-measurable lesion. However, if a lesion previously treated with radiotherapy has clearly progressed since the radiotherapy, it can be considered as a measurable lesion.

Imaging assessments during treatment period as described in Table 22 should be performed using the same imaging modality used at baseline, irrespective of study treatment interruption or actual dosing (Table 22). Imaging assessments for response evaluation will be performed every 8 weeks (+/−7 days) until C9D1 (Day 225), and every 12 weeks (+/−7 days) thereafter until disease progression, death, lost to follow-up or withdrawal of consent. Imaging assessments should be scheduled using the C1D1 date as the reference date (not the date of the previous tumor assessment), and should be respected regardless of whether treatment with study treatment is temporarily withheld or unscheduled assessments performed. Additional imaging assessments may be performed at any time during the study at the investigator's discretion to support the efficacy evaluations for a subject, as necessary. Clinical suspicion of disease progression at any time requires a physical examination and imaging assessments to be performed promptly rather than waiting for the next scheduled imaging assessment.

Each lesion that is measured at baseline must be measured by the same method (either same imaging method or by photography, including a metric ruler) and when possible, the same local radiologist/physician throughout the study so that the comparison is consistent. If an off-schedule imaging assessment is performed because progression is suspected, subsequent imaging assessments should be performed in accordance with the original imaging schedule. Combined PET/CT may be used only if the CT is of similar diagnostic quality as a CT performed without PET, including the utilization of IV contrast media. At the discretion of the Investigators, FDG-PET scans may be performed to document progressive disease per RECIST 1.1.

Partial response (PR) or complete response (CR), per both RECIST v1.1 and iRECIST, will be confirmed by a new assessment after at least 4 weeks. Also disease progression, as per iRECIST, will be confirmed after at least 4 weeks. Subjects who continue study treatment beyond initial disease progression as per RECIST criteria will continue the regular efficacy assessments. In case of clinical deterioration or suspicion of disease progression, an imaging assessment should be performed promptly rather than waiting for the next scheduled imaging assessment. For subjects who discontinue treatment for reasons other than documented disease progression, death, lost to follow-up, or withdrawal of consent, tumor assessments must continue to be performed every 12 weeks until documented disease progression, death, lost to follow-up, or withdrawal of consent.

TABLE 22
Tumor assessment collection plan

| Procedure | Screening/Baseline | During Treatment/Follow-up |
|---|---|---|
| CT or MRI with | Mandated | Treatment period: Every 8 weeks (±7 days) from |
| contrast | | C3D1(Day 57) until C9D1 (Day 225), then every 12 |
| enhancement | | weeks (±7 days) |
| (Chest, | | Following EOT, during disease progression f/u, every 12 |
| Abdomen, | | weeks until documented clinical or radiological disease |
| Pelvis,) | | progression. |
| CT or MRI with | if clinically indicated | Treatment period: Every 8 weeks (±7 days) from |
| contrast | | C3D1(Day 57) until C9D1 (Day 225), then every 12 |
| enhancement | | weeks (±7 days) |
| (Neck) | | Following EOT, during disease progression f/u, every 12 |
| | | weeks until documented clinical or radiological disease |
| | | progression or until new anti-neoplastic therapy. |
| Brain CT or | For subjects with | Treatment period: Every 8 weeks (±7 days) from |
| MRI with | known CNS | C3D1(Day 57) until C9D1 (Day 225), then every 12 |
| contrast | metastases, or if | weeks (±7 days) |
| | clinically indicated | Following EOT, during disease progression f/u, every 12 |
| | | weeks until documented clinical or radiological disease |
| | | progression or till new anti-neoplastic therapy. |
| Bone scan and/or | If clinically indicated | When clinically indicated |
| bone x-ray | | |

The efficacy assessments are standard approach to solid tumor measurement. Safety assessments are specified below with the assessment schedule detailing when each assessment is to be performed.

TABLE 23
Physical Assessments

| Assessment | Specification |
|---|---|
| Physical | Physical examination will be performed according to visit evaluation schedule. |
| examination | At Screening and Cycle 1 Day 1 prior to treatment, a complete physical examination |
| | will be performed and will include the examination of general appearance, skin, neck |
| | (including thyroid), eyes, ears, nose, throat, lungs, heart, abdomen, back, lymph |
| | nodes, extremities and neurological. |
| | After Cycle 1 Day 1 onwards, a short physical examination will be performed (it will |
| | include the examination of general appearance, vital signs). |
| | Clinically relevant findings that were present prior to signing informed consent must |
| | be recorded on the appropriate eCRF that captures medical history. Significant new |
| | findings that begin or worsen after informed consent which meet the definition of an |
| | Adverse Event must be recorded as an adverse event. |
| | Oxygen saturation (SpO₂) will be measured by pulse oximetry for subjects in Japan |
| | every time a physical examination is performed. The results of SpO_{₂} will be recorded |
| | only in the source documentation. |
| Vital signs | Vital signs include blood pressure (supine position preferred when ECG is collected), |
| | pulse measurement, and body temperature. |
| Height and | Height will be measured at screening. |
| weight | Body weight (in indoor clothing, but without shoes) will be measured at screening and |
| | at subsequent time points. |

ECOG Performance status scale will be used as described in the Table 24 and measured at the various timepoints.

TABLE 24
ECOG performance status

| Grade | ECOG Status |
|---|---|
| 0 | Fully active, able to carry on all pre-disease performance |
| | without restriction |
| 1 | Restricted in physically strenuous activity but ambulatory |
| | and able to carry out work of a light or sedentary nature |
| | (e.g., light house work, office work) |
| 2 | Ambulatory and capable of all self-care but unable to carry |
| | out any work activities. Up and about more than 50% of |
| | waking hours |
| 3 | Capable of only limited self-care, confined to bed or chair |
| | more than 50% of waking hours |
| 4 | Completely disabled. Cannot carry on any self-care. Totally |
| | confined to bed or chair |

Note:
Grade 5 (death) was removed from this table. This information will be collected on Death eCRF

### 3.40 Laboratory Evaluations

All laboratory parameters assessed for safety purposes will be evaluated locally, with the exception of cytokines, which will be analyzed centrally. Refer to Table 25 for a summary of the parameters to be evaluated according to the assessment schedule. On days of dosing, samples for these parameters will be collected prior to the administration of study drugs. More frequent evaluations may be performed at the investigator's discretion if medically indicated; results should be recorded as unscheduled laboratory assessments. Novartis will be provided with a copy of the laboratory certification and tabulation of the nomal ranges for each parameter required. In addition, ifat any time a subject has laboratory parameters obtained from a different outside laboratory, Novartis must be provided with a copy ofthe certification and a tabulation ofthe normal ranges for that laboratory. Urinalysis will be performed locally.

TABLE 25
Laboratory Assessments

| Test Category | Test Name |
|---|---|
| Hematology | Hemoglobin, Platelets, White blood Cells, Differential |
| | (Basophils, Eosinophils, Lymphocytes, Monocytes, |
| | Neutrophils, Bands, other (absolute value preferred, |
| | percentages are acceptable) |
| Chemistry | Albumin, Alkaline phosphatase, ALT, AST, Bicarbonate, |
| | Calcium, Magnesium, Phosphorus, Sodium, Potassium, |
| | Creatinine, Creatine kinase, Direct Bilirubin, Total |
| | Bilirubin, Blood Urea Nitrogen (BUN) or Urea, |
| | Amylase, Lipase, Glucose (non-fasting) |
| Urinalysis | Macroscopic Panel (Dipstick) (Bilirubin, Blood, Glucose, |
| | Ketones, Leukocytes esterase, Nitrite, pH, Protein, |
| | Specific Gravity) If macroscopic panel is abnormal then |
| | perform microscopic panel (Red Blood Cells, White |
| | blood Cells, Casts, Crystals, Bacteria, Epithelial cells) |
| Lipid Panel | Total Cholesterol, LDL, HDL and triglycerides |
| (fasting) | |
| Coagulation | Prothrombin time (PT) , Prothrombin time (quick test) or |
| | International normalized ratio [INR], Activated |
| | partial thromboplastin time (APTT) |
| Thyroid | Free T4 and/or T3, TSH (Thyroid Stimulation Hormone) |
| Virology | HIV, HBV, HCV |
| Pregnancy Test | Serum/Urine pregnancy test |

### 3.41 Electrocardiogram (ECG)

A standard 12-lead triplicate ECGs or 12-lead Holter-ECG will be performed at each ECG collection time point indicated in Section 3.42 and Section 3.43. For the standard 12-lead triplicate ECG, the ECG will be performed after the subject has been resting (supine) for approximately 10 minutes prior to each ECG collection time point. Blood samples scheduled at the same time point should be taken after the ECGs are completed or the data is collected by Holter. The individual ECGs should be recorded approximately 2 minutes apart. The mean QTcF value for each time point will be calculated from the triplicate ECG for each subject. For any ECGs with subject safety concerns, two additional ECGs must be performed to confirm the safety finding and copies forwarded to the central ECG laboratory for assessment. A monitoring or review process should be in place for clinically significant ECG findings throughout the study and especially at baseline before administration of study treatment.

Subject's eligibility for the study should be assessed by the investigator based on the local interpretation of results from the triplicate ECG recordings performed on the machine provided. Clinically significant abnormalities present at screening should be reported as Medical History in the eCRF. Clinically significant findings must be discussed with Novartis prior to assigning the subject in the study. All ECG evaluations will be independently reviewed at central labs. Instructions for collection and transmission of ECGs to the central ECG laboratory will be provided in the ECG manual. Interpretation of the tracing must be made by a qualified physician and documented on the appropriate CRF. Each ECG tracing should be labeled with the study number, subject initials (where regulations permit), subject number, date, and kept in the source documents at the study site. New or worsened clinically significant findings occurring after informed consent must be recorded as adverse events. In the event that a QTcF value of >500 ms is observed or if an unscheduled ECG is performed for safety reasons, it is recommended to collect a time-matched PK sample and record the time and date of the last study drug intake to determine the drug exposure.

Dose adjustments in case of QT prolongation should be performed per Sections 3.31-3.33 Additional, unscheduled, safety ECGs may be repeated at the discretion of the investigator at any time during the study as clinically indicated. Local cardiologist ECG assessment may also be performed at any time during the study at the discretion of the investigator.

### 3.42 I-57 Single Agent Escalation Part 12-Lead Holter-ECG

For the I-57 single agent dose escalation part, Holter-ECGs with 12-lead devices will be recorded as specified in Table 26. The central ECG laboratory will provide Holter ECG recorders and will assess the recording. During ECG collection time windows, ECGs will be recorded while strictly controlled conditions are maintained for 15 minutes. ECG replicates will be extracted during the terminal portion of the predefined collection time window. The extraction will be three times on each time point. Holter extracts for determination of QTc and other ECG parameters will be taken at the time points described in Table 26. Additional time points may be reviewed based on preliminary safety data. A standard 12-lead triplicate ECGs will be performed at the remaining time points described in Table 27.

TABLE 26
Central Holter-ECG extraction time points
for I-57 single agent dose escalation

| Cycle | Day | Time | ECG Type |
|---|---|---|---|
| Screening | −21 to −1 days | Anytime during the | 12 Lead |
| | | screening period | Holter ECG |
| 1 | 1 | Pre-dose | 12 Lead |
| | | (baseline ECG) | Holter ECG |
| 1 | 1 | 2-hr post-I-57 dose | 12 Lead |
| | | (±15 min) | Holter ECG |
| 1 | 1 | 4-hr post-I-57 dose | 12 Lead |
| | | (±30 min) | Holter ECG |
| 1 | 1 | 8-hr post-I-57 dose | 12 Lead |
| | | (±30 min) | Holter ECG |
| 1 | 2 | 24-hr post-I-57 dose | 12 Lead |
| | | (±1 h) | Holter ECG |
| 1 | 15 | Pre-dose | 12 Lead |
| | | (baseline ECG) | Holter ECG |
| 1 | 15 | 2-hr post-I-57 dose | 12 Lead |
| | | (±15 min) | Holter ECG |
| 1 | 15 | 4-hr post-I-57 dose | 12 Lead |
| | | (±30 min) | Holter ECG |
| 1 | 15 | 8-hr post-I-57 dose | 12 Lead |
| | | (±30 min) | Holter ECG |

All measurement times are relative to dose of I-57 unless otherwise specified.
If both ECG and PK sample are scheduled at the same time, the PK sample should be taken immediately (within 1 h) after ECG assessment.

TABLE 27
Central 12-Lead ECG for I-57 single agent dose escalation

| Cycle | Day | Time | ECG Type |
|---|---|---|---|
| Screening | −21 to −1 days | Anytime during the | 12 Lead |
| | | screening period | Holter ECG |
| 1 | 8 | Pre-dose | 12 Lead, |
| | | (baseline ECG) | triplicate |
| 2 | 1 | Pre-dose | 12 Lead |
| | | (baseline ECG) | Holter ECG |
| 2 | 1 | 4-hr post-I-57 dose | 12 Lead |
| | | (±30 min) | Holter ECG |
| 3 | 1 | Pre-dose | 12 Lead, |
| | | (baseline ECG) | triplicate |
| 4-6 | 1 | Pre-dose | 12 Lead, |
| | | (baseline ECG) | triplicate |
| EOT | | Within 14 days of | 12 Lead, |
| | | last dose | triplicate |
| Unscheduled | | As clinically | 12 Lead |
| | | indicated | |

All measurement times are relative to dose of I-57 unless otherwise specified.
If both ECG and PK sample are scheduled at the same time, the PK sample should be taken immediately (within 1 h) after ECG assessment.

### 3.43 ECGs for I-57+PDR001 Escalation and all Expansion Parts

The following tables describe the ECG schedule for subjects receiving 1-57 single agent in expansion, and the combination of I-57+PDR001 in both the escalation and expansion part.

TABLE 28
Central ECG collection plan for single
agent dose expansion of I-57

| Cycle | Day | Time | ECG Type |
|---|---|---|---|
| Screening | −21 to −1 days | Anytime during the | 12 Lead, |
| | | screening period | triplicate |
| 1 | 1 | Pre-dose | 12 Lead, |
| | | (baseline ECG) | triplicate |
| 1 | 1 | 2-hr post-I-57 dose | 12 Lead, |
| | | (±15 min) | triplicate |
| 1 | 1 | 4-hr post-I-57 dose | 12 Lead, |
| | | (±30 min) | triplicate |
| 1 | 1 | 8-hr post-I-57 dose | 12 Lead, |
| | | (±30 min) | triplicate |
| 1 | 2 | 24-hr post-I-57 dose | 12 Lead, |
| | | (±1 h, pre-dose for | triplicate |
| | | next day) | |
| 1 | 8 | Pre-dose | 12 Lead, |
| | | (baseline ECG) | triplicate |
| 1 | 15 | Pre-dose | 12 Lead, |
| | | (baseline ECG) | triplicate |
| 1 | 15 | 4-hr post-I-57 dose | 12 Lead, |
| | | (±30 min) | triplicate |
| 2 | 1 | Pre-dose | 12 Lead, |
| | | (baseline ECG) | triplicate |
| 2 | 1 | 4-hr post-I-57 dose | 12 Lead, |
| | | (±30 min) | triplicate |
| 3 | 1 | Pre-dose | 12 Lead, |
| | | (baseline ECG) | triplicate |
| 4-6 | 1 | Pre-dose | 12 Lead, |
| | | (baseline ECG) | triplicate |
| EOT | | Within 14 days of | 12 Lead, |
| | | last dose | triplicate |
| Unscheduled | | As clinically | 12 Lead |
| | | indicated | |

All measurement times are relative to dose of I-57 unless otherwise specified.
If both ECG and PK sample are scheduled at the same time, the PK sample should be taken immediately (within 1 h) after ECG assessment.

TABLE 29
Central ECG collection plan for I-57 + PDR001 dose expansion

| Cycle | Day | Time | ECG Type |
|---|---|---|---|
| Screening | −21 to −1 days | Anytime during the | 12 Lead, |
| | | screening period | triplicate |
| 1 | 1 | Pre-dose | 12 Lead, |
| | | (baseline ECG) | triplicate |
| 1 | 1 | End of Infusion | 12 Lead, |
| | | (EOI) | triplicate |
| 1 | 1 | 2-hr post-I-57 dose | 12 Lead, |
| | | (±15 min) | triplicate |
| 1 | 1 | 4-hr post-I-57 dose | 12 Lead, |
| | | (±30 min) | triplicate |
| 1 | 1 | 8-hr post-I-57 dose | 12 Lead, |
| | | (±30 min) | triplicate |
| 1 | 2 | 24-hr post-I-57 dose | 12 Lead, |
| | | (±1 h, pre-dose for | triplicate |
| | | next day) | |
| 1 | 8 | Pre-dose | 12 Lead, |
| | | (baseline ECG) | triplicate |
| 1 | 15 | Pre-dose | 12 Lead, |
| | | (baseline ECG) | triplicate |
| 2 | 1 | Pre-dose | 12 Lead, |
| | | (baseline ECG) | triplicate |
| 2 | 1 | 4-hr post-I-57 dose, ±15 min | 12 Lead, |
| | | | triplicate |
| 2 | 15 | Pre-dose | 12 Lead, |
| | | (baseline ECG) | triplicate |
| 3 | 1 | Pre-dose | 12 Lead, |
| | | (baseline ECG) | triplicate |
| 4-6 | 1 | Pre-dose | 12 Lead, |
| | | (baseline ECG) | triplicate |
| EOT | | Within 14 days of | 12 Lead, |
| | | last dose | triplicate |
| Unscheduled | | As clinically | 12 Lead |
| | | indicated | |

All measurement times are relative to dose of I-57 unless otherwise specified.
If both ECG and PK sample are scheduled at the same time, the PK sample should be taken immediately (within 1 h) after ECG assessment.

### 3.44 Pregnancy and Assessments of Fertility

All pre-menopausal women who are not surgically sterile will have pregnancy testing. Additional pregnancy testing might be performed if requested by local requirements. At screening, for all females of child-bearing potential a serum pregnancy test must be performed within 72 hours before the first dose. During the study (Day 1 of every cycle starting with Cycle 1) and at End of Treatment, a serum pregnancy test must be performed. Until safety follow-up completion, urine or serum pregnancy test should be performed at every month during and at the end of the safety follow-up period. If the subject is not coming to the clinic during the safety follow-up, it can be performed at home or at a local doctor's office, and the results will be communicated to the site staff. These follow-up pregnancy tests will be recorded only in the source documentation, not in the CRF.

In case of a positive urine pregnancy test, additional tests must be performed to confirm pregnancy and if confirmed the reporting requirements as described in Section 3.60, must be followed. If a subject becomes pregnant, study treatment must be stopped immediately. If a pregnancy test (urine or serum) is positive, but the subject is thought not to be pregnant, study drug should be stopped until it is determined that the test was falsely positive, and pregnancy is excluded.

### 3.45 Assessments of Fertility

Medical documentation of oophorectomy, hysterectomy, or tubal ligation must be retained as source documents. Subsequent hormone level assessment to confirm the woman is not of child bearing potential must also be available as source documentation in the following cases: (1) surgical bilateral oophorectomy without a hysterectomy; (2) reported 12 months of natural (spontaneous) amenorrhea with an appropriate clinical profile. In the absence of the above medical documentation, FSH testing is required of any female subject, regardless of reported reproductive/menopausal status at screening/baseline.

### 3.46 Chest X-Ray (Japan Only)

For Japanese subjects, a 2-view chest X-ray will be performed at screening and on Day 15 of cycle 1.

### 3.47 Hearing Assessment

At screening, an audiometry assessment, including distortion product otoacoustic emissions (DPOAE) if available and feasible, will be performed. After Cycle 1 Day 1, an audiometry assessment will be performed as clinically indicated for signs and symptoms of ototoxicity. If a subject already has significant hearing loss or has cochlear implants, no baseline hearing assessment should be performed.

### 3.48 Appropriateness of Safety Measurements

The inclusion/exclusion criteria, dose modification guidelines and safety assessments in this FIH trial account for both the disease indications and preclinical safety profile of I-57 and PDR001. Of note are the provisions put in place for the risk of autoimmune events for both PDR001 and potentially I-57. Additionally, due to the preclinical observation of QTc prolongation, particular safety measure have been implemented such as stringent exclusion criteria, dose modification guidelines, robust ECG schedule and prohibited medications. Similarly, regular urinalysis are required due to the preclinical observation of hematuria and proteinuria.

### 3.49 Pharmacokinetics and Immunogenicity Assessments

Serial blood samples will be collected in all subjects at the visits defined in the pharmacokinetic log (Table 30 and Table 31). Follow instructions outlined in the laboratory manual regarding sample collection, numbering, processing and shipment. Urine samples will also be collected in dose level 1 and a subsequent dose level close to the RD at the visits defined in the pharmacokinetic urine log (Table 32). The number of urine samples/blood draws and total blood volume collected will not exceed those stated in the protocol.

I-57 plasma and urine concentration will be determined by a validated LC-MS/MS method, and PDR001 serum concentrations will be measured by a validated ELISA method. Concentrations will be expressed in mass per volume units and will refer to the free base; concentrations below the LLOQ will be reported as “zero” and missing data will be labeled as such in the Bioanalytical Data Report. Pharmacokinetic parameters will be determined for I-57 and for PDR001. PK profiles to assess PK properties of single agent I-57 and I-57 in combination with PDR001 will be collected from all enrolled subjects. Please refer to Table 30 and Table 31 for details on PK and anti-drug antibodies (ADA) sample collections.

PK and ADA samples will be collected also at the End of Treatment Visit and in the event of a clinically significant AE (such as infusion reaction/anaphylaxis) or if ADA is suspected, at which time those samples could be used to measure any relevant biomarkers, to understand the infusion reaction/adverse event better. After the primary CSR data cut-off date is reached, no additional PK and ADA samples will be collected for the subjects still on-going on the study. Residual PK and ADA serum samples for PK and ADA analysis may also be used for exploratory PK and/or PD analyses related to I-57 treatment alone as well as combination therapy with PDR001. This could include but not limited to using leftover plasma for protein binding analysis, exploratory metabolite profiling, exploratory biomarker analysis, or alternative PK assay development and analysis.

TABLE 30
Pharmacokinetic blood collection log for I-57 alone (Arm A)

| Cycle | Day | Scheduled Time Point (h) | Analytes ^{a} |
|---|---|---|---|
| 1 | 1 | Pre-treatment of Cycle 1 | I-57 |
| 1 | 1 | 0.5 h post dose I-57 (±5 min) | I-57 |
| 1 | 1 | 1 h post dose I-57 (±15 min) | I-57 |
| 1 | 1 | 2 h post dose I-57 (±15 min) | I-57 |
| 1 | 1 | 4 h post dose I-57 (±30 min) | I-57 |
| 1 | 1 | 6 h post dose I-57 (±30 min) | I-57 |
| 1 | 1 | 8 h post dose I-57 (±30 min) | I-57, |
| 1 | 2 | Pre-treatment I-57 (24 h post I-57, ±2 h) | I-57 |
| 1 | 8 | Pre-treatment I-57 | I-57 |
| 1 | 8 | 4 h post dose I-57 (±30 min) | I-57 |
| 1 | 15 | Pre-treatment I-57 | I-57 |
| 1 | 15 | 0.5 h post dose I-57 (±15 min) | I-57 |
| 1 | 15 | 1 h post dose I-57 (±15 min) | I-57 |
| 1 | 15 | 2 h post dose I-57 (±15 min) | I-57 |
| 1 | 15 | 4 h post dose I-57 (±30 min) | I-57 |
| 1 | 15 | 6 h post dose I-57 (±30 min) | I-57 |
| 1 | 15 | 8 h post dose I-57 (±30 min) | I-57 |
| 2 | 1 | Pre-treatment I-57 | I-57 |
| 2 | 8 | Pre-treatment I-57 | I-57 |
| 2 | 8 | 4 h post dose I-57 (±30 min) | I-57 |
| 2 | 15 | Pre-treatment I-57 | I-57 |
| 2 | 15 | 4 h post dose I-57 (±30 min) | I-57 |
| 3 | 1 | Pre-treatment of Cycle 3 | I-57 |
| 4 | 1 | Pre-treatment of Cycle 4 | I-57 |
| 5 | 1 | Pre-treatment of Cycle 5 | I-57 |
| 6 | 1 | Pre-treatment of Cycle 6 | I-57 |
| EOT | | | I-57 |
| Unscheduled | | | I-57 |

^{a} Residual PK and IG serum samples used for PK and IG analysis may also be used for exploratory PK and/or PD analyses related to I-57 treatment alone
Collection of these samples may be stopped if sufficient data have been collected

TABLE 31
Pharmacokinetic blood collection log for
I-57 in combination with PDR001 (Arm B)

| Cycle | Day | Scheduled Time Point (h)^{a b} | Analytes^{c d} |
|---|---|---|---|
| 1 | 1 | Pre-treatment of Cycle 1 | I-57, PDR001, ADA |
| 1 | 1 | 0.5 h post dose I-57 (±5 min) | I-57 |
| 1 | 1 | 1 h post dose I-57 (±15 min) | I-57, PDR001 |
| | | Post infusion PDR001 (±15 min) | |
| 1 | 1 | 2 h post dose I-57 (±15 min) | I-57 |
| 1 | 1 | 4 h post dose I-57 (±30 min) | I-57 |
| 1 | 1 | 6 h post dose I-57 (±30 min) | I-57 |
| 1 | 1 | 8 h post dose I-57 (±30 min) | I-57 |
| 1 | 2 | Pre-treatment I-57 (24 h post I-57, ± 2h) | I-57, PDR001 |
| | | 24 h post infusion PDR001 (±2 h) | |
| 1 | 3 | 48 h post infusion PDR001 (±8 h) | PDR001 |
| 1 | 8 | Pre-treatment I-57 | I-57 |
| | | 4 h post dose I-57 (±30 min) | I-57 |
| | | 168 h post infusion PDR001 (±12 h) | PDR001 |
| 1 | 15 | Pre-treatment I-57 | I-57, PDR001, ADA |
| | | 336 h post infusion PDR001 (±24 h) | |
| 1 | 15 | 0.5 h post dose I-57 (±15 min) | I-57 |
| 1 | 15 | 1 h post dose I-57 (±15 min) | I-57 |
| 1 | 15 | 2 h post dose I-57 (±15 min) | I-57 |
| 1 | 15 | 4 h post dose I-57 (±30 min) | I-57 |
| 1 | 15 | 6 h post dose I-57 (±30 min) | I-57 |
| 1 | 15 | 8 h post dose I-57 (±30 min) | I-57 |
| 2 | 1 | Pre-treatment of Cycle 2 | I-57, PDR001, ADA |
| | | 672 h post infusion PDR001 (±24 h) | |
| 2 | 8 | Pre-treatment I-57 | I-57 |
| 2 | 8 | 4 h post dose I-57 (±30 min) | I-57 |
| 2 | 15 | Pre-treatment I-57 | I-57, PDR001 |
| | | 336 h post infusion PDR001 (±24 h) | |
| 2 | 15 | 4 h post dose I-57 (±30 min) | I-57 |
| 3 | 1 | Pre-treatment of Cycle 3 | I-57, PDR001, ADA |
| | | 672 h post infusion PDR001 (±24 h) | |
| 3 | 1 | Post infusion PDR001 (±15 min) | PDR001 |
| 3 | 2 | 24 h post infusion PDR001 (±2 h) | PDR001 |
| 3 | 15 | 336 h post infusion PDR001 (±24 h) | PDR001, ADA |
| 4 | 1 | Pre-treatment of Cycle 4 | I-57, PDR001, ADA |
| | | 672 h post infusion PDR001 (±24 h) | |
| 5 | 1 | Pre-treatment of Cycle 5 | I-57, PDR001, ADA |
| | | 672 h post infusion PDR001 (±24 h) | |
| 6 | 1 | Pre-treatment of Cycle 6 | I-57, PDR001, ADA |
| EOT | | | I-57, PDR001, ADA |
| Unscheduled | | | I-57, PDR001, ADA |

^{a}Residual PK and IG serum samples used for PK and IG analysis may also be used for exploratory PK and/or PD analyses related to I-57 treatment alone
^{b} Time points are taken from the infusion completion time.
^{c}PK samples are to be collected from the arm opposite from infusion site, or alternatively, infusion site will need to be flushed with 10 mL of saline.
^{d} Residual PK and IG serum samples used for PK and IG analysis may also be used for exploratory PK and/or PD analyses related to I-57 treatment alone as well as combination therapy with PDR001.
Collection of these samples may be stopped if sufficient data have been collected

TABLE 32
Pharmacokinetic urine collection log

| Cycle | Day | Scheduled Time Point (h) |
|---|---|---|
| 1 | 1 | 0-4 h post dose I-57 |
| 1 | 1 | 4-8 h post dose I-57 |
| 1 | 1-2 | 8-24 h post dose I-57 (24 h post day 1 dose) |
| 1 | 15 | 0-8 h post dose I-57 |

Collection of these samples may be stopped if sufficient data have been collected

### 3.50 Pharmacokinetic Blood Collection and Handling

Urine samples will be collected in clinic or at home. Please see the [CI-57A12101C Laboratory Manual] for detailed instructions about collection, handling and shipment of samples. The actual collection date and time of each sample will be entered on the Pharmacokinetics Urine Collection eCRF pages.

### 3.51 Analytical Method

Bioanalysis for pharmacokinetic samples will employ the following validated assays:
1. The assay to quantify I-57 and PDR001 will be a validated LCMS or another validated method.
2. The assay to quantify and assess the IG against PDR001 will be using a validated homogeneous ELISA.

### 3.52 Biomarkers

In this study, biomarker analyses will be used to investigate the effect of I-57 as a single agent and in combination with PDR001 at the molecular and cellular level as well as to determine how changes in the markers may relate to exposure and clinical outcomes. In addition, potential predictive markers of efficacy, as well as mechanisms of resistance to I-57 as a single agent and in combination with PDR001, may be explored.

While the goal of the biomarker assessments is to provide supportive data for the clinical study, there may be circumstances when a decision is made to stop a collection, or not perform or discontinue an analysis due to either practical or strategic reasons (e.g., inadequate sample number, issues related to the quality of the sample or issues related to the assay that preclude analysis, impossibility to perform correlative analyses, etc.). Therefore, depending on the results obtained during the study, sample collection and/or analysis may be omitted at the discretion of Novartis. Similarly, if a particular assay is not available for samples collected in a particular the region/country, sample collection and/or analysis may be omitted at the discretion of Novartis. In such case, the samples collections are not required.

The sample collection information must be entered on the appropriate sample collection eCRF page(s) and requisition form(s). Detailed instructions for the collection, processing, and shipment of all biomarker samples are outlined in the laboratory manual for the study. Sample should be collected at the visit/time point(s) defined in the biomarker table; Table 33.

TABLE 33
Biomarker sample collection plan

| | | | | |
|---|---|---|---|---|
| Sample Type | Visit/Time point | Approx. volume | Marker | Purpose |

Tumor samples

| | | | | |
|---|---|---|---|---|
| Newly obtained | Screening¹ | Newly obtained | IHC expression | Assess expression |
| tumor sample | C2D15² | tumor: (3-6 passes | of markers such | status of |
| (mandatory)¹ | 2-8 hours post-dose | of core needle | as: | pharmacodynamic |
| | | biopsy) | IKZF2 | and immune |
| | | Archival tumor: | PD-L1 | markers |
| | | Block (preferred) or | CD8 | Assess expression |
| | | a minimum of 20 | FOXP3 | of potential |
| | | newly cut sections | Nucleic Acid | predictors of |
| | | (4 um thickness) | sequencing | efficacy and |
| | | | (RNA/DNA) of | pharmacodynamic |
| | | | immune and | markers |
| | | | cancer related | |
| | | | genes | |

Blood samples

| | | | | |
|---|---|---|---|---|
| Blood for | C1D1 pre-dose | 10 mL per visit | Expression of | Assess |
| pharmacodynamics | C1D1 post-dose | | IKZF2 protein | pharmacodynamic |
| | C1D2 pre-dose | | | effect |
| | C1D15 pre-dose | | | |
| | C1D15 post-dose | | | |
| | C2D15 pre-dose³ | | | |
| | C2D15post-dose³ | | | |
| | Unscheduled (e.g. | | | |
| | in case of dose | | | |
| | interruption or | | | |
| | modifications) | | | |
| Blood for | C1D1 pre-dose | 10 mL per visit | Phenotypic | Assess |
| immunomonitoring | C1D2 pre-dose | | characterization | pharmacodynamic |
| | C1D15 pre-dose | | of immune cells | effect |
| | C2D15 pre-dose | | (e.g. FOXP3, | |
| | | | CD3, CD8) | |
| Blood for cytokines | C1D1 pre-dose | 3 mL per visit | Cytokine panel | Assess |
| | C1D2 | | (e.g. CCL22, | pharmacodynamic |
| | C1D15 | | IFN-γ, IL-2, | effect |
| | C2D15 | | IL-4, IL-6, IL-8) | |
| Blood for cfDNA | C1D1 pre-dose | 10 mL per visit | cfDNA as a | Assess tumor |
| | | | surrogate marker | mutational burden |
| | | | of tumor | |
| | | | mutation burden | |
| Blood for soluble | C1D1 pre-dose | 7 mL per visit | IgG markers of | Assess |
| immune factors | C1D15 | | immune response | immunological |
| | C3D1 | | to antigen (e.g. | response |
| | | | latent CMV, | |
| | | | EBV) | |

Note:
On days and time points when biomarker and pharmacokinetic blood samples are being collected, the PK sample must be drawn first.
¹Archival tumor [+pathology report] may be permitted if it meets the inclusion criteria outlined in Section 3.14
² ±3 days
³±3 days but must occur on same day as tumor biopsy

### 3.53 Tumor Collection

Newly obtained pre- and on-treatment paired tumor samples are required and are collected at screening and on-treatment as indicated in Table 33. An archival tumor and a copy of the corresponding pathology report may be submitted at the screening visit in place of a new biopsy provided the archival biopsy meets the criteria outlined in Section 3.14. Otherwise, a new biopsy at screening is required. The timing for the on-treatment biopsy may be adjusted based on emerging data.

To the extent possible, tumor sample collection for biomarker analysis should occur on the same day as the tumor evaluation when scheduled coincidentally. Table 33 summarizes the biomarker collection plan for this study. Collection of newly obtained paired tumor samples is critical to assessing the PD effect of I-57 directly in tumor. If a core needle biopsy is performed, 3-6 tumor biopsy passes are requested at both the screening and post-treatment visits. Tumor specimens from subjects treated with I-57 will be examined to assess several markers, including IKZF2 protein levels.

The status of several immune checkpoint targets, target modulation and cell populations may be analyzed in tumor tissue. Expression and localization of biomarkers including but not limited to CD8 and PD-L1 may be measured by IHC or using additional techniques deemed suitable. Target modulation and pharmacodynamic effect of treatment will be assessed by RNA/DNA expression analysis of immunomodulatory and cancer-related genes. Other related biomarkers may also be analyzed from this sample, depending on sample availability, resources, and subject's outcomes and as new scientific evidence becomes available.

### 3.54 Blood Collection

Peripheral blood will be collected pre- & during treatment as indicated in Table 33. An unscheduled blood sample for IKZF2 protein measurement may be collected in the event there is a dose interruption or dose modification, to be collected immediately prior to resumption of treatment. Collection of peripheral blood for PD assessments will allow for the assessment of IKZF2 and downstream pathway modulation by measuring IKZF2 protein levels, biomarkers of activation in circulating immune cells, and soluble cytokines (e.g. IFN-γ, IL-2, IL-4, IL-6, IL-8) in the blood. Additionally, peripheral blood sample will allow for the assessment of humoral immunological response by measurement of IgG against common latent viral antigens (e.g. CMV, EBV, HSV1). Collection of blood samples for PD effects is mandatory for all subjects. Blood will be collected at baseline to allow for sequence analysis of cfDNA. This analysis will explore the presence of mutations in tumor and cfDNA, and investigate their relationship with clinical response.

### 3.55 Additional Exploratory Analysis

During the study, in addition to the biomarkers specified above, exploratory biomarker research may be conducted on any remaining biomarker and/or PK samples. These studies would extend the search for other potential relevant biomarkers for study treatment and/or study treatment effect and/or safety. This may also include the development of ways to detect, monitor or treat cancer. These additional investigations would be dependent upon clinical outcome, reagent and sample availability.

### 3.56 Optional Additional Biomarker Studies

If the subject agrees, the biomarker samples that remain after analysis is completed (tumor and blood) may be kept for up to 15 years to be used for additional studies related to study treatment or cancer, including research to help develop ways to detect, monitor or treat cancer. A decision to perform such exploratory biomarker research studies would be based on outcome data from this study or from new scientific findings related to the drug class or disease, as well as assay availability.

### 3.57Adverse Events

An adverse event (AE) is any untoward medical occurrence (e.g. any unfavorable and unintended sign [including abnormal laboratory findings], symptom or disease) in a subject or clinical investigation subject after providing written informed consent for participation in the study. Therefore, an AE may or may not be temporally or causally associated with the use of a medicinal (investigational) product. The investigator has the responsibility for managing the safety of individual subject and identifying adverse events. Novartis qualified medical personnel will be readily available to advise on trial related medical questions or problems. The occurrence of adverse events must be sought by non-directive questioning of the subject at each visit during the study. Adverse events also may be detected when they are volunteered by the subject during or between visits or through physical examination findings, laboratory test findings, or other assessments. Adverse events must be recorded under the signs, symptoms, or diagnosis associated with them, accompanied by the following information (as far as possible) (if the event is serious refer to Section 3.58):
1. The severity grade (CTCAE Grade 1-5); Adverse events will be assessed and graded according to the Common Terminology Criteria for Adverse Events (CTCAE) version 5.
2. Its relationship to the study treatment. If the event is due to lack of efficacy or progression of underlying illness (i.e. progression of the study indication) the assessment of causality will usually be ‘Not suspected.’ The rationale for this guidance is that the symptoms of a lack of efficacy or progression of underlying illness are not caused by the trial drug, they happen in spite of its administration and/or both lack of efficacy and progression of underlying disease can only be evaluated meaningfully by an analysis of cohorts, not on a single subject
3. Its duration (start and end dates) or if the event is ongoing, an outcome of not recovered/not resolved must be reported
4. Whether it constitutes a SAE (see Section 3.58 for definition of SAE) and which seriousness criteria have been met
5. Action taken regarding with study treatment All adverse events must be treated appropriately. Treatment may include one or more of the following: (a) Dose not changed; (b) Dose Reduced/increased; and/or (3) Drug interrupted/withdrawn
6. its outcome

If the event worsens the event should be reported a second time in the CRF noting the start date when the event worsens in toxicity. For grade 3 and 4 adverse events only, if improvement to a lower grade is determined a new entry for this event should be reported in the CRF noting the start date when the event improved from having been Grade 3 or Grade 4. Conditions that were already present at the time of informed consent should be recorded in medical history of the subject. Adverse events (including lab abnormalities that constitute AEs) should be described using a diagnosis whenever possible, rather than individual underlying signs and symptoms.

Adverse event monitoring should be continued for at least 30 days for I-57 single agent or 150 days after the last dose of PDR001 or 30 days after the last dose of I-57, whichever is latest, for the combination of I-57 and PDR001. After initiation of new post-treatment antineoplastic therapy, only AEs suspected to be related to study treatment will be collected in the Adverse Event CRF. Once an adverse event is detected, it must be followed until its resolution or until it is judged to be permanent (e.g. continuing at the end of the study), and assessment must be made at each visit (or more frequently, if necessary) of any changes in severity, the suspected relationship to the interventions required to treat it, and the outcome.

Progression of malignancy (including fatal outcomes), if documented by use of appropriate method (for example, as per RECIST criteria for solid tumors or as per Cheson's guidelines for hematological malignancies), should not be reported as a serious adverse event. Adverse events separate from the progression of malignancy (i.e. deep vein thrombosis at the time of progression or hemoptysis concurrent with finding of disease progression) will be reported as per usual guidelines used for such events with proper attribution regarding relatedness to the drug.

Abnormal laboratory values or test results constitute adverse events only if they fulfill at least one of the following criteria: (a) they induce clinical signs or symptoms; (b) they are considered clinically significant; and/or (c) they require therapy. Clinically significant abnormal laboratory values or test results must be identified through a review of values outside of normal ranges/clinically notable ranges, significant changes from baseline or the previous visit, or values, which are considered to be non-typical in subjects with the underlying disease.

### 3.58 Serious Adverse Events

An SAE is defined as any adverse event [appearance of (or worsening of any pre-existing)] undesirable sign(s), symptom(s) or medical conditions(s)) which meets any one of the following criteria: (a) fatal; and/or (b) life-threatening. Life-threatening in the context of a SAE refers to a reaction in which the subject was at risk of death at the time of the reaction; it does not refer to a reaction that hypothetically might have caused death if it were more severe (See ICH-E2D Guidelines).
results in persistent or significant disability/incapacity
constitutes a congenital anomaly/birth defect
requires inpatient hospitalization or prolongation of existing hospitalization, unless hospitalization is for:
   routine treatment or monitoring of the studied indication, not associated with any deterioration in condition
   elective or pre-planned treatment for a pre-existing condition that is unrelated to the indication under study and has not worsened since signing the informed consent
   social reasons and respite care in the absence of any deterioration in the subject's general condition
   treatment on an emergency outpatient basis for an event not fulfilling any of the definitions of a SAE given above and not resulting in hospital admission
is medically significant, e.g. defined as an event that jeopardizes the subject or may require medical or surgical intervention to prevent one of the outcomes listed above

Medical and scientific judgment should be exercised in deciding whether other situations should be considered serious reactions, such as important medical events that might not be immediately life threatening or result in death or hospitalization but might jeopardize the subject or might require intervention to prevent one of the other outcomes listed above. Such events should be considered as “medically significant”. Examples of such events are intensive treatment in an emergency room or at home for allergic bronchospasm, blood dyscrasias or convulsions that do not result in hospitalization or development of dependency or abuse (See ICH-E2D Guidelines). All malignant neoplasms will be assessed as serious under “medically significant” if other seriousness criteria are not met and the malignant neoplasm is not a disease progression of the study indication. Any suspected transmission via a medicinal product of an infectious agent is also considered a serious adverse reaction. All reports of intentional misuse and abuse of the product are also considered serious adverse event irrespective if a clinical event has occurred.

### 3.59 SAE Reporting

To ensure subject safety, every SAE, regardless of suspected causality, occurring after the subject has provided informed consent and until at least 30 days for I-57 single agent and for the combination of I-57+PDR001, 150 days after the last dose of PDR001 and 30 days after the last dose of I-57, must be reported to Novartis within 24 hours of learning of its occurrence. If a subject starts a post-treatment antineoplastic therapy, then only SAEs suspected to be related to study treatment will be reported. Any additional information for the SAE including complications, progression of the initial SAE, and recurrent episodes must be reported as follow-up to the original episode within 24 hours of the investigator receiving the follow-up information. An SAE occurring at a different time interval or otherwise considered completely unrelated to a previously reported one should be reported separately as a new event.

Any SAEs experienced after the completion of the safety evaluation follow-up period (as described above) should only be reported to Novartis if the investigator suspects a causal relationship to the study treatment. Information about all SAEs is collected and recorded on the Serious Adverse Event Report Form; all applicable sections of the form must be completed in order to provide a clinically thorough report. The investigator must assess and record the relationship of each SAE to each specific study treatment (if there is more than one study treatment), complete the SAE Report Form in English, and submit the completed form within 24 hours to Novartis. Detailed instructions regarding the SAE submission process and requirements for signatures are to be found in the investigator folder provided to each site.

Follow-up information is submitted in the same way as the original SAE Report. Each re-occurrence, complication, or progression of the original event should be reported as a follow-up to that event regardless of when it occurs. The follow-up information should describe whether the event has resolved or continues, if and how it was treated, whether the blind was broken or not, and whether the patient continued or withdrew from study participation. SAEs occurring after the subject has provided informed consent until the time the subject is deemed a Screen Failure must be reported to Novartis.

If the SAE is not previously documented in the Investigator's Brochure or Package Insert (new occurrence) and is thought to be related to the study treatment, a CMO & PS Department associate may urgently require further information from the investigator for health authority reporting. Novartis may need to issue an Investigator Notification (IN) to inform all investigators involved in any study with the same study treatment that this SAE has been reported. Suspected Unexpected Serious Adverse Reactions (SUSARs) will be collected and reported to the competent authorities and relevant ethics committees in accordance with EU Guidance 2011/C 172/01 or as per national regulatory requirements in participating countries.

3.60 Pregnancy Reporting

To ensure subject safety, each pregnancy occurring after signing the informed consent must be reported to Novartis within 24 hours of learning of its occurrence. The pregnancy should be followed up to determine outcome, including spontaneous or voluntary termination, details of the birth, and the presence or absence of any birth defects, congenital abnormalities, or maternal and/or newborn complications. After the mother has provided consent, the newborn will be followed-up for 12 months. Pregnancy outcomes should be collected for the female partners of any males who took study treatment in this study and the newborn will be followed up to 12 months after delivery date. Consent to report information regarding these pregnancy outcomes should be obtained from the mother.

Pregnancy should be recorded and reported by the investigator to the Novartis Medical Office and Patient Safety (CMO&PS). Pregnancy follow-up should be recorded on the same form and should include an assessment of the possible relationship of the study treatment to any pregnancy outcome. Any SAE experienced during pregnancy must be reported. Pregnancy outcomes should be collected for the female partners of any males who took study treatment in this study. Consent to report information regarding these pregnancy outcomes should be obtained from the mother.

### 3.61 Reporting of Study Treatment Errors Including Misuse/Abuse

Medication errors are unintentional errors in the prescribing, dispensing, administration or monitoring of a medicine while under the control of a healthcare professional, subject or consumer (EMA definition). Misuse refers to situations where the medicinal product is intentionally and inappropriately used not in accordance with the protocol. Abuse corresponds to the persistent or sporadic, intentional excessive use of a medicinal product, which is accompanied by harmful physical or psychological effects. Study treatment errors and uses outside of what is foreseen in the protocol will be recorded on the appropriate CRF irrespective of whether or not associated with an AE/SAE and reported to Safety only if associated with an SAE. Misuse or abuse will be collected and reported in the safety database irrespective of it being associated with an AE/SAE within 24 hours of Investigator's awareness.

TABLE 34
Guidance for capturing the study treatment
errors including misuse/abuse

| | Document in | | |
|---|---|---|---|
| Treatment | Dosing CRF | Document in | Complete |
| error type | (Yes/No) | AE eCRF | SAE form |
| Unintentional | Yes | Only if associated | Only if associated |
| study treatment | | with an AE | with an SAE |
| error | | | |
| Misuse/Abuse | Yes | Yes | Yes, even if not |
| | | | associated with |
| | | | a SAE |

### 3.62 Data Analysis and Statistical Methods

Data from participating centers in this protocol will be combined, so that an adequate number of subjects will be available for analysis. Data will be summarized using descriptive statistics (continuous data) and/or contingency tables (categorical data) for demographic and baseline characteristics, and efficacy, safety, pharmacokinetic and pharmacodynamics measurements. Study data will be analyzed and reported in a primary CSR based on all subjects' data up to the time when all subjects have completed at least six cycles of study treatment or discontinued treatment. Any additional data for subjects continuing to receive study treatment past the data cutoff date for the primary CSR, as allowed by the protocol, will be reported at completion of the study in a final CSR.

Categorical data will be presented as frequencies and percentages. For continuous data, mean, standard deviation, median, minimum, and maximum will be presented. For selected parameters, 25th and 75th percentiles will also be presented. Dose escalation decisions will be based on a synthesis of all relevant data available from all dose levels evaluated in the ongoing study, including safety information, PK, and available PD and preliminary anti-cancer activity data. In particular, the following core data are required to be reviewed for each subject in each cohort to be discussed in the dose escalation meeting, prior to deciding the next steps: (a) Safety: AEs, SAEs, DLTs; (b) Laboratory parameters: hematological parameters (hemoglobin, platelets, WBC, Neutrophils), renal function tests (BUN/urea, creatinine, sodium, potassium), liver function tests (AST, ALT, total bilirubin, ALP)' (c) Dosing information collected during the DLT observation period, prior and concomitant medications, demographics and diagnosis and extent of cancer (which are relevant to inclusion/exclusion criteria), date of visit and end of treatment phase disposition (if end of treatment has already occurred for a subject who is in a cohort to be discussed in the dose escalation meeting).

The following rules will be followed for reporting results unless stated otherwise:
For the escalation part, data will be analyzed by Arm and cohort and/or by indications treated with the same dose or dose combination (dose levels and schedules) will be pooled into a single treatment group by Arm. All summaries, listings, figures and analyses will be performed by treatment group and Arm. Arms to be analyzed are:
   Arm A: Single agent I-57
   Arm B: Combination I-57+PDR001
For the expansion part, all summaries, listings, figures for primary efficacy analysis and safety analyses will be presented by disease group and/or by dose group for one or more disease groups whenever applicable. Subjects from the expansion part will be classified according to the disease group to which they were assigned at baseline based on the disease type.
For PK analyses, PK data from the food effect cohort run-in period (if conducted) will be reported separately.

Screen failure subjects and the reasons for not starting the study treatment will be reported in a listing, but will not be included in any analyses. Details of the statistical analysis and data reporting will be provided in the Statistical Analysis Plan (SAP). Any data analysis carried out independently by the investigator should be submitted to Novartis before publication or presentation.

### 3.63 Analysis Sets

The Full Analysis Set (FAS) and Safety Set (SS) comprise all subjects who received at least one dose of any study drug. Subjects will be analyzed according to the study treatment received where treatment received is defined as the treatment most frequently taken between Study Day 1 and the end of cycle 1, the onset of a DLT or treatment discontinuation whichever occurs first. The Dose-Determining Set (DDS) includes all subjects from the FAS (escalation part) who met the minimum exposure criterion and had sufficient safety evaluations, or experienced a dose limiting toxicity (DLT) during cycle 1. A subject is considered to have a minimum exposure criterion if having received at least 75% of the planned dose of I-57 during Cycle 1 (i.e. at least 21 doses in the QD regimen) for both single agent and combination cohorts, and a full dose of PDR001 for the combination cohorts. Subjects who do not experience a DLT during cycle 1 are considered to have sufficient safety evaluations if they have been observed for more than 1 cycle, and are considered by both the sponsor and investigators to have enough safety data to conclude that a DLT did not occur. Subjects will be analyzed according to the study treatment received as defined for FAS.

The Pharmacokinetic analysis set (PAS) for Treatment period includes all subjects who provide an evaluable PK profile. A profile is considered evaluable if all of the following conditions are satisfied:
Subject receives one of the planned treatments
Subject provides at least one evaluable post-dose concentration
Subject does not vomit within 4 hours after dosing of I-57

### 3.64 Subject Demographics and Other Baseline Characteristics

Demographic and other baseline data including disease characteristics will be listed and summarized descriptively by treatment arm for the FAS. Relevant medical histories and current medical conditions at baseline will be summarized by system organ class and preferred term, by treatment group.

### 3.65 Treatments

The Safety set will be used for the analyses below. Categorical data will be summarized as frequencies and percentages. For continuous data, mean, standard deviation, median, 25th and 75th percentiles, minimum, and maximum will be presented. The duration of exposure to each treatment arm as well as the dose intensity (computed as the ratio of actual cumulative dose received and actual duration of exposure) and the relative dose intensity (computed as the ratio of dose intensity and planned dose intensity) will be summarized by means of descriptive statistics using the safety set.

Concomitant medications and significant non-drug therapies prior to and after the start of the study treatment will be listed by treatment group. The number of subjects with dose adjustments (reductions, interruption, or permanent discontinuation) and the reasons will be summarized by treatment group and all dosing data will be listed.

The primary objective is to characterize the safety and tolerability of single agent I-57 and I-57 in combination with PDR001 and identify a recommended dose for future studies for each treatment arm. The primary endpoints are described in Table 35.

TABLE 35
Primary endpoints

| Objective | Primary endpoint |
|---|---|
| Safety | Incidence and severity of AEs and SAEs, |
| | including changes in laboratory values, |
| | vital signs, and ECGs |
| Tolerability | Dose interruptions, reductions, and dose |
| | intensity |
| Identification of | Incidence of dose limiting toxicities (DLTs) |
| recommended dose | in Cycle 1 |

Estimation of the MTD in the dose-escalation part of the study will be based upon the estimation of the probability of DLT in Cycle 1 for subjects in the dose-determining set. This probability is estimated by the statistical models in Section 3.66. A dose-limiting toxicity (DLT) is defined as an adverse event or abnormal laboratory value assessed as clinically relevant, occurring ≤28 days following the first administration of study treatment as defined in Section 3.30, Table 20.

### 3.66 Statistical Model, Hypothesis, and Method of Analysis

Separate adaptive BHLRMs (single agent and combination) guided by the EWOC criteria will be used to make dose recommendations and estimate the appropriate MTD during the dose escalation part of the study. The BHLRM will be fit on the dose-limiting toxicity data (i.e. absence or presence of DLT) during the DLT window accumulated throughout the dose escalation to model the dose-toxicity relationship.

I-57 Single agent BHLRM (Arm A)—The Arm A dose escalation will be guided by a BHLRM based on the first Cycle DLT data of the study treatment. This model estimates the relationship between dose and the probability of a subject experiencing a DLT in fasted conditions following a QD regimen (stratum 1), and in fed condition (stratum 2). The latter stratum will be introduced if a clinically relevant food effect is observed in food effect cohort during the dose escalation part. The BHLRM will allow for both full exchangeability between the strata parameters and non-exchangeability between strata parameters. In this way the model has the flexibility to allow for the case in which the dose-toxicity relationship is either similar (i.e. exchangeable) or different (i.e. non-exchangeable) for the two strata. In other words, the model structure will allow partially borrowing of information between the two strata, which in turn leads to improved estimation of the dose toxicity relationship.

Combination BHLRM model (Arm B)— The Arm B dose escalation will be guided by a meta-analytic-combined (MAC) BHLRM based on the first Cycle DLT data of the study treatment. The model will integrate both single-agent and combination toxicity parts under either fasted or fed conditions. Both historical data and concurrent data are incorporated into the model. For different regimens and/or fed conditions, a plausible between-cohorts heterogeneity will be assumed, allowing for non-exchangeability across trial parameters. The strata under fed condition will be introduced if a clinically relevant food effect is observed in food effect cohort during the dose escalation part.

### 3.67 Assessment of Subject Risk

After each cohort of subjects, the posterior distribution for the risk of DLT for new subjects at doses of interest will be evaluated. The posterior distributions will be summarized to provide the posterior probability that the risk of DLT lies within the following intervals: (a) [0%, 16%] under-dosing; (b) [16%, 33%] targeted toxicity; and (c) [33%, 100%] excessive toxicity. Dosing decisions are guided by the escalation with overdose control principal (Babb et al, Stat Med. 1998). A dose may only be used for newly enrolled subjects if the risk of excessive toxicity at that dose is less than 25%. Decisions on dose escalation will follow the procedure as outlined in Section 3.27.

### 3.68 Listing/Summary of DLTs

DLTs will be listed and their incidence summarized by primary system organ class, worst grade based on the CTCAE version 5.0, type of adverse event, and by treatment group in the dose escalation. The dose-determining set will be used for these summaries.

### 3.69 Safety Objectives

Analysis set and grouping for the analyses. For summaries of DLTs the DDS will be used, for all other safety analyses the safety set will be used. All listings and tables will be presented by treatment group. The overall observation period will be divided into three mutually exclusive segments:
1. pre-treatment period: from day of subject's informed consent to the day before first administration of study treatment
2. on-treatment period: from day of first administration of study treatment to 30 days after date of last administration of study treatment (including start and stop date)
3. post-treatment period: from 31 days after date of last administration of study treatment

Safety summaries will primarily be based on all data from the on-treatment period. Following last administration of PDR001 in the combination arm, adverse events (including serious adverse events), and new antineoplastic therapies are collected for a period of 150 days. Following start of new antineoplastic therapy only treatment related adverse events will be collected. Select summaries of related adverse events will be produced for the combined on-treatment and post-treatment periods for subjects receiving PDR001.

### 3.70 Adverse Events

All information obtained on adverse events will be displayed by treatment group and subject. The number (and percentage) of subjects with treatment emergent adverse events (events started after the first dose of study medication or events present prior to start of treatment but increased in severity based on preferred term) will be summarized in the following ways: (a) by treatment, primary system organ class and preferred term and (b) by treatment, primary system organ class, preferred term and maximum severity. Separate summaries will be provided for study medication related adverse events, death, serious adverse events and other significant adverse events leading to discontinuation.

A subject with multiple adverse events within a primary system organ class is only counted once towards the total of the primary system organ class. Summary tables for adverse events (AEs) will include only AEs that started or worsened during the on-treatment period, the treatment-emergent AEs. Additional select summaries will be produced using all related AEs that started or worsened during the combined on-treatment and post-treatment periods. The incidence of treatment-emergent adverse events (new or worsening from baseline) will be summarized by system organ class and or preferred term, severity (based on CTCAE grades), type of adverse event, relation to study treatment. Serious adverse events, non-serious adverse events and adverse events of special interest (AESI) during the on-treatment period will be tabulated. All deaths (on-treatment and post-treatment) will be summarized. All AEs, deaths and serious adverse events (including those from the pre and post-treatment periods) will be listed and those collected during the pre-treatment and post-treatment period will be flagged.

### 3.71 ECG

All ECG data will be listed by treatment group, subject and visit/time, abnormalities will be flagged. Summary statistics will be provided by treatment and visit/time.

### 3.72 Vital Signs

All vital signs data will be listed by treatment group, subject and visit/time, abnormalities will be flagged. Summary statistics will be provided by treatment and visit/time.

### 3.73 Clinical Laboratory Evaluations

All laboratory data will be listed by treatment group, subject, and visit/time and if normal ranges are available abnormalities will be flagged. Summary statistics will be provided by treatment and visit/time. Shift tables using the low/normal/high/(low and high) classification will be used to compare baseline to the worst on-treatment value.

Grading of laboratory values will be assigned programmatically as per NCI Common Terminology Criteria for Adverse Events (CTCAE) version 5.0. The calculation of CTCAE grades will be based on the observed laboratory values only, clinical assessments will not be taken into account.

CTCAE Grade 0 will be assigned for all non-missing values not graded as 1 or higher. Grade 5 will not be used.

For laboratory tests where grades are not defined by CTCAE v.5.0, results will be categorized as low/normal/high based on laboratory normal ranges.

The following summaries will be generated separately for hematology, and biochemistry tests:
Listing of all laboratory data with values flagged to show the corresponding CTCAE v.5.0 grades if applicable and the classifications relative to the laboratory normal ranges

For laboratory tests where grades are defined by CTCAE v.5.0
Worst post-baseline CTCAE grade (regardless of the baseline status). Each subject will be counted only once for the worst grade observed post-baseline.
Shift tables using CTCAE v.5.0 grades to compare baseline to the worst on-treatment value For laboratory tests where grades are not defined by CTCAE v.5.0,
Shift tables using the low/normal/high/(low and high) classification to compare baseline to the worst on-treatment value.

### 3.74 Tolerability

Tolerability of study drug will be assessed by summarizing the number of and reasons for dose delays and dose reductions. Dose intensity will also be tabulated by treatment group.

### 3.75 Handling of Missing Values/Censoring/Discontinuations

Subjects who are ineligible for the DDS will be removed from the DLT analysis and additional subjects may be recruited. Their data will be used for all remaining analyses.

### 3.76 Efficacy and or Pharmacodynamic Endpoint(s)

Tumor response will be determined per local investigator's assessment. All efficacy endpoints will be defined and analyzed based on tumor assessment by RECIST 1.1 and iRECIST. iRECIST defines progression on the basis of RECIST 1.1; however, progression requires confirmation. The event date to be used for calculation of PFS is the first date at which progression criteria are met provided that progression is confirmed at the next assessment. If progression is followed by a better response (SD, PR or CR) then the bar is reset and progression needs to occur again and then be confirmed. If progression is not confirmed and there is no subsequent better response, then the first progression date should still be used in the following scenarios:
the subject stops protocol treatment because they were not judged to be clinically stable
no further response assessments are done (because of subject refusal, protocol noncompliance, or subject death)
the next timepoint responses are all unconfirmed progression and confirmation never occurs
the subject dies from their cancer.

Individual lesion measurements and overall response assessments will be listed by subject and assessment date. BOR and PFS will be listed by subject.

The following analyses will be presented by treatment group for subjects treated in each treatment arm expansion part. BOR and ORR will also be summarized by treatment group for all subjects treated in each treatment arm dose escalation part.
BOR will be summarized
ORR and DOR will be summarized with an accompanying 90% exact binomial confidence interval (CI).
For PFS the survival function will be estimated using the Kaplan-Meier (KM) product limit method and displayed graphically. Median duration, with a two-sided 90% CI, and 25th and 75th percentiles (Brookmeyer and Crowley, 1982) (Klein and Moeschberger, 1997) will be presented. KM estimates of survival proportions at specified time points, along with corresponding 90% CIs (Kalbfleisch and Prentice, 1980) will also be provided.

### 3.77 Pharmacokinetics

All subjects who have evaluable PK data will be included in the PK data analysis. PK parameters will be determined using non-compartmental method(s). PK parameters such as those listed in Table 36 will be estimated and reported, when applicable. Exploratory PK and PK/PD analysis may be conducted based on preliminary data prior to database lock, and nominal time instead of actual elapsed time may be used. Concentration data of each component of the combination treatments will be listed by treatment, subject, and visit/sampling time point. Descriptive summary statistics will be provided by treatment and visit/sampling time point, including the frequency (n, %) of concentrations below the LLOQ and reported as zero.

Summary statistics will include mean (arithmetic and geometric), SD, CV (arithmetic and geometric), median, minimum and maximum. Concentrations below LLOQ will be treated as zero in summary statistics and for PK parameter calculations. Descriptive graphical plots of individual concentration versus time profiles and mean concentration versus time profiles will be generated. Pharmacokinetic parameters will be listed by treatment and subject. Descriptive summary statistics will include mean (arithmetic and geometric), SD, and CV (arithmetic and geometric), median, minimum and maximum. An exception to this is Tmax where median, minimum and maximum will be presented. The effect of food on PK profiles of I-57 single agent under fasted and fed conditions will be evaluated based on analysis plan defined in Statistical Analysis Plan.

TABLE 36
Non-compartmental pharmacokinetic parameters

| | |
|---|---|
| AUClast | The AUC from time zero to the last measurable concentration |
| | sampling time (tlast) (mass × time × volume-1) |
| AUCinf | The AUC from time zero to infinity (mass × time × |
| | volume-1) |
| AUCtau | The AUC calculated to the end of a dosing interval (tau) |
| | at steady-state (amount × time × volume-1) |
| AUC₀₋₄₈ | Area under the concentration-time curve during a dosing |
| | interval [mass × time × volume-1] (for |
| | cohorts with food effect run-in period and 48 hour |
| | PK profiles) |
| Cmax | The maximum (peak) observed plasma, blood, serum, or |
| | other body fluid drug concentration after single dose |
| | administration (mass × volume-1) |
| Ctrough | The observed plasma, blood, serum, or other body fluid |
| | drug concentration before the next dose is administered |
| | (pre-dose concentration) (mass × volume-1) |
| Tmax | The time to reach maximum (peak) plasma, blood, serum, |
| | or other body fluid drug concentration after single |
| | dose administration (time) |
| T½ | The elimination half-life associated with the terminal |
| | slope (λz) of a semi logarithmic concentration-time |
| | curve (time). Use qualifier for other half-lives |
| CL/F | The total body clearance of drug from the plasma |
| | (volume × time-1) |
| Vz/F | The apparent volume of distribution during terminal phase |
| | (associated with λz) (volume) |
| Racc | Cmax (multiple Dose)/Cmax (single dose) or AUC (multiple |
| | Dose)/AUC (single dose) |

### 3.78 Advanced Data Analysis and Exploratory Analysis

If feasible, exploratory PK analysis may be performed using non-linear mixed effects modeling to characterize the population PK and to estimate inter-individual variability in the study population. In addition, the emerging PK data may be compared to historical data from relevant studies or clinical reports to assess DDI potential.

Any analyses performed will be specified either in the Statistical Analysis Plan (SAP) prior to clinical database lock in an amendment to the SAP or in a stand-alone analysis plan document. All analyses will be reported either in the CSR or a stand-alone report.

### 3.79 PK/PD Relationships

The concentration-PD relationship will be evaluated by a linear mixed effects model in order to characterize the relationship between PD changes from baseline and the plasma concentration of I-57. If feasible, effects of covariates (such as age, gender, race, presence of concomitant medications) will be evaluated as well. In addition, the potential correlation between I-57 exposure and other endpoints (major safety, efficacy and biomarker parameters) will be evaluated. This will be done in two steps. First, a descriptive analysis will be performed graphically between PK exposure values and major safety, efficacy, and biomarker parameters (either as categories or continuous variables). If any potential correlation is identified, further investigation will be performed using a mechanism-based modeling approach, as appropriate.

### 3.80 Biomarkers

The objectives include the assessment of the pharmacodynamics (PD) effect of each treatment arm in tumor tissue and PBMCs. For assessment of PD effects of each treatment arm in tumor, pre- and post-treatment tumor biopsies will be examined for expression of immunological markers such as, but not restricted to IKZF2, TILs (e.g. CD8), PD-L1 (see Section 3.1). All biomarker data summary and analysis will be based on the Full Analysis Set (FAS). All biomarker data collected will be at the very least listed for each subject by treatment group. If enough data are collected for any parameter, then summary statistics will be provided.

### 3.81 Doses Escalation Part

Cohorts of 3 to 6 evaluable subjects with NSCLC and melanoma, and NPC will be enrolled in the dose escalation part including at least six subjects at the MTD/RD level, as described in the dose escalation section (Section 3.2). Multiple cohorts may be sequentially enrolled to the same dose level. Additional cohorts of 1 to 6 subjects may be enrolled at any dose level at or below a previously tested dose level, and satisfying EWOC, for further elaboration of safety and pharmacokinetic parameters as required. At least 21 subjects should be treated in the dose escalation part for the single agent I-57 arm and 12 subjects for the combination of I-57 and PDR001 arm, for the model to have reasonable operating characteristics relating to its MTD recommendation.

The MTD declaration will occur when the following conditions are met:
1) at least 6 treated subjects at the dose to be determined as the MTD
2) this dose satisfies one of the following conditions:
   a) the posterior probability of targeted toxicity at this dose exceeds 50% and is the highest among potential doses, or
   b) minimum of 21 treated subjects for the single agent I-57 arm and 12 subjects for the combination of I-57 and PDR001 arm on the trial.

### 3.82 Dose Expansion Part

For the dose expansion part, single agent I-57 and the I-57 combination with PDR001 part will initially enroll 20 subjects for indications of NSCLC, melanoma and NPC for each part. Additional subjects may be enrolled into the NSCLC and melanoma expansion arms to capture an adequate number of subjects with tumor infiltration (defined as subject with a CD8≥2%), where high degradation is expected. For the cohorts of NSCLC and melanoma, up to 40 subjects may be enrolled in order to enroll adequate subjects with infiltrated tumors. Analysis will be done in the pooled and CD8≥2% subgroups. Cohorts of TNBC and/or mssCRC with 15 subjects each may be enrolled. The probability to detect a special interest of adverse event (AE) with a true incidence rate of 10% is 79.4% with 15 subjects, 87.8% with 20 subjects, and 98.5% with 40 subjects (Table 37).

TABLE 37
Probability to detect at least one special adverse event of interest

Number of subjects

| AE rate | 15 | 20 | 30 | 40 |
|---|---|---|---|---|
| 2.5% | 0.316 | 0.397 | 0.532 | 0.637 |
| 5% | 0.537 | 0.642 | 0.785 | 0.871 |
| 10% | 0.794 | 0.878 | 0.958 | 0.985 |
| 15% | 0.913 | 0.961 | 0.992 | 0.998 |

3.83 Efficacy Assessment for Dose Expansion Groups of I-57 Single Agent and in Combination with PDR001

Table 38 shows the 95% credible intervals for a sample size of N=15, 20 and 40 for different number of responses using a minimally informative beta distribution as prior distribution with parameters b=1 and a=0.34 and prior mean 0.253. Note that the final interval will depend on the final sample size and the number of responses observed in each group.

TABLE 38
95% credible intervals for I-57 in combination
with PDR001 (N = 15, 20 and 40)

| | | | |
|---|---|---|---|
| Observed | Observed | | 95% credible |
| responses | response rate | Posterior mean | interval |

N = 15

| | | | |
|---|---|---|---|
| 0 | 0.00 | 0.021 | (0, 0.122) |
| 1 | 0.07 | 0.082 | (0.005, 0.25) |
| 2 | 0.13 | 0.143 | (0.024, 0.343) |
| 3 | 0.20 | 0.204 | (0.053, 0.424) |
| 4 | 0.27 | 0.266 | (0.089, 0.496) |
| 5 | 0.33 | 0.327 | (0.13, 0.564) |
| 6 | 0.40 | 0.388 | (0.175, 0.627) |
| 7 | 0.47 | 0.449 | (0.224, 0.686) |
| 8 | 0.53 | 0.51 | (0.277, 0.741) |

N = 20

| | | | |
|---|---|---|---|
| 0 | 0 | 0.016 | (0, 0.094) |
| 1 | 0.05 | 0.063 | (0.004, 0.195) |
| 2 | 0.10 | 0.11 | (0.018, 0.269) |
| 3 | 0.15 | 0.157 | (0.039, 0.334) |
| 4 | 0.20 | 0.203 | (0.066, 0.393) |
| 5 | 0.25 | 0.25 | (0.096, 0.449) |
| 6 | 0.30 | 0.297 | (0.128, 0.502) |
| 7 | 0.35 | 0.344 | (0.163, 0.552) |
| 8 | 0.40 | 0.391 | (0.201, 0.600) |

N = 40

| | | | |
|---|---|---|---|
| 0 | 0 | 0.008 | (0, 0.049) |
| 1 | 0.025 | 0.032 | (0.002, 0.103) |
| 2 | 0.05 | 0.057 | (0.009, 0.144) |
| 3 | 0.075 | 0.081 | (0.019, 0.18) |
| 4 | 0.1 | 0.105 | (0.032, 0.214) |
| 5 | 0.125 | 0.129 | (0.047, 0.246) |
| 6 | 0.15 | 0.153 | (0.062, 0.276) |
| 7 | 0.175 | 0.178 | (0.078, 0.306) |
| 8 | 0.2 | 0.202 | (0.096, 0.335) |
| 9 | 0.225 | 0.226 | (0.114, 0.363) |
| 10 | 0.25 | 0.25 | (0.132, 0.391) |

If assuming a 20% ORR, the chance of no subjects responding is 1.2%, 3.5% and 6.8% with 20 subjects, 15 subjects and 12 subjects respectively. Assuming a 35% ORR, the chance of no subjects responding is <0.1%, 0.16% with 20 subjects and 15 subjects respectively.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of treating or preventing cancer comprising administering to a patient in need thereof a combination comprising,
(a) a compound of Formula (Ic): 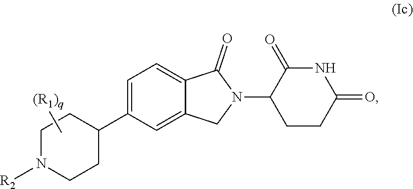
or a pharmaceutically acceptable salt, hydrate, solvate, prodrug, stereoisomer, or tautomer thereof:
wherein:
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R_{6′}, are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more Rn, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S; and
q is 0, 1, 2, 3, or 4; and
(b) a second therapeutic agent.

2. The method according to claim 1, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

3. The method according to claim 1 or 2, wherein the cancer is selected from non-small cell lung cancer (NSCLC), melanoma, triple-negative breast cancer (TNBC), nasopharyngeal cancer (NPC), and microsatellite stable colorectal cancer (mssCRC).

4. The method according to any one of claims 1-3, wherein the compound and the second agent are administered simultaneously, separately, or over a period of time.

5. The method according to any one of claims 1-4, wherein the amount of the compound of Formula (Ic), or a pharmaceutically acceptable salt, hydrate, solvate, prodrug, stereoisomer, or tautomer thereof, administered to the patient in need thereof is effective to treat or prevent the cancer.

6. The method according to any one of claims 1-5, wherein the amounts of: (a) compound of Formula (Ic), or a pharmaceutically acceptable salt, hydrate, solvate, prodrug, stereoisomer, or tautomer thereof; and (b) the second therapeutic agent, administered to the patient in need thereof are effective to treat or prevent the cancer.

7. The method according to any one of claims 1-6, wherein the compound of Formula (Ic) is selected from 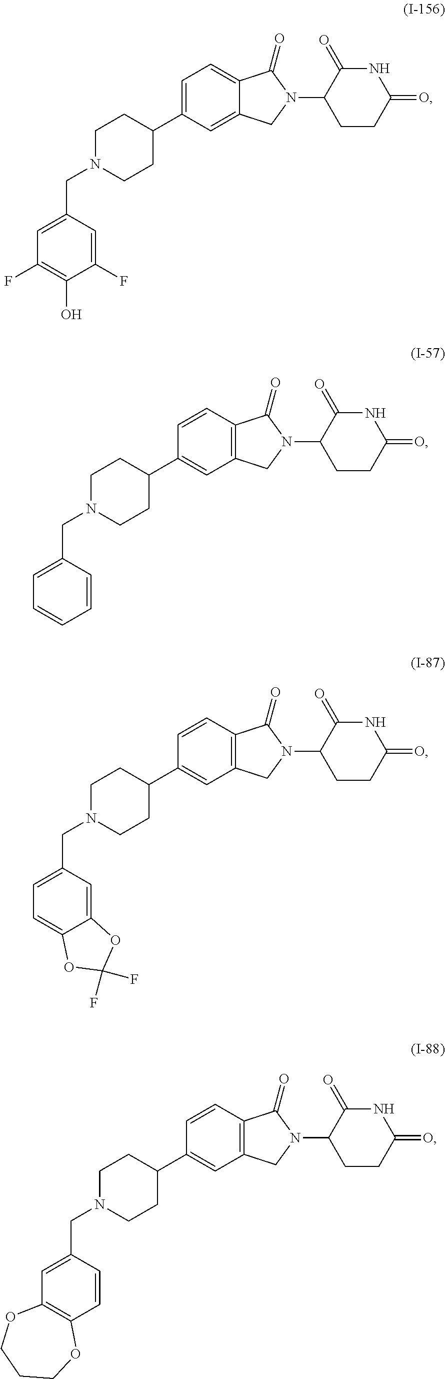 [SMILES: O=C1CCC(N2Cc3cc(C4CCN(Cc5cc(F)c(O)c(F)c5)CC4)ccc3C2=O)C(=O)N1 ; O=C1CCC(N2Cc3cc(C4CCN(Cc5ccccc5)CC4)ccc3C2=O)C(=O)N1 ; O=C1CCC(N2Cc3cc(C4CCN(Cc5ccc6c(c5)OC(F)(F)O6)CC4)ccc3C2=O)C(=O)N1 ; O=C1CCC(N2Cc3cc(C4CCN(Cc5ccc6c(c5)OCCCO6)CC4)ccc3C2=O)C(=O)N1] 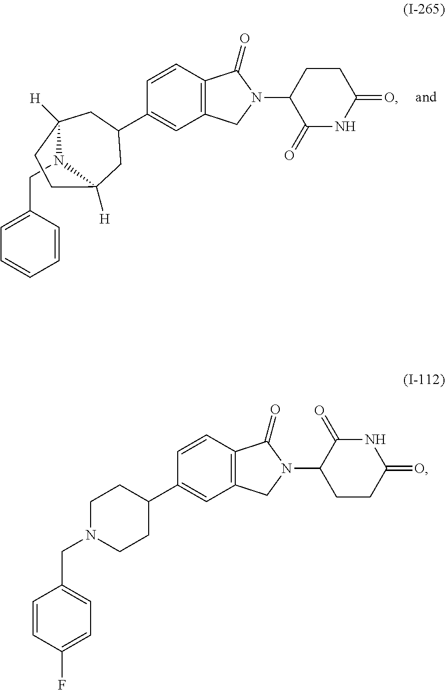 [SMILES: O=C1CCC(N2Cc3cc(C4C[C@H]5CC[C@@H](C4)N5Cc4ccccc4)ccc3C2=O)C(=O)N1 ; O=C1CCC(N2Cc3cc(C4CCN(Cc5ccc(F)cc5)CC4)ccc3C2=O)C(=O)N1]
or a pharmaceutically acceptable salt, hydrate, solvate, prodrug, stereoisomer, or tautomer thereof.

8. The method according to any one of claims 1-7, wherein the compound of Formula (Ic) is Compound I-156.

9. The method according to any one of claims 1-7, wherein the compound of Formula (Ic) is Compound I-57.

10. The method according to any one of claims 1-7, wherein the compound of Formula (Ic) is Compound I-87.

11. The method according to any one of claims 1-7, wherein the compound of Formula (Ic) is Compound I-88.

12. The method according to any one of claims 1-7, wherein the compound of Formula (Ic) is Compound I-265.

13. The method according to any one of claims 1-7, wherein the compound of Formula (Ic) is Compound I-112.

14. The method according to any one of claims 1-13, wherein the second therapeutic agent is an immunomodulator.

15. The method according to claim 14, wherein the second therapeutic agent is an immune checkpoint inhibitor.

16. The method according to claim 15, wherein the second therapeutic agent is a PD-1 inhibitor.

17. The method according to claim 16, wherein the PD-1 inhibitor is PDR001, Nivolumab, Pembrolizumab, Pidilizumab, MEDI0680, REGN2810, TSR-042, PF-06801591, BGB-A317, BGB-108, INCSHR1210, or AMP-224.

18. The method according to claim 17, wherein the PD-1 inhibitor is PDR001.

19. The method according to any one of claims 1-18, wherein the compound is administered at a dose of about 2 mg per day, or about 4 mg per day, or about 10 mg per day, or about 20 mg per day, or about 40 mg per day, or about 80 mg per day, or about 160 mg per day, or about 320 mg per day.

20. The method according to any one of claims 1-19, wherein the compound is administered orally.

21. The method according to any one of claims 1-20, wherein the second therapeutic agent is administered at a dose of about 100 mg once every four weeks, or about 200 mg once every four weeks, or about 300 mg once every four weeks, or about 400 mg once every four weeks, or about 500 mg once every four weeks.

22. The method according to any one of claims 1-21, wherein the second therapeutic agent is administered at a dose of about 400 mg once every four weeks.

23. The method according to any one of claims 1-22, wherein the second therapeutic agent is administered intravenously.

24. The method according to any one of claims 1-23, wherein the compound is administered orally at a dose of about 2 mg per day, or about 4 mg per day, or about 10 mg per day, or about 20 mg per day, or about 40 mg per day, or about 80 mg per day, or about 160 mg per day, or about 320 mg per day; and the second therapeutic agent is administered intravenously at a dose of about 100 mg once every four weeks, or about 200 mg once every four weeks, or about 300 mg once every four weeks, or about 400 mg once every four weeks, or about 500 mg once every four weeks.

25. A method of treating or preventing cancer comprising administering to a patient in need thereof a compound of Formula (Ic): 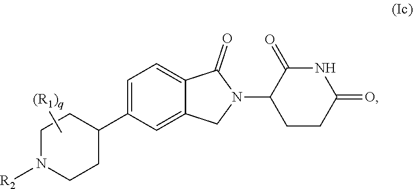
or a pharmaceutically acceptable salt, hydrate, solvate, prodrug, stereoisomer, or tautomer thereof:
wherein:
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₅)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₃-C₇)cycloalkyl ring or a 5- to 7-membered heterocycloalkyl ring comprising 1 to 3 heteroatoms selected from O, N, and S optionally substituted with one or more R₁₀;
R₆ and R_{6′} are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S; and
q is 0, 1, 2, 3, or 4;
wherein the compound of Formula (Ic) is administered orally at a dose of about 2 mg per day, or about 4 mg per day, or about 10 mg per day, or about 20 mg per day, or about 40 mg per day, or about 80 mg per day, or about 160 mg per day, or about 320 mg per day.

26. The method according to claim 25, wherein the amount of the compound of Formula (Ic), or a pharmaceutically acceptable salt, hydrate, solvate, prodrug, stereoisomer, or tautomer thereof, is effective to treat or prevent the cancer.

27. The method according to claim 25 or 26, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

28. The method according to any one of claims 25-27, wherein the cancer is selected from non-small cell lung cancer (NSCLC), melanoma, triple-negative breast cancer (TNBC), nasopharyngeal cancer (NPC), and microsatellite stable colorectal cancer (mssCRC).

29. The method according to any one of claims 25-28, wherein the compound of Formula (Ic) is selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof.

30. The method according to any one of claims 25-29, wherein the compound of Formula (Ic) is Compound I-156.

31. The method according to any one of claims 25-29, wherein the compound of Formula (Ic) is Compound I-57.

32. The method according to any one of claims 25-29, wherein the compound of Formula (Ic) is Compound I-87.

33. The method according to any one of claims 25-29, wherein the compound of Formula (Ic) is Compound I-88.

34. The method according to any one of claims 25-29, wherein the compound of Formula (Ic) is Compound I-265.

35. The method according to any one of claims 25-29, wherein the compound of Formula (Ic) is Compound I-112.

36. The method according to any one of claims 25-35 further comprising a second therapeutic agent.

37. The method according to claim 36, wherein the compound and the second agent are administered simultaneously, separately, or over a period of time.

38. The method according to claim 36 or 37, wherein the second therapeutic agent is an immunomodulator.

39. The method according to claim 38, wherein the immunomodulator is an immune checkpoint inhibitor.

40. The method according to claim 39, wherein the immune checkpoint inhibitor is a PD-1 inhibitor.

41. The method according to claim 40, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

42. The method according to claim 41, wherein the PD-1 inhibitor is PDR001.

43. The method according to any one of claims 36-42, wherein the second therapeutic agent is administered at a dose of about 100 mg once every four weeks, or about 200 mg once every four weeks, or about 300 mg once every four weeks, or about 400 mg once every four weeks, or about 500 mg once every four weeks.

44. The method according to any one of claims 36-43, wherein the second therapeutic agent is administered at a dose of about 400 mg once every four weeks.

45. The method according to any one of claims 36-44, wherein the second therapeutic agent is administered intravenously.

46. The method according to any one of claims 36-45, wherein the amounts of: (a) the compound of Formula (Ic), or a pharmaceutically acceptable salt, hydrate, solvate, prodrug, stereoisomer, or tautomer thereof; and (b) the second therapeutic agent, are effective to treat or prevent the cancer.

47. The method according to any one of claims 36-46, wherein the amounts of: (a) Compound I-156, Compound I-57, Compound I-87, Compound I-88, Compound I-265, or Compound I-112, or a pharmaceutically acceptable salt, hydrate, solvate, prodrug, stereoisomer, or tautomer thereof; and (b) the second therapeutic agent, are effective to treat or prevent the cancer.

48. The method according to any one of claims 1-47, wherein the method further comprises measuring the level of at least one biomarker selected from IKZF2, PD-L1, CD8, and FOXP3.

49. The method according to claim 48, wherein the level of IKZF2 is reduced.

50. The method according to any one of claims 1-49, wherein the patient was previously treated with an anti-PD-1/PD-L1 therapy.

51. The method according to any one of claims 1-50, wherein the patient being treated for NSCLC or melanoma, or a combination thereof, was primarily refractory to anti-PD-1/PD-L1 therapy agent showing no significant radiologic response during treatment with an anti-PD-1/PD-L1 agent <6 months prior to disease progression.

52. The method according to any one of claims 1-50, wherein the patient being treated for NPC, mssCRC, or TNBC, or a combination thereof, was naive to anti-PD-1/PD-L1 therapy.

53. The method according to any one of claims 1-52, wherein the patient has not been treated with an IKZF2 targeting agent.

54. The method according to any one of claims 1-53, wherein the patient does not show the presence of symptomatic central nervous system (CNS) metastases, or CNS metastases requiring local CNS-directed therapy (such as radiotherapy or surgery), or increasing doses of corticosteroids within 2 weeks prior to the time of the first administration of the compound or the combination comprising the compound and a second agent.

55. The method according to any one of claims 1-54, wherein the patient does not have a history of severe hypersensitivity reactions to any ingredient of study drug(s) and other mAbs and/or their excipients.

56. The method according to any one of claims 1-55, wherein the patient does not have clinically significant cardiac disease or impaired cardiac function.

57. The method according to any one of claims 1-56, wherein the patient does not have any one of the following clinically significant cardiac disease or impaired cardiac function:
(i) clinically significant and/or uncontrolled heart disease such as congestive heart failure requiring treatment with NYHA grade ≥2;
(ii) uncontrolled hypertension or clinically significant arrhythmia;
(iii) QT interval corrected by Fridericia's formula (QTcF) >450 msec in male patients, or >460 msec female patients;
(iv) QTc that is not assessable;
(v) congenital long QT syndrome;
(vi) history of familial long QT syndrome or known family history of as Torsades de Pointes; and
(vii) acute myocardial infarction or unstable angina pectoris ≤3 months prior to the time of the first administration of the compound or the combination comprising the compound and a second agent.

58. The method according to any one of claims 1-57, wherein the patient does not have HIV infection.

59. The method according to any one of claims 1-58, wherein the patient does not have hepatitis B virus (HBV) infection.

60. The method according to any one of claims 1-59, wherein the patient does not have hepatitis C virus (HCV) infection.

61. The method according to any one of claims 1-60, wherein the patient does not have active, known, or suspected autoimmune disease.

62. The method according to any one of claims 1-61, wherein the patient does not have the presence or history of interstitial lung disease or interstitial pneumonitis, including clinically significant radiation or drug-induced pneumonitis.

63. The method according to any one of claims 1-62, wherein the patient has not been treated with
(i) a cytotoxic or targeted antineoplastics within 3 weeks prior to the time of the first administration of the compound or the combination comprising the compound and a second agent;
(ii) systemic chronic steroid therapy (>10 mg/day prednisone or equivalent) or any other immunosuppressive therapy within 7 days prior to the time of the first administration of the compound or the combination comprising the compound and a second agent;
(iii) radiotherapy within 2 weeks prior to the time of the first administration of the compound or the combination comprising the compound and a second agent; or
(iv) any immunosuppressive medication that would interfere with the action of the compound or the combination comprising the compound and a second agent;
or a combination thereof.

64. The method according to any one of claims 1-63, wherein the patient has not been using any live vaccines against infectious diseases within 4 weeks prior to the time of the first administration of the compound or the combination comprising the compound and a second agent; or using hematopoietic colony-stimulating growth factors thrombopoietin mimetics or erythroid stimulating agents within ≤2 weeks prior to the time of the first administration of the compound or the combination comprising the compound and a second agent.

65. A pharmaceutical combination comprising,
(a) a compound of Formula (Ic): 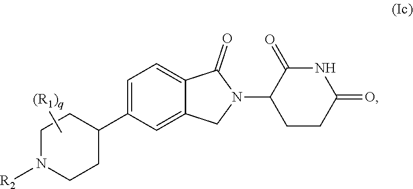
or a pharmaceutically acceptable salt, hydrate, solvate, prodrug, stereoisomer, or tautomer thereof:
wherein:
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is H, (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
each R₄ is independently selected from —C(O)OR₆, —C(O)NR₆R_{6′}, —NR₆C(O)R_{6′}, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R₆, are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S; and
q is 0, 1, 2, 3, or 4; and
(b) a second therapeutic agent.

66. The combination according to claim 65, wherein the compound of Formula (Ic) is selected from Compound I-156, Compound I-57, Compound I-87, Compound I-88, Compound I-265, and Compound I-112, or a pharmaceutically acceptable salt, hydrate, solvate, prodrug, stereoisomer, or tautomer thereof.

67. The combination according to claim 65 or 66, wherein the compound of Formula (Ic) is Compound I-156.

68. The combination according to claim 65 or 66, wherein the compound of Formula (Ic) is Compound I-57.

69. The combination according to claim 65 or 66, wherein the compound of Formula (Ic) is Compound I-87.

70. The combination according to claim 65 or 66, wherein the compound of Formula (Ic) is Compound I-88.

71. The combination according to claim 65 or 66, wherein the compound of Formula (Ic) is Compound I-265.

72. The combination according to claim 65 or 66, wherein the compound of Formula (Ic) is Compound I-112.

73. The combination according to any one of claims 65-72, wherein the second therapeutic agent is an immunomodulator.

74. The combination according to claim 73, wherein the immunomodulator is an immune checkpoint inhibitor.

75. The combination according to claim 74, wherein the immune checkpoint inhibitor is a PD-1 inhibitor.

76. The combination according to claim 75, wherein the PD-1 inhibitor PDR001, Nivolumab, Pembrolizumab, Pidilizumab, MEDI0680, REGN2810, TSR-042, PF-06801591, BGB-A317, BGB-108, INCSHR1210, or AMP-224.

77. The combination according to claim 76, wherein the PD-1 inhibitor is PDR001.

78. The combination according to any one of claims 65-77, wherein the combination comprises about 2 mg, or about 10 mg, or about 20 mg, or about 40 mg, or about 80 mg, or about 160 mg, or about 320 mg of the compound.

79. The combination according to any one of claims 65-78, wherein the combination comprises about 100 mg, or about 200 mg, or about 300 mg, or about 400 mg, or about 500 mg of the second therapeutic agent.

80. The combination according to any one of claims 65-79, wherein the combination comprises about 2 mg, or about 10 mg, or about 20 mg, or about 40 mg, or about 80 mg, or about 160 mg, or about 320 mg of the compound; and about 100 mg, or about 200 mg, or about 300 mg, or about 400 mg, or about 500 mg of the second therapeutic agent.

81. A combination according to any one of claims 65-80 for use in the treatment or prevention of cancer.

82. Use of the combination according to any one of claims 65-80 for the manufacture of a medicament for treating or preventing cancer.

83. Use of the combination according to any one of claims 65-80 for the treatment or prevention of cancer.

84. The combination according to claim 81 or the use according to claims 82 or 83, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST).

85. A method of treating or preventing cancer comprising administering to a patient in need thereof a compound that has degrader activity for IKZF2 in combination with one or more therapeutic agents, wherein the therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof.

86. The method of claim 85, wherein the one or more therapeutic agents is selected from a PD-1 inhibitor, a LAG-3 inhibitor, a cytokine, an A2A antagonist, a GITR agonist, a TIM-3 inhibitor, a STING agonist, and a TLR7 agonist.

87. The method of claim 85, wherein the one or more therapeutic agents is a PD-1 inhibitor.

88. The method of claim 85, wherein the one or more therapeutic agents is a LAG-3 inhibitor.

89. The method of claim 85, wherein the one or more therapeutic agents is a cytokine.

90. The method of claim 85, wherein the one or more therapeutic agents is an A2A antagonist.

91. The method of claim 85, wherein the one or more therapeutic agents is a GITR agonist.

92. The method of claim 85, wherein the one or more therapeutic agents is a TIM-3 inhibitor.

93. The method of claim 85, wherein the one or more therapeutic agents is a STING agonist.

94. The method of claim 85, wherein the one or more therapeutic agents is a TLR7 agonist.
